(19) United States
(12) Patent Application Publication
(10) Pub. No.: US 2024/0075004 A1
(43) Pub. Date: Mar. 7, 2024

(54) VERY-LONG-CHAIN POLYUNSATURATED FATTY ACIDS, ELOVANOID HYDROXYLATED DERIVATIVES, AND METHODS OF USE

(71) Applicant: The Board of Supervisors of Louisiana State University and Agricultural and Mechanical College, Baton Rouge, LA (US)

(72) Inventor: Nicolas G. BAZAN, New Orleans, LA (US)

(21) Appl. No.: 18/268,926

(22) PCT Filed: Dec. 21, 2021
(86) PCT No.: PCT/US2021/064654

§ 371 Date: Jun. 21, 2023

Related U.S. Application Data
(60) Provisional application No. 63/128,590, filed on Dec. 21, 2020.

(51) Int. Cl. A61K 31/202; A61P 39/00

(52) U.S. Cl. CPC A61K 31/202; A61P 39/00

(57) ABSTRACT

This disclosure relates to methods of use related to omega-3 very-long-chain polyunsaturated fatty acids (n-3 VLC-PUFA) and their hydroxylated derivatives known as elovanoids to alleviate a symptom of, treat, or prevent disease. Furthermore, this invention is directed to compositions and methods for modulating VLC-PUFA bioactivity and availability.

This application is an International Application which claims priority from U.S. patent application No. 63/128,590 filed on Dec. 21, 2020, the entire contents of which are incorporated herein by reference.

### STATEMENT REGARDING FEDERALLY SPONSORED RESEARCH OR DEVELOPMENT

This invention was made with government support under contracts EY005121, NS104117 and NS109221 awarded by the National Institutes of Health. The Government has certain rights in the invention.

### FIELD OF THE DISCLOSURE

This disclosure relates to methods of use related to omega-3 very-long-chain polyunsaturated fatty acids (n-3 VLC-PUFA) and their hydroxylated derivatives known as elovanoids to alleviate a symptom of, treat, or prevent disease. Furthermore, this invention is directed to compositions and methods for modulating VLC-PUFA bioactivity and availability.

### BACKGROUND

Long chain polyunsaturated fatty acids (LC-PUFAs) can include the omega-3 (n-3) and omega-6 (n6) polyunsaturated fatty acids containing 18-22 carbons including: arachidonic acid (ARA, C20:4n6, i.e. 20 carbons, 4 double bonds, omega-6), eicosapentaenoic acid (EPA, C20:5n-3, 20 carbons, 5 double bonds, omega-3), docosapentaenoic acid (DPA, C22:5n-3, 22 carbons, 5 double bonds, omega-3), and docosahexaenoic acid (DHA, C22:6n-3, 22 carbons, 6 double bonds, omega-3). LC-PUFAs are converted via lipoxygenase-type enzymes to biologically active hydroxylated PUFA derivatives that function as biologically active lipid mediators that play important roles in inflammation and related conditions. Most important among these are hydroxylated derivatives generated in certain inflammation-related cells via the action of a lipoxygenase (LO or LOX) enzyme (e.g. 15-LO, 12-LO), and result in the formation of mono-, di- or tri-hydroxylated PUFA derivatives with potent actions including anti-inflammatory, pro-resolving, neuroprotective or tissue-protective actions, among others. For example, neuroprotectin D1 (NPD1), a dihydroxy derivative from DHA formed in cells via the enzymatic action of 15-lipoxygenase (15-LO) was shown to have a defined R/S and Z/E stereochemical structure (10R,17S-dihydroxy-docosa-4Z,7Z,11E, 13E,15Z,19Z-hexaenoic acid) and a unique biological profile that can includes stereoselective potent anti-inflammatory, homeostasis-restoring, pro-resolving, bioactivity. NPD1 has been shown to modulate neuroinflammatory signaling and proteostasis, and to promote nerve regeneration, neuroprotection, and cell survival.

### SUMMARY OF THE DISCLOSURE

Aspects of the disclosure are drawn to methods of delaying or preventing aging of a subject. In embodiments, the method comprises administering to the subject a therapeutically effective amount of a senolytic agent, wherein the senolytic agent comprises a VLC-PUFA or a hydroxylated derivative thereof. In embodiments, the aging is not skin aging. In embodiments, aging is indicated by the presence or absence of senescent cells, the presence or absence of inflammation, or both. In a further embodiment, the presence or absence of pro-inflammatory cytokines and chemokines is indicative of inflammation. In a further embodiment, the pro-inflammatory cytokines and chemokines comprise at least one of IL-6, IL-10, IL-8/CXCL8, CCL2/MCP-1, CXCL1/KC/GRO, VEGF, ICAM1(CD54). In an embodiment the VLC-PUFA abrogates the production of pro-inflammatory cytokines and chemokines.

In embodiments, preventing aging comprises reducing the number or activity of senescent cells.

In embodiments, the VLC-PUFA can be selected from the group consisting of the formula A or B:

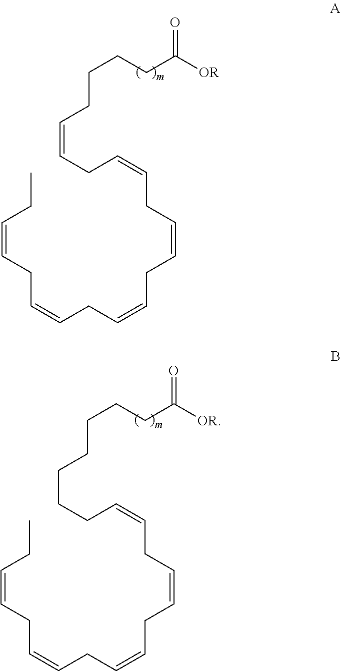

In embodiments, the hydroxylated derivative compound can be selected from the group consisting of:

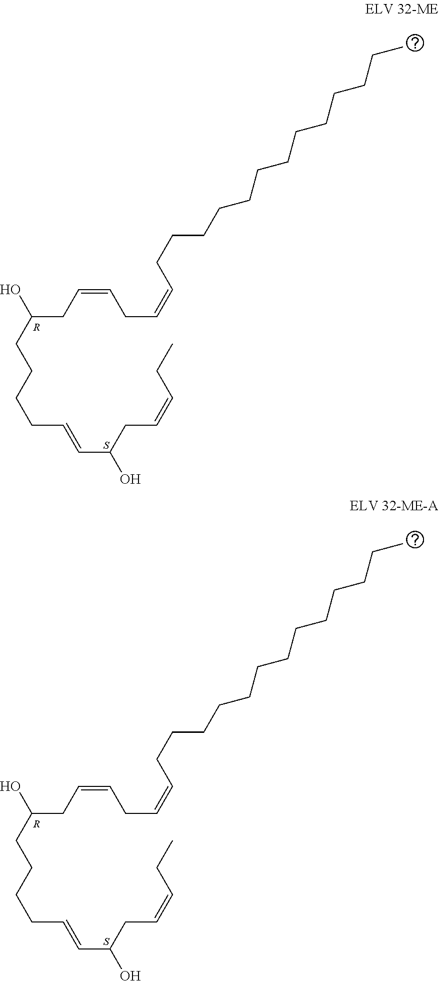

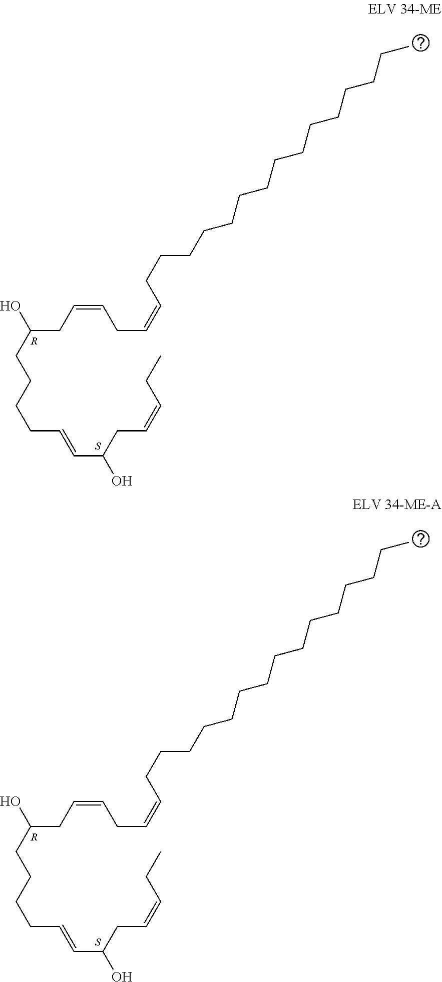

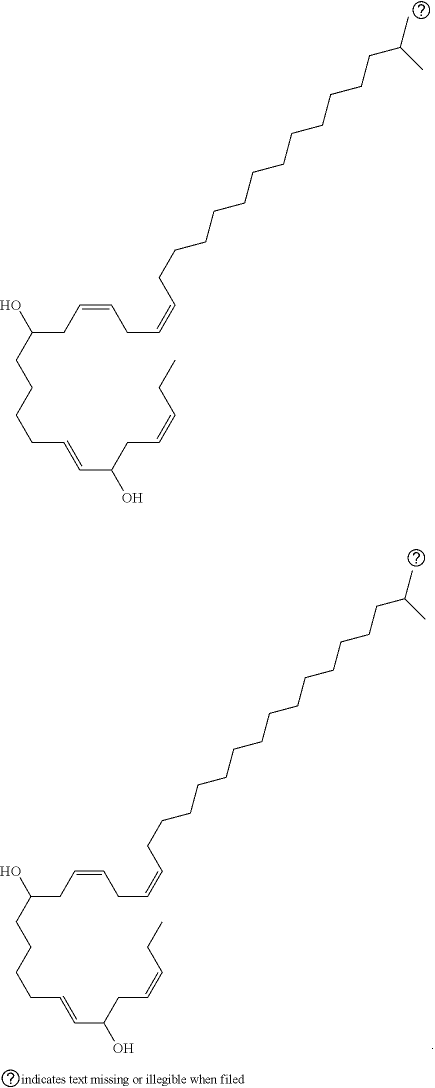 [SMILES: CC/C=C\CC(O)/C=C/CCCCC(O)C/C=C\C/C=C\CCCCCCCCCCCCC(C)C ; CC/C=C\CC(O)/C=C/CCCCC(O)C/C=C\C/C=C\CCCCCCCCCCCCCCC(C)C]

In embodiments, the VLC-PUFA or hydroxylated derivative thereof is provided as a pharmaceutical composition. In embodiments, the therapeutically effective amount comprises about 500 nM concentration, greater than about 500 nM concentration, or less than about 500 nM concentration. In embodiments, the subject is a human.

Further, aspects of the disclosure are drawn to a method of alleviating a symptom of, treating, or preventing a age-related disease or dysfunction by using a senolytic such as a VLC-PUFA or hydroxylated derivative thereof to modulate cellular senescence, ferroptosis, or cellular senescence and ferroptosis. For example, the method can comprise administering to the subject a therapeutically effective amount of a VLC-PUFA.

In embodiments, the VLC-PUFA is provided as a pharmaceutical composition. For example, the pharmaceutical composition comprises a composition for topical administration, a composition for intranasal administration, a composition for oral administration, or a composition for parenteral administration.

In embodiments, the VLC-PUFA or pharmaceutical composition is administered topically, orally, intranasally, or parenterally.

In embodiments, the therapeutically effective amount comprises about 500 nM concentration, greater than about 500 nM concentration, or less than about 500 nM concentration.

In embodiments, the VLC-PUFA abrogates the production of pro-inflammatory cytokines and chemokines, enhances the production of anti-inflammatory molecules, or both.

Provided herein are compounds and pharmaceutical compositions comprising omega-3 very-long-chain polyunsaturated fatty acids (n-3 VLC-PUFA) and/or their endogenous hydroxylated derivatives thereof, known as elovanoids.

n-3 VLC-PUFAs are converted in vivo to several previously unknown types of VLC-PUFA hydroxylated derivatives named elovanoids (ELVs) that are able to protect and prevent the progressive damage to tissues and organs, whose functional integrity has been disrupted. Without wishing to be bound by theory, the ELVs can abrogate the production of pro-inflammatory cytokines and chemokines and thus alleviate a symptom of, treat, or prevent an allergic inflammatory disease in a subject.

The production of pro-inflammatory cytokines and chemokines can be effectively suppressed by providing certain compounds related to n-3 VLC-PUFA and their corresponding elovanoids (ELVs).

The present disclosure provides compounds, compositions and methods that can promote the protection, prevention, and treatment of disturbances in many organs triggered by pro-inflammatory cytokines.

Accordingly, one aspect of the disclosure encompasses embodiments of a composition comprising at least one omega-3 very long chain polyunsaturated fatty acid having at least 23 carbon atoms in its carbon chain.

In some embodiments of this aspect of the disclosure, the composition comprises at least one n-3 VLC-PUFA having at least 23 carbon atoms in its carbon chain, wherein the n-3 VLC-PUFA can be in the form of a carboxylic acid, carboxylic ester, carboxylate salt, or phospholipid derivative.

In some embodiments of this aspect of the disclosure, the n-3 VLC-PUFA compound can be selected from the group consisting of the formulas A or B:

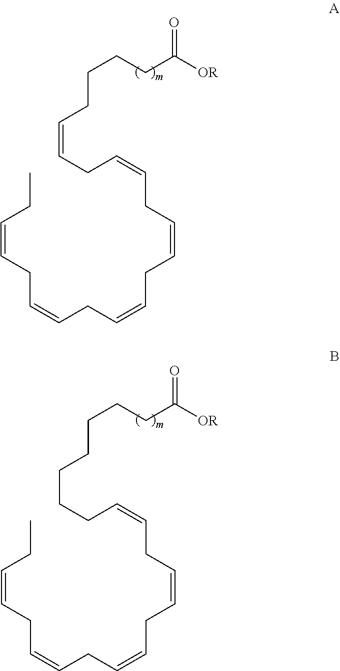

wherein: m can be 0 to 19 and —CO—OR can be a carboxylic acid group, or a salt or an ester thereof, and wherein: if —CO—OR can be a carboxylic acid group and the compound A or B can be a salt thereof, the cation of the salt can be a pharmaceutically acceptable cation, and if —CO—OR can be an ester, then R can be an alkyl group.

In some other embodiments, the disclosure, the n-3 VLC-PUFA compound can be in the form of a phospholipid selected from the group consisting of the formulas C, D, E or F, wherein m can be 0 to 19:

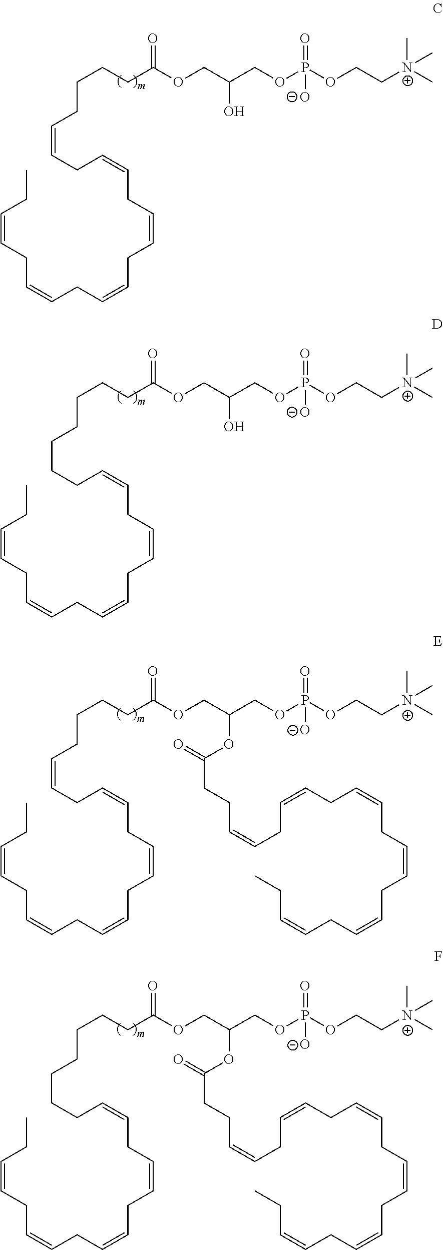

In some embodiments of this aspect of the disclosure, the composition can further comprise a pharmaceutically-acceptable carrier and formulated for delivery of an amount of the at least one omega-3 very long chain polyunsaturated fatty acid effective in reducing a pathological condition of a tissue of a recipient subject or the onset of a pathological condition of a tissue of a recipient subject.

In some embodiments of this aspect of the disclosure, the composition can be formulated for topical delivery of the at least one very long chain polyunsaturated fatty acid to the skin of a recipient subject or to the eye of a recipient subject, such as in an eye drop.

In some embodiments of this aspect of the disclosure, the composition can be formulated for intranasal delivery of the at least one very long chain polyunsaturated fatty acid to the nasal tissue of a recipient subject.

In some embodiments of this aspect of the disclosure, the composition can be formulated for oral delivery or parenteral delivery of the at least one very long chain polyunsaturated fatty acid to a recipient subject.

In some embodiments of this aspect of the disclosure, the at least one omega-3 very long chain polyunsaturated fatty acid can have from about 26 to about 42 carbon atoms in its carbon chain.

In some embodiments of this aspect of the disclosure, the at least one omega-3 very long chain polyunsaturated fatty acid can have 32 or 34 carbon atoms in its carbon chain.

In some embodiments of this aspect of the disclosure, the omega-3 very long chain polyunsaturated fatty acid can have in its carbon chain five or six alternating double bonds with cis geometry.

In some embodiments of this aspect of the disclosure, the omega-3 very long chain polyunsaturated fatty acid is 14Z,17Z,20Z,23Z,26Z,29Z)-dotriaconta-14,17,20,23,26,29-hexaenoic acid or (16Z,19Z,22Z,25Z,28Z,31Z)-tetratriaconta-16,19,22,25,28,31-hexaenoic acid.

In some embodiments of this aspect of the disclosure, the at least one omega-3 very long chain polyunsaturated fatty acid can be 14Z,17Z,20Z,23Z,26Z,29Z)-dotriaconta-14,17,20,23,26,29-hexaenoic acid or (16Z,19Z,22Z,25Z,28Z,31Z)-tetratriaconta-16,19,22,25,28,31-hexaenoic acid.

Another aspect of the disclosure encompasses embodiments of a composition comprising at least one elovanoid having at least 23 carbon atoms in its carbon chain.

In some embodiments of this aspect of the disclosure, the composition can further comprise a pharmaceutically-acceptable carrier and can be formulated for delivery of an amount of the at least one elovanoid effective in alleviating a symptom of, preventing, or reducing a pathological condition of a tissue of a recipient subject.

In some embodiments of this aspect of the disclosure, the composition can be formulated for topical delivery of the at least one elovanoid to the skin of a recipient subject or the eye of a recipient subject, such as by an eye drop.

In some embodiments of this aspect of the disclosure, the composition can be formulated for intranasal delivery of the at least one elovanoid to the nasal tissue of a recipient subject.

In some embodiments of this aspect of the disclosure, the composition can be formulated for oral delivery or parenteral delivery of the at least one elovanoid to a recipient subject.

In some embodiments of this aspect of the disclosure, the at least one elovanoid can be selected from the group consisting of: a mono-hydroxylated elovanoid, a di-hydroxylated elovanoid, an alkynyl mono-hydroxylated elovanoid, and an alkynyl di-hydroxylated elovanoid, or any combination thereof.

In some embodiments of this aspect of the disclosure, the at least one elovanoid can be a combination of elovanoids, wherein the combination is selected from the group consisting of: a mono-hydroxylated elovanoid and a di-hydroxylated elovanoid; a mono-hydroxylated elovanoid and an alkynyl mono-hydroxylated elovanoid; a mono-hydroxylated elovanoid and an alkynyl di-hydroxylated elovanoid; a di-hydroxylated elovanoid and an alkynyl mono-hydroxylated elovanoid; a di-hydroxylated elovanoid and an alkynyl di-hydroxylated elovanoid; a mono-hydroxylated elovanoid, a di-hydroxylated elovanoid, and an alkynyl mono-hydroxylated elovanoid; a mono-hydroxylated elovanoid, a di-hydroxylated elovanoid, and an alkynyl di-hydroxylated elovanoid; and a mono-hydroxylated elovanoid, a di-hydroxylated elovanoid, and an alkynyl mono-hydroxylated elovanoid an alkynyl di-hydroxylated elovanoid, wherein each elovanoid is independently a racemic mixture, an isolated enantiomer, or a combination of enantiomers wherein the amount of one enantiomer greater than the amount of another enantiomer; and wherein each elovanoid is independently a diastereomeric mixture, an isolated diastereomer, or a combination of diastereomers wherein the amount of one diastereomer is greater than the amount of another diastereomer.

In some embodiments of this aspect of the disclosure, the mono-hydroxylated elovanoid can be selected from the group consisting of the formulas G, H, I or J:

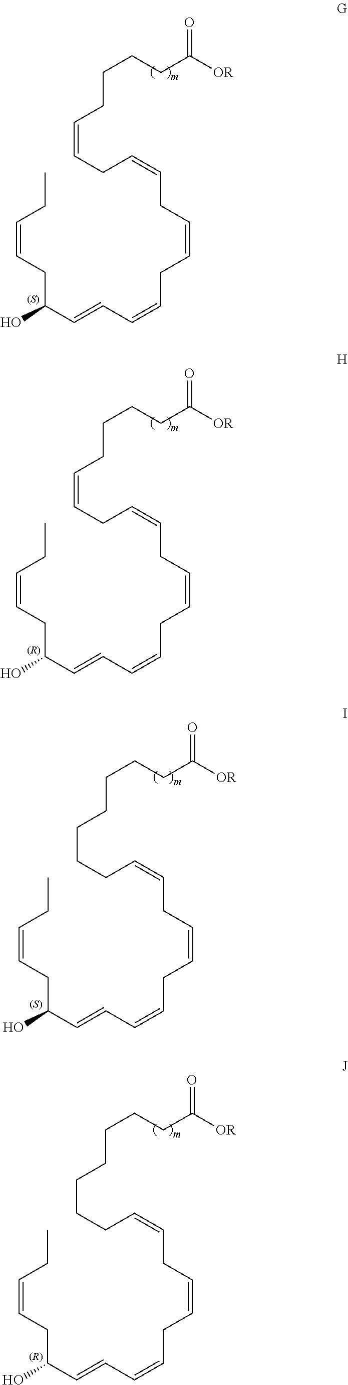

wherein: m can be 0 to 19 and —CO—OR can be a carboxylic acid group, or a salt or an ester thereof, and wherein: if —CO—OR can be a carboxylic acid group and the compound G, H, I or J can be a salt thereof, the cation of the salt can be a pharmaceutically acceptable cation, and if —CO—OR can be an ester, then R can be an alkyl group.

In some embodiments of this aspect of the disclosure, the composition can comprise equimolar amounts of the enantiomers G and H wherein the enantiomers have (S) or (R) chirality at the n-6 carbon bearing the hydroxyl group.

In some embodiments of this aspect of the disclosure, the composition can comprise amounts of the enantiomers I and J wherein the enantiomers have (S) or (R) chirality at the n-6 carbon bearing the hydroxyl group.

In some embodiments of this aspect of the disclosure, the composition can comprise one of the enantiomers of G or H in an amount exceeding the amount of the other enantiomer of G or H.

In some embodiments of this aspect of the disclosure, the composition can comprise one of the enantiomers of I or J in an amount exceeding the amount of the other enantiomer of I or J.

In some embodiments of this aspect of the disclosure, the di-hydroxylated elovanoid can be selected from the group consisting of the formulas K, L, M, and N:

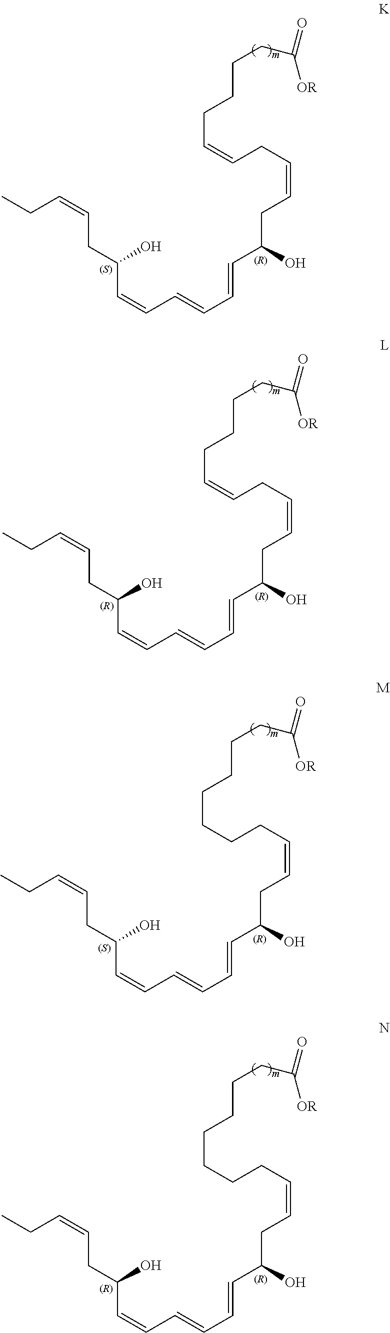

wherein: m can be 0 to 19 and —CO—OR can be a carboxylic acid group, or a salt or an ester thereof, and wherein: if —CO—OR can be a carboxylic acid group and the compound K, L, M, or N can be a salt thereof, the cation of the salt can be a pharmaceutically acceptable cation, and if —CO—OR can be an ester, then R can be an alkyl group, and wherein: compounds K and L each have a total from 23 to 42 carbon atoms in the carbon chain, with 4 cis carbon-carbon double bonds starting at positions n-3, n-7, n-15 and n-18, and 2 trans carbon-carbon double bonds starting at positions n-9 and n-11; and compounds M and N each have a total from 23 to 42 carbon atoms in the carbon chain, with 3 cis carbon-carbon double bond starting at positions n-3, n-7 and n-15; and 2 trans carbon-carbon double bonds starting at positions n-9 and n-11.

In some embodiments of this aspect of the disclosure, the composition can comprise equimolar amounts of diastereomers K and L wherein the diastereomers have either (S) or (R) chirality at position n-6, and (R) chirality at position n-13.

In some embodiments of this aspect of the disclosure, the composition can comprise equimolar amounts of one or more diastereomers K and L wherein the diastereomers have either (S) or (R) chirality at position n-6, and either (S) or (R) chirality at position n-13.

In some embodiments of this aspect of the disclosure, the composition can comprise one of the diastereomers of K or L in an amount exceeding the amount of the other diastereomers of K or L.

In some embodiments of this aspect of the disclosure, the composition can comprise equimolar amounts of diastereomers M and N wherein the diastereomers have either (S) or (R) chirality at position n-6, and (R) chirality at position n-13.

In some embodiments of this aspect of the disclosure, the composition can comprise equimolar amounts of one or more diastereomers M and N wherein the diastereomers have either (S) or (R) chirality at position n-6, and either (S) or (R) chirality at position n-13.

In some embodiments of this aspect of the disclosure, the composition can comprise one of the diastereomers of M or N in an amount exceeding the amount of the other diastereomers of M or N.

In some embodiments of this aspect of the disclosure, the alkynyl mono-hydroxylated elovanoid can be selected from the group consisting of the formulas 0, P, Q or R:

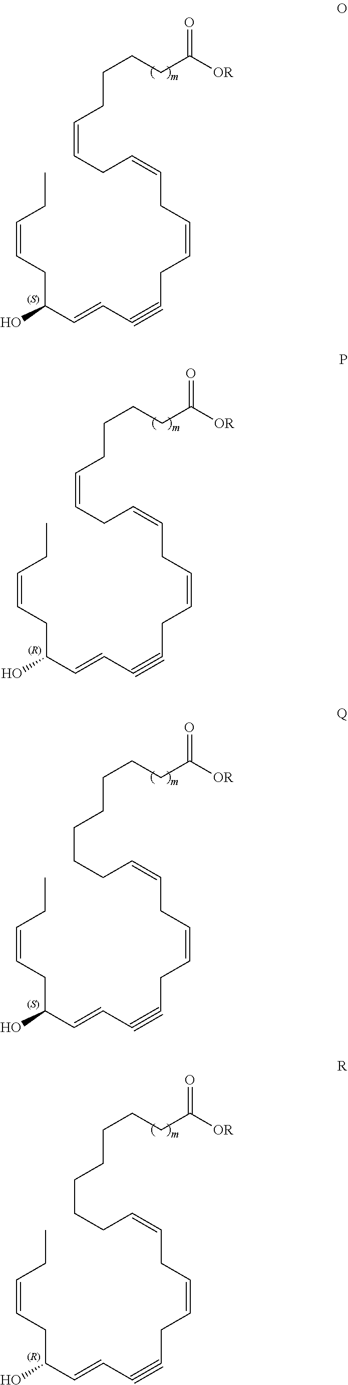

wherein: m can be 0 to 19 and —CO—OR can be a carboxylic acid group, or a salt or an ester thereof, and wherein: if —CO—OR can be a carboxylic acid group and the compound O, P, Q or R can be a salt thereof, the cation of the salt can be a pharmaceutically acceptable cation, and if —CO—OR can be an ester, then R can be an alkyl group, and wherein: compounds O and P each have a total from 23 to 42 carbon atoms in the carbon chain, with 4 cis carbon-carbon double bonds located at positions starting at n-3, n-12, n-15 and n-18; with a trans carbon-carbon double bond at position starting at n-7, and a carbon-carbon triple bond starting at position n-9; and compounds Q and R each have a total from 23 to 42 carbon atoms in the carbon chain, with 3 cis carbon-carbon double bond starting at positions n-3, n-12 and n-15, with a trans carbon-carbon double bond at position starting at n-7, and a carbon-carbon triple bond starting at position n-9.

In some embodiments of this aspect of the disclosure, the composition can comprise equimolar amounts of the enantiomers O and P wherein the enantiomers have (S) or (R) chirality at the n-6 carbon bearing the hydroxyl group.

In some embodiments of this aspect of the disclosure, the composition can comprise equimolar amounts of the enantiomers Q and R wherein the enantiomers have (S) or (R) chirality at the n-6 carbon bearing the hydroxyl group.

In some embodiments of this aspect of the disclosure, the composition can comprise one of the enantiomers of O or P in an amount exceeding the amount of the other enantiomer of O or P.

In some embodiments of this aspect of the disclosure, the composition can comprise one of the enantiomers of Q or R in an amount exceeding the amount of the other enantiomer of Q or R.

In some embodiments of this aspect of the disclosure, the elovanoid can be an alkynyl di-hydroxylated elovanoid selected from the group consisting of the formulas S, T, U or V:

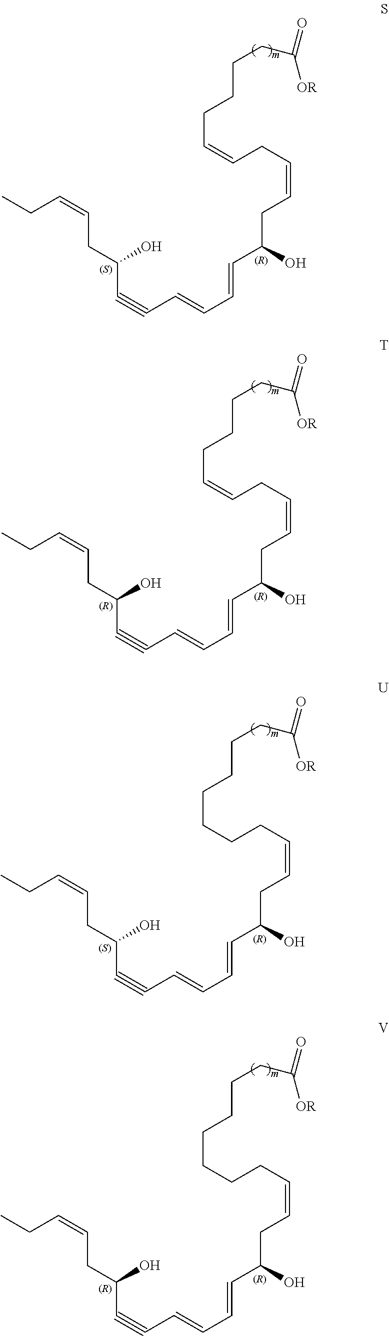

wherein: m can be 0 to 19 and —CO—OR can be a carboxylic acid group, or a salt or an ester thereof, and wherein: if —CO—OR can be a carboxylic acid group and the compound S, T, U or V can be a salt thereof, the cation of the salt can be a pharmaceutically acceptable cation, and if —CO—OR can be an ester, then R can be an alkyl group, and wherein: compounds S and T each have a total from 23 to 42 carbon atoms in the carbon chain, with 3 cis carbon-carbon double bonds located at positions starting at n-3, n-15 and n-18, with 2 trans carbon-carbon double bonds located at positions starting at n-9 and n-11, and a carbon-carbon triple bond starting at position n-7; and compounds U and V each have a total from 23 to 42 carbon atoms in the carbon chain, with 2 cis carbon-carbon double bond starting at positions n-3 and n-15, with 2 trans carbon-carbon double bonds located at positions starting at n-9, n-11, and a carbon-carbon triple bond starting at position n-7.

In some embodiments of this aspect of the disclosure, the composition can comprise equimolar amounts of diastereomers S and T wherein the diastereomers have either (S) or (R) chirality at position n-6, and (R) chirality at position n-13.

In some embodiments of this aspect of the disclosure, the composition can comprise equimolar amounts of one or more diastereomers S and T wherein the diastereomers have either (S) or (R) chirality at position n-6, and either (S) or (R) chirality at position n-13.

In some embodiments of this aspect of the disclosure, the composition can comprise one of the diastereomers of S or T in an amount exceeding the amount of the other diastereomers of S or T.

In some embodiments of this aspect of the disclosure, the composition can comprise equimolar amounts of diastereomers U and V wherein the diastereomers have either (S) or (R) chirality at position n-6, and (R) chirality at position n-13.

In some embodiments of this aspect of the disclosure, the composition can comprise equimolar amounts of one or more diastereomers U and V wherein the diastereomers have either (S) or (R) chirality at position n-6, and either (S) or (R) chirality at position n-13.

In some embodiments of this aspect of the disclosure, the composition can comprise one of the diastereomers of U or V in an amount exceeding the amount of the other diastereomers of U or V.

Aspects of the invention are further drawn towards a peptide analog that binds to an epitope of a molecular target of the Table included herein.

In embodiments, the peptide analog modulates cellular senescence, ferroptosis, or cellular senescence and ferroptosis.

Still further, aspects of the invention are drawn towards a method of treating a disease by administering to a subject the peptide analog as described herein. For example, embodiments are drawn towards a method of treating a disease by modulating cellular senescence, ferroptosis, or cellular senescence and ferroptosis. In embodiments, the method comprises the steps of administering to a subject afflicted with or at risk of a disease an elovanoid or a peptide analog thereof. In embodiments, the elovanoid or peptide analog thereof binds to an epitope of a molecular target as described herein.

Aspects of the invention are further drawn towards a method of treating a disease associated with cellular senescence, ferroptosis, or cellular senescence and ferroptosis. For example, the method comprises targeting at least one molecular target with an elovanoid or peptide analog thereof.

### BRIEF DESCRIPTION OF THE DRAWINGS

The present disclosure is focused on compounds, compositions and methods for applications in delaying or preventing aging and/or age-related dysfunctions in a subject by administering to the subject a therapeutically effective amount of a senolytic agent, wherein the senolytic agent comprises a VLC-PUFE or a hydroxylated derivative thereof. Further aspects of the present disclosure will be readily appreciated upon review of the detailed description of its various embodiments, described herein, when taken in conjunction with the accompanying drawings.

FIG. 1 is a scheme illustrating the biosynthesis of elovanoids (ELV) from omega-3 (n-3 or n-3) very long chain polyunsaturated fatty acids (n-3 VLC-PUFA).

FIG. 2 is a scheme illustrating the biosynthesis of n-3 VLC-PUFA.

FIGS. 3A-3K illustrate the generation and structural characterization of elovanoids ELV-N-32 and ELV-N-34 from cultured primary human retinal pigment epithelial cells (RPE).

FIG. 3A is a scheme illustrating ELV-N-32 and ELV-N-34 synthesis from the intermediates (1, 2, and 3), each of which was prepared in stereochemically-pure form. The stereochemistry of intermediates 2 and 3 was pre-defined by using enantiomerically-pure epoxide starting materials. The final ELVs (4) were assembled via iterative couplings of intermediates 1, 2, and 3, and were isolated as the methyl esters (Me) or sodium salts (Na).

FIG. 3B illustrates the elution profile of C32:6n-3, endogenous mono-hydroxy-C32:6n-3, and ELV-N-32 shown with ELV-N-32 standard. MRM of ELV-N-32 shows two large peaks eluted earlier than the peak when standard ELV-N-32 is eluted, displaying the same fragmentation patterns (shown in the insert spectra), indicating that they are isomers.

FIG. 3C illustrates the chromatogram for full daughter scans for ELV-N-32 and ELV-N-34.

FIG. 3D illustrates the fragmentation pattern of ELV-N-32.

FIG. 3E illustrates the elution profile of C34:6n-3 and ELV-N-34.

FIG. 3F illustrates the UV spectrum of endogenous ELV-N-34 showing triene features analogous to NPD1, with λmax at 275 nm and shoulders at 268 and 285 nm.

FIG. 3G illustrates the fragmentation pattern of ELV-N-32.

FIG. 3H illustrates the full fragmentation spectra of endogenous ELV-N-32.

FIG. 3I illustrates the ELV-N-32 standard shows that all major peaks from standard match to the endogenous peaks. However, endogenous ELV-N-32 has more fragments that don't show up in the standard, indicating that it can includes different isomers.

FIG. 3J illustrates the full fragmentation spectra of endogenous ELV-N-34 peaks match to standard ELV-N-34.

FIG. 3K illustrates the existence of ELV-N-34 isomers.

FIGS. 4A-4K illustrate the structural characterization of elovanoids ELV-N-32 and ELV-N-34 from neuronal cell cultures. Cerebral cortical mixed neuronal cells were incubated with 32:6n-3 and 34:6n-3 10 μM each under OGD conditions.

FIG. 4A is a scheme illustrating ELV-N-32 and ELV-N-34 synthesis from the intermediates (a, b, and c), each of which was prepared in stereochemically-pure form. The stereochemistry of intermediates b and c was pre-defined by using enantiomerically-pure epoxide starting materials. The final ELVs (d) were assembled via iterative couplings of intermediates a, b, and c, and were isolated as the methyl esters (Me) or sodium salts (Na).

FIG. 4B illustrates the 32:6n-3, endogenous mono-hydroxy-32:6, ELV-N-32, and ELV-N-32 standard in the insert. MRM of ELV-N-32 shows two large peaks eluted earlier than the peak when standard ELV-N-32 is eluted, but they show the same fragmentation patterns, indicating that they are isomers.

FIG. 4C illustrates the same features as in FIG. 4A, were shown in 34:6n-3 and ELV-N-34.

FIG. 4D illustrates the UV spectrum of endogenous ELV-N-32 shows triene features, but these are not definite at this concentration.

FIG. 4E illustrates the full fragmentation spectra of endogenous ELV-N-32.

FIG. 4F illustrates the UV spectrum of endogenous ELV-N-34 showing triene features analogous to NPD1, with λmax at 275 nm and shoulders at 268 and 285 nm.

FIG. 4G illustrates the fragmentation pattern of endogenous ELV-N-34.

FIG. 4H illustrates the full fragmentation pattern of endogenous ELV-N-32.

FIG. 4I illustrates the ELV-N-34 standard shows that all major peaks from the standard match to the endogenous peaks, but not perfectly matched; endogenous ELV-N-34 has more fragments that do not show up in the standard. Without wishing to be bound by theory, this indicates that it can contain isomers.

FIG. 4J illustrates the ELV-N-34 full fragmentation spectra; the endogenous ELV-N-34 peaks match to the standard ELV-N-34

FIG. 4K illustrates the existence of ELV-N-34 isomers.

FIGS. 5A and 5B illustrate the detection of ELV-N-32 and ELV-N-34 in neuronal cell cultures. Cells were incubated with C32:6n-3 and C34:6n-3 5 μM each, under OGD conditions.

FIG. 5A illustrates the VLC-PUFA C32:6n-3, endogenous 27-hydroxy-32:6n-3, endogenous 27,33-dihydroxy-32:6n-3 (ELV-N-32), and synthetic ELV-N-32 prepared in stereochemical pure form via stereocontrolled total organic synthesis. MRM of endogenous ELV-N-32 matches well with the MRM of the synthetic ELV-N-32 standard.

FIG. 5B illustrates the same features as in FIG. 5A were shown in C34:6n-3 and ELV-N-34, with more peaks in ELV-N-34 MRMs, which indicates isomers.

FIG. 6 illustrates Scheme 1 for the total synthesis of mono-hydroxylated elovanoids G, H, I, J, O, P, Q, R. Reagents & Conditions: (a) Catechol borane, heat; (b) N-iodo-succinimide, MeCN; (c) 4-chlorobut-2-yn-1-ol, Cs₂CO₃, NaI, CuI, DMF; (d) CBr₄, PPh₃, CH₂Cl₂, 0° C.; (e) ethynyl-trimethylsilane, CuI, NaI, K₂CO₃, DMF; (f) Lindlar cat., H₂, EtOAc; (g) Na₂CO₃, MeOH; (h) Pd(PPh₃)₄, CuI, Et₃N: (i) ^{t}Bu₄NF, THF; (j) Lindlar cat., H₂, EtOAc or Zn(Cu/Ag), MeOH; (k) NaOH, THF, H₂O, then acidification with HCl/H₂O; (l) NaOH, KOH, or the like. or amine, imine, etc.

FIG. 7 illustrates Scheme 2 for the total synthesis of di-hydroxylated elovanoids K, L, S, and T. Reagents & Conditions: (a) CuI, NaI, K₂CO₃, DMF; (b) camphorsulfonic acid (CSA), CH₂Cl₂, MeOH, rt; (c) Lindlar cat., H₂, EtOAc; (d) DMSO, (COCl)₂, Et₃N, −78° C.; (e) Ph₃P═CHCHO, PhMe, reflux; (f) CHI₃, CrCl₂, THF, 0° C.; (g) cat. Pd(Ph₃)₄, CuI, PhH, rt; (h) ^{t}Bu₄NF, THF, rt; (i) Zn(Cu/Ag), MeOH, 40° C.; (j) NaOH, THF, H₂O, then acidification with HCl/H₂O; (k) NaOH, KOH, etc. or amine, imine, etc.

FIG. 8 illustrates Scheme 3 for the total synthesis of di-hydroxylated elovanoids M, N, U, and V. Reagents & Conditions: (a) cyanuric chloride, Et₃N, acetone, rt; (b) (3-methyloxetan-3-yl)methanol, pyridine, CH₂Cl₂, 0° C.; (c) BF₃·OEt₂, CH₂Cl₂; (d) ^{t}BuLi, BF₃·OEt₂, THF, −78° C., then 1; (e) ^{t}BuPh₂SiCl, imidazole, DMAP, CH₂Cl₂, rt; (f) camphorsulfonic acid, CH₂Cl₂, ROH, rt; (g) Lindlar cat., H₂, EtOAc; (h) DMSO, (COCl)₂, Et₃N, −78° C.; (i) Ph₃P═CHCHO, PhMe, reflux; (j) CHI₃, CrCl₂, THF, 0° C.; (k) cat. Pd(Ph₃)₄, CuI, PhH, rt; (l) ^{t}Bu₄NF, THF, rt; (m) Zn(Cu/Ag), MeOH, 40° C.; (n) NaOH, THF, H₂O, then acidification with HCl/H₂O; (o) NaOH, KOH, etc. or amine, imine, etc.

FIG. 9 illustrates Scheme 4 for the total synthesis of 32-carbon di-hydroxylated elovanoids.

FIG. 10 illustrates Scheme 5 for the total synthesis of 34-carbon di-hydroxylated elovanoids.

FIG. 11 shows bright field images showing morphology of HNEpC—10× and 20×.

FIG. 12 shows house dust mite allergenicity. The various components of HDM and their associated fecal pellets and dust activate the immune system. See, Trends in Immunology, September, 2011, Vol. 32, No. 9. Gregory, 2011.

FIG. 13 shows structures of LPS and Poly(I:C).

FIG. 14 shows experimental design of challenging HNEpC using several stressors (aeroallergens)

FIG. 15 shows different elovanoids (ELVs) that were used at 500 nM concentration for verifying lipid specificity against HNEpCs stressed with different aeroallergens.

FIG. 16 shows cytotoxicity assay using CyQuant LDH assay. Damage to the plasma membrane releases LDH into the surrounding cell culture media. The extracellular LDH in the media can be quantified by a coupled enzymatic reaction in which LDH catalyzes the conversion of lactate to pyruvate via NAD+ reduction to NADH. Oxidation of NADH by diaphorase leads to the reduction of a tetrazolium salt (INT) to a red formazan product that can be measured spectrophotometrically at 490 nm. The level of formazan formation is directly proportional to the amount of LDH released into the medium, which is indicative of cytotoxicity.

FIG. 17A and FIG. 17B shows cytotoxicity assay using CyQuant LDH assay.

FIG. 18A and FIG. 18B shows cell viability assay using Presto Blue HS reagent.

FIG. 19A and FIG. 19B shows ELISA (IL-6).

FIG. 20A and FIG. 20B shows ELISA (IL-10).

FIG. 21A and FIG. 21B shows ELISA (IL-8).

FIG. 22A and FIG. 22B shows ELISA (CCL2).

FIG. 23A and FIG. 23B shows ELISA (CXCL1).

FIG. 24A and FIG. 24B shows ELISA (VEGF).

FIG. 25A and FIG. 25B shows ELISA (ICAM1).

FIG. 26A and FIG. 26B shows ELISA (IL-10)

FIG. 27 shows representative drawing from Trends in Molecular Medicine; October, 2011; Vol. 17, No. 10. Jacquet, 2011.

FIG. 28 shows the converging mechanisms that regulate senescence for the development of new synthetic non-lipidic analogs to mimic the bioactivity of the lipid mediators.

FIG. 29 shows oxidative-stress and Erastin induced cell deaths counteracted by NPD1 and ELV-32:6 in human RPE cells.

FIG. 30 shows elovanoids attenuates Erastin-mediated phosphorylation of PEBP-1 (adapted from Wenzel et al., 2017, Cell, 171:628-641).

FIG. 31 shows upstream regulation by elovanoids of ferroptosis and senescence.

FIG. 32 shows deletion of MFRP (Membrane Frizzled-Related Protein) leads to progressive PRC degeneration.

FIG. 33 shows selective loss of PC44:12 and 56:12 in AdipoR1^{-/-} and MFRP^{rd6}.

FIG. 34 shows targets in human retina with AMD.

FIG. 35 shows AMD shows PCs selective differences in cone-rich macula and rod-rich periphery; MALDI MS molecular imaging displays distinct layering.

FIG. 36 shows heat maps of 168 GPCR targets and of 73 orphan GPRCs (antagonists or partial agonists) screened by PathHunter β-arrestin enzyme fragment complementation/β-galactosidase against the orphanMAX Panel (DiscoverX, Eurofins, Fremonst, CA). Without wishing to be bound by theory, GPCR targets (blue arrows) display activity above threshold. Assays with NPD1, ELV-N-32 or ELV-N-34 (5 μM) or vehicle, incubated with cells expressing GPCR panels, 37° C., 90 min. Microplates read with PerkinElmer EnVision multimode for Chemiluminescence. Activity analyzed using CBIS data suite (ChemInnovation, CA). Screening performed twice in a blind manner; results were identical both times. Rainbow colored Heat maps generated with GraphPad Prism 8.2 using % activity (agonists) and % Inhibition (antagonists) values. Color mapping done with a uniform legend having the smallest value being 0, and the largest value being 60. Only GPCR that had high cutoff values (green to red) color intensities are considered candidates (blue arrows).

FIG. 37 shows induction of the expression and activation of a specific AdipoR1 molecule will compensate deficits of neuroprotective mediators (1) deficiencies in precursors and intermediates of lipid mediators pathways and of ELOVL-4 during the early development of retina pathology in the 5×FAD mouse. (A) The biosynthetic pathway of NPD1, 32:6n-3 and 34:6n-3 elovanoids from their PC 54-12 and PC56-12 precursors. For the mass spectrometry detection, the stable monohydroxy products of VLC-PUFAs are used. (B) The bar chart for free 32:6n-3 and 34:6n-3 VLC-PUFAs, 27-monohydroxy 32:6n-3 and 29-monohydroxy 34:6n-3 VLC-PUFAs, free DHA, and NPD1 in the retina (top) and RPE layers (bottom). (C-D) Western blots and quantification of 15-lypoxygenase-1 expression in RPE and Retina. In RPE, the expression of 15-lypoxygenase-1 in 5×FAD is less than WT RPE. In the retina, there is no difference between two groups. This explains why the level of NPD1 is lower in 5×FAD, but no change in the retina. ELOVL4 is only express in retina and its expression is lower in 5×FAD. As a consequence, there was less free 32:6n-3 and 34:6n-3 as well as less abundant monohydroxy molecules. (NS: non-significant, *P<0.05, using student t-test comparison).

FIG. 38 shows interactions between NPD1, ELV32 and ELV34 with positive screened GPCRs by Path Hunter β-arrestin complementation. Receptors for which the lipids showed antagonism (red) and agonist activity (blue) are depicted. The circled GPCRs showed activity over threshold, while the rest are borderline. Because path-hunter is a heterologous system, as an alternative approach, we can also test GPCR that did not meet the limit activity but were close to it.

FIG. 39 shows UOS up-regulates pro-inflammatory transcriptome and down regulates pro-homeostatic pathways in RPE single-cells. NPD1 and ELV32-6 suppress these changes. Dots in box plots represent expression of the gene for a single RPEC (total of 96 per sample). P<0.001 (***), P<0.01 (**), One-way Anova, with Post-Hoc Tukey HSD test for multiple comparisons.

FIG. 40 shows structure of glutathione reductase based on the x-ray structure of human glutathione reductase.

FIG. 41 shows analysis of target candidate protein TXNRD1. Structure shown is based on the x-ray structure of human NADP(H) thioredoxin reductase I.

FIG. 42 shows the converging mechanisms that regulate senescence for the development of new synthetic non-lipidic analogs to mimic the bioactivity of the lipid mediators.

FIG. 43 shows population level electrical activity was recorded from different hypothalamic neurons, using the MEA system.

FIG. 44 shows protection by Elovanoids of hypothalamic neuronal cell death (by Fluoro-Jade B staining) in adult obese diabetic mice (db/db).

FIG. 45 shows senescense associated β-galactosidase activity measured in human neuronal-glial (HNG) cells exposed to oligomeric amyloid beta (Oaβ) (10 μM). (A-G) SA-β-Gal activity in HNG cells treated with Oaβ (10 μM) and different Elovanoids (ELVs) or neuroprotectin D1 (NPD1) at a concentration of 500 nM. Micrographs were obtained with bright field microscopy. (H) Quantification of SA-β-Gal+ cells shown in (A-G). SA-β-Gal+ cells were scored in 3 random fields of at least 150 total cells. Results are expressed as percentage of stained SA-β-Gal+ cells (mean±SEM). Statistical analysis were done using Graphpad Prism software 8.3. Results compared with one-way ANOVA, followed by Holm's Sidak post hoc tests and p<0.05 was considered statistically significant.

FIG. 46 shows senescense associated β-galactosidase activity measured in human neuronal-glial (HNG) cells exposed to Erastin (10 μM). (A-G) SA-β-Gal activity in HNG cells treated with Erastin (10 μM) and different Elovanoids (ELVs) or neuroprotectin D1 (NPD1) at a concentration of 500 nM. Micrographs were obtained with bright field microscopy. (H) Quantification of SA-β-Gal+ cells shown in (A-G). SA-β-Gal+ cells were scored in 3 random fields of at least 150 total cells. Results are expressed as percentage of stained SA-β-Gal+ cells (mean±SEM). Statistical analysis were done using Graphpad Prism software 8.3. Results compared with one-way ANOVA, followed by Holm's Sidak post hoc tests and p<0.05 was considered statistically significant.

FIG. 47 shows experimental design: Human neuronal glial (HNG) cells were challenged using Oaβ or Erastin.

FIG. 48 shows ELV34 revert the effect of IL1β in human diabetic adipocytes. A) experimental design. B) expression levels of TP53 and IL8 in human diabetic and non-diabetic adipocytes by the means of Tagman Real time PCR.

FIG. 49 shows ELV34 reduced the levels of IL6 (marker of SASP) induced by IL1β in Diabetic db/db mice hypothalamus indicating that hypothalamic neurons and astrocytes undergo SP. Different effects have been observed in female and male mice.

FIG. 50 shows characteristics of the db/db mice versus WT.

FIG. 51 shows ELV34 treatment increased the levels of Adiponectin an anti-diabetic systemic hormone secreted by adipocytes and other tissues (hypothalamus) that promotes insulin sensitivity. A) Diabetic hypothalamus treated with ELV34 showed a trend of increase in adiponectin in female and males. B) Differential effect of ELV34 in subcutaneous adipose tissue (SAT), and visceral adipose tissue (VAT). SAT and VAT possess differential ability to browning 19.

FIG. 52 shows deficiency of VLC-PUFAs in phosphatidylcholine molecular species in the retina of the 5×FAD. (A) PCs heatmap analysis of 6-month-old 5×FAD (n=6) and wild-type (n=6). Two main clusters of PCs evolved with distinct features. Group 1 depicts abundant PCs in 5×FAD while Group 2 shows PCs prevalent in WT, with most PCs containing VLC-PUFAs. (B) PCA analysis for PCs illustrates two populations (WT-black and 5×FAD-red) scatter across the principal component 1. Thus, the loading score for the principal component 1 is essential to identify distinct PCs for the difference between WT and 5×FAD. (C) The loading score (absolute values) of PCs to the principal component 1. The higher loading score, the more contribution of the PCs into principal component to distinguish WT and 5×FAD (SI Appendix, FIG. S1A). Ten short chain PUFAs (<48 C) contained in PCs are found in the top 12 most loading score PCs (C) while the VLCPUFAs contained in PCs contribute two (58:1 and 58:12). (D) The time used in random forest classification for PCs of WT and 5×FAD. The higher time used, the more valuable of the PCs in WT and 5×FAD difference (SI Appendix, FIG. S1B). VLC-PUFAs contained PCs contribute seven of 12 top time used in this classification (D). (E-G) Box plot for VLC-PUFAs (E), DHA (F), and AA (G) containing PCs. The WT has more VLC-PUFAs and DHA containing PCs while the 5×FAD has more AA contained PCs. (*P<0.05, student t-test)

FIG. 53 shows deficiency of VLC-PUFAs in phosphatidylcholine molecular species of the RPE of the 5×FAD. (A) Heatmap analysis for PCs of 6-month-old 5×FAD (n=6) and WT (n=6). VLC-PUFAs contained PCs are less abundant in 5×FAD. (B) PCA for PCs in RPE of 5×FAD and WT. There are two populations scatter across the Principal component 1. However, it is not as clear as in the distribution in the retina (FIG. 52). For this reason, the loading score for Principal Component 1 is essential to identify the distinct PCs for the difference between WT and 5×FAD. (C) The loading score (absolute values) of PCs to the Principal Component 1. The higher loading score, the more contribution of PCs into Principal Component 1 to distinguish WT and 5×FAD (FIG. 59, panel C). Six short chain PUFAs (<48 C) contained PCs are found in the top 12 most loading score PCs (C) while the VLC-PUFAs contained PCs contributes six (50:8, 50:12, 52:8, 54:12, 56:12 and 58:12). (D) The time used in random forest classification for PCs of WT and 5×FAD. The higher time used, the more valuable of the PCs in WT and 5×FAD difference (FIG. 59, panel D). VLC-PUFAs contained PCs contribute nine of 12 top time used in this classification (D). (E) Violin plot for % of PC38-6, PC40-6, and PC44-12 in the WT and 5×FAD retina (upper) and eyecup (RPE, lower). Surprisingly, the eyecup PCs show that 5×FAD has more PC38:6, less PC44-12, and equal PC40-6 to WT. (NS: non-significant, *P<0.05, student t-test).

FIG. 54 shows deficiencies in precursors and intermediates of lipid mediator pathways and of ELOVL-4 during the early development of retina pathology in the 5×FAD. (A) Biosynthetic pathways of NPD1 and of 32:6n-3 and 34:6n-3 ELVs from PC 54-12 and PC56-12. (B) Free 32:6n-3 and 34:6n-3. (C) 27-monohydroxy 32:6n-3 and 29-monohydroxy 34:6n-3, stable derivatives of the hydroperoxyl-precursors of ELV N-32 and ELV N-34, respectively. (D) Free DHA. (E) NPD1. In B-E, retina (top) and RPE (bottom) (n=6/group). (F and H) 15 LOX1 and ELOVL4 Western blots and quantification in RPE and retina (n=6/group). In RPE, 15-lipoxygenase-1 in 5×FAD is less than WT. In retina, there is no difference between two groups. This is in agreement with lower NPD1 pool size in 5×FAD, but no change in retina (E). ELOVL4 is only expressed in retina and is lower in 5×FAD. As a consequence, there was less free 32:6n-3 and 34:6n-3 as well as less abundant monohydroxy stable precursor derivatives. (NS: non-significant, *P<0.05, student t-test).

FIG. 55 shows morphology and function of the 5×FAD retina. (A) V log I plot showing maximum ERG b-wave amplitudes for light flashes from 0 to 0.075 cd·s/m2. 5×FAD achieved maximum amplitude of about 100 μV, approximately half that recorded for WT (n=6/group). (B) Electron microscopy of 6-month-old 5×FAD retinas illustrating similarities to WT. (Bi) Basal side of a 5×FAD RPE cell showing membrane infoldings along Bruch's membrane (Br). (Bii) Disk synthesis region (arrow) at basal portion of a rod outer segment showing newly formed disks from the connecting cilium (CC) membrane (WT). (Biii) Similar region in a 5×FAD displaying new disk formation (arrow). (Biv) The outer limiting membrane (OLM, arrow) at the scleral edge of the cell body layer (N, photoreceptor nucleus) in the 5×FAD. The cytoplasm of Müller cells (M) are lighter than that of PRC. (Bv) The interface between a 5×FAD RPE cell and a rod PRC tip (PR). Two phagosomes (Ph) within the RPE cytoplasm; the lower Ph is held within the RPE apical processes, while the upper, darker Ph is older and just entering the RPE cell body, illustrating normal phagocytic function. (Bvi) Inner segment mitochondria (M) of 5×FAD retain the very elongate PRC. (C) Five-month old WT and 5×FAD retinal sections illustrating normal PRC profiles within the 5×FAD retina. (D) Fluorescent staining of the retina from WT and 5×FAD. The blue (DAPI) is the nuclei and red A3 in the RPE layer at 6 months old in 5×FAD. (*P<0.05, student t-test).

FIG. 56 shows ELVs restore RPE morphology and reduce gene expression after subretinal injection of OAβ in WT mice. (A) In vivo experimental design: 6-month-old C57BL/6J WT mice were divided into 7 groups (n=12/group): non-injected, PBS, OAβ only, OAβ+ELV-N-32, OAβ+ELV-N-34, ELV-N-32 only and ELV-N-34 only. On day 3, mRNA were isolated for Real-Time PCR. On day 7, mice were subjected to OCT and then eyes enucleated and processed for whole mount RPE staining and Western blots. (B) Whole flat mount of RPE with tight junction marker, Zonula occludens-1 (ZO-1) antibody. OAβ disrupted RPE morphology. (C) OAβ effects on retina and RPE by OCT. (D) Thickness of PRC layer was thinner in OAβ injected group. (E) RPE gene expression after Oaβ (1-42) injection and treatment with ELVs. (E-G) Gene expression in the same functional group were plotted. Senescence- and AMD-related genes (E), and collagenases, gelatinase, stromelysins and others matrix metalloproteinases (MMP) (F) and autophagy (G). (H) p16INK4a, a key marker for senescence, western blots of RPE. (I) Retina apoptosis gene expression after OAβ (1-42) injection and treatment with ELVs. (*P<0.05, student t-test).

FIG. 57 shows OA-β-mediated activation of senescence-associated secretory phenotype (β-galactosidase, SA-β-Gal) and of gene expression in human RPE cells in primary culture are counteracted by ELVs. (A) In vitro experimental design: Primary human RPE cells were treated with 10 μM OAβ+/−ELVs. After 3 days, RNA was isolated and qPCR analyzed. After 7 days, cells were subjected to β-Galactosidase staining. (B) Live cell images under bright field microscopy after 7 days. (C) β-Gal staining+/−ELVs. Quantitation of % for the β-Gal positive cells. ELVs decreased positive senescent cells. (D) Gene transcription of senescence, AMD-related and autophagy genes after OAβ (1-42) exposure+/−ELVs. (*P<0.05, student t-test).

FIG. 58 shows summary of ELVs effects on OAβ-induced RPE and PRC damage. (A) OAβ induces a senescence program and disrupts RPE tight junctions. Without wishing to be bound by theory, OAβ penetrates the retina, causing PRC cell death in our in vivo WT mice study reflected in less cell body layer (CBL) nuclei. ELVs restore RPE morphology and PRC integrity. (B) OAβ induces expression of senescence, autophagy, matrix metalloproteinases, and AMD-related genes in the RPE and of apoptosis genes in retina. ELVs downregulated the OAβ-gene inductions. Pathways for the ELVs synthesis are depicted.

FIG. 59 shows principal component analysis loading score and random forest time used for all PC species in retina and RPE. (A) The full loading score (absolute values) of all PCs to the Principle Component 1 in the retina. The higher loading score, the more contribution of the PCs into Principle Component 1 to distinguish the retinas from wild type and 5×FAD. (B) The time used in random forest classification for all retinal PCs of wild type and 5×FAD mouse. The higher time used, the more valuable of the PCs in wild type and 5×FAD difference. (C) The loading score (absolute values) of all PCs to the Principle Component 1 in the RPE. The higher loading score, the more contribution of the PCs into Principle Component 1 to distinguish the RPEs from wild type and 5×FAD. (D) The time used in random forest classification for all RPE's PCs of wild type and 5×FAD mouse. The higher time used, the more valuable of the PCs in wild type and 5×FAD difference.

FIG. 60 shows the fatty acid compositions of PCs are obtained by full fragmentation. The negative ion modes were used for LC/MS/MS data acquisition. The VLC-PUFA-containing PCs are depicted; (A) PC54:12 (m/z 1076 (M+CH₃COO—) corresponds to m/z 1018 (M+H+) in positive mode) is composed of FA32:6 (m/z 467) and FA22:6 (m/z 327). (B) PC56:12 (m/z 1104 (M+CH₃COO—) corresponds to m/z 1046 (M+H+) in positive mode) is composed of FA34:6 (m/z 495) and FA22:6 (m/z 327). (C) PC58:12 (m/z 1132 (M+CH₃COO—) corresponds to m/z 1074 (M+H+) in positive mode) is composed of FA36:6 (m/z 523) and FA22:6 (m/z 327). DHA-containing PCs are on the second row; (D) PC38:6 (m/z 864 (M+CH₃COO—) corresponds to m/z 806 (M+H+) in positive mode) is composed of FA16:0 (m/z 255) and FA22:6 (m/z 327). (E) PC40:6 (m/z 892 (M+CH₃COO—) corresponds to m/z 834 (M+H+) in positive mode) is composed of FA18:0 (m/z 283) and FA22:6 (m/z 327.0). (F) PC44:12 (m/z 936 (M+CH₃COO—) corresponds to m/z 878 (M+H+) in positive mode) is composed of two FA22:6s (m/z 327.0). AA-containing PCs are at the third row; (G) PC36:4 (m/z 840 (M+CH₃COO—) corresponds to m/z 782 (M+H+) in positive mode) is composed of FA16:0 (m/z 255) and FA20:4 (m/z 303). (H) PC38:4 (m/z 868 (M+CH₃COO—) corresponds to m/z 810 (M+H+) in positive mode) is composed of FA18:0 (m/z 283) and FA20:4 (m/z 303). (I) PC38:5 (m/z 866 (M+CH₃COO—) corresponds to m/z 808 (M+H+) in positive mode) is composed of FA18:1 (m/z 281) and FA20:4 (m/z 303). This peak is also from PC38:6 isotopes (two carbons being naturally C13 labeled). That will produce FA16:0 (m/z 255) and FA22:6 (m/z 329 when two carbons are C13), or FA16:0 (m/z 256 when one carbon is C13) and FA22:6 (m/z 328 when one carbon is C13).

FIG. 61 shows full fragmentation spectra of ELVs and pre-cursor molecules and NPD1. (A) Full fragmentation spectra of elovanoid precursors, free fatty acids FA32:6 n-3 and FA34:6-n-3, shows the molecular structure and fragmentation patterns. (B) Stable precursors of elovanoids, Endogenous 27-monohydroxy 32:6 and 29-monohydroxy 34:6, show good matching to the theoretical fragmentation patterns, shown in the structure. (C) Full fragmentation of elovanoids, ELV32 and ELV34. The standards exhibit all of the peaks shown in the structure and fragmentation patterns. (D) NPD1 fragmentation spectrum is described with theoretical values.

FIG. 62 shows fundus and OCT analysis. (A) Fundus and Optical Coherence Tomography (OCT) images of Wild type (WT) and 5×FAD mice. Mice are about 6 months old. (B) The analysis of the thickness of the photoreceptor layers. There is no difference between WT and 5×FAD.

FIG. 63 shows inflammation signal is activated by 5×FAD. (A) The relative normalized expression of AMD-related genes of the RPE in WT and 5×FAD. The RNA from eye cup/choroid of WT and 5×FAD (6 months old) were isolated and reverse transcribed into cDNA and subjected to RT-PCR with AMD-related genes. Briefly, there is the activation of different genes, related to the AMD in 5×FAD. (B) The relative normalized expression of inflammatory genes in the retina. The RNA from retina of WT and 5×FAD (6 months old) were isolated and reverse transcribed into cDNA and subjected to RT-PCR with different inflammatory genes. Briefly, there is the activation of inflammation signaling in 5×FAD.

FIG. 64 shows western blot for Oligomers Aβ. After 24 hours of oligomerization, the 2 μl of the Aβ stock was loaded in the tricine gel, without the denaturation.

FIG. 65 shows the unfolded protein response (UPR) genes. After 3 days injection, the RNA from RPE and retina were isolated and reverse transcribed into cDNA and subjected to RT-PCR with UPR primers. There was no change in both RPE (A) and retina (B) for these genes.

FIG. 66 shows antibodies as utilized herein.

FIG. 67 shows gene primer sequences as utilized herein.

FIG. 68 shows dust mites under a microscope. Under a microscope, dust mites can look like white bugs.

FIG. 69 shows an illustration of HDM allergenicity: the various components and their associated fecal pellets and dust, which activate the immune system to initiate inflammatory responses (see, Gregory et al., Orchestrating house dust mite-associated allergy in the lung. Trends in Immunology, vol. 32, issue 9, p. 402-411, 2011).

FIG. 70 shows a schematic of lipopolysaccharide (LPS) of a gram negative bacteria and the structure of polyinosinic-polycytidylic acid, poly(I:C).

FIG. 71 shows a non-limiting, exemplary experimental design of human nasal epithelial cells challenged by aeroallergens.

FIG. 72 shows a schematic representation of the reported and potential hidden functions of Der p 1 in the HDM induced inflammatory response. Der p 1 triggers a proteolytic activation cascade in the digestive tract of the mite Dermatophagoides pteronyssinus, leading to maturation of the serine protease allergens Der p 3, Der p 6, and Der p 9 and other mite protein targets. Within mite fecal pellets, Der p 1 could selectively cleave and/or extensively degrade several proteins, including HDM allergens and endosymbiotic bacteria. These activities could modulate the respective stability of HDM allergens and facilitate their release from the peritrophic matrix, as well as affecting generation of peptides for antigen processing. When fecal pellets reach the airway mucosa, elution of the microbial compounds and intact and fragmented allergens leads to initiation of the HDM-induced allergic response. Active Der p 1 could exert direct and indirect pleiotropic effects at the level of the airway epithelium, lung microbiome, and secretome, leading to release of proinflammatory cytokines (IL-6, IL-8, GM-CSF, thymic stromal lymphopoietin, and IL-25), alarmins (IL-lan and IL-33), and chemo-attractants (CCL2 and CCL20), which activate DCs, basophils, and ILC2s to initiate the TH2-biased HDM-induced allergic response. Red and black arrows or parentheses represent the different hypothetical and experimentally evidenced effects of active Der p 1, respectively. AEC, Airway epithelial cell; AMG, anterior midgut; cDC, conventional DC; cDC2c, type 2 conventional DC; HG, hindgut; IL-33FL, full length IL-33; PM, peritrophic matrix; TJ, tight junction protein. See, Chevigne A, Jacquet A. Emerging roles of the protease allergen Der p 1 in house dust mite-induced airway inflammation. J Allergy Clin Immunol. 2018 August; 142(2):398-400, Epub 2018.

FIG. 73 shows graphs of HDMs, D. ptero (Dermatophagoides pteronyssinus) or D. fari (Dermatophagoides farina) on cell survival and IL-6 (senescence activator).

FIG. 74 shows HDMs, D.P. (Dermatophagoides pteronyssinus) or D.F. Dermatophagoides farina) on IL-1β and IL-8 (senescence inducers).

FIG. 75 shows LPS and Poly(I:C) on cell survival.

FIG. 76 shows the effects of LPS or Poly(I:C).

FIG. 77 shows schematics of senescence initiation, early senescence, and late senescence (Herranz and Gil, Mechanisms and Functions of Cellular Senescence, J. Clin Invest. 2018; 128(4): 1238-1246).

FIG. 78 shows a schematic of a quiescent and senescent cell (see Maciel-Baron et al., Cellular Senescence, Neurological Function, and Redox State, Antioxid Redox Signal. 2018 Jun. 20; 28(18): 1704-1723).

FIG. 79 shows a schematic of cellular senescence and the senescence associated secretory phenotype (SASP) (see Parikh et al., Colonic epithelial cell diversity in health and inflammatory bowel disease, nature 2019 March; 567(7746): 49-55.

FIG. 80 shows a graph of expression data. HDM induces MMPs gene expression: ELV 34 inhibits these effects.

FIG. 81 shows graphs of senescence gene programming data and inflammation/senescence. Elovanoid N-34 as a senolytic. HDM induces senescence (except p53) and senescence-associated cytokines gene expression in human nasal epithelial cells: Elovanoids 34 inhibits these effects.

FIG. 82 shows assessment of 30 μg/ml+/−ELV-34:6 (500 nM) as a Senolytic Compound Cellular senescence in cells challenged with HDM Staining with Spider β-gal (Dojindo) (see Cellular Senescence Detection Kit-SPiDER-βGal Technical Manual Issued Mar. 30, 2017).

FIG. 83 shows images of human nasal cells stained with spider β-gal.

FIG. 84 shows a graph of β-gal positive cells (%).

FIG. 85 shows a model of ELVs in OAβ-induced RPE and PRC damage. ELVs downregulated the OAβ-gene inductions. Pathways for the synthesis of ELVs are depicted.

FIG. 86 shows a non-limiting, exemplary schematic elovanoids as senolytics for allergy.

FIG. 87 shows a schematic representation of the link between cellular senescence and aging (see Thoppil H, et al: A Translational Bridge Between Cellular Senescence and Organismal Aging. Front Cell Dev Biol. 2020; 7:367).

FIG. 88 shows a schematic diagram showing multiple locations of Aβ synthesis, secretion, and aggregation in the ageing retina (red asterisk) that is reported in the literature to date. BrM, Bruch's Membrane; POS, photoreceptor outer segments; RGC, retinal ganglion cells; RPE, retinal pigment epithelium (from Ratnayaka J A, et al. Eye Lond Engl. 2015; 29:1013. doi:10.1038/eye.2015.100).

FIG. 89 shows a non-limiting, exemplary scheme for synthesis of elovanoids.

FIG. 90 shows a diagram of the ELOVL4 protein (see Ozaki et al., JAMA Neurol. 2015; 72:797-805) and diagrams of mutations in DNA and protein (see Logan et al., 2013 PNAS USA). The brown boxes indicate transmembrane domains as predicted in previous reports and the Uniprot Knowledgebase (see Magrane M; UniProt Consortium. UniProt Knowledgebase: a hub of integrated protein data. Database. 2011; 2011:bar009 and Zhang K, Kniazeva M, Han M, et al. A 5-bp deletion in ELOVL4 is associated with two related forms of autosomal dominant macular dystrophy. Nat Genet. 2001; 27(1):89-93); the green box indicates the dioxy iron-binding motif (HXXHH); the yellow box indicates the dilysine motif for the retention of transmembrane proteins in the endoplasmic reticulum. The mutations (p.W246G and p.L168F) lead to spinocerebellar ataxia (black arrowheads). The p.W246G mutation was identified in our study (the red rectangle). Two recessive mutations (p.R216X and p.I230MfsX22 [blue arrowheads]) were reported to cause ichthyosis, spastic quadriplegia, and mental retardation (Aldahmesh M A, Mohamed J Y, Alkuraya H S, et al. Recessive mutations in ELOVL4 cause ichthyosis, intellectual disability, and spastic quadriplegia. Am J Hum Genet. 2011; 89(6):745-750). Three mutations (N264LfsX9, N264TfsX10, and Y270X [orange arrowheads]) cause autosomal dominant Stargardt-like macular dystrophy (Vasireddy V, Wong P, Ayyagari R. Genetics and molecular pathology of Stargardt-like macular degeneration. Prog Retin Eye Res. 2010; 29(3):191-207). (K Ozaki et al., JAMA Neurol. 2015; 72:797-805).

FIG. 91 shows non-limiting, exemplary structures, UV spectra, and mass spectra of elovanoids (see Bhattacharjee S, et al. Sci. Adv. 2017:3:e1700735 and Bazan N. Mol. Aspects Med. 2018; 64:18-33.doi:10.1016).

FIG. 92 shows a non-limiting experimental design where mice were divided into 7 groups: non-injected, PBS, OAβ only, OAβ+ELV-N32, OAβ+ELV-N34, ELV-N32 only and ELV-N34 only. On day 3, mRNA were isolated for RT-PCR. On day 7, mice were subjected to OCT and then eyes were enucleated and processed to whole mount RPE staining and Western blot.

FIG. 93 shows Whole flat mount of RPE. OAβ disrupted RPE morphology. However, RPE were less damage in the ELVs treatment group as well as PBS, ELVs alone did not induce changes (see K. Do et al, PNAS, 2019).

FIG. 94 shows non-limiting, exemplary evaluation the OAβ effects on retina and RPE by OCT and thickness of PRC was thin in the OAβ injected group. OAβ cause the cell death of PRC, as the thinner in OCT measurement.

FIG. 95 shows graphs of non-limiting, exemplary data. RPE gene expression after OAβ (1-42) injection and treatment with ELVs. 3 days after injection, the RNAs from RPE were isolated, reverse transcribed into cDNA and subjected to RT-PCR with different primers. Genes in the same functional group were plotted in the same chart, including senescence- and AMD-related genes (Panel A), and collagenases, gelatinase, stromelysins and others matrix metalloproteinases (MMP) (Panel B) and autophagy (Panel C). (Panel D) p16INK4a western blots of RPE/Choroid. ELV-N32 and ELV-N34 down regulated the expression of the senescence marker, p16INK4a, which was elevated by OAβ injection. (Panel E) Retina gene expression after OAβ (1-42) injection and treatment with ELVs. OAβ activates apoptosis genes in the retina. With ELVs co-injection, these genes were down-regulated. (*P<0.05, using student t-test comparison).

FIG. 96 shows non limiting-exemplary data of lipid mediator pathways and ELOVL-4 during early retina pathology in the 5×FAD mouse. Panel A shows the biosynthetic pathway of NPD1, 32:6n3 and 34:6n3 elovanoids from their PC 54-12 and PC56-12 precursors. For the mass spectrometry detection, the stable monohydroxy products of VLC-PUFAs are used. Panel B shows the bar chart for free 32:6n3 and 34:6n3 VLC-PUFAs, 27-monohydroxy 32:6n3 and 29-monohydroxy 34:6n3 VLC-PUFAs, free DHA, and NPD1 in the retina (top) and RPE layers (bottom). Panel C shows western blots and quantification of 15-lypoxygenase-1 expression in RPE and Retina. In RPE, the expression of 15-lypoxygenase-1 in 5×FAD is less than WT RPE. In the retina, there is no difference between two groups. This explains why the level of NPD1 is lower in 5×FAD, but no change in the retina. ELOVL4 is only express in retina and its expression is lower in 5×FAD. As a consequence, there was less free 32:6n3 and 34:6n3 as well as less abundant monohydroxy molecules. (NS: non-significant, *P<0.05, using student t-test comparison).

FIG. 97 shows OA-β-mediated activation of senescence-associated secretory phenotype (β-galactosidase, SA-β-Gal) and of gene expression in human RPE cells in primary culture are counteracted by ELVs. (Panel A) In vitro experimental design: Primary human RPE cells were treated with 10 μM OAβ+/−ELVs. After 3 days, RNA was isolated and qPCR analyzed. After 7 days, cells were subjected to β-Galactosidase staining. (Panel B) Live cell images under bright field microscopy after 7 days. (Panel C) β-Gal staining+/−ELVs. Quantitation of % for the β-Gal positive cells. ELVs decreased positive senescent cells. (Panel D) Gene transcription of senescence, AMD-related and autophagy genes after OAβ (1-42) exposure+/−ELVs. (*P<0.05, student t-test).

FIG. 98 shows (Panel A) a schematic of healthy condition and retinal degeneration tight junctions and panel B shows non-limiting examples of the effects of elovanoids on OAβ

FIG. 99 shows a schematic of omega-3 PUFA in AMD (Das Y, et al Peroxisomal Multifunctional Protein 2 Deficiency Perturbs Lipid Homeostasis in the Retina and Causes Visual Dysfunction in Mice. Front Cell Dev Biol. 2021).

FIG. 100 shows ELV-N32 and ELV-N34 enhance abundance of pro-homeostatic proteins and decrease abundance of cell damaging proteins in RPE cells under UOS. ELV-N32 or ELV-N34 indicates the sodium salt forms, and ELV-N34-Me or ELV-N32-Me indicates the methyl ester forms. ELVs induces the following effects in ARPE-19 cells undergoing UOS: (panel a) Concentration-dependent (100 and 250 nM) upregulation of SIRT1. The results are the averages of three independent experiments; (panel b) upregulation of Iduna abundance; (panel c) increased abundance of anti-apoptotic proteins Bcl-2 and Bcl-xL; (panel d) decreased abundance of pro-apoptotic proteins Bax, (panel e) Bid and (panel f) Bim. (panel g) Concentration-dependent (100 and 250 nM) upregulation of Prohibitin (type-1) by ELVs takes place. (panel h) Concentration-dependent (50, 100, 250, and 500 nM) reduction of UOS-induced apoptosis. Error bars, SEM; *p<0.05. See Jun B, et al Sci. Rep 7

FIG. 101 shows data and schematics proteins and elovanoids.

FIG. 102 shows structures and schematics for very long-chain PUFA Phosphatidyl Choline Molecular Species.

FIG. 103 shows non-limiting, exemplary images and data of ELV-34. ELVN-34 protects HNEpC from the senescence-associated secretory phenotype (SASP response). Representative images of HNEpC stained with Hoechst 33342 and spider β-galactosidase—(Panel A) untreated (Control), (Panel B) challenged with HDM (30 μg/ml), and (Panel C) HNEpC treated with HDM and ELVN-34:6 Na (500 nM). (Panel D) Quantitative assessment of increased SASP response (measured by the spider β-galactosidase staining) upon HDM exposure (30 μg/ml). ELVN-34:6 Na (500 nM) treatment reduces the number of beta-gal positive HNEpC induced by HDM. The results showed the averages of three independent experiments. (*p<0.05).

FIG. 104 shows graphs of non-limiting, exemplary expression data. HDM triggered multiple signaling in human nasal epithelial cells. HNEpC challenged with HDM (D. farinae+D. pteronyssinus) (30 μg/ml) induces the expression of several genes related with autophagy (ATG3, ATG5, ATG7, BECLIN-1, and P62), unfolded protein response (UPR) (ATF6, CHOP, and IRE1) and matrix metalloproteinases (MMPs) (MMP8, MMP2, MMP9, MMP3, MMP12, TIMP1, and TIMP2). HDM stressors also induce the expression of senescence (P21, P16, P27, and P53) and inflammation genes (IL-1a, IL-6, and IL-10) on HNEpC. The treatment with ELVN-34 Na (500 nM) reduces the expression of autophagy, UPR, MMP, senescence (except P53), and inflammation genes induced by HDM extracts. The results showed the averages of three independent experiments. (*p<0.0001).

FIG. 105 shows non-limiting, exemplary structures of elovanoids. ELVN-34:6 Na, ELVN-34:6 Me, and ELVN-34:6 Me-A at (500 nM) were used for experiments in this study.

FIG. 106 shows ELISA Standard curves. For the ELISA assays mentioned in this example, the standard curves for each assay are given herein.

FIG. 107 shows a schematic of allergens triggering an inflammatory immune response which can be attenuated by elovanoid N34.

### DETAILED DESCRIPTION OF THE DISCLOSURE

Before the present disclosure is described in greater detail, this disclosure is not limited to particular embodiments described, and as such can, of course, vary. The terminology used herein is for the purpose of describing particular embodiments only, and is not intended to be limiting, since the scope of the present disclosure will be limited only by the appended claims.

Where a range of values is provided, each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges can independently be can included in the smaller ranges and are also encompassed within the disclosure, subject to any specifically excluded limit in the stated range. Where the stated range can include one or both of the limits, ranges excluding either or both of those can included limits are also can included in the disclosure.

Unless defined otherwise, all technical and scientific terms used herein have the same meaning as understood by one of ordinary skill in the art to which this disclosure belongs. Although any methods and materials similar or equivalent to those described herein can also be used in the practice or testing of the present disclosure, the advantageous methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that can need to be independently confirmed.

As will be apparent to those of skill in the art upon reading this disclosure, each of the individual embodiments described and illustrated herein has discrete components and features which can be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, toxicology, and the like, which are within the skill of the art. Such techniques are explained fully in the literature.

It must be noted that, as used in the specification and the appended claims, the singular forms “a,” “an,” and “the” can include plural referents unless the context clearly dictates otherwise. Thus, for example, reference to “a support” can includes a plurality of supports. In this specification and in the claims that follow, reference will be made to a number of terms that can have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “can includes,” “including,” and the like; “consisting essentially of” or “consists essentially” or the like, when applied to methods and compositions encompassed by the present disclosure can refer to compositions like those disclosed herein, but which can contain additional structural groups, composition components or method steps (or analogs or derivatives thereof as discussed herein). Such additional structural groups, composition components or method steps, etc., however, do not materially affect the characteristic(s) of the compositions or methods, compared to those of the corresponding compositions or methods disclosed herein.

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

As used herein, the term “about” can be approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Prior to describing the various embodiments, the following exemplary descriptions are provided.

As used herein, the nomenclature alkyl, alkoxy, carbonyl, etc. is used as is understood by those of skill in the chemical art. As used in this specification, alkyl groups can include straight-chained, branched and cyclic alkyl radicals containing up to about 20 carbons, or 1 to 16 carbons, and are straight or branched. Alkyl groups herein can include, but are not limited to, methyl, ethyl, propyl, isopropyl, isobutyl, n-butyl, sec-butyl, tert-butyl, isopentyl, neopentyl, tert-pentyl and isohexyl.

As used herein, lower alkyl can refer to carbon chains having from about 1 or about 2 carbons up to about 6 carbons. Suitable alkyl groups can be saturated or unsaturated. Further, an alkyl can also be substituted one or more times on one or more carbons with substituents selected from a group consisting of C1-C15 alkyl, allyl, allenyl, alkenyl, C3-C7 heterocycle, aryl, halo, hydroxy, amino, cyano, oxo, thio, alkoxy, formyl, carboxy, carboxamido, phosphoryl, phosphonate, phosphonamido, sulfonyl, alkylsulfonate, arylsulfonate, and sulfonamide. Additionally, an alkyl group can contain up to 10 heteroatoms, in certain embodiments, 1, 2, 3, 4, 5, 6, 7, 8 or 9 heteroatom substituents. Suitable heteroatoms can include nitrogen, oxygen, sulfur and phosphorous.

As used herein, “cycloalkyl” can refer to a mono- or multicyclic ring system, in certain embodiments of 3 to 10 carbon atoms, in other embodiments of 3 to 6 carbon atoms. The ring systems of the cycloalkyl group can be composed of one ring or two or more rings which can be joined together in a fused, bridged or spiro-connected fashion.

As used herein, “aryl” can refer to aromatic monocyclic or multicyclic groups containing from 3 to 16 carbon atoms. As used in this specification, aryl groups are aryl radicals, which can contain up to 10 heteroatoms, in certain embodiments, 1, 2, 3 or 4 heteroatoms. An aryl group can also be substituted one or more times, in certain embodiments, 1 to 3 or 4 times with an aryl group or a lower alkyl group and it can be also fused to other aryl or cycloalkyl rings. Suitable aryl groups can include, for example, phenyl, naphthyl, tolyl, imidazolyl, pyridyl, pyrroyl, thienyl, pyrimidyl, thiazolyl and furyl groups.

As used in this specification, a ring can have up to 20 atoms that can include one or more nitrogen, oxygen, sulfur or phosphorous atoms, provided that the ring can have one or more substituents selected from the group consisting of hydrogen, alkyl, allyl, alkenyl, alkynyl, aryl, heteroaryl, chloro, iodo, bromo, fluoro, hydroxy, alkoxy, aryloxy, carboxy, amino, alkylamino, dialkylamino, acylamino, carboxamido, cyano, oxo, thio, alkylthio, arylthio, acylthio, alkylsulfonate, arylsulfonate, phosphoryl, phosphonate, phosphonamido, and sulfonyl, and further provided that the ring can also contain one or more fused rings, including carbocyclic, heterocyclic, aryl or heteroaryl rings.

As used herein, alkenyl and alkynyl carbon chains, if not specified, contain from 2 to 20 carbons, or 2 to 16 carbons, and are straight or branched. Alkenyl carbon chains of from 2 to 20 carbons, in certain embodiments, contain 1 to 8 double bonds, and the alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds.

As used herein, “heteroaryl” can refer to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group can be fused to a benzene ring. Heteroaryl groups can include, but are not limited to, furyl, imidazolyl, pyrrolidinyl, pyrimidinyl, tetrazolyl, thienyl, pyridyl, pyrrolyl, N-methylpyrrolyl, quinolinyl and isoquinolinyl.

As used herein, “heterocyclyl” can refer to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur. In embodiments where the heteroatom(s) is(are) nitrogen, the nitrogen is substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, or the nitrogen can be quaternized to form an ammonium group where the substituents are selected as described herein.

As used herein, “aralkyl” can refer to an alkyl group in which one of the hydrogen atoms of the alkyl is replaced by an aryl group.

As used herein, “halo”, “halogen” or “halide” can refer to F, Cl, Br or I.

As used herein, “haloalkyl” can refer to an alkyl group in which one or more of the hydrogen atoms are replaced by halogen. Such groups can include, but are not limited to, chloromethyl and trifluoromethyl.

As used herein, “aryloxy” can refer to RO—, in which R is aryl, including lower aryl, such as phenyl.

As used herein, “acyl” can refer to a —COR group, including for example alkylcarbonyl, cycloalkylcarbonyl, arylcarbonyl, or heteroarylcarbonyls, all of which can be substituted.

As used herein, “n-3” or “n-3”, “n-6” or “n6”, etc. can refer to the customary nomenclature of polyunsaturated fatty acids or their derivatives, wherein the position of a double bond (C═C) is at the carbon atom counted from the end of the carbon chain (methyl end) of the fatty acid or fatty acid derivative. For example, “n-3” means the third carbon atom from the end of the carbon chain of the fatty acid or fatty acid derivative. Similarly, “n-3” or “n-3”, “n-6” or “n6”, etc. also can refer to the position of a substituent such as a hydroxyl group (OH) located at a carbon atom of the fatty acid or fatty acid derivative, wherein the number (e.g. 3, 6, etc.) is counted from the end of the carbon chain of the fatty acid or fatty acid derivative.

As used herein, the abbreviations for any protective groups and other compounds, are, unless indicated otherwise, in accord with their common usage, recognized abbreviations, or the IUPAC-IUB Commission on Biochemical Nomenclature (see, (1972) Biochem. 11:942-944).

As used herein, wherein in chemical structures of the compounds of the disclosure are shown having a terminal carboxyl group “—COOR,” the “R” can represent a group covalently bonded to the carboxyl such as an alkyl group. In the alternative, the carboxyl group can further have a negative charge as “—COO⁻” and R is a cation including a metal cation, an ammonium cation and the like.

The term “subject” or “patient” can refer to any organism to which aspects of the invention can be administered, e.g., for experimental, diagnostic, prophylactic, and/or therapeutic purposes. The term “subject” can include a mammal, for example, a human at any age suffering from pathology. In another embodiment, the term encompasses a subject at risk of developing pathology. Subjects to which compounds of the present disclosure can be administered will be animals, for example mammals, such as primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals for example pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” can refer to a subject noted herein or another organism that is alive. The term “living subject” can refer to the entire subject or organism and not just a part excised (e.g., a liver or other organ) from the living subject.

A “subject afflicted with a condition” or “a subject having a condition” can refer to a subject with an existing condition or a known or suspected predisposition toward developing a condition. In embodiments, the condition can be an inflammatory disease, such as an allergic inflammatory disease. In another embodiment, the condition can be a disease associated with cellular senescence, ferroptosis, or both, such as a disease associated with Aβ, including age-related macular degeneration or Alzheimer's disease. As still another embodiment, the disease can be a metabolic disorder, such as diabetes, obesity, or both.

As an example, a “subject having an allergic condition” can refer to a subject with an existing allergic condition or a known or suspected predisposition toward developing an allergic condition. Thus, the subject can have an active allergic condition or a latent allergic condition. It is not necessary that the allergen be known. However, certain allergic conditions are associated with seasonal or geographical environmental factors, which can but need not be apparent to the subject. In one embodiment the allergic condition is intentionally induced in the subject for experimental purposes.

As used herein, “pharmaceutically acceptable derivatives” of a compound can include salts, esters, enol ethers, enol esters, acetals, ketals, orthoesters, hemiacetals, hemiketals, acids, bases, solvates, hydrates or prodrugs thereof. Such derivatives can be readily prepared by those of skill in this art using known methods for such derivatization. The compounds produced can be administered to animals or humans without substantial toxic effects and either are pharmaceutically active or are prodrugs.

Pharmaceutically acceptable salts can include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamineand other alkylamines, piperazine and tris(hydroxymethyl) aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates.

Pharmaceutically acceptable esters can include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids.

Pharmaceutically acceptable enol ethers can include, but are not limited to, derivatives of formula C═C(OR) where R is hydrogen, alkyl, alkenyl, alkynyl, aryl, heteroaryl, aralkyl, heteroaralkyl, cycloalkyl, or heterocyclyl. Pharmaceutically acceptable enol esters can include, but are not limited to, derivatives of formula C═C(OC(O)R) where R is hydrogen, alkyl, alkenyl, alkynyl, aryl, heteroaryl, aralkyl, heteroaralkyl, cycloalkyl ar heterocyclyl.

Pharmaceutically acceptable solvates and hydrates are complexes of a compound with one or more solvent or water molecules, or 1 to about 100, or 1 to about 10, or one to about 2, 3 or 4, solvent or water molecules.

“Formulation” as used herein can refer to any collection of components of a compound, mixture, or solution selected to provide optimal properties for a specified end use, including product specifications and/or service conditions. The term formulation shall can include liquids, semi-liquids, colloidal solutions, dispersions, emulsions, microemulsions, and nanoemulsions, including oil-in-water emulsions and water-in-oil emulsions, pastes, powders, and suspensions. The formulations of the present invention can also be can included, or packaged, with other non-toxic compounds, such as cosmetic carriers, excipients, binders and fillers, and the like. Specifically, the acceptable cosmetic carriers, excipients, binders, and fillers for use in the practice of the present invention are those which render the compounds amenable to oral delivery and/or provide stability such that the formulations of the present invention exhibit a commercially acceptable storage shelf life.

As used herein, the term “administering” can refer to introducing a substance, such as a VLC-PUFA, into a subject. Any route of administration can be utilized including, for example, intranasal, topical, oral, parenteral, intravitreal, intraocular, ocular, subretinal, intrathecal, intravenous, subcutaneous, transcutaneous, intracutaneous, intracranial and the like administration. In embodiments, “administering” can also refer to providing a therapeutically effective amount of a formulation or pharmaceutical composition to a subject. The formulation or pharmaceutical compound of the present invention can be administered alone, but can be administered with other compounds, excipients, fillers, binders, carriers or other vehicles selected based upon the chosen route of administration and standard pharmaceutical practice. Administration can be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; other proteins and peptides; synthetic polymers; microspheres; nanoparticles; and the like.

The formulations or pharmaceutical composition can also be can included, or packaged, with other non-toxic compounds, such as pharmaceutically acceptable carriers, excipients, binders and fillers including, but not limited to, glucose, lactose, gum acacia, gelatin, mannitol, xanthan gum, locust bean gum, galactose, oligosaccharides and/or polysaccharides, starch paste, magnesium trisilicate, talc, corn starch, starch fragments, keratin, colloidal silica, potato starch, urea, dextrans, dextrins, and the like. Specifically, the pharmaceutically acceptable carriers, excipients, binders, and fillers for use in the practice of the present invention are those which render the compounds of the invention amenable to intranasal delivery, oral delivery, parenteral delivery, intravitreal delivery, intraocular delivery, ocular delivery, subretinal delivery, intrathecal delivery, intravenous delivery, subcutaneous delivery, transcutaneous delivery, intracutaneous delivery, intracranial delivery, topical delivery and the like. Moreover, the packaging material can be biologically inert or lack bioactivity, such as plastic polymers or silicone, and can be processed internally by the subject without affecting the effectiveness of the composition/formulation packaged and/or delivered therewith.

Different forms of the present inventive formulation can be calibrated in order to adapt both to different individuals and to the different needs of a single individual. However, the present formulation need not counter every cause in every individual. Rather, by countering the necessary causes, the present formulation will restore the body and brain to their normal function. Then the body and brain themselves will correct the remaining deficiencies.

The term “therapeutically effective amount” as used herein can refer to that amount of an embodiment of the composition or pharmaceutical composition being administered that will relieve to some extent one or more of the symptoms of the disease or condition being treated, and/or that amount that will prevent, to some extent, one or more of the symptoms of the condition or disease that the subject being treated has or is at risk of developing. As used interchangeably herein, “subject,” “individual,” or “patient,” can refer to a vertebrate, for example, a mammal, such as a human. Mammals can include, but are not limited to, murines, simians, humans, farm animals, sport animals, and pets. The term “pet” can include a dog, cat, guinea pig, mouse, rat, rabbit, ferret, and the like. The term farm animal can include a horse, sheep, goat, chicken, pig, cow, donkey, llama, alpaca, turkey, and the like.

A “pharmaceutically acceptable excipient,” “pharmaceutically acceptable diluent,” “pharmaceutically acceptable carrier,” or “pharmaceutically acceptable adjuvant” can refer to an excipient, diluent, carrier, and/or adjuvant that are useful in preparing a pharmaceutical composition that are safe, non-toxic and neither biologically nor otherwise undesirable, and can include an excipient, diluent, carrier, and adjuvant that are acceptable for veterinary use and/or human pharmaceutical use. “A pharmaceutically acceptable excipient, diluent, carrier and/or adjuvant” as used herein can include one and more such excipients, diluents, carriers, and adjuvants.

The phrase “pharmaceutical composition” or a “pharmaceutical formulation” can refer to a composition or pharmaceutical composition suitable for administration to a subject, such as a mammal, especially a human and that can refer to the combination of an active agent(s), or ingredient with a pharmaceutically acceptable carrier or excipient, making the composition suitable for diagnostic, therapeutic, or preventive use in vitro, in vivo, or ex vivo. In a “pharmaceutical composition” can refer to the composition being sterile, and free of contaminants that can elicit an undesirable response within the subject (e.g., the compound(s) in the pharmaceutical composition is pharmaceutical grade). Pharmaceutical compositions can be designed for administration to subjects or patients in need thereof via a number of different routes of administration including oral, intranasal, topical, intravenous, buccal, rectal, parenteral, intraperitoneal, intradermal, intracheal, intramuscular, subcutaneous, by stent-eluting devices, catheters-eluting devices, intravascular balloons, inhalational and the like.

In embodiments, the pharmaceutical composition can comprise a therapeutically effective amount of an elovanoid and a therapeutically effective amount of one or more additional active agents (such as one or more anti-oxidants, anti-allergenics, anti-inflammatory agents, or pain relievers). For example, the one or more anti-oxidants can be synthetic antioxidants, natural antioxidants, or a combination thereof. In embodiments, the anti-oxidants can protect the double bonds of the elovanoids.

The term “administration” can refer to introducing a composition of the present disclosure into a subject. Advantageous route of administration of the composition is topical administration, oral administration, or intranasal administration. However, any route of administration, such as intravenous, subcutaneous, peritoneal, intra-arterial, inhalation, vaginal, rectal, introduction into the cerebrospinal fluid, intravascular either veins or arteries, or instillation into body compartments can be used.

As used herein, “treatment” and “treating” can refer to the management and care of a subject for the purpose of combating a condition, disease or disorder, in any manner in which one or more of the symptoms of a disease or disorder are ameliorated or otherwise beneficially altered. The term can include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” can refer to the management and care of a patient for the purpose of hindering the development of the condition, disease or disorder, and can includes the administration of the active compounds to prevent or reduce the risk of the onset of symptoms or complications. Skilled artisans will appreciate a variety of methodologies and assays can be used to assess the development of pathology, and similarly, a variety of methodologies and assays can be used to reduce pathology, driveway, or regression.

As used herein, the term “preventing” can refer to preventing a disease, disorder, or condition from occurring in a subject that may be at risk for the disease, but is not yet diagnosed as having the disease. Prevention (and effective dose to prevent) can be demonstrated in population studies. For example, an amount effective to prevent a given disease or condition is an amount effective to reduce the incidence in the treated population, compared to an untreated control population.

The phrase “alleviating a symptom of” can refer to ameliorating, reducing, or eliminating any condition or symptom associated with an allergic inflammatory disease. For example, symptoms of allergic inflammatory conditions can include tingling or itching in the mouth; hives, itching, or eczema; swelling of the lips, face, tongue and throat or other parts of the body; wheezing nasal congestion, or trouble breathing; abdominal pain, diarrhea, nausea or vomiting; and dizziness, lightheadedness, or fainting.

The patient to be treated can be a mammal, such as a human being. Treatment also encompasses any pharmaceutical use of the compositions herein, such as use for treating a disease as provided herein.

In embodiments, the composition can further comprise one or more “nutritional components”. The term “nutritional component” as used herein can refer to such as protein, a carbohydrate, vitamins, minerals and other beneficial nutrients including functional ingredients of the disclosure, that is, ingredients that can produce specific benefits to a person consuming the food. The carbohydrate can be, but is not limited to, glucose, sucrose, fructose, dextrose, tagatose, lactose, maltose, galactose, xylose, xylitol, dextrose, polydextrose, cyclodextrins, trehalose, raffinose, stachyose, fructooligosaccharide, maltodextrins, starches, pectins, gums, carrageenan, inulin, cellulose based compounds, sugar alcohols, sorbitol, mannitol, maltitol, xylitol, lactitol, isomalt, erythritol, pectins, gums, carrageenan, inulin, hydrogenated indigestible dextrins, hydrogenated starch hydrolysates, highly branched maltodextrins, starch and cellulose.

Commercially available sources of nutritional proteins, carbohydrates, and the like and their specifications are known, or can be ascertained easily, by those of ordinary skill in the art of processed food formulation.

The compositions described herein that can include nutritional components can be food preparations that can be, but are not limited to, “snack sized”, or “bite sized” compositions that is, smaller than what might normally be considered to be a food bar. For instance, the food bar can be indented or perforated to allow the consumer to break off smaller portions for eating, or the food “bar” can be small pieces, rather than a long, bar-shaped product. The smaller pieces can be individually coated or enrobed. They can be packaged individually or in groups.

The food can include solid material that is not ground to a homogeneous mass, such as, without limitation. The food can be coated or enrobed, such as, and without limitation, with chocolate, including dark, light, milk or white chocolate, carob, yogurt, other confections, nuts or grains. The coating can be a compounded confectionary coating or a non-confectionary (e.g., sugar free) coating. The coating can be smooth or can contain solid particles or pieces.

An allergy is when one's immune system reacts to a foreign substance, called an allergen. For example, the allergen can be eaten, inhaled into one's lungs, injected into a subject or touched. This allergic reaction could cause coughing, sneezing, itchy eyes, a runny nose and a scratchy throat. In severe cases, it can cause rashes, hives, low blood pressure, breathing trouble, asthma attacks and even death. There is no cure for allergies even though it is among the country's most common diseases.

Allergic reactions are treated using anti-allergy medications, such as Brompheniramine (Dimetane), Cetirizine (Zyrtec), Chlorpheniramine (Chlor-Trimeton), Clemastine (Tavist), Diphenhydramine (Benadryl), and Fexofenadine (Allegra). Many such anti-allergy medications are associated with unwanted side effects such as drowsiness, dizziness, dry mouth/nose/throat, headache, upset stomach, constipation, or trouble sleeping. Pharmaceutical compositions provided herein comprising VLC-PUFAs do not cause such unwanted side effects, and therefore are an improvement over the known anti-allergy medications.

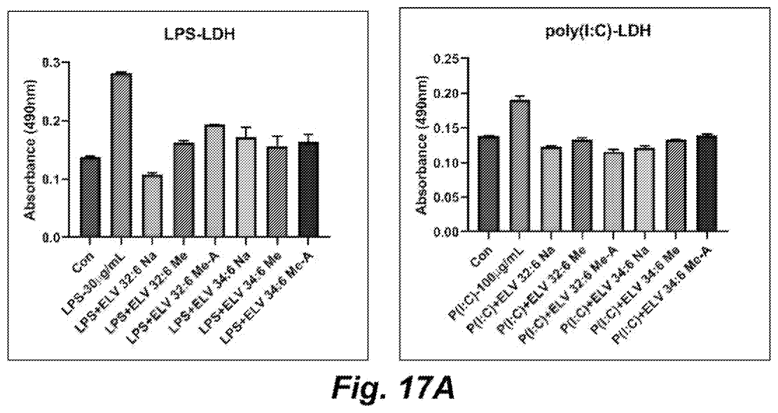

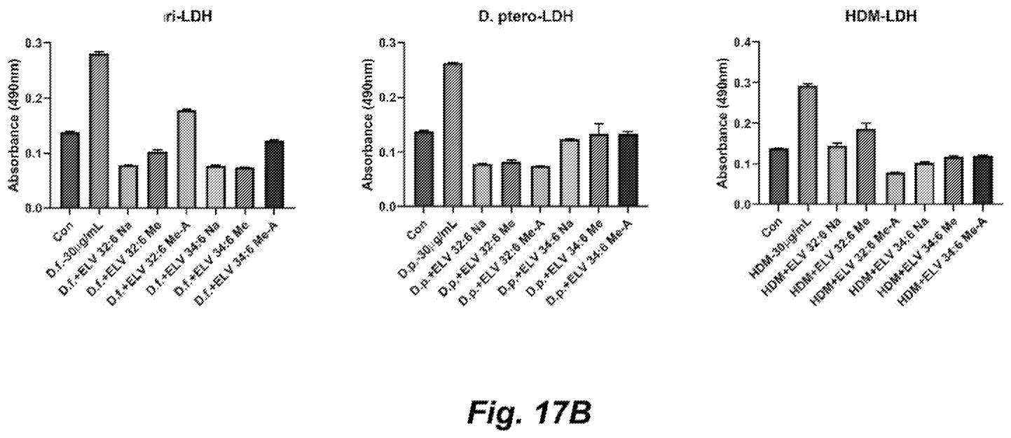

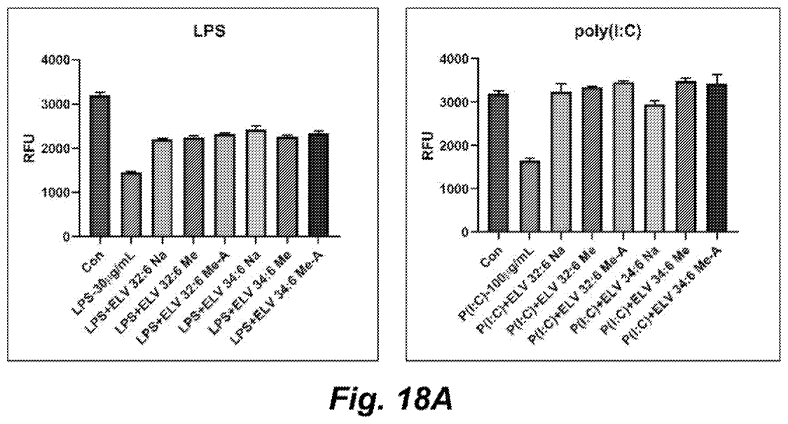

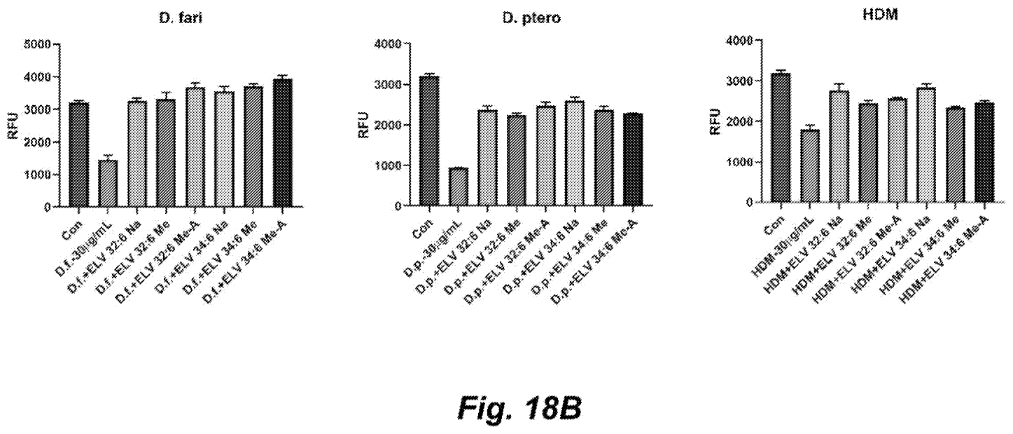

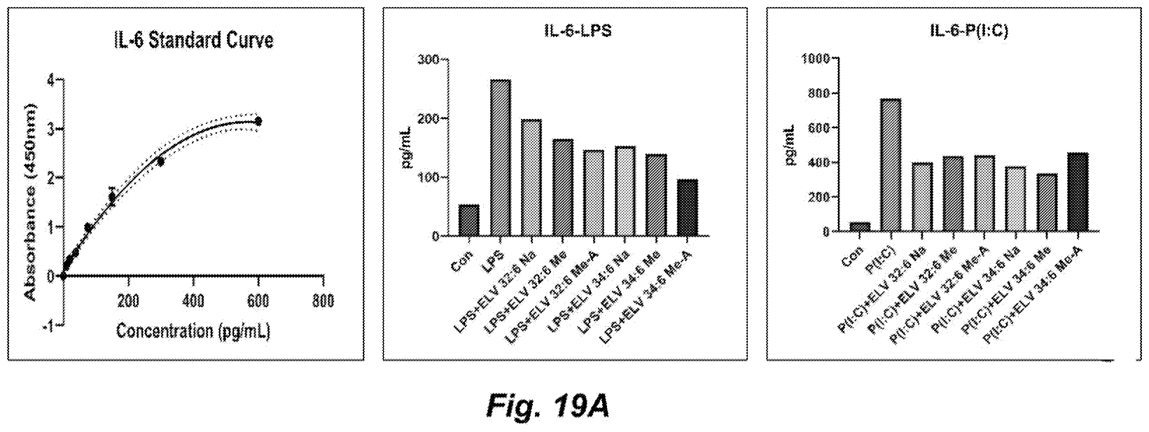

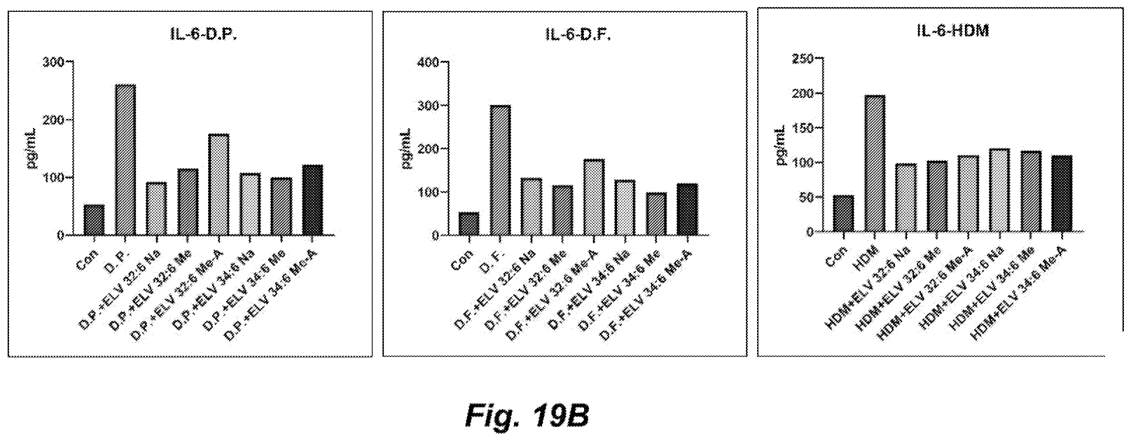

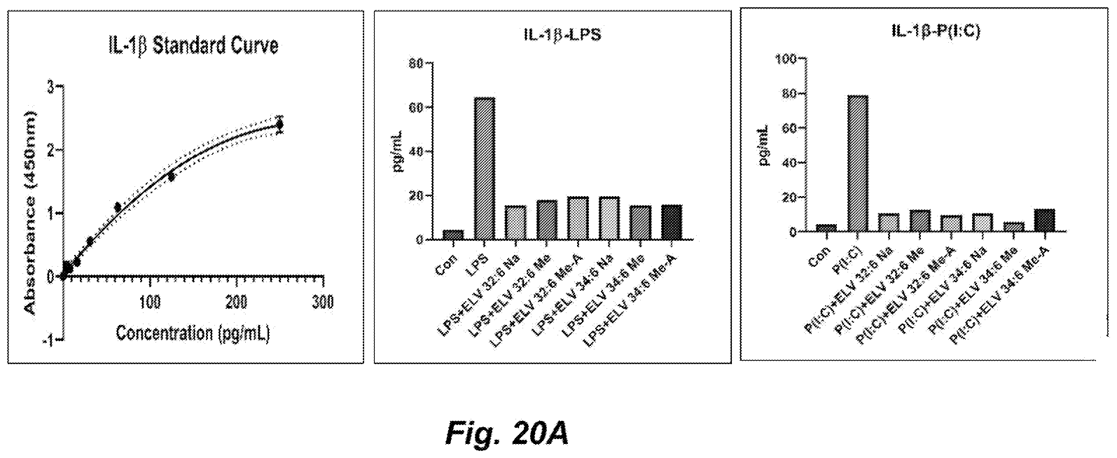

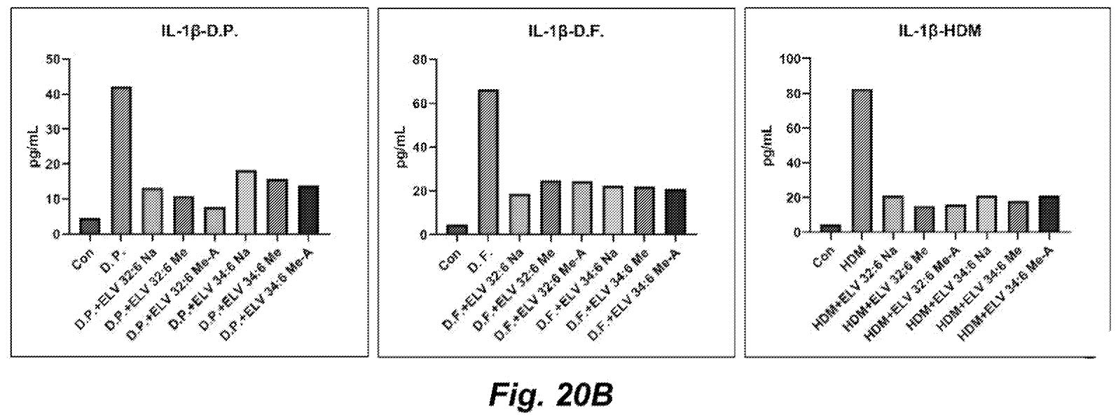

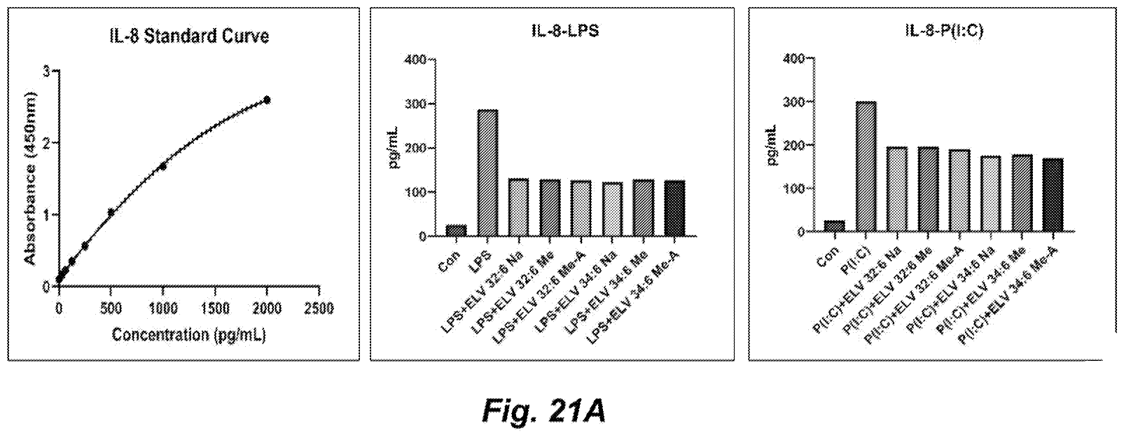

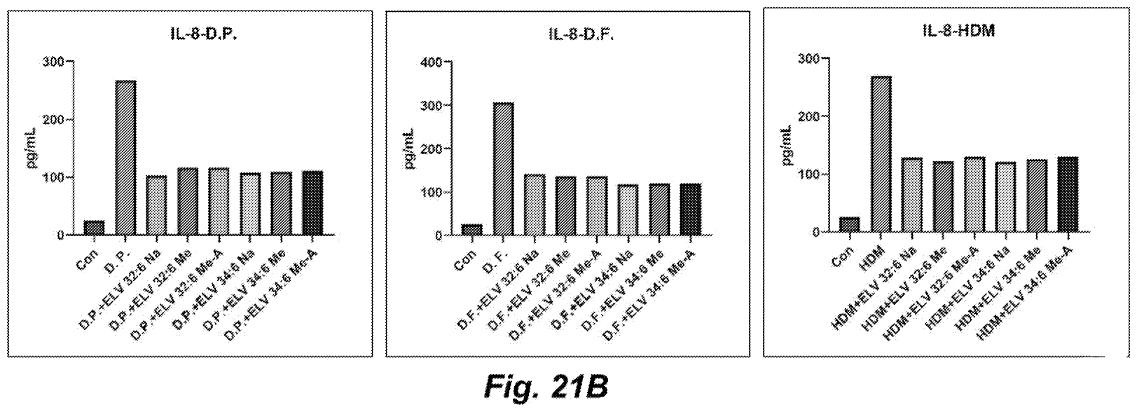

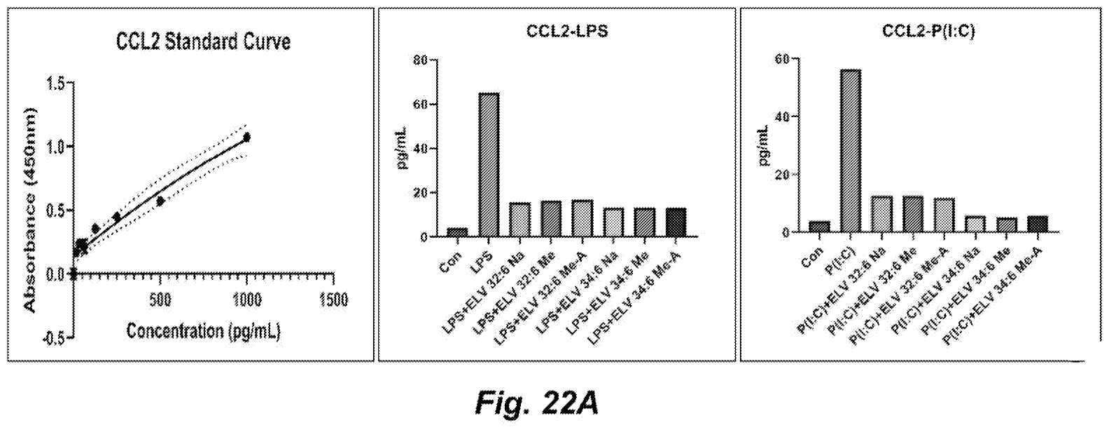

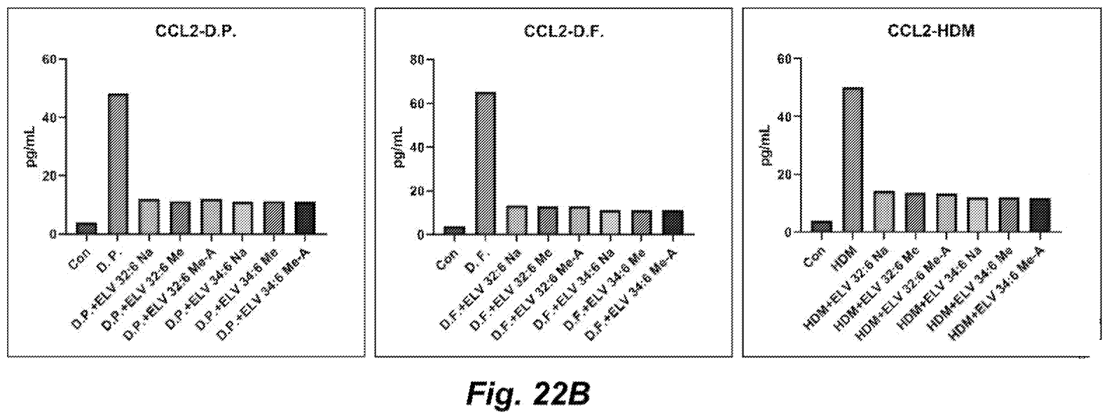

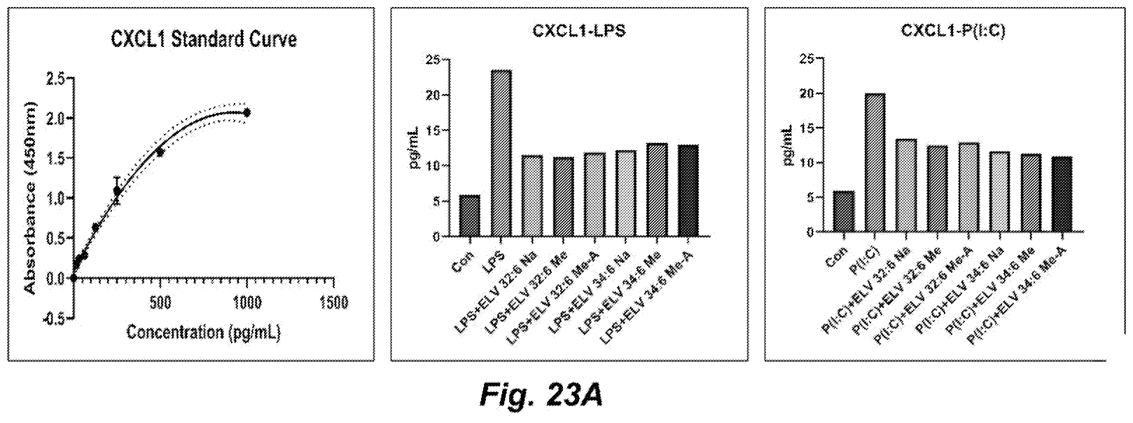

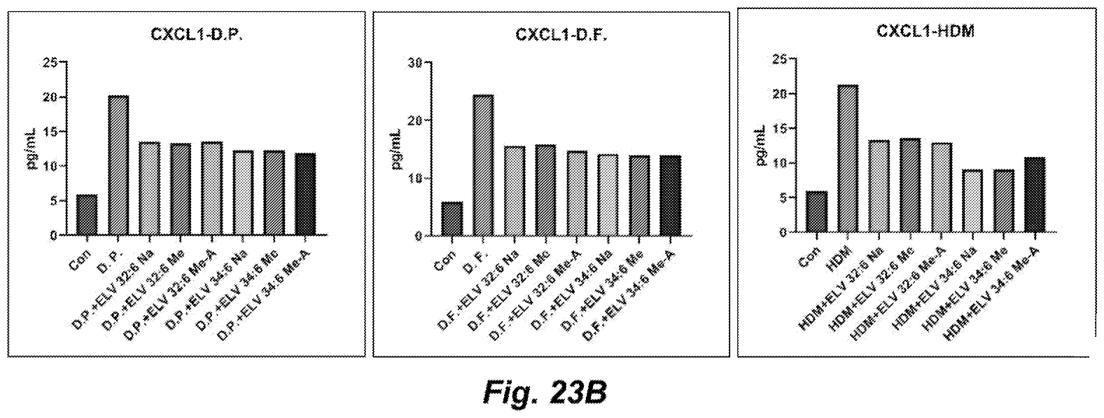

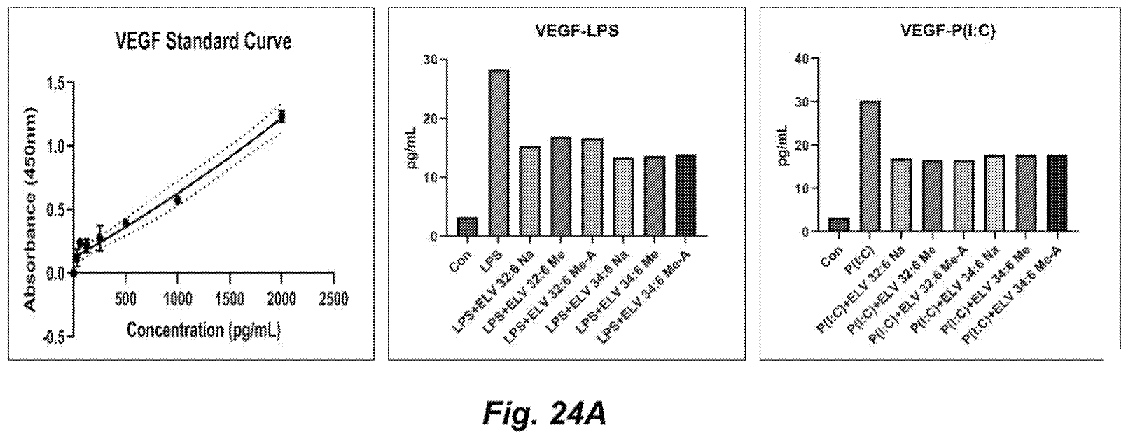

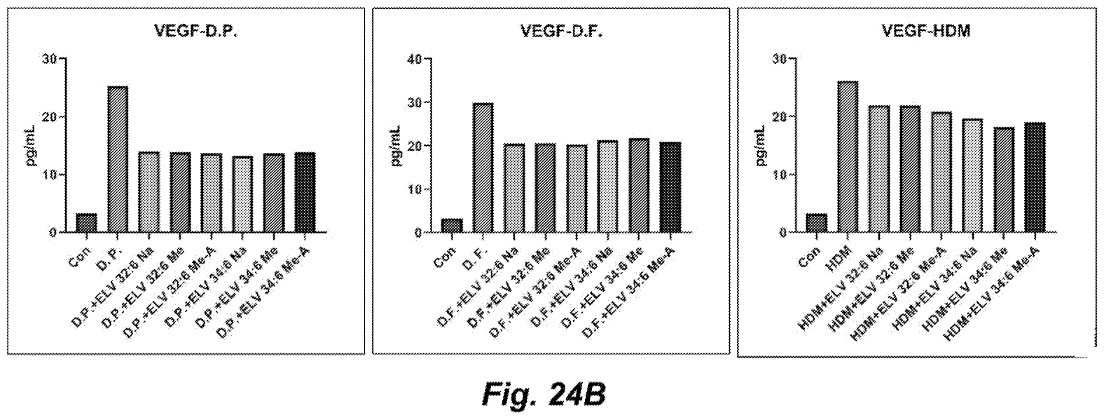

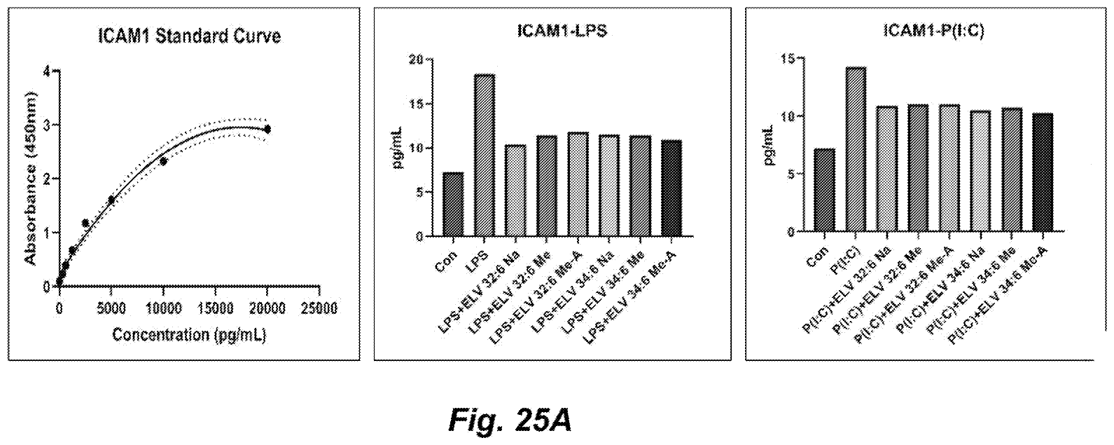

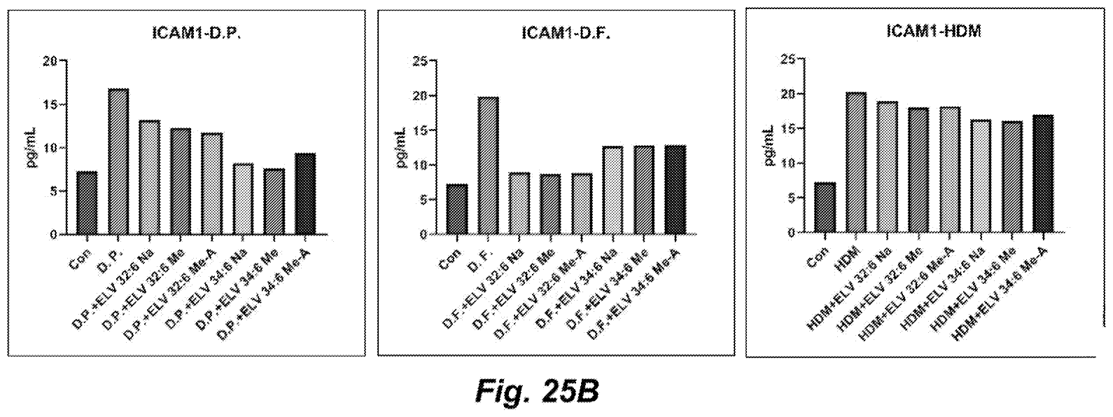

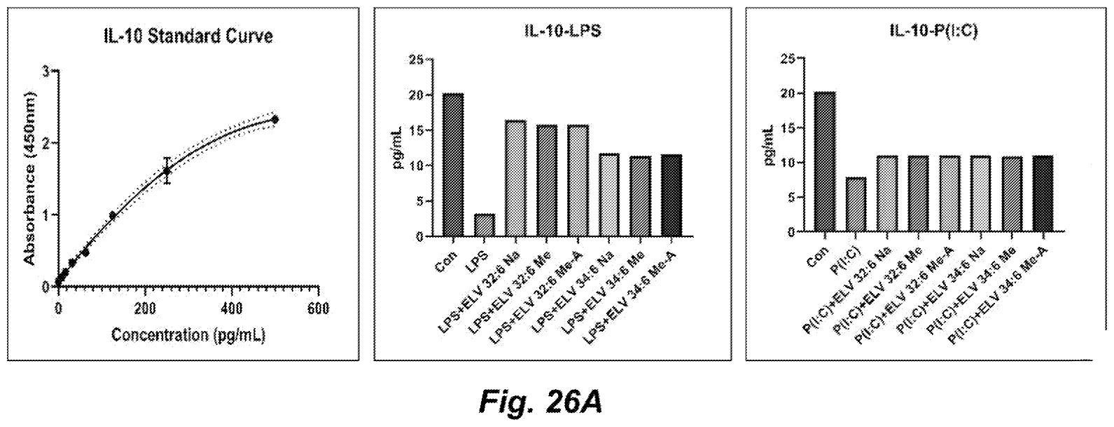

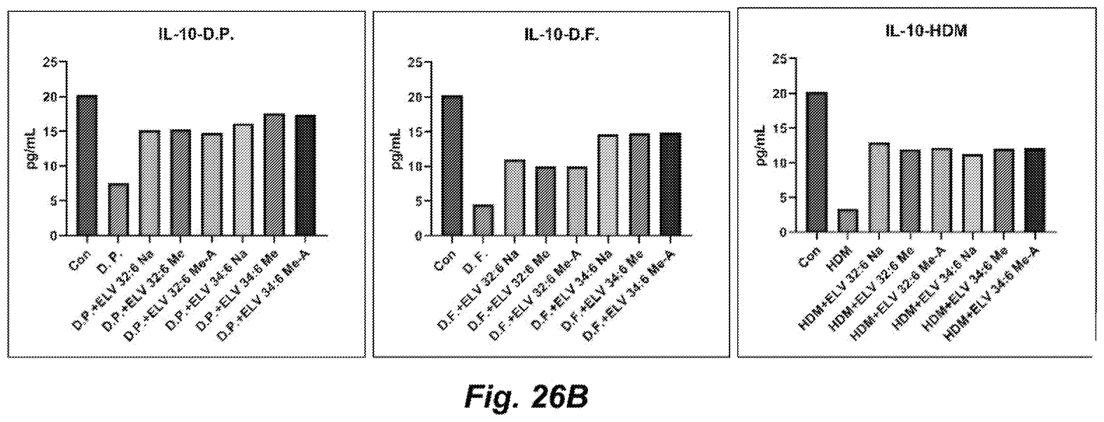

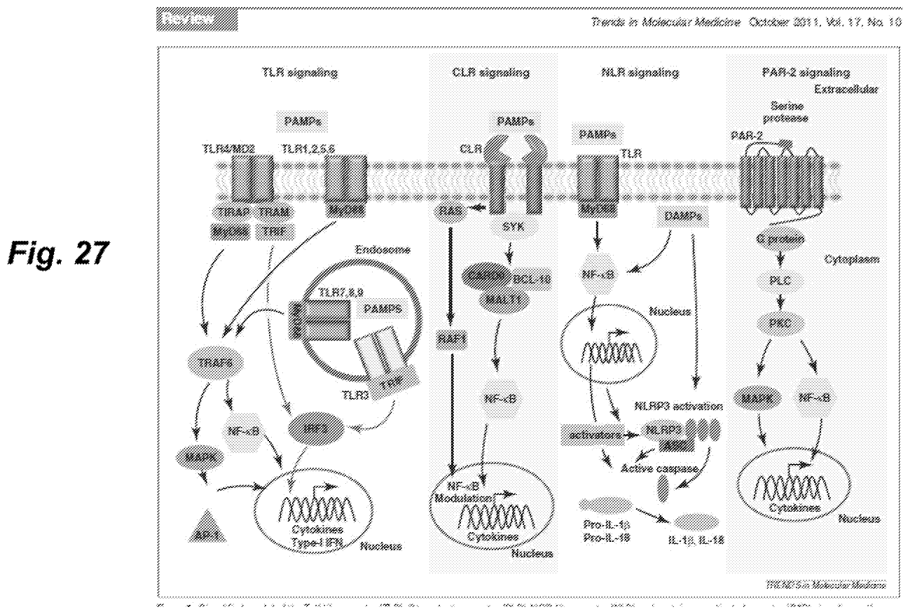

Aspects of this invention are drawn to compositions and methods for alleviating a symptom of, preventing, or treating allergic inflammatory diseases. Referring to the Examples included herein, results from a cytotoxicity assay (LDH) show that upon addition of the stressors to a culture of nasal epithelial cells, there is pronounced increase in the formation of red formazan indicating cytotoxicity, which are reduced by the addition of ELVs. (FIG. 17A and FIG. 17B). Further, cell viability assay using PrestoBlue HS reagent also shows more resorufin production in control cells as compared to cells challenged with the different stressors, and that addition of ELVs increases cell viability and gives protection to the HNEpC (FIG. 18A and FIG. 18B). Still further, when HNEpC were challenged with the different stressors, there is a pronounced production of pro-inflammatory cytokines and chemokines compared to controls and a pronounced decrease in the release of anti-inflammatory cytokines. This increased production of pro-inflammatory cytokines and chemokines are abrogated by the addition of ELVs at a concentration of 500 nM, 30 min post challenge with the respective stressor (FIG. 19A and FIG. 19B), while the decrease in production of anti-inflammatory cytokines is reversed (FIG. 26A and FIG. 26B).

Aspects of the invention are also drawn to compositions and methods for alleviating a symptom of, preventing, or treating a disease associated with cellular senescence, ferroptosis, or both. For example, the disease is a disease associated with Ap. Non-limiting examples of such diseases include age-related macular degeneration or alzheimer's disease.

Aspects of the invention are still further drawn to compositions and methods for alleviating a symptom of, preventing, or treating a metabolic disorder. Non-limiting examples of metabolic disorders include diabetes and obesity.

Aspects of the invention are further drawn to compositions and methods for delaying and/or preventing aging of a subject, and/or sustaining healthy cellular and molecular responses that counteract age-related dysfunctions.

“Aging” can refer to any time-dependent change to which biological entities, from molecules to ecosystems, are subject, though the mechanisms and consequences to function may be vastly different (Medawar, “An Unsolved Problem of Biology”, H. K. Lewis, London, (1952). Additionally or alternatively, aging can refer to the process of growing old or sentencing. Aging is associated with phenotypes in a cell or organism. For example, aging of an organism refers to the deterioration of the organism associated with changes in structures and functions (e.g., phenotype) that are characteristic of the organism. Such characteristics can include, for example, hair loss, graying of hair, osteoporosis, cataracts, atherosclerosis, loss of skin elasticity and a propensity for certain cancers. Thus, the compositions and methods described herein can be used to delay and/or prevent aging of a subject. In embodiments, aging is not skin aging.

The disclosure encompasses embodiments of compounds, compositions, and methods for the alleviation of a symptom of, the prevention of, and treatment of diseases.

This is based on new findings described herein regarding surprising biological activities of certain very long chain-polyunsaturated fatty acids (VLC-PUFA) and their related hydroxylated derivatives. For example, such biological activities include anti-inflammatory role of certain VLC-PUFAs, among others.

Long chain polyunsaturated fatty acids (LC-PUFAs) can include the omega-3 (n-3) and omega-6 (n6) polyunsaturated fatty acids containing 18-22 carbons including: arachidonic acid (ARA, C20:4n6, i.e. 20 carbons, 4 double bonds, omega-6), eicosapentaenoic acid (EPA, C20:5n-3, 20 carbons, 5 double bonds, omega-3), docosapentaenoic acid (DPA, C22:5n-3, 22 carbons, 5 double bonds, omega-3), and docosahexaenoic acid (DHA, C22:6n-3, 22 carbons, 6 double bonds, omega-3). LC-PUFAs are converted via lipoxygenase-type enzymes to biologically active hydroxylated PUFA derivatives that function as biologically active lipid mediators that play important roles in inflammation and related conditions. Most important among these are hydroxylated derivatives generated in certain inflammation-related cells via the action of a lipoxygenase (LO or LOX) enzyme (e.g. 15-LO, 12-LO), and result in the formation of mono-, di- or tri-hydroxylated PUFA derivatives with potent actions including anti-inflammatory, pro-resolving, neuroprotective or tissue-protective actions, among others. For example, neuroprotectin D1 (NPD1), a dihydroxy derivative from DHA formed in cells via the enzymatic action of 15-lipoxygenase (15-LO) was shown to have a defined R/S and Z/E stereochemical structure (10R,17S-dihydroxy-docosa-4Z,7Z,11E, 13E,15Z,19Z-hexaenoic acid) and a unique biological profile that can includes stereoselective potent anti-inflammatory, homeostasis-restoring, pro-resolving, bioactivity. NPD1 has been shown to modulate neuroinflammatory signaling and proteostasis, and to promote nerve regeneration, neuroprotection, and cell survival.

Other important types of fatty acids are the n-3 and n6 very-long-chain polyunsaturated fatty acids (n-3 VLC-PUFA, n6 VLC-PUFA) that are produced in cells containing elongase enzymes that elongate n-3 and n6 LC-PUFA to n-3 and n6 VLC-PUFA containing from 24 to 42 carbons (C24-C42). The most important among these seem to be VLC-PUFA with 28-38 carbons (C28-C38). Representative types of VLC-PUFA can include C32:6n-3 (32 carbons, 6 double bonds, omega-3), C34:6n-3, C32:5n-3, and C34:5n-3. These VLC-PUFA are biogenically-derived through the action of elongase enzymes, such as ELOVL4 (ELOngation of Very Long chain fatty acids 4). VLC-PUFA are also acylated in complex lipids including sphingolipids and phospholipids such as in certain molecular species of phosphatidyl choline.

The biosynthetic role of ELOVL4 and the biological functions of VLC-PUFA have been the subject of a number of recent investigations. See, for example, PCT/US2016/017112, PCT/US2018/023082, and U.S. Ser. No. 16/576,456, each of which are can included herein by reference in their entireties. These VLC-PUFA display functions in membrane organization, and their significance to health is increasingly recognized.

The compounds, compositions and methods encompassed by the embodiments of the disclosure involve the use of n-3 VLC-PUFA for alleviating a symptom of, preventing, or treating a disease.

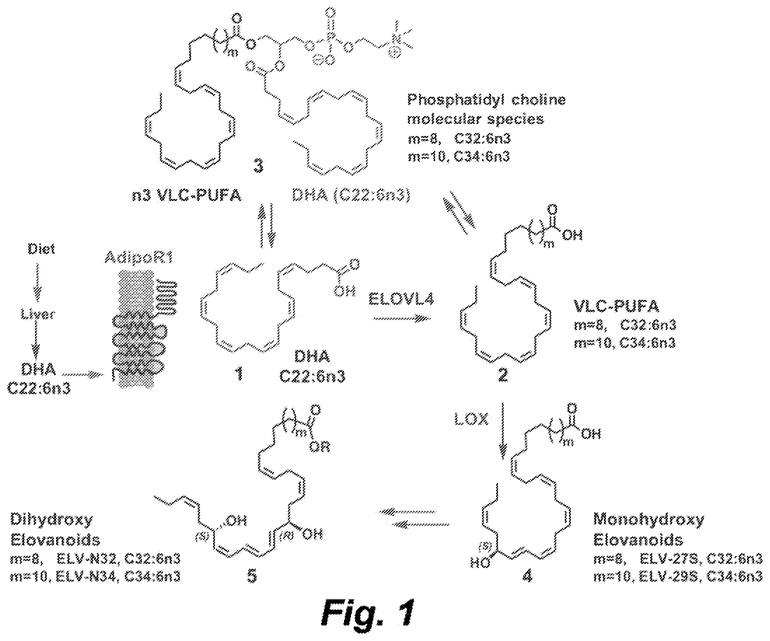

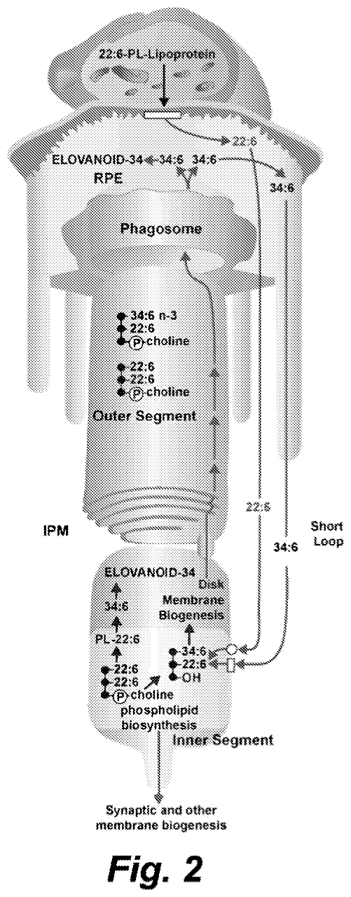

Biosynthetic pathways for n-3 VLC-PUFA: The biosynthesis of n-3 VLC-PUFA begins from lower-carbon PUFA that contain only an even number of carbons in their carbon chain, such as docosahexaenoic acid (DHA) that contains 22 carbons and 6 alternating C═C bonds (C22:6n-3), and docosapentaenoic acid (DPA) that contains 22 carbons and 5 alternating C═C bonds (C22:5n-3). The biosynthesis of n-3 VLC-PUFA requires the availability of DHA or other shorter-chain PUFA as substrates, and the presence and actions of certain elongase enzymes, e.g. ELOVL4. As summarized in FIGS. 1 and 2, these 22-carbon omega-3 long-chain fatty acids (n-3 LC-PUFA) are substrates to elongase enzymes, such as ELOVL4, which adds a 2-carbon CH₂CH₂ group at a time to the carboxylic end, forming n-3 VLC-PUFA that contain carbon chains with at least 24 carbons of up to at least 42 carbons.

Docosahexaenoic acid (DHA, C22:6n-3, 1 is incorporated at the 2-position of phosphatidyl choline molecular species (3) and is converted by elongase enzymes to longer-chain n-3 VLC-PUFA. Elongation by the elongase enzyme ELOVL4 (ELOngation of Very Long chain fatty acids-4) leads to the formation of very long chain omega-3 polyunsaturated fatty acids (n-3 VLC-PUFA, 2, including C32:6n-3 and C34:6n-3 that are then incorporated at the 1-position of phosphatidyl choline molecular species, 3. The presence of DHA at the 2-position and n-3 VLC-PUFA at the 1-position can offer redundant, complementary, and synergistic cytoprotective and neuroprotective actions that amplify the survival of neurons and other key cell types when challenged with pathological conditions.

Lipoxygenation of n-3-VLC-PUFA, 3 leads to the formation of enzymatically-hydroxylated derivatives of n-3-VLC-PUFA, termed elovanoids, which can include monohydroxy compounds (e.g. ELV-27S and ELV-29S, 4, and dihydroxy derivatives, e.g. ELV-N-32 and ELV-N-34, 5. Elovanoid ELV-N-32 is the 20R,27S-dihydroxy 32:6 derivative (32-carbon, 6 double bond elovanoid with a neuroprotectin-like 20(R),27(S)-dihydroxy pattern). Elovanoid ELV-N-34 is the 22R,29S-dihydroxy 34:6 derivative (34-carbon, 6 double bond elovanoid with a 22(R),29(S)-dihydroxy pattern).

FIG. 2 illustrates the delivery of docosahexaenoic acid (DHA, C22:6n-3) to photoreceptors, photoreceptor outer segment membrane renewal, and the synthesis of elovanoids. DHA or precursor C18:3n-3 are obtained by diet, as is DHA itself (FIG. 1). The systemic circulation (mainly the portal system) brings them to the liver. Once within the liver, hepatocytes incorporate DHA into DHA-phospholipid (DHA-PL), which is then transported as lipoproteins to the choriocapillaries, neurovascular unit, and to the capillaries of other tissues.

DHA crosses Bruch's membrane from the choriocapillaries (FIG. 2) and is taken up by the retinal pigment epithelium (RPE) cells lining the back of the retina to be sent to the inner segment of photoreceptors. This targeted delivery route from the liver to the retina is referred to as the DHA long loop.

DHA then passes through the interphotoreceptor matrix (IPM) and to the photoreceptor inner segment, where it is incorporated into phospholipids for the photoreceptor outer segments, cell membrane and organelles. The majority is used in disk membrane biogenesis (outer segments). As new DHA-rich disks are synthesized at the base of the photoreceptor outer segment, older disks are pushed apically toward the RPE cells. Photoreceptor tips are phagocytized by the RPE cells each day, removing the oldest disks. The resulting phagosomes are degraded within the RPE cells, and DHA is recycled back to photoreceptor inner segments for new disk membrane biogenesis. This local recycling is referred to as the 22:6 short loop.

Elovanoids are formed from omega-3 very long chain polyunsaturated fatty acids (n-3 VLC-PUFA) biosynthesized by ELOVL4 (ELOngation of Very Long chain fatty acids-4) in the photoreceptor inner segments. Thus, a phosphatidylcholine molecular species in the inner segment that contains VLC Omega-3 FA at C1 (C34:6n-3 is depicted) and DHA (C22:6n-3) at C2 is used for photoreceptor membrane biogenesis. This phospholipid has been found tightly associated to rhodopsin. Once the discs are phagocytized in RPE cells as a daily physiological process, upon homeostatic disturbances, a phospholipase A1 (PLA1) cleaves the acyl chain at sn-1, releasing C34:6n-3 and leads to the formation of elovanoids (e.g. elovanoid-34, ELV-N-34). VLC omega-3 fatty acids that are not used for elovanoid synthesis are recycled through the short loop.

Therefore, for biosynthetic reasons, the naturally occurring and biogenetically derived n-3 VLC-PUFA contain only an even number of carbons, ranging from at least 24 carbons to at least 42 carbons (i.e. 24, 26, 28, 30, 32, 34, 36, 38, 40, 42 carbons). Thus, n-3 VLC-PUFA that contain only an odd number of carbons ranging from at least 23 of up to at least 41 carbons (i.e. 23, 25, 27, 29, 31, 33, 35, 37, 39, 41 carbons) are not naturally occurring, but they can be synthesized and manufactured using synthetic chemical methods and strategies.

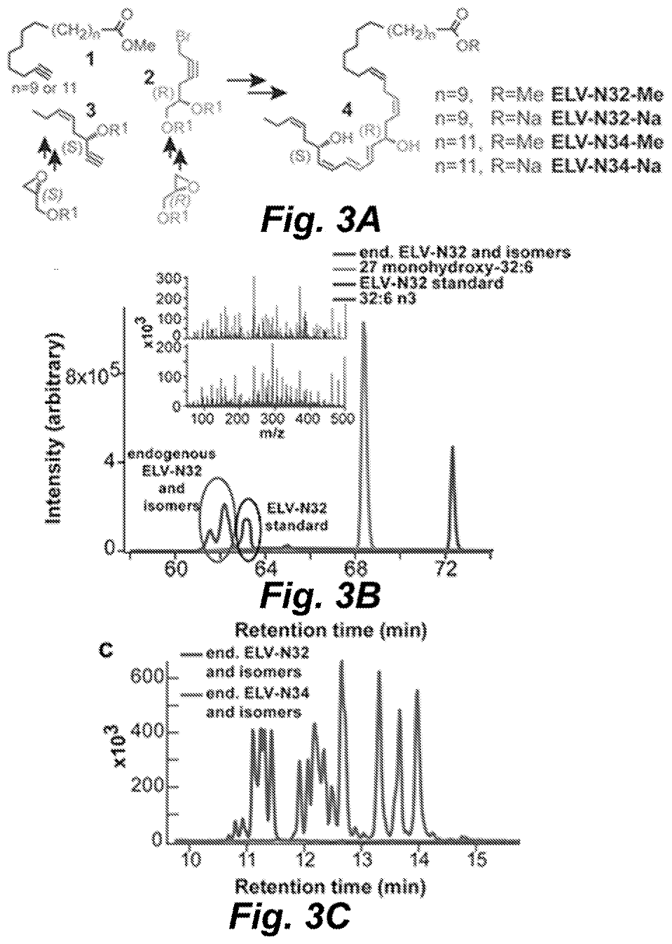

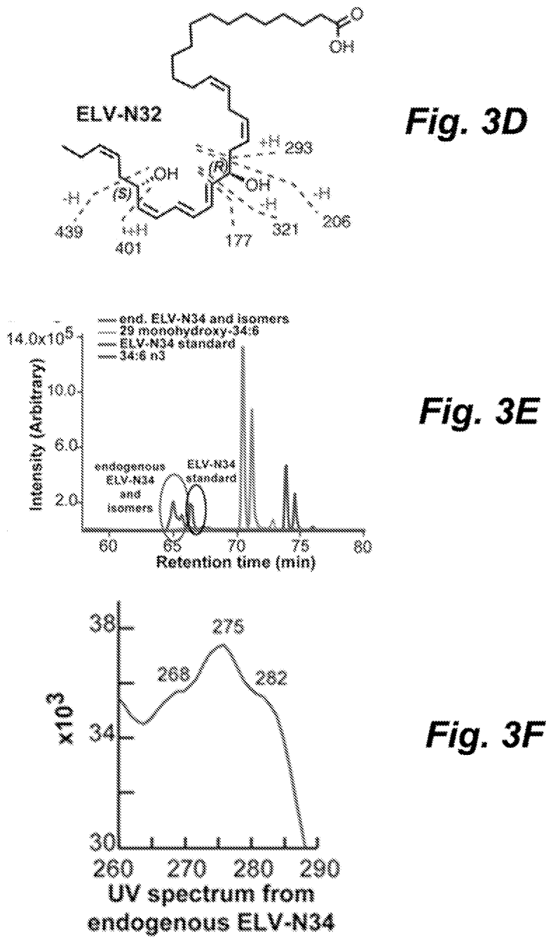

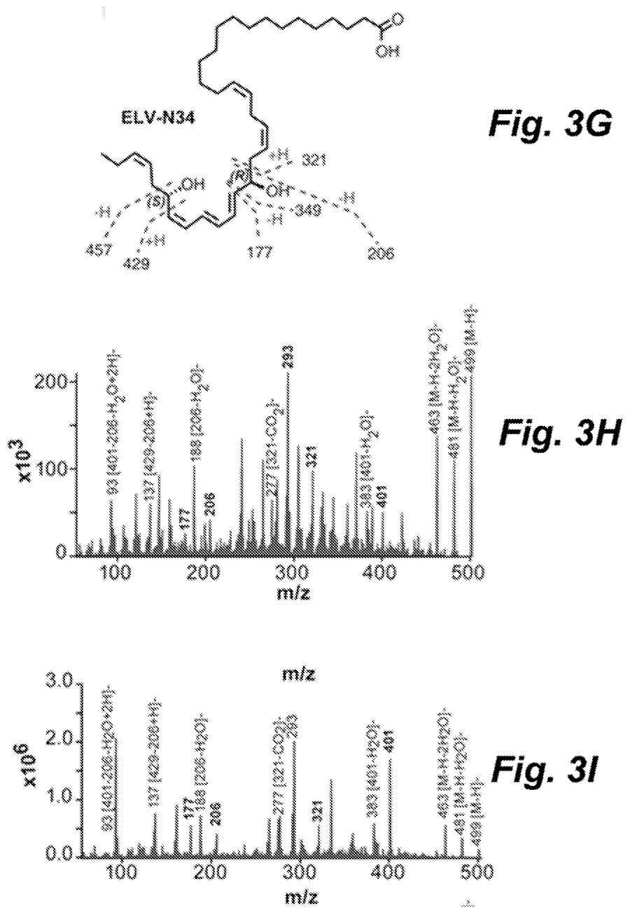

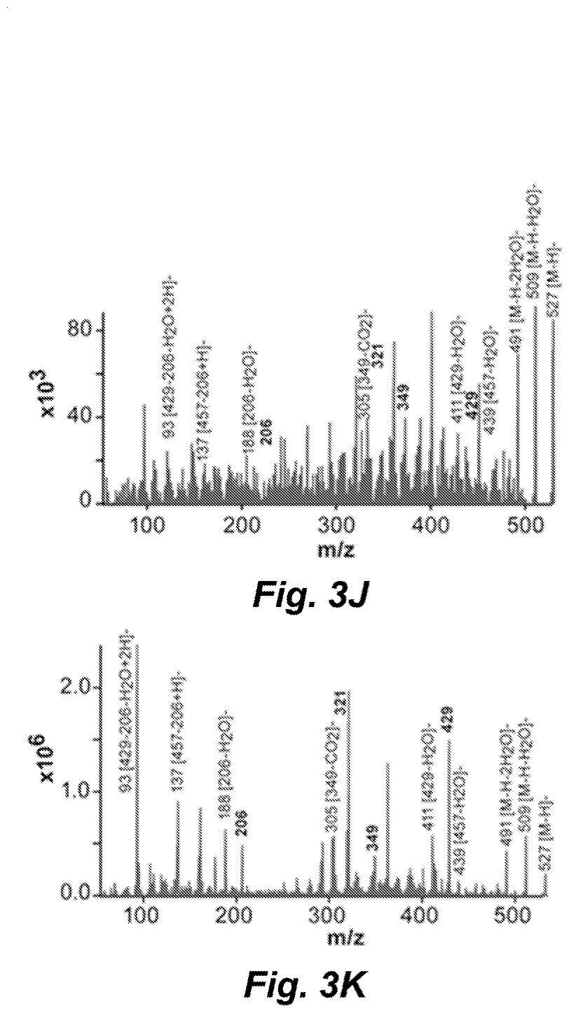

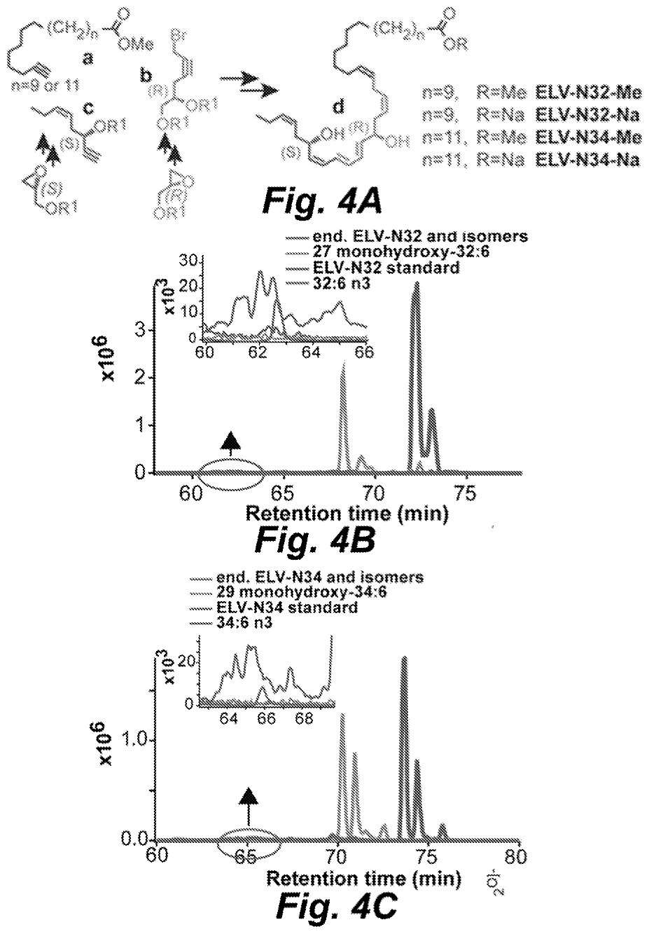

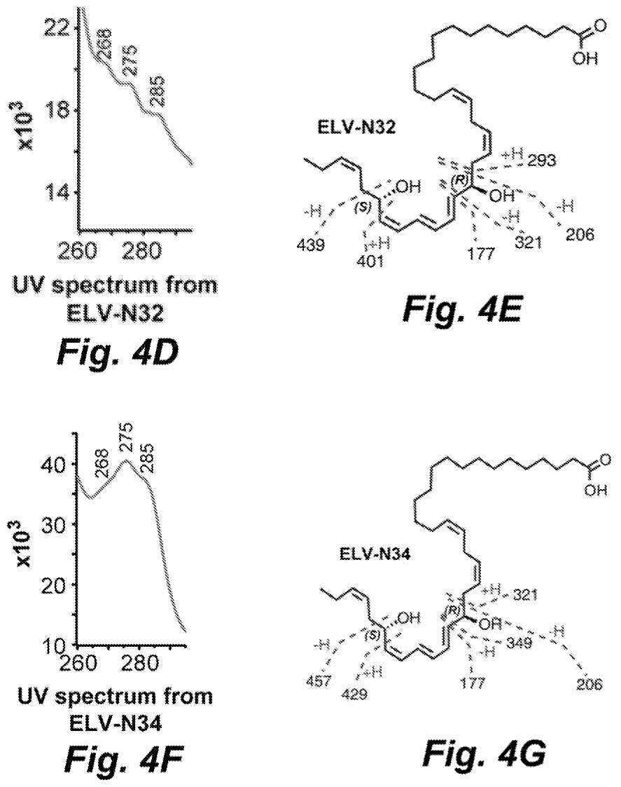

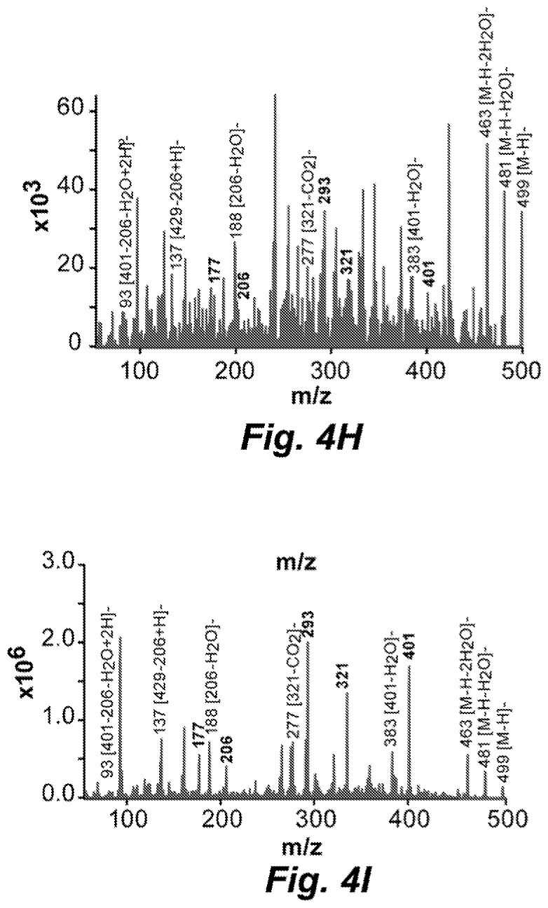

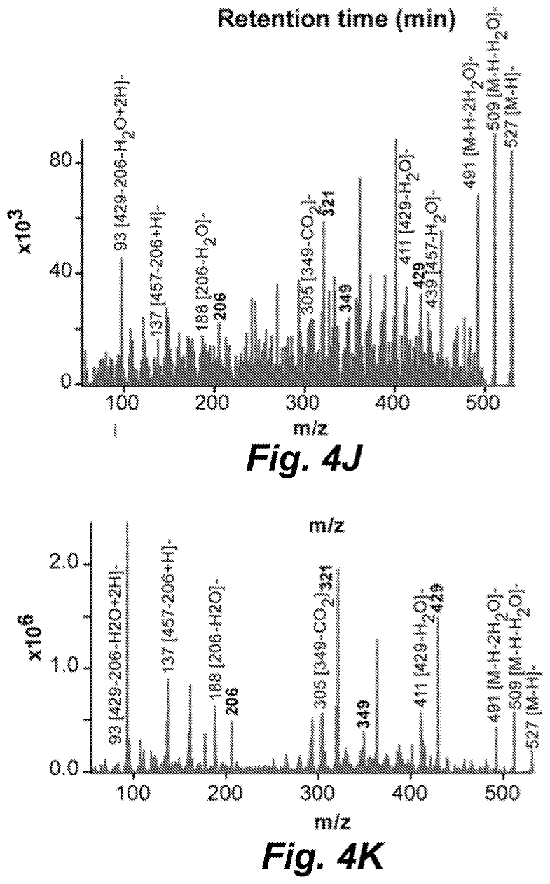

Stereocontrolled total synthesis and structural characterization of elovanoids ELV-N-32 and ELV-N-34 in the retina and the brain: As summarized in FIG. 3 and FIG. 4, ELV-N-32 (27S- and ELV-N-34 were synthesized from three key intermediates (1, 2, and 3), each of which was prepared in stereochemically-pure form. The stereochemistry of intermediates 2 and 3 was pre-defined by using enantiomerically pure epoxide starting materials. Iterative couplings of intermediates 1, 2, and 3, led to ELV-N-32 and ELV-N-34 (4) that were isolated as the methyl esters (Me) or sodium salts (Na). The synthetic materials ELV-N-32 and ELV-N-34 were matched with endogenous elovanoids with the same number of carbons on their carbon chain, obtained from cultured human retinal pigment epithelial cells (RPE) (FIG. 3), and neuronal cell cultures (FIG. 4).

Experimental detection and characterization of the Elovanoids: Experimental evidence documents the biosynthetic formation of the elovanoids, which are mono-hydroxy and di-hydroxy n-3 VLC-PUFA derivatives with molecular structures that are analogous to DHA-derived 17-hydroxy-DHA and the di-hydroxy compound NPD1 (10R,17S-dihydroxy-docosa-4Z,7Z,11E,13E,15Z,19Z-hexaenoic acid). The elovanoids are enzymatically generated hydroxylated derivatives of 32-carbon (ELV-N-32) and 34-carbon (ELV-N-34) n-3 VLC-PUFA in that were first identified in cultures of primary human retinal pigment epithelial cells (RPE) (FIG. 3A-3K) and in neuronal cell cultures (FIG. 4A-4K).

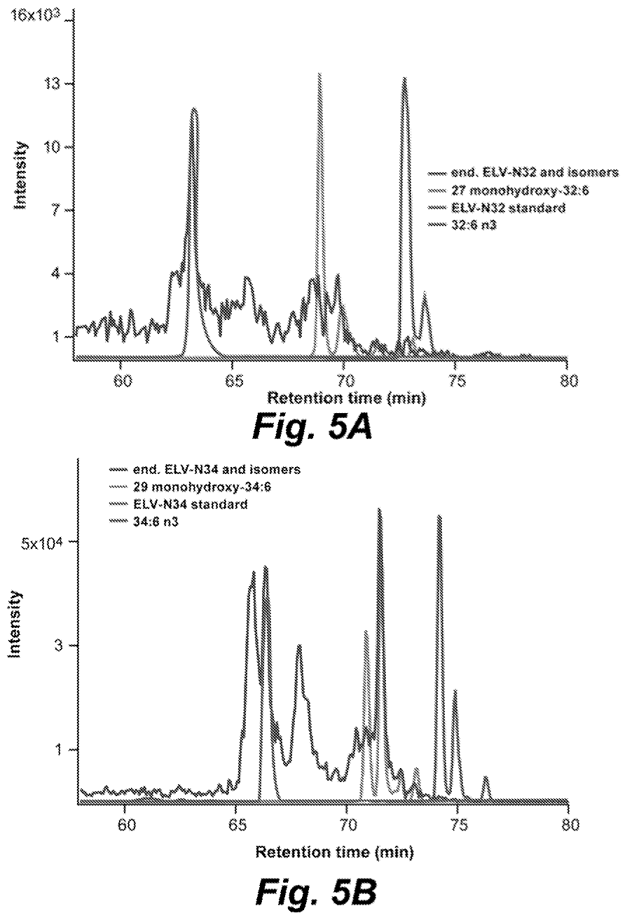

The disclosure provides compounds having carbon chains related to n-3 VLC-PUFA that in addition to having 6 or 5 C═C bonds, they also contain one, two or more hydroxyl groups. Considering that compounds of this type can be responsible for the protective and neuroprotective actions of n-3 VLC-PUFA, we sought to identify their existence in human retinal pigment epithelial cells in culture, with added 32:6n-3 and 34:6n-3 VLC-PUFA fatty acids. Our results indicated mono-hydroxy- and di-hydroxy elovanoid derivatives from both 32:6n-3 and 34:6n-3 VLC-PUFA fatty acids. The structures of these elovanoids (ELV-N-32, ELV-N-34) were compared with standards prepared in stereochemical pure form via stereocontrolled total organic synthesis (FIG. 5A and FIG. 5B).

Beneficial Roles of n-3 VLC-PUFA as Therapeutics: The data described herein provided support for the beneficial use of the provided n-3 VLC-PUFA and/or elovanoid compounds, as therapeutics for the prevention and treatment of diseases.

The phrase “allergic disease” or “allergic inflammatory disease” can refer to a disease with an allergic reaction. More specifically, an “allergic disease” can be characterized by a strong correlation between exposure to an allergen and the development of pathological changes, and that the pathological changes have an immune mechanism (i.e., an allergic inflammatory disease). For example, the immune mechanism can refer to leukocytes exhibit an immune response to allergen stimulation. For example, the immune response can refer to increased production of pro-inflammatory cytokines and chemokines. Examples of allergens can include mite antigens and pollen antigens. Representative allergic diseases can include bronchial asthma, allergic rhinitis, atopic dermatitis or allergic dermatitis, allergic conjunctivitis, and pollen and insect allergies. Allergic predisposition is a genetic factor that can be inherited by a parent child of an allergic predisposition. Familial allergic diseases are also called atopic diseases, and the causative genetic factor is atopic constitution. “Atopic dermatitis” is a term for atopic diseases, such as diseases associated with skin inflammation. Non-limiting examples can include allergic conditions selected from the group consisting of eczema, allergic rhinitis, hay fever, urticaria, and food allergies. Allergic conditions can include eczema, allergic rhinitis or nasal cold, hay fever, bronchial asthma, urticaria (urticaria (hives), and food allergies, as well as other atopic conditions.

“Asthma” can refer to a disorder of the respiratory system characterized by inflammation, airway narrowing, and increased airway responsiveness to inhaled substances or allergens. Asthma is often but not exclusively associated with atopic or allergic symptoms. Symptoms of asthma are widely recognized to can include dyspnea, cough, and wheezing; while all three symptoms coexist, their coexistence is not required to make a diagnosis of asthma.

The term “allergic asthma” can refer to the allergic aspect of asthma among asthma symptoms and can includes, for example, mixed type asthma and atopic asthma. Allergic asthma is discriminated from non-allergic asthma such as aspirin asthma. A “therapeutic agent for asthma”, for example, can exert a therapeutic effect via the action on the allergic reaction of asthma. Furthermore, the therapeutic agent of asthma for example exerts an effect on chronic bronchitis or airway hypersensitivity. For example, the therapeutic agent of asthma has an effect on chronic bronchitis and airway hypersensitivity. The therapeutic agent of asthma exerts an effect on the late phase response, the delayed-type response, or the late phase and the delayed-type responses of the allergic reaction. For example, the therapeutic agent of asthma exerts an effect on the late phase response, the delayed-type response or the late phase and the delayed-type responses, in addition to the immediate-type response.

“Allergic rhinitis” can refer to any allergic reaction in the nasal mucosa, which can include hay fever (seasonal allergic rhinitis) and perennial rhinitis (non-seasonal allergic rhinitis). Symptoms of allergic rhinitis can be sneezing, rhinorrhea, nasal congestion, pruritis, eye itching, redness and tearing.

The phrase “skin disorder” can include the skin reactions of urticaria and angioedema. These skin disorders can be triggered by exposure to certain foods, medications, or virus infections. Urticarira (referred to as hives or welts) are red, itchy, raised areas of the skin of varying shapes and sizes. Urticarira are the result of release of histamine and other compounds from mast cells that cause serum to leak from local blood vessels and thereby cause swelling in the skin. Angioedema is a form of tissue swelling similar to urticaria, but involving deeper skin tissues (i.e., “deep hives”) and lasting longer than urticarial

The term “allergic dermatitis” can refer to dermatitis related with allergic reaction and can includes, for example, atopic dermatitis. Allergic dermatitis is discriminated from non-allergic dermatitis such as dermatitis due to injuries or wounds. As a “therapeutic agent for atopic dermatitis”, those that show therapeutic effect by acting on the allergic reaction occurring in atopic dermatitis are useful. Furthermore, the therapeutic agent of atopic dermatitis has an effect on the late phase response, the delayed-type response, or the late phase and delayed-type responses of the allergic reaction. For example, the therapeutic agent of atopic dermatitis has an effect on the late phase response, the delayed-type response or the late phase response and the delayed-type response, in addition to the immediate-type response.

The phrase “allergic conjunctivitis” can refer to an irritation by an allergen of the clear, thin membrane called the conjunctiva that covers the eyeball and the inside of the eyelids. Symptoms can include swollen eyes, itchy/burning eyes, tearing, and ocular redness. Some allergens can include pollen from trees, grass and ragweed, animal skin and secretions such as saliva, perfumes and cosmetics, skin medicines, air pollution and smoke.

An “allergen” can refer to a substance that can induce an allergic inflammatory disease in a subject. The list of allergens is enormous and can include pollens, insect venoms, animal dander, house dust mite, dust, fungal spores, latex, and drugs (e.g., penicillin). Examples of natural, animal and plant allergens can include proteins specific to the following genera: Canis (Canis familiaris); Dermatophagoides (e.g., Dermatophagoides farinae); Felis (Felis domesticus); Ambrosia (Ambrosia artemiisfolia); Lolium (e.g., Lolium perenne or Lolium multiflorum); Cryptomeria (Cryptomeria japonica); Alternaria (Alternaria alternata); Alder; Alnus (Alnus gultinosa); Betula (Betula verrucosa); Quercus (Quercus alba); Olea (Olea europa); Artemisia (Artemisia vulgaris); Plantago (e.g., Plantago lanceolata); Parietaria (e.g., Parietaria officinalis or Parietaria judaica); Blattella (e.g., Blattella germanica); Apis (e.g., Apis multiflorum); Cupressus (e.g., Cupressus sempervirens, Cupressus arizonica and Cupressus macrocarpa); Juniperus (e.g., Juniperus sabinoides, Juniperus virginiana, Juniperus communis and Juniperus ashei); Thuya (e.g., Thuya orientalis); Chamaecyparis (e.g., Chamaecyparis obtusa); Periplaneta (e.g., Periplaneta americana); Agropyron (e.g., Agropyron repens); Secale (e.g., Secale cereale); Triticum (e.g., Triticum aestivum); Dactylis (e.g., Dactylis glomerata); Festuca (e.g., Festuca elatior); Poa (e.g., Poa pratensis or Poa compressa); Avena (e.g., Avena sativa); Holcus (e.g., Holcus lanatus); Anthoxanthum (e.g., Anthoxanthum odoratum); Arrhenatherum (e.g., Arrhenatherum elatius); Agrostis (e.g., Agrostis alba); Phleum (e.g., Phleum pratense); Phalaris (e.g., Phalaris arundinacea); Paspalum (e.g., Paspalum notatum); Sorghum (e.g., Sorghum halepensis); and Bromus (e.g., Bromus inermis). Allergens also can include peptides and polypeptides such as are used in experimental animal models of allergy and asthma, including ovalbumin (OVA) and Schistosoma mansoni egg antigen.

“Metabolic disorder” can refer to a disorder or disease that results in disruption of the normal physiological state of homeostasis due to changes in metabolism (anabolism and/or catabolism). Metabolic changes fail to degrade (catabolize) the substance to be degraded (eg, phenylalanine), resulting in increased levels of the substance and/or intermediate substance, or some essential substances (eg, insulin) cannot be produced (anabolic).

“Metabolic syndrome” can refer to the concept of a grouping of metabolic risk factors that gather in a single individual and lead to a high risk of developing diabetes and/or cardiovascular disease. The main features of metabolic syndrome include insulin resistance, hypertension (hypertension), cholesterol abnormalities, dyslipidemia, triglyceride abnormalities, increased risk for coagulation and especially in the abdomen and overweight or obesity. Metabolic syndrome is also known as Syndrome X, Insulin Resistance Syndrome, Obesity Syndrome, Abnormal Metabolic Syndrome and Reaven's Syndrome. The interrelationship of the various risk factors of metabolic syndrome is shown in FIG. The presence of three or more risk factors in a single individual is indicative of metabolic syndrome. The American Heart Association states that metabolic syndrome is diagnosed by the presence of three or more of the following factors: (1) Increased waist circumference (male, 40 inches (102 cm) or greater; female, 35 inches (88 cm or more), (2) triglyceride elevation (150 mg/dL or more), (3) high density lipid or HDL reduction (male, less than 40 mg/dL; female, less than 50 mg/dL); (4) Increased blood pressure (130/85 mmHg or higher); and (5) increased fasting blood glucose (100 mg/dL or higher).

“Metabolic syndrome related metabolic disorders” can refer to metabolic syndrome and obesity, insulin resistance, type 2 diabetes, atherosclerosis and cardiomyopathy.

“Diabetes” can refer to a group of metabolic disorders characterized by hyperglycemia (glucose) levels resulting from deficiencies in insulin secretion or action or both.

“Type 2 diabetes” is one of the two major types of diabetes, at least in the early stages of the disease, because the R cells of the pancreas produce insulin, but the cells of the body are resistant to the action of insulin. Later in the disease, beta cells may stop producing insulin. Type 2 diabetes is also known as insulin resistant diabetes, non-insulin dependent diabetes and adult-onset diabetes.

“Prediabetes” can refer to one or more early diabetic conditions including impaired glucose utilization, abnormal or impaired fasting plasma glucose, impaired glucose tolerance, impaired insulin sensitivity and insulin resistance.

“Insulin resistance” means that a cell has become resistant to the action of insulin (a hormone that regulates glucose uptake into cells) or the amount of insulin produced maintains normal glucose levels. Cells can have a reduced ability to respond to the effects of insulin (ie, loss of sensitivity to insulin) in facilitating the transport of sugar glucose from blood to muscle and other tissues. Eventually, the pancreas produces much more insulin than normal, and the cells remain resistant. As long as enough insulin is produced to overcome this resistance, blood glucose levels remain normal. When the pancreas can no longer be maintained, blood sugar begins to rise, leading to diabetes. Insulin resistance varies from normal (insulin sensitivity) to insulin resistance (IR).

“A-beta associated diseases” can refer to those diseases or conditions characterized by A-beta protein aggregates. A primary component of amyloid plaques characteristic of A-beta associated diseases is beta amyloid peptide (A-beta), a highly insoluble peptide 39-43 amino acids (aa) in length that has a strong propensity to adopt beta sheet structures, oligomerize and form protein aggregates. Non-limiting examples of A-beta associated diseases include neurodegenerative diseases or disorders, Alzheimer's disease, dementia of the Alzheimer type, cerebral amyloid angiopathy (CAA), trisomy 21 (Down's Syndrome), adult Down syndrome, hereditary cerebral hemorrhage with amyloidosis of the Dutch-type (HCHWA-D), dementia with Lewy Bodies, frontotemporal lobar degeneration, glaucoma, age-related macular degeneration, amyotrophic lateral sclerosis, sporadic inclusion body myositis, and anxiety disorder in an elderly human subject,

Origin of the compounds of the disclosure: The provided compounds were not isolated from tissues naturally occurring in nature, but from the result of an artificial experiment combining a human cell and a chemically synthesized n-3-VLC-PUFA. The structures of our synthetic elovanoid compounds were matched using HPLC and mass spectrometry with compounds biosynthesized in human retinal pigment epithelial cells or detected in neuronal cell cultures. However, the natural occurrence of the provided mono- and di-hydroxylated elovanoids with specifically defined stereochemistry is not known at this time. Moreover, the provided compounds are not obtained from natural sources, but they are prepared by adapting stereocontrolled synthetic methods known in the art, starting with commercially available materials. The provided preparation methods were designed to be suitable to the unique hydrophobic properties of n-3 VLC-PUFA, which differ significantly from compounds that have a total number of carbons of 22 carbons or less.

The present disclosure encompasses compounds that have stereochemically pure structures and are chemically synthesized and modified to have additional structural features and properties that allow them to exert pharmacological activity. The provided compounds are chemically modified pharmaceutically acceptable derivatives in the form of carboxylic esters or salts that enhance their chemical and biological stability and allow for their use in therapeutic applications involving various forms of drug delivery.

The disclosure also provides pharmacologically effective compositions of the provided compounds that enhance their ability to be delivered to a subject in a manner that can reach the targeted cells and tissues.

The data described herein also provides support for the beneficial use of the provided n-3 VLC-PUFA and/or elovanoid compounds, as therapeutics for the prevention and treatment of diseases, such as diseases associated with allergies or allergic reactions, by abrogating the production of pro-inflammatory cytokines and chemokines by a cell, such as an epithelial cell.

Epithelium lines both the outside (skin) and the inside cavities and lumina of bodies. Epithelial tissue is scutoid shaped, tightly packed and form a continuous sheet. It has almost no intercellular spaces. Epithelia is separated from underlying tissues by an extracellular fibrous basement membrane. The lining of the mouth, lung alveoli and kidney tubules are all made of epithelial tissue. The lining of the blood and lymphatic vessels are of a specialized form of epithelium called endothelium.

The term “epithelial cell” can refer to cells that line the outside (skin), mucous membranes, and the inside cavities and lumina of the body. Most epithelial cells exhibit an apical-basal polarization of cellular components. Epithelial cells are classified by shape and by their specialization.

The epidermis (i.e., skin) consists of keratinized stratified squamous epithelium. Four cell types are present: keratinocytes produce keratin, a protein that hardens and waterproofs the skin. Mature keratinocytes at the skin surface are dead and filled almost entirely with keratin. Melanocytes produce melanin, a pigment that protects cells from ultraviolet radiation. Melanin from the melanocytes is transferred to the keratinocytes. Langerhans cells are phagocytic macrophages that interact with white blood cells during an immune response. Merkel cells occur deep in the epidermis at the epidermal-dermal boundary. They form Merkel discs, which, in association with nerve endings, serve a sensory function.

There are several layers making up the epidermis. “Thick skin,” found on the palms of the hands and soles of the feet, consists of five layers while “thin skin” consists of only four layers. The five layers can include the stratum corneum contains many layers of dead, anucleate keratinocytes completely filled with keratin. The outermost layers are constantly shed. The stratum lucidum contains two to three layers of anucleate cells. This layer is found only in “thick skin” such as the palm of the hand and the sole of the foot. The stratum granulosum contains two to four layers of cells held together by desmosomes. These cells contain keratohyaline granules, which contribute to the formation of keratin in the upper layers of the epidermis. The stratum spinosum contains eight to ten layers of cells connected by desmosomes. These cells are moderately active in mitosis. The stratum basale (stratum germinativum) contains a single layer of columnar cells actively dividing by mitosis to produce cells that migrate into the upper epidermal layers and ultimately to the surface of the skin.

Nasal epithelial cells, for example, form the outermost protective layer against environmental factors. They clean, humidify, and warm inhaled air. They produce mucus, which bind particles that are subsequently transported to the pharynx by cilia on the epithelial cells.

The corneal epithelium, for example, is made up of epithelial tissue and covers the front of the cornea. It acts as a barrier to protect the cornea, resisting the free flow of fluids from the tears, and prevents bacteria from entering the epithelium and corneal stroma.

Respiratory epithelium, or airway epithelium, is a type of ciliated columnar epithelium found lining most of the respiratory tract as respiratory mucosa. The cells in the respiratory epithelium are of four main types: a) ciliated cells, b) goblet cells, and c) club cells, and d) basal cells. The respiratory epithelium functions to moisten and protect the airways. It acts as a physical barrier to pathogens and foreign particles, as well as the removal of pathogens in the mechanism of mucociliary clearance, thus preventing infection and tissue injury by the secretion of mucus and the action of mucociliary clearance.

Compounds

Described herein are compounds based on omega-3 very long chain polyunsaturated fatty acids and their hydroxylated derivatives, termed “elovanoids”.

The omega-3 very long chain polyunsaturated fatty acids have the structures of A or B, or derivatives thereof:

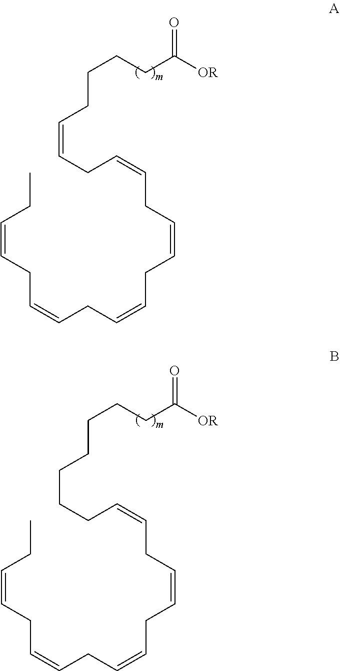

wherein: A contains a total from 23 to 42 carbon atoms in the carbon chain, and with 6 alternating cis carbon-carbon double bonds starting at positions n-3, n-6, n-9, n-12, n-15 and n-18, and wherein B contains a total from 23 to 42 carbon atoms in the carbon chain, and with 5 alternating cis carbon-carbon double bonds starting at positions n-3, n-6, n-9, n-12 and n-15. R can be hydrogen, methyl, ethyl, alkyl, or a cation such as an ammonium cation, an iminium cation, or a metal cation including, but not limited to, sodium, potassium, magnesium, zinc, or calcium cation, and wherein m is a number from 0 to 19.

The omega-3 very long chain polyunsaturated fatty acids of the disclosure can have a terminal carboxyl group “—COOR” wherein “R” can represent a group covalently bonded to the carboxyl such as an alkyl group. In the alternative, the carboxyl group can further have a negative charge as “—COO⁻” and R is a cation including a metal cation, an ammonium cation and the like.

In some omega-3 very long chain polyunsaturated fatty acids, m is a number selected from a group consisting of 0 to 15. Thus, can be a number selected from 1, 3, 5, 7, 9, 11, 13, or 15 where the fatty acid component contains a total of 24, 26, 28, 30, 32, 34, 36 or 38 carbon atoms in its carbon chain. In other omega-3 very long chain polyunsaturated fatty acids, m is a number selected from a group consisting of 0, 2, 4, 6, 8, 10, 12 or 14, where the fatty acid component contains a total of 23, 25, 27, 19, 31, 33, 35 or 37 carbon atoms in its carbon chain. In some omega-3 very long chain polyunsaturated fatty acids, m is a number selected from a group consisting of 5 to 15, where the fatty acid component contains a total of 28, 29, 30, 31, 32, 33, 34, 35, 36, 37 or 38 carbon atoms in its carbon chain. In some omega-3 very long chain polyunsaturated fatty acids, m is a number selected from a group consisting of 9 to 11, where the fatty acid component contains a total of 32 or 34 carbon atoms in its carbon chain.

In some embodiments the omega-3 very long chain polyunsaturated fatty acids is a carboxylic acid, i.e. R is hydrogen. In other embodiments the omega-3 very long chain polyunsaturated fatty acids is a carboxylic ester, wherein R is methyl, ethyl or alkyl. When the omega-3 very long chain polyunsaturated fatty acid is a carboxylic ester, R can be, but is not limited to, methyl or ethyl. In some embodiments the omega-3 very long chain polyunsaturated fatty acid is a carboxylic ester, wherein R is methyl.

In some embodiments the omega-3 very long chain polyunsaturated fatty acid can be a carboxylate salt, wherein R is an ammonium cation, iminium cation, or a metal cation selected from a group consisting of sodium, potassium, magnesium, zinc, or calcium cation. In some advantageous embodiments, R is ammonium cation or iminium cation. R can be a sodium cation or a potassium cation. In some embodiments, R is a sodium cation.

The omega-3 very long chain polyunsaturated fatty acid or derivative of the disclosure can have 32- or 34 carbons in its carbon chain and 6 alternating cis double bonds starting at the n-3 position, and have the formula A1 (14Z,17Z,20Z,23Z,26Z,29Z)-dotriaconta-14,17,20,23,26,29-hexaenoic acid) or formula A2 (16Z,19Z,22Z,25Z,28Z,31Z)-tetratriaconta-16,19,22,25,28,31-hexaenoic acid):

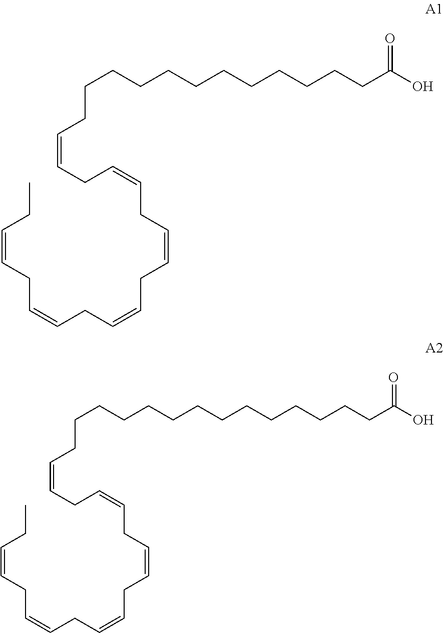 [SMILES: CC/C=C\C/C=C\C/C=C\C/C=C\C/C=C\C/C=C\CCCCCCCCCCCCC(=O)O ; CC/C=C\C/C=C\C/C=C\C/C=C\C/C=C\C/C=C\CCCCCCCCCCCCCCC(=O)O]

In some embodiments of the omega-3 very long chain polyunsaturated fatty acids, the carboxyl derivative is part of a glycerol-derived phospholipid, which can be readily prepared starting with the carboxylic acid form of the n-3 VLC-PUFA of structure A or B, by utilizing methods known in the art, and represented by structures C, D, E, or F:

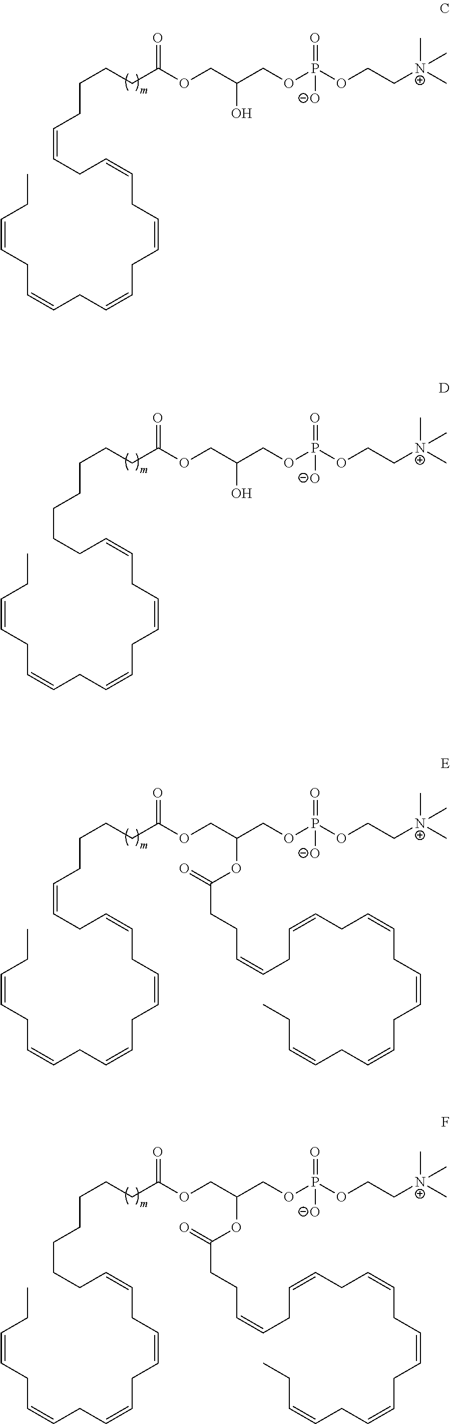

wherein C or E contains a total from 23 to 42 carbon atoms in the carbon chain, and with 6 alternating cis carbon-carbon double bonds starting at positions n-3, n-6, n-9, n-12, n-15 and n-18, and wherein D or E contains a total from 23 to 42 carbon atoms in the carbon chain, and with 5 alternating cis carbon-carbon double bonds starting at positions n-3, n-6, n-9, n-12 and n-15. In advantageous embodiments, m is a number selected from a group consisting of 0 to 15. In other embodiments, m is a number selected from 1, 3, 5, 7, 9, 11, 13, or 15 where the fatty acid component contains a total of 24, 26, 28, 30, 32, 34, 36 or 38 carbon atoms in its carbon chain. In additional advantageous embodiments, m is a number selected from a group consisting of 0, 2, 4, 6, 8, 10, 12 or 14, where the fatty acid component contains a total of 23, 25, 27, 19, 31, 33, 35 or 37 carbon atoms in its carbon chain.

In some embodiments, m is a number selected from a group consisting of 5 to 15, where the fatty acid component contains a total of 28, 29, 30, 31, 32, 33, 34, 35, 36, 37 or 38 carbon atoms in its carbon chain. In some embodiments, m is a number selected from a group consisting of 5, 7, 9, 11, 13, or 15, where the fatty acid component contains a total of 28, 30, 32, 34, 36 or 38 carbon atoms in its carbon chain. In other embodiments, m is a number selected from a group consisting of 4, 6, 8, 10, 12 or 14, where the fatty acid component contains a total of 27, 29, 31, 33, 35 or 37 carbon atoms in its carbon chain. In advantageous embodiments, m is a number selected from a group consisting of 9 to 11, where the fatty acid component contains a total of 32 or 34 carbon atoms in its carbon chain.

The mono-hydroxylated elovanoids of the disclosure can have the structures of G, H, I or J:

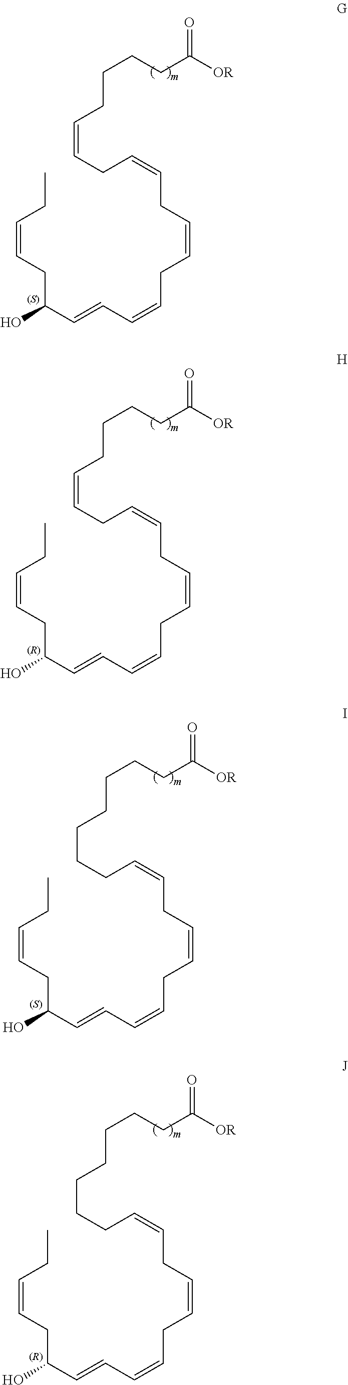

wherein compounds G and H have a total from 23 to 42 carbon atoms in the carbon chain, with 5 cis carbon-carbon double bonds starting at positions n-3, n-9, n-12, n-15 and n-18 and a trans carbon-carbon double bond starting at positions n-7; and wherein compounds I and J have a total from 23 to 42 carbon atoms in the carbon chain, and with 4 cis carbon-carbon double bonds starting at positions n-3, n-9, n-12 and n-15, and a trans carbon-carbon double bond starting at positions n-7; wherein R is hydrogen, methyl, ethyl, alkyl, or a cation selected from a group consisting of: ammonium cation, iminium cation, or a metal cation selected from a group consisting of sodium, potassium, magnesium, zinc, or calcium cation, and wherein m is a number selected from a group consisting of 0 to 19; wherein compounds G and H can exist as an equimolar mixture; wherein compounds I and J can exist as an equimolar mixture; wherein, the provided compounds G and H are predominately one enantiomer with a defined (S) or (R) chirality at the carbon bearing the hydroxyl group; and wherein, the compounds G and H are predominately one enantiomer with a defined (S) or (R) chirality at the carbon bearing the hydroxyl group.

As used herein and in other structures of the present disclosure, the compounds of the disclosure are shown having a terminal carboxyl group “—COOR” the “R” can represent a group covalently bonded to the carboxyl such as an alkyl group. In the alternative, the carboxyl group can further have a negative charge as “—COO⁻” and R is a cation including a metal cation, an ammonium cation and the like.

In some embodiments of the mono-hydroxylated elovanoids of the disclosure, m is a number selected from a group consisting of 0 to 15. In other advantageous embodiments, m is a number selected from 1, 3, 5, 7, 9, 11, 13, or 15 where the fatty acid component contains a total of 24, 26, 28, 30, 32, 34, 36 or 38 carbon atoms in its carbon chain. In other embodiments, m is a number selected from a group consisting of 0, 2, 4, 6, 8, 10, 12 or 14, where the fatty acid component contains a total of 23, 25, 27, 19, 31, 33, 35 or 37 carbon atoms in its carbon chain.

In some embodiments, m is a number selected from a group consisting of 5 to 15, where the fatty acid component contains a total of 28, 29, 30, 31, 32, 33, 34, 35, 36, 37 or 38 carbon atoms in its carbon chain. In some embodiments, m is a number selected from a group consisting of 5, 7, 9, 11, 13, or 15, where the fatty acid component contains a total of 28, 30, 32, 34, 36 or 38 carbon atoms in its carbon chain. In other embodiments, m is a number selected from a group consisting of 4, 6, 8, 10, 12 or 14, where the fatty acid component contains a total of 27, 29, 31, 33, 35 or 37 carbon atoms in its carbon chain. In advantageous embodiments, m is a number selected from a group consisting of 9 to 11, where the fatty acid component contains a total of 32 or 34 carbon atoms in its carbon chain.

In some embodiments the mono-hydroxylated elovanoids of the disclosure are a carboxylic acid, i.e. R is hydrogen. In other embodiments the compound is a carboxylic ester, wherein R is methyl, ethyl or alkyl. In advantageous embodiments the compound is a carboxylic ester, wherein R is methyl or ethyl. In advantageous embodiments the compound is a carboxylic ester, wherein R is methyl. In other advantageous embodiments the compound is a carboxylate salt, wherein R is an ammonium cation, iminium cation, or a metal cation selected from a group consisting of sodium, potassium, magnesium, zinc, or calcium cation. In some advantageous embodiments, R is ammonium cation or iminium cation. In other advantageous embodiments, R is a sodium cation or a potassium cation. In advantageous embodiments, R is a sodium cation.

The di-hydroxylated elovanoids of the disclosure can have the structures K, L, M, or N

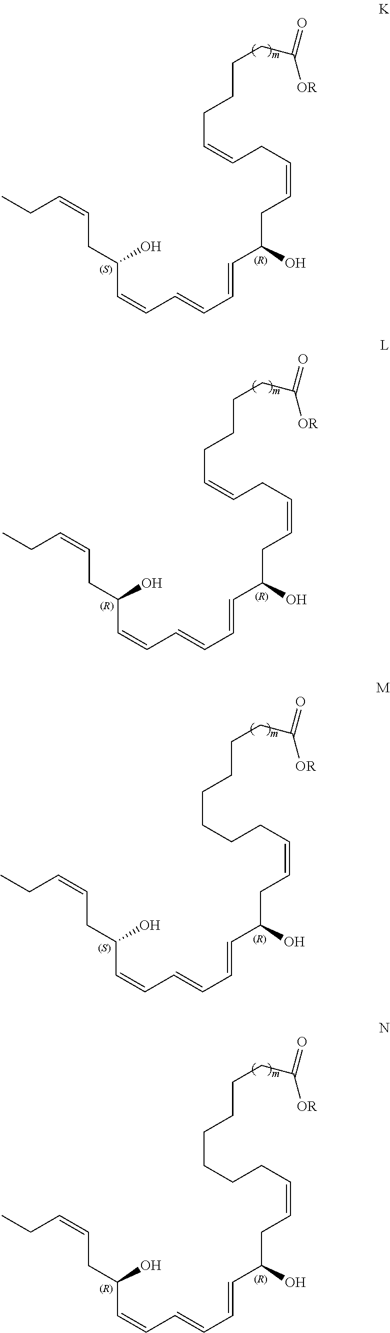

wherein compounds K and L have a total from 23 to 42 carbon atoms in the carbon chain, with 4 cis carbon-carbon double bonds starting at positions n-3, n-7, n-15 and n-18, and 2 trans carbon-carbon bonds starting at positions n-9, n-11; and wherein compounds M and N have a total from 23 to 42 carbon atoms in the carbon chain, with 3 cis carbon-carbon double bonds starting at positions n-3, n-7, n-12 and n-15, and 2 trans carbon-carbon bonds starting at positions n-9, n-11, wherein R is hydrogen, methyl, ethyl, alkyl, or a cation selected from a group consisting of: ammonium cation, iminium cation, or a metal cation selected from a group consisting of sodium, potassium, magnesium, zinc, or calcium cation, and wherein m is a number selected from a group consisting of 0 to 19; wherein compounds K and L can exist as an equimolar mixture; wherein compounds M and N can exist as an equimolar mixture, wherein the compounds K and L are predominately one enantiomer with a defined (S) or (R) chirality at the carbon bearing the hydroxyl group; and wherein, the provided compounds M and N are predominately one enantiomer with a defined (S) or (R) chirality at the carbon bearing the hydroxyl group.

As used herein and in other structures of the present disclosure, the compounds of the disclosure are shown having a terminal carboxyl group “—COOR” the “R” can represent a group covalently bonded to the carboxyl such as an alkyl group. In the alternative, the carboxyl group can further have a negative charge as “—COO⁻” and R is a cation including a metal cation, an ammonium cation and the like.

In some embodiments of the di-hydroxylated elovanoids of the disclosure, m is a number selected from a group consisting of 5 to 15, where the fatty acid component contains a total of 28, 29, 30, 31, 32, 33, 34, 35, 36, 37 or 38 carbon atoms in its carbon chain. In useful embodiments, m is a number selected from a group consisting of 5, 7, 9, 11, 13, or 15, where the fatty acid component contains a total of 28, 30, 32, 34, 36 or 38 carbon atoms in its carbon chain. In other embodiments, m is a number selected from a group consisting of 4, 6, 8, 10, 12 or 14, where the fatty acid component contains a total of 27, 29, 31, 33, 35 or 37 carbon atoms in its carbon chain. In useful embodiments, m is a number selected from a group consisting of 9 to 11, where the fatty acid component contains a total of 32 or 34 carbon atoms in its carbon chain.

Some di-hydroxylated elovanoids of the disclosure are carboxylic acid, i.e. R is hydrogen. In other embodiments the di-hydroxylated elovanoid of the disclosure is a carboxylic ester, wherein R is methyl, ethyl or alkyl. In useful embodiments the compound is a carboxylic ester, wherein R is methyl or ethyl. In useful embodiments the compound is a carboxylic ester, wherein R is methyl.

In other embodiments the di-hydroxylated elovanoid of the disclosure is a carboxylate salt, wherein R is an ammonium cation, iminium cation, or a metal cation selected from a group consisting of sodium, potassium, magnesium, zinc, or calcium cation. In some useful embodiments, R is ammonium cation or iminium cation. In other useful embodiments, R is a sodium cation or a potassium cation. In useful embodiments, R is a sodium cation.

The alkynyl mono-hydroxylated elovanoids of the disclosure can have the structures of O, P, Q or R:

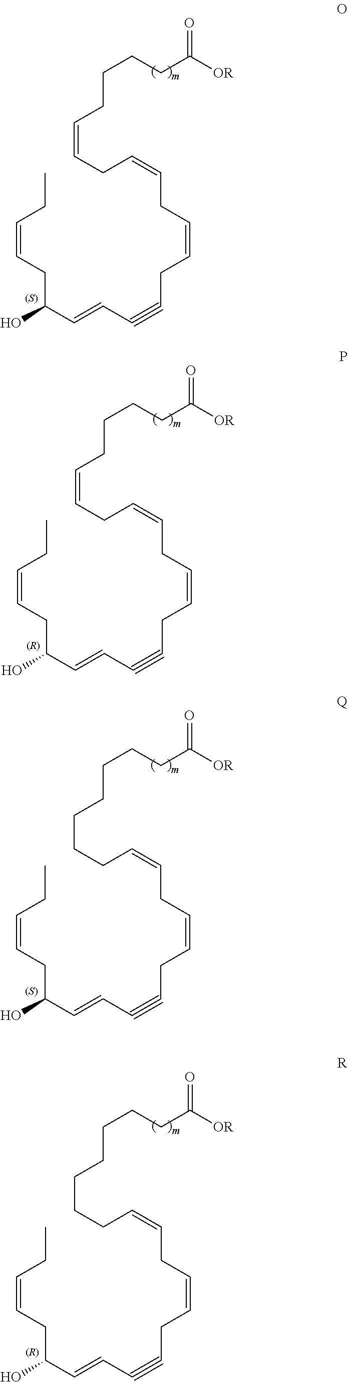

wherein compounds O and P have a total from 23 to 42 carbon atoms in the carbon chain, with 4 cis carbon-carbon double bonds starting at positions n-3, n-12, n-15 and n-18, a trans carbon-carbon bond starting at position n-7, and a carbon-carbon triple bond starting at position n-9; and wherein compounds I and J have a total from 23 to 42 carbon atoms in the carbon chain, with 3 cis carbon-carbon double bonds starting at positions n-3, n-12 and n-15, a trans carbon-carbon bond starting at position n-7, and a carbon-carbon triple bond starting at position n-9; wherein R is hydrogen, methyl, ethyl, alkyl, or a cation selected from a group consisting of: ammonium cation, iminium cation, or a metal cation selected from a group consisting of sodium, potassium, magnesium, zinc, or calcium cation, and wherein m is a number selected from a group consisting of 0 to 19; wherein compounds O and P can exist as an equimolar mixture; wherein compounds Q and R can exist as an equimolar mixture; wherein, the provided compounds O and P are predominately one enantiomer with a defined (S) or (R) chirality at the carbon bearing the hydroxyl group; and wherein, the provided compounds O and P are predominately one enantiomer with a defined (S) or (R) chirality at the carbon bearing the hydroxyl group.

As used herein and in other structures of the present invention, the alkynyl mono-hydroxylated elovanoids of the disclosure are shown having a terminal carboxyl group “—COOR” the “R” can represent a group covalently bonded to the carboxyl such as an alkyl group. In the alternative, the carboxyl group can further have a negative charge as “—COO⁻” and R is a cation including a metal cation, an ammonium cation and the like.

In some embodiments, m is a number selected from a group consisting of 0 to 15. In other embodiments, m is a number selected from 1, 3, 5, 7, 9, 11, 13, or 15 where the fatty acid component contains a total of 24, 26, 28, 30, 32, 34, 36 or 38 carbon atoms in its carbon chain.

In additional embodiments, m is a number selected from a group consisting of 0, 2, 4, 6, 8, 10, 12 or 14, where the fatty acid component contains a total of 23, 25, 27, 19, 31, 33, 35 or 37 carbon atoms in its carbon chain. In some embodiments, m is a number selected from a group consisting of 5 to 15, where the fatty acid component contains a total of 28, 29, 30, 31, 32, 33, 34, 35, 36, 37 or 38 carbon atoms in its carbon chain. In embodiments, m is a number selected from a group consisting of 5, 7, 9, 11, 13, or 15, where the fatty acid component contains a total of 28, 30, 32, 34, 36 or 38 carbon atoms in its carbon chain. In other embodiments, m is a number selected from a group consisting of 4, 6, 8, 10, 12 or 14, where the fatty acid component contains a total of 27, 29, 31, 33, 35 or 37 carbon atoms in its carbon chain. In some embodiments, m is a number selected from a group consisting of 9 to 11, where the fatty acid component contains a total of 32 or 34 carbon atoms in its carbon chain.

In some embodiments the alkynyl mono-hydroxylated elovanoids of the disclosure are carboxylic acids, i.e. R is hydrogen. In other embodiments the alkynyl mono-hydroxylated elovanoids of the disclosure are carboxylic esters, wherein R is methyl, ethyl or alkyl. In embodiments the alkynyl mono-hydroxylated elovanoids of the disclosure are carboxylic esters, wherein R is methyl or ethyl.

In some embodiments R is methyl. In other embodiments, alkynyl mono-hydroxylated elovanoids of the disclosure can be a carboxylate salt, wherein R is an ammonium cation, iminium cation, or a metal cation selected from a group consisting of sodium, potassium, magnesium, zinc, or calcium cation. In some embodiments, R is ammonium cation or iminium cation. In other embodiments, R is a sodium cation or a potassium cation. In embodiments, R is a sodium cation.

The alkynyl di-hydroxylated elovanoids can have the structures of S, T, U or V:

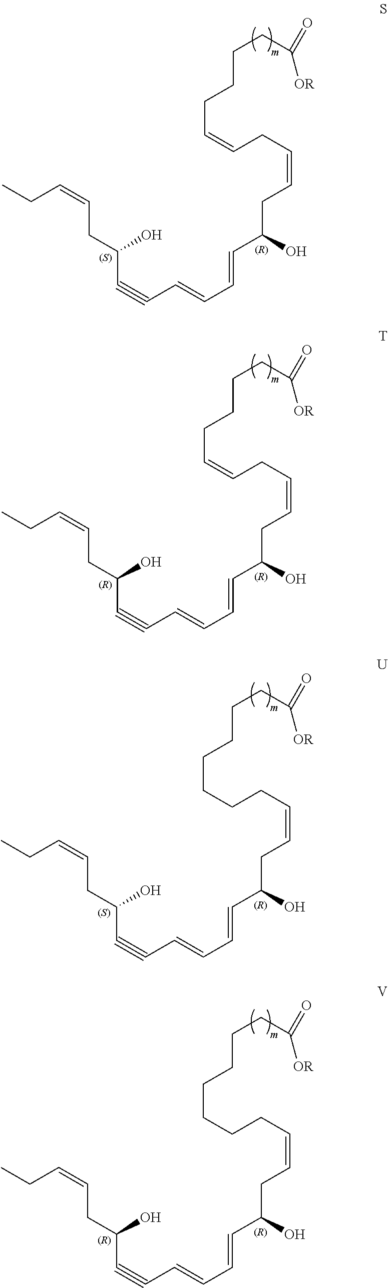

wherein compounds S and T have a total from 23 to 42 carbon atoms in the carbon chain, with 3 cis carbon-carbon double bonds starting at positions n-3, n-12, n-15 and n-18, with 2 trans carbon-carbon double bonds starting at positions n-9 and n-11, and a carbon-carbon triple bond starting at position n-7; and wherein compounds U and V have a total from 23 to 42 carbon atoms in the carbon chain, and with 2 cis carbon-carbon double bonds starting at positions n-3 and n-15, with 2 trans carbon-carbon double bonds starting at positions n-9 and n-11, and a carbon-carbon triple bond starting at position n-7; wherein R is hydrogen, methyl, ethyl, alkyl, or a cation selected from a group consisting of: ammonium cation, iminium cation, or a metal cation selected from a group consisting of sodium, potassium, magnesium, zinc, or calcium cation, and wherein m is a number selected from a group consisting of 0 to 19; wherein compounds S and T can exist as an equimolar mixture; wherein compounds U and V can exist as an equimolar mixture.

In some embodiments, the provided compounds S and T are predominately one enantiomer with a defined (S) or (R) chirality at the carbon bearing the hydroxyl group; and wherein, the provided compounds U and V are predominately one enantiomer with a defined (S) or (R) chirality at the carbon bearing the hydroxyl group.

As used herein and in other structures of the present invention, the compounds of the invention are shown having a terminal carboxyl group “—COOR” the “R” can represent a group covalently bonded to the carboxyl such as an alkyl group. In the alternative, the carboxyl group can further have a negative charge as “—COO⁻” and R is a cation including a metal cation, an ammonium cation and the like.

In some embodiments, m is a number selected from a group consisting of 5 to 15, where the fatty acid component contains a total of 28, 29, 30, 31, 32, 33, 34, 35, 36, 37 or 38 carbon atoms in its carbon chain. In embodiments, m is a number selected from a group consisting of 5, 7, 9, 11, 13, or 15, where the fatty acid component contains a total of 28, 30, 32, 34, 36 or 38 carbon atoms in its carbon chain. In other embodiments, m is a number selected from a group consisting of 4, 6, 8, 10, 12 or 14, where the fatty acid component contains a total of 27, 29, 31, 33, 35 or 37 carbon atoms in its carbon chain. In embodiments, m is a number selected from a group consisting of 9 to 11, where the fatty acid component contains a total of 32 or 34 carbon atoms in its carbon chain.

In some embodiments the provided compound is a carboxylic acid, i.e. R is hydrogen.

In other embodiments the provided compound is a carboxylic ester, wherein R is methyl, ethyl or alkyl. In embodiments the provided compound is a carboxylic ester, wherein R is methyl or ethyl. In embodiments the provided compound is a carboxylic ester, wherein R is methyl. In other embodiments the provided compound is a carboxylate salt, wherein R is an ammonium cation, iminium cation, or a metal cation selected from a group consisting of sodium, potassium, magnesium, zinc, or calcium cation. In some embodiments, R is ammonium cation or iminium cation. In other embodiments, R is a sodium cation or a potassium cation. In embodiments, R is a sodium cation.

In embodiments, the present disclosure provides a mono-hydroxylated 32-carbon methyl ester of formula G1, having the name: methyl (S,14Z,17Z,20Z,23Z,25E,29Z)-27-hydroxydotriaconta-14,17,20,23,25,29-hexaenoate; a mono-hydroxylated 32-carbon sodium salt of formula G2, having the name: sodium (S,14Z,17Z,20Z,23Z,25E,29Z)-27-hydroxydotriaconta-14,17,20,23,25,29-hexaenoate; a mono-hydroxylated 34-carbon methyl ester of formula G3, having the name: methyl (S,16Z,19Z,22Z,25Z,27E,31Z)-29-hydroxytetratriaconta-16,19,22,25,27,31-hexaenoate; or a mono-hydroxylated 34-carbon sodium salt of formula G4, having the name sodium (S,16Z,19Z,22Z,25Z,27E,31Z)-29-hydroxytetratriaconta-16,19,22,25,27,31-hexaenoate:

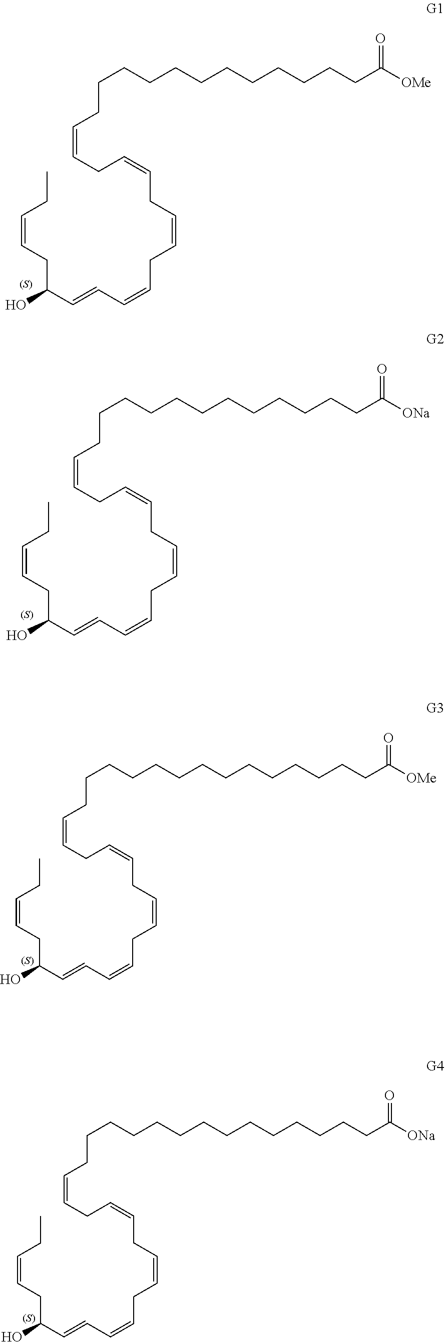

In other embodiments, the present disclosure provides a di-hydroxylated 32-carbon methyl ester of formula K1, having the name: methyl (14Z,17Z,20R,21E,23E,25Z,27S,29Z)-20,27-dihydroxydotriaconta-14,17,21,23,25,29-hexaenoate; a di-hydroxylated 32-carbon sodium salt of formula K2, having the name: sodium (14Z,17Z,20R,21E,23E,25Z,27S,29Z)-20,27-dihydroxydotriaconta-14,17,21,23,25,29-hexaenoate; or a di-hydroxylated 34-carbon methyl ester of formula K3, having the name: methyl (16Z,19Z,22R,23E,25E,27Z,29S,31Z)-22,29-dihydroxytetratriaconta-16,19,23,25,27,31-hexaenoate; or a di-hydroxylated 34-carbon sodium salt of formula K4, having the name: sodium (16Z,19Z,22R,23E,25E,27Z,29S,31Z)-22,29-dihydroxytetratriaconta-16,19,23,25,27,31-hexaenoate:

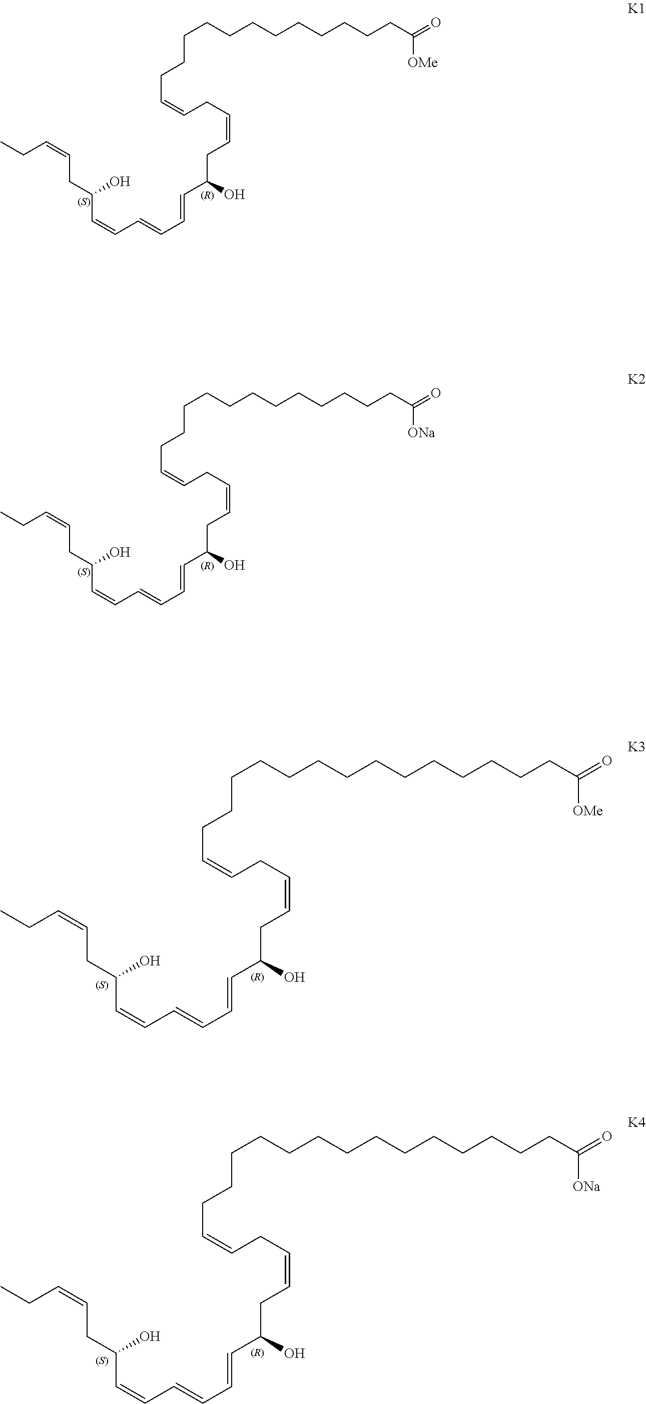

In other embodiments, the present invention provides an alkynyl mono-hydroxylated 32-carbon methyl ester of formula O1, having the name: methyl (S,14Z,17Z,20Z,25E,29Z)-27-hydroxydotriaconta-14,17,20,25,29-pentaen-23-ynoate; an alkynyl mono-hydroxylated 32-carbon sodium salt of formula O2, having the name: sodium (S,17Z,20Z,25E,29Z)-27-hydroxydotriaconta-17,20,25,29-tetraen-23-ynoate; an alkynyl mono-hydroxylated 34-carbon methyl ester of formula O3, having the name: methyl (S,16Z,19Z,22Z,27E,31Z)-29-hydroxytetratriaconta-16,19,22,27,31-pentaen-25-ynoate; an alkynyl mono-hydroxylated 34-carbon sodium salt of formula O4, having the name: sodium (S,16Z,19Z,22Z,27E,31Z)-29-hydroxytetratriaconta-16,19,22,27,31-pentaen-25-ynoate:

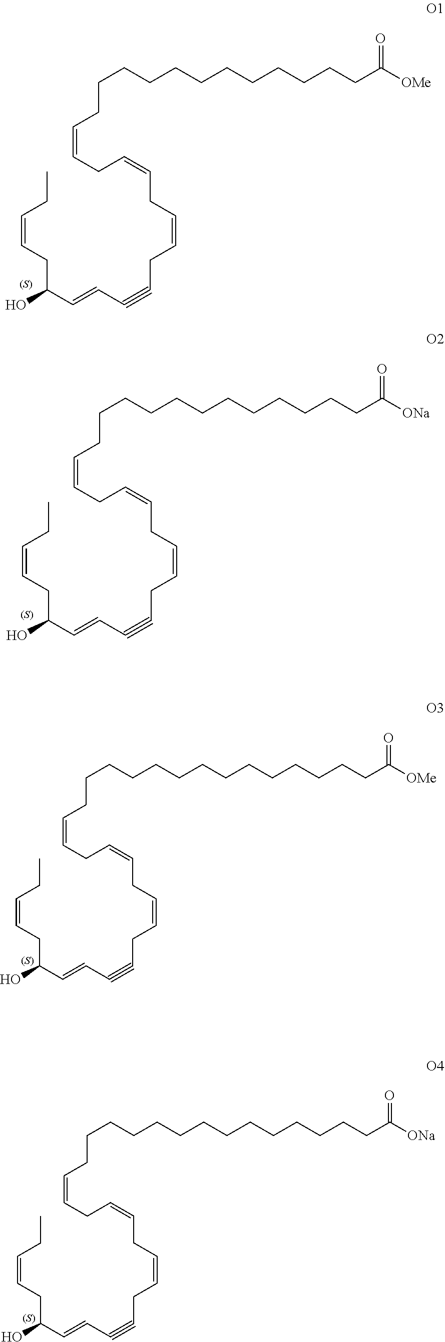

In other advantageous embodiments, the present invention provides an alkynyl di-hydroxylated 32-carbon methyl ester of formula S1, having the name: methyl (14Z,17Z,20R,21E,23E,27S,29Z)-20,27-dihydroxydotriaconta-14,17,21,23,29-pentaen-25-ynoate; an alkynyl di-hydroxylated 32-carbon sodium salt of formula S2, having the name: sodium (14Z,17Z,20R,21E,23E,27S,29Z)-20,27-dihydroxydotriaconta-14,17,21,23,29-pentaen-25-ynoate; or an alkynyl di-hydroxylated 34-carbon methyl ester of formula S3, having the name: methyl (16Z,19Z,22R,23E,25E,29S,31Z)-22,29-dihydroxytetratriaconta-16,19,23,25,31-pentaen-27-ynoate; or an alkynyl di-hydroxylated 34-carbon sodium salt of formula S4, having the name: sodium (16Z,19Z,22R,23E,25E,29S,31Z)-22,29-dihydroxytetratriaconta-16,19,23,25,31-pentaen-27-ynoate.

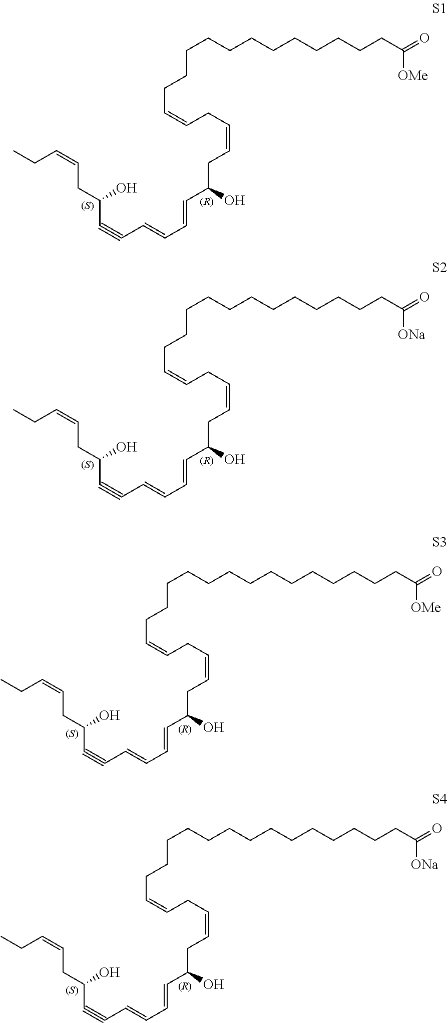

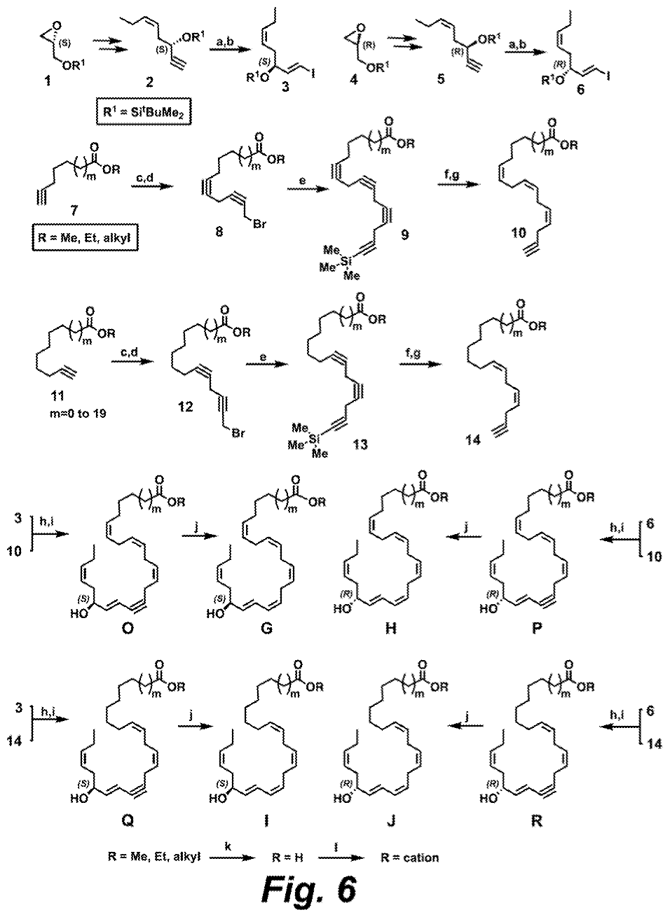

Methods of preparation and manufacturing of provided compounds: The provided compounds of the disclosure can be readily prepared by adapting methods known in the art, starting with commercially available materials as summarized in Schemes 1-5 as shown in FIGS. 6-10.

Scheme 1 (FIG. 6) shows the detailed approach for the stereocontrolled total synthesis of compounds of type O, wherein n is 9, and the fatty acid chain contains a total of 32 carbon atoms, and the R group is methyl or sodium cation. For example, Scheme 1 shows the synthesis of compounds ELV-N-32-Me and ELV-N-32-Na, starting with methyl pentadec-14-ynoate (S1). By starting with heptadec-16-ynoate (T1), this process affords compounds ELV-N-34-Me and ELV-N-34-Na. The alkynyl precursors of ELV-N-32-Me and ELV-N-32-Na, namely 13a, 13b, 15a, and 15b are also among the provided compounds X and Z in this disclosure. Scheme 1 provides the reagents and conditions for the preparations of the provided compounds, by employing reaction conditions that are typical for this type of reactions.

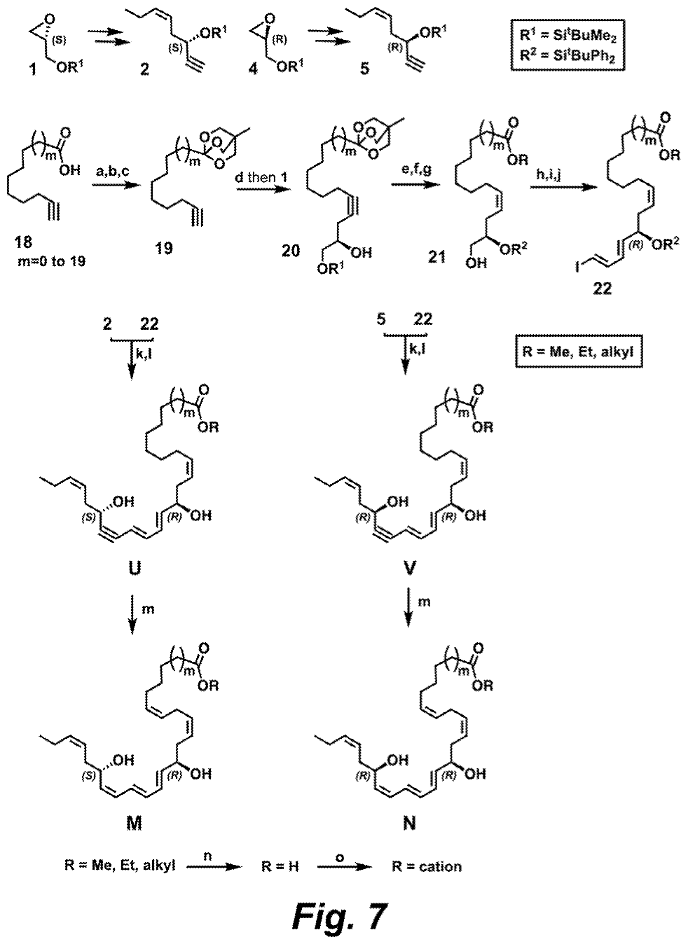

Scheme 2 (FIG. 7) describes the total synthesis of the di-hydroxylated elovanoids K and L and their alkyne precursors S and T, by starting with intermediates 2, 5, and 7 that were also used in Scheme 1. The conversion of the protected (R) epoxide 4 to intermediate 15, and the coupling of 7 and 15 followed by conversion into intermediate 17 can be done according to literature procedures (Tetrahedron Lett. 2012; 53(14):1695-8).

Catalytic cross-coupling between intermediates 2 or 17 or between intermediates 5 or 17, followed by deprotection, leads to the formation of alkynyl compounds S and T, which are then selectively reduced to form di-hydroxylated elovanoids K and L. Hydrolysis and acidification affords the corresponding carboxylic acids, which can be converted into carboxylate salts with the addition of equivalent amounts of the corresponding base. Di-hydroxylated elovanoids of types K, L, S and T with at least 23 carbons and up to 42 carbons in their carbon chain, can be similarly prepared by varying the number of carbons in the alkyne starting material 7.

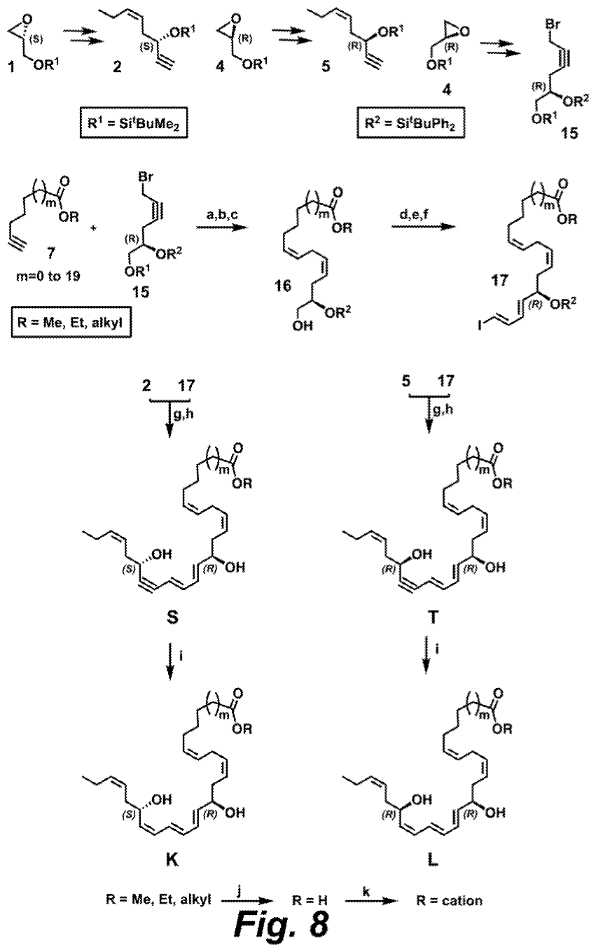

Scheme 3 (FIG. 8) describes the total synthesis of di-hydroxylated elovanoids with five unsaturated double bonds of types M and N, as well as their alkyne precursors U and V, by utilizing the same alkynyl intermediates 2 and 5, which were also used in Scheme 1. (Tetrahedron Lett. 2012; 53(14):1695-8).

The synthesis of the intermediate 22 begins with the carboxylic acid 18, which is converted into orthoester 19, using known methodologies (Tetrahedron Lett. 1983, 24 (50), 5571-4). Reaction of the lithiated alkyne with epoxide 1 affords intermediate 21, which is converted into the iodide intermediate 22, similarly to the conversion of 16 to 17. Catalytic cross-coupling between intermediates 2 or 5 with 22, followed by deprotection, leads to the formation of alkynyl di-hydroxy elovanoids U and V, which are then selectively reduced to form di-hydroxylated elovanoids M and N.

Hydrolysis and acidification affords the corresponding carboxylic acids, which can be converted into carboxylate salts with the addition of equivalent amounts of the corresponding base. Di-hydroxylated elovanoids of types M, N, U and V with at least 23 carbons and up to 42 carbons in their carbon chain, can be similarly prepared by varying the number of carbons in the alkyne carboxylic acid 18.

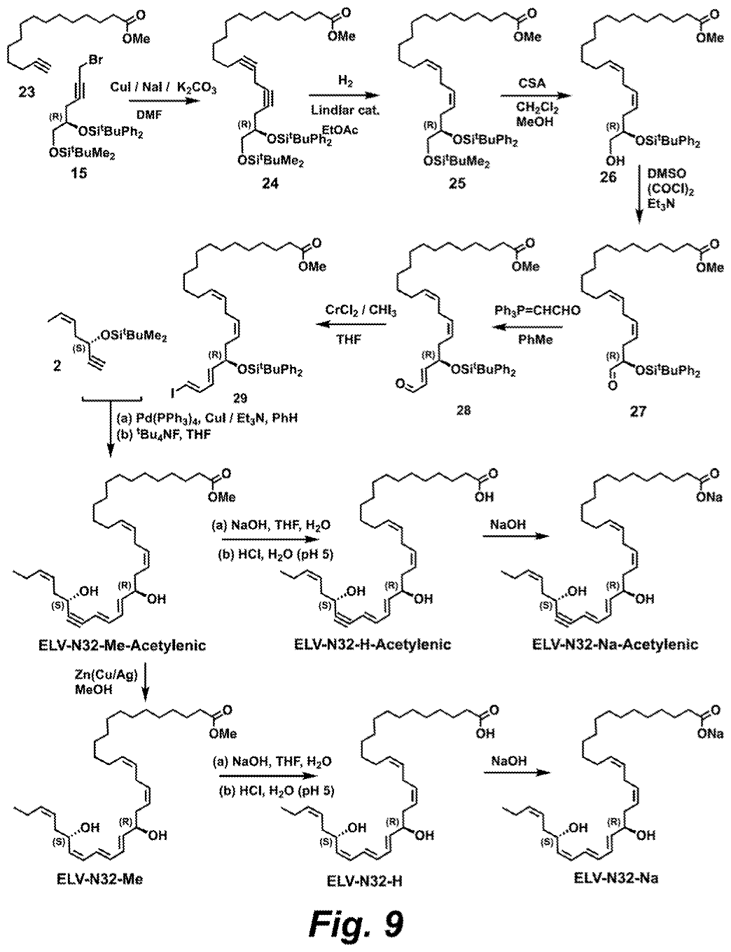

Scheme 4 (FIG. 9) shows the stereocontrolled total synthesis of 32-carbon dihydroxylated elovanoids, starting with alkyne methyl ester 23, intermediate 15, and alkyne intermediate 2. For example, this scheme shows the total synthesis of the 32-carbon alkynyl elovanoid compound ELV-N-32-Me-Acetylenic, and its conversion to elovanoid methyl ester ELV-N-32-Me, the elovanoid carboxylic acid ELV-N-32-H, and the elovanoid sodium salt ELV-N-32-Na.

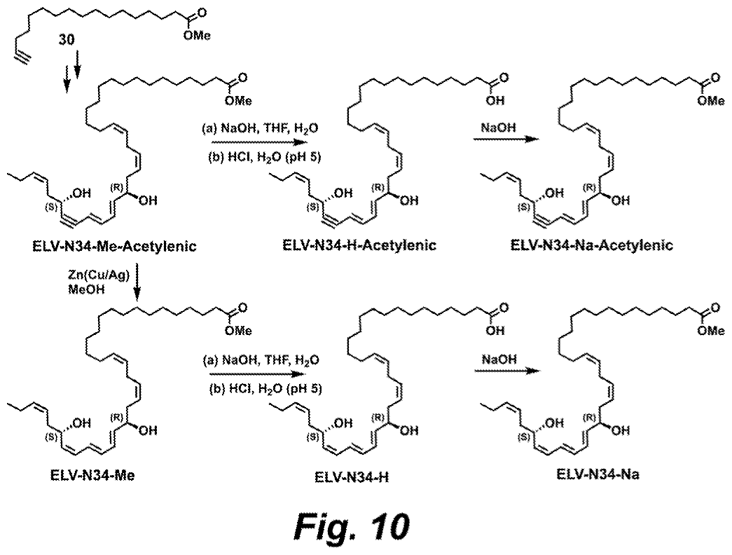

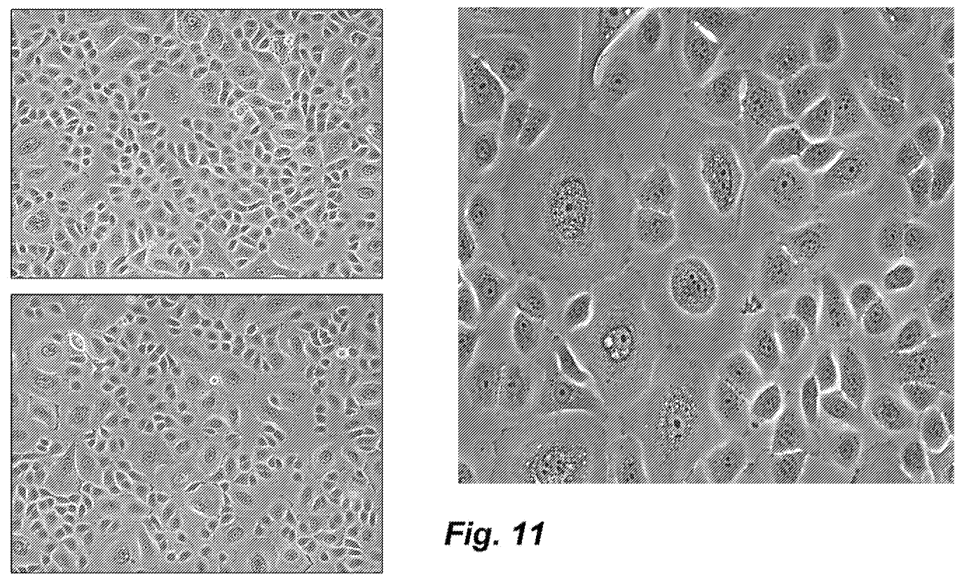

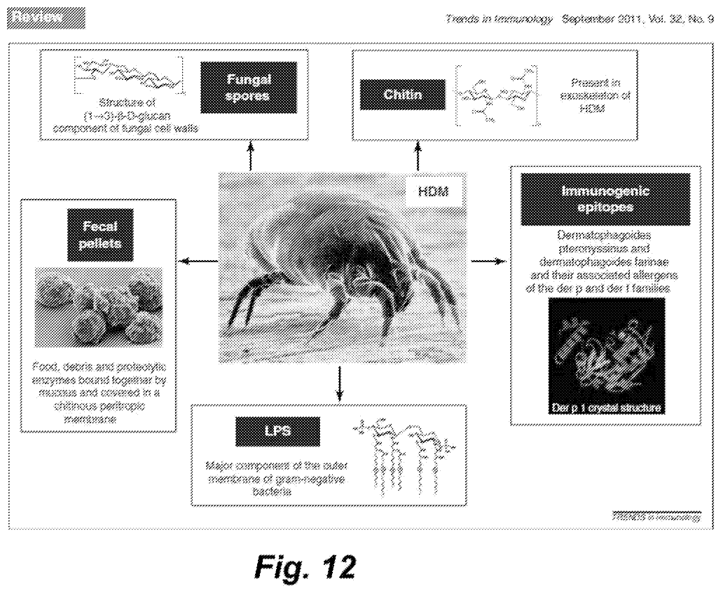

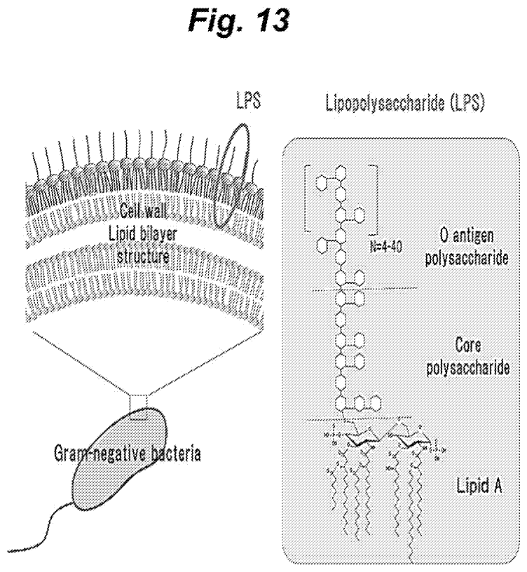

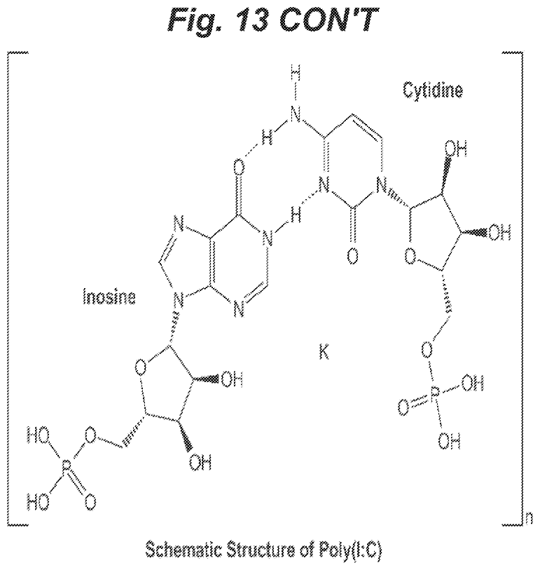

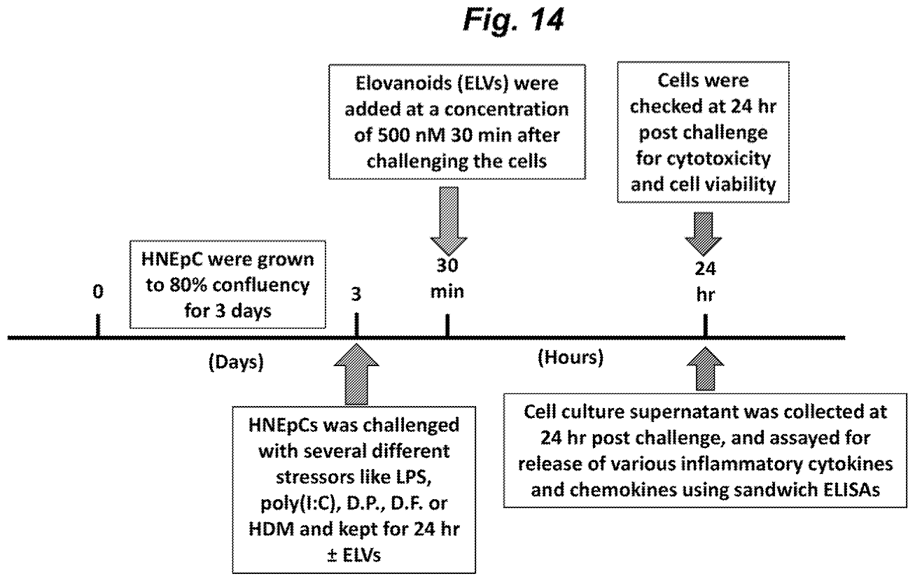

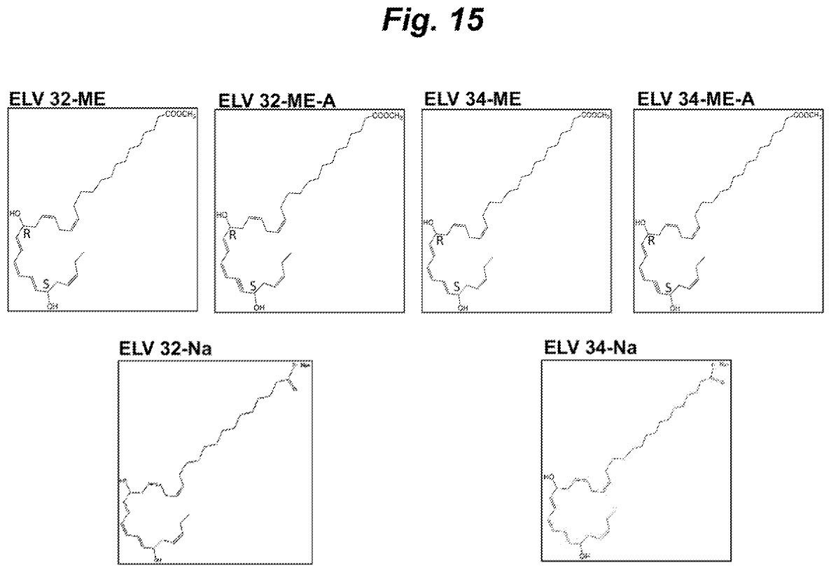

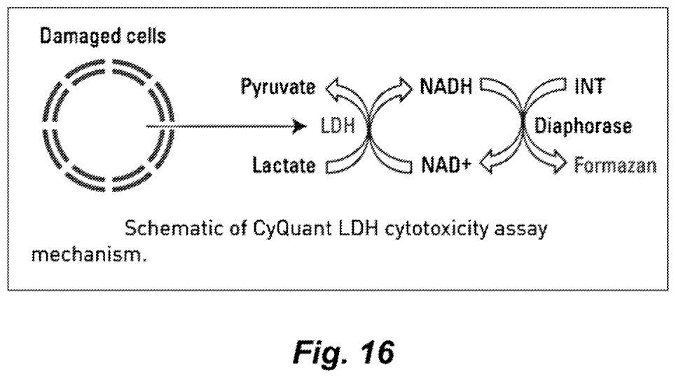

Scheme 5 (FIG. 10) shows the stereocontrolled total synthesis of 34-carbon dihydroxylated elovanoids, starting with alkyne methyl ester 30, and by employing the same sequence of reactions as in Scheme 4.

For example, this scheme shows the total synthesis of the 34-carbon alkynyl elovanoid compound ELV-N-34-Me-Acetylenic, and its conversion to elovanoid methyl ester ELV-N-34-Me, the elovanoid carboxylic acid ELV-N-34-H, and the elovanoid sodium salt ELV-N-34-Na.

The chemistry presented in Schemes 1-5 (FIGS. 6-10) can be also adapted for the total synthesis of additional mono-hydroxylated and di-hydroxylated elovanoids, having at least 23 carbons and up to 42 carbons in their carbon chain.

In embodiments, any isomer of a compound described herein is encompassed by the disclosure. In certain embodiments, the elovanoid compounds comprise a cis-trans-trans triene (ω-7, ω-9, and ω-11, respectively).

Pharmaceutical compositions for the treatment of diseases: In other embodiments, the present disclosure provides formulations of pharmaceutical compositions containing therapeutically effective amounts of one or more of compounds provided herein or their salts thereof in a pharmaceutically acceptable carrier.

The provided compositions contain one or more compounds provided herein or their salts thereof, and a pharmaceutically acceptable excipient, diluent, carrier and/or adjuvant. The compounds can be formulated into suitable pharmaceutical preparations such as solutions, suspensions, tablets, dispersible tablets, pills, capsules, powders, sustained release formulations or elixirs, for oral, buccal, intranasal, vaginal, rectal, ocular administration, sustained release from intravitreal implanted reservoirs or nano-devices or dendrimers, embedded in collagen or other materials on the eye surface, or in sterile solutions or suspensions for parenteral administration, dermal patches as well as transdermal patch preparation and dry powder inhalers. The provided formulations can be in the form of a drop, such as an eye drop, and the pharmaceutical formulation can further contain antioxidants and/or known agents for the treatment of eye diseases. The compounds described herein are formulated into pharmaceutical compositions using techniques and procedures well known in the art (see, e.g., Ansel Introduction to Pharmaceutical Dosage Forms, Fourth Edition 1985, 126).

Embodiments of the disclosure provide pharmaceutical compositions containing various forms of the provided compounds, as the free carboxylic acids or their pharmaceutically acceptable salts, or as their corresponding esters or their phospholipid derivatives. In other useful embodiments, the disclosure provides pharmaceutical compositions containing one or more elovanoid that contains one or two hydroxyl groups at positions located between n-3 to n-18 of the very long chain polyunsaturated fatty acids, as the free carboxylic acids or their pharmaceutically acceptable salts, or as their corresponding esters.

In a further embodiment, the disclosure provides a pharmaceutical composition for alleviating the symptom of, treating, or preventing a disease. For example, the disease is an allergic inflammatory disease, a disease associated with cellular senescence and/or ferroptosis, or a metabolic disorder.

In the provided compositions, effective concentrations of one or more compounds or pharmaceutically acceptable derivatives is (are) mixed with a suitable pharmaceutical carrier or vehicle. The compounds can be derivatized as the corresponding salts, esters, enol ethers or esters, acids, bases, solvates, hydrates or prodrugs prior to formulation, as described herein. The concentrations of the compounds in the compositions are effective for delivery of an amount, upon administration, that treats, prevents, or ameliorates one or more of the symptoms of a disease, disorder or condition.

As described herein, the compositions can be prepared by adapting methods known in the art. The compositions can be a component of a pharmaceutical formulation. The pharmaceutical formulation can further contain known agents for the treatment of inflammatory or degenerative diseases, including neurodegenerative diseases. The provided compositions can serve as pro-drug precursors of the fatty acids and can be converted to the free fatty acids upon localization to the site of the disease.

The present disclosure also provides packaged composition(s) or pharmaceutical composition(s) for prevention, restoration, or use in treating the disease or condition. Other packaged compositions or pharmaceutical compositions provided by the present disclosure further can include indicia including at least one of: instructions for using the composition to treat the disease or condition. The kit can further include appropriate buffers and reagents known in the art for administering various combinations of the components listed herein to the host.

Pharmaceutical formulations: Embodiments of the present disclosure can include a composition or pharmaceutical composition as identified herein and can be formulated with one or more pharmaceutically acceptable excipients, diluents, carriers, naturally occurring or synthetic antioxidants, and/or adjuvants. In addition, embodiments of the present disclosure can include a composition or pharmaceutical composition formulated with one or more pharmaceutically acceptable auxiliary substances. For example, the composition or pharmaceutical composition can be formulated with one or more pharmaceutically acceptable excipients, diluents, carriers, and/or adjuvants to provide an embodiment of a composition of the present disclosure.

A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc. The pharmaceutically acceptable excipients, such as vehicles, adjuvants, carriers or diluents, are available to the public. Moreover, pharmaceutically acceptable auxiliary substances, such as pH adjusting and buffering agents, tonicity adjusting agents, stabilizers, wetting agents and the like, are readily available to the public.

In an embodiment of the present disclosure, the composition or pharmaceutical composition can be administered to the subject using any means capable of resulting in the desired effect. Thus, the composition or pharmaceutical composition can be incorporated into a variety of formulations for therapeutic administration. For example, the composition or pharmaceutical composition can be formulated into pharmaceutical compositions by combination with appropriate, pharmaceutically acceptable carriers or diluents, and can be formulated into preparations in solid, semi-solid, liquid or gaseous forms, such as tablets, capsules, powders, granules, ointments, solutions, suppositories, injections, inhalants, creams, and aerosols.

Suitable excipient vehicles for the composition or pharmaceutical composition are, for example, water, saline, dextrose, glycerol, ethanol, or the like, and combinations thereof. In addition, the vehicle can contain minor amounts of auxiliary substances such as wetting or emulsifying agents, antioxidants or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pennsylvania, 17th edition, 1985. The composition or formulation to be administered will, in any event, contain a quantity of the composition or pharmaceutical composition adequate to achieve the desired state in the subject being treated.

Compositions of the present disclosure can include those that comprise a sustained release or controlled release matrix. In addition, embodiments of the present disclosure can be used in conjunction with other treatments that use sustained-release formulations. As used herein, a sustained-release matrix is a matrix made of materials, for example polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices can include a polylactide matrix, a polyglycolide matrix, and a polylactide co-glycolide (co-polymers of lactic acid and glycolic acid) matrix. In another embodiment, the pharmaceutical composition of the present disclosure (as well as combination compositions) can be delivered in a controlled release system. For example, the composition or pharmaceutical composition can be administered using intravenous infusion, an implantable osmotic pump, a transdermal patch, liposomes, or other modes of administration. In one embodiment, a pump can be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials are used. In yet another embodiment a controlled release system is placed in proximity of the therapeutic target thus requiring only a fraction of the systemic dose. In yet another embodiment, a controlled release system is placed in proximity of the therapeutic target, thus requiring only a fraction of the systemic. Other controlled release systems are discussed in the review by Langer (1990). Science 249:1527-1533.

In another embodiment, the compositions of the present disclosure (as well as combination compositions separately or together) can include those formed by impregnation of the composition or pharmaceutical composition described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the disclosure.

In another embodiment, the compositions or pharmaceutical compositions of the present disclosure (as well as combination compositions separately or together) can be part of a delayed-release formulation. Delayed-release dosage formulations can be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, M D, 2000, and “Pharmaceutical dosage forms and drug delivery systems”, 6th Edition, Ansel et al., (Media, PA: Williams and Wilkins, 1995). These references provide information on excipients, materials, equipment and process for preparing tablets and capsules and delayed release dosage forms of tablets, capsules, and granules. These references provide information on carriers, materials, equipment and process for preparing tablets and capsules and delayed release dosage forms of tablets, capsules, and granules.

Embodiments of the composition or pharmaceutical composition can be administered to a subject in one or more doses. Those of skill will appreciate that dose levels can vary as a function of the specific the composition or pharmaceutical composition administered, the severity of the symptoms and the susceptibility of the subject to side effects. Useful dosages for a given compound are readily determinable by those of skill in the art by a variety of means.

In an embodiment, multiple doses of the composition or pharmaceutical composition are administered. The frequency of administration of the composition or pharmaceutical composition can vary depending on any of a variety of factors, e.g., severity of the symptoms, and the like. For example, in an embodiment, the composition or pharmaceutical composition can be administered once per month, twice per month, three times per month, every other week (qow), once per week (qw), twice per week (biw), three times per week (tiw), four times per week, five times per week, six times per week, every other day (qod), daily (qd), twice a day (qid), three times a day (tid), or four times a day. As discussed herein, in an embodiment, the composition or pharmaceutical composition is administered 1 to 4 times a day over a 1 to 10-day time period.

The duration of administration of the composition or pharmaceutical composition analogue, e.g., the period of time over which the composition or pharmaceutical composition is administered, can vary, depending on any of a variety of factors, e.g., patient response, etc. For example, the composition or pharmaceutical composition in combination or separately, can be administered over a period of time of about one day to one week, about one day to two weeks.

The amount of the compositions and pharmaceutical compositions of the present disclosure that can be effective in treating the condition or disease can be determined by standard clinical techniques. In addition, in vitro or in vivo assays can be employed to help identify optimal dosage ranges. The precise dose to be employed can also depend on the route of administration, and can be decided according to the judgment of the practitioner and each patient's circumstances.

Routes of Administration: Embodiments of the present disclosure provide methods and compositions for the administration of the active agent(s) to a subject (e.g., a human) using any available method and route suitable for drug delivery, including in vivo and ex vivo methods, as well as systemic and localized routes of administration. Routes of administration can include intranasal, intramuscular, intratracheal, subcutaneous, intradermal, intravitreal, topical application, intravenous, rectal, nasal, oral, and other enteral and parenteral routes of administration. Routes of administration can be combined or adjusted depending upon the agent and/or the desired effect. An active agent can be administered in a single dose or in multiple doses.

In embodiments, aspects of the invention can be administered by a nebulizer. The term “nebulizer” can refer to any device known in the art that produces small droplets or an aerosol from a liquid. For example, the composition can be administered in the form of a mist, inhaled into the lungs.

The n-3 VLC-PUFA and their biogenic derivatives are formed in cells and are not a component of human diet. Advantageous routes of administration of the compounds provided herein will can include topical, oral, intranasal, and parenteral administration. For example, the provided formulations can be delivered in the form of a drop, such as an eye drop, or any other customary method for the treatment of an allergic inflammatory disease of the eye. For example, the provided formulations can be delivered in the form of an intranasal spray or any other customary method for the treatment of an allergic inflammatory disease of the nasal passage or lungs. For example, the provided formulations can be delivered in the form of a cream or gel or any other customary method for the treatment of an allergic inflammatory disease of the skin.

Parenteral routes of administration other than inhalation administration can include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to affect systemic or local delivery of the composition. Where systemic delivery is desired, administration involves invasive or systemically absorbed topical or mucosal administration of pharmaceutical preparations. In an embodiment, the composition or pharmaceutical composition can also be delivered to the subject by enteral administration. Enteral routes of administration can include, but are not limited to, oral and rectal (e.g., using a suppository) delivery.

Methods of administration of the composition or pharmaceutical composition through the skin or mucosa can include, but are not limited to, topical application of a suitable pharmaceutical preparation, transdermal transmission, injection and epidermal administration. For transdermal transmission, absorption promoters or iontophoresis are suitable methods. Iontophoretic transmission can be accomplished using commercially available “patches” that deliver their product continuously via electric pulses through unbroken skin for periods of several days or more.

The compounds and compositions provided by this disclosure are able to restore homeostasis and induce survival signaling in certain cells undergoing oxidative stress or other homeostatic disruptions. The disclosure also provides methods of use of the provided compounds and compositions containing a hydroxylated derivative of very long chain polyunsaturated fatty acids, as the free carboxylic acids or their pharmaceutically acceptable salts, or as their corresponding esters or other prodrug derivatives. The provided compounds can be readily prepared by adapting methods known in the art, starting with commercially available materials.

The bioactivity of the provided compounds, as exemplified by the elovanoid derivatives ELV-N-32-Me, ELV-N-32-Na, ELV-N-34-Me and ELV-N-34-Na, is attributed to their ability to reach the targeted human cells and exert their biological actions either by entering into the cell or/and by acting at a membrane bound receptor. Alternatively, the provided compounds can act via intracellular receptors (e.g. nuclear membrane), and thus they would work specifically by affecting key signaling events. Administration of a pharmaceutical composition, containing a provided compound and a pharmaceutically acceptable carrier, restores the homeostatic balance and promotes the survival of certain cells that are essential for maintaining normal function. The provided compounds, compositions, and methods can be used for the preventive and therapeutic treatment of inflammatory, degenerative, and neurodegenerative diseases. This disclosure targets critical steps of the initiation and early progression of these conditions by mimicking the specific biology of intrinsic cellular/organs responses to attain potency, selectivity, devoid of side effects and sustained bioactivity.

Accordingly, one aspect of the disclosure encompasses embodiments of a composition comprising at least one very long chain polyunsaturated fatty acid having at least 23 carbon atoms in its carbon chain.

In some embodiments of this aspect of the disclosure, the composition can further comprise a pharmaceutically-acceptable carrier and formulated for delivery of an amount of the at least one very long chain polyunsaturated fatty acid effective in reducing a pathological condition of a tissue of a recipient subject or the onset of a pathological condition of a tissue of a recipient subject.

In some embodiments of this aspect of the disclosure, the pathological condition can be an allergic inflammatory disease or allergic inflammatory condition of a tissue of the recipient subject.

In some embodiments of this aspect of the disclosure, the pathological condition can be associated with cellular senescence, ferroptosis, or both.

In some embodiments of this aspect of the disclosure, the pathological condition can be associated with a metabolic disorder.

In some embodiments of this aspect of the disclosure, the composition can be formulated for topical delivery of the at least one very long chain polyunsaturated fatty acid tissue to the skin or eye of a recipient subject.

In some embodiments of this aspect of the disclosure, the composition can be formulated for intranasal delivery of the at least one very long chain polyunsaturated fatty acid tissue to the nasal passage and/or lungs of a recipient subject.

In some embodiments of this aspect of the disclosure, the composition can further comprise at least one nutritional component, and, for example, the composition can be formulated for the oral or parenteral delivery of the at least one very long chain polyunsaturated fatty acid to a recipient subject.

In some embodiments of this aspect of the disclosure, the at least one very long chain polyunsaturated fatty acid can have from about 26 to about 42 carbon atoms in its carbon chain.

In some embodiments of this aspect of the disclosure, the at least one very long chain polyunsaturated fatty acid can have 32 or 34 carbon atoms in its carbon chain.

In some embodiments of this aspect of the disclosure, the very long chain polyunsaturated fatty acid can have in its carbon chain five or six double bonds with cis geometry.

In some embodiments of this aspect of the disclosure, the very long chain polyunsaturated fatty acid is 14Z,17Z,20Z,23Z,26Z,29Z)-dotriaconta-14,17,20,23,26,29-hexaenoic acid or (16Z,19Z,22Z,25Z,28Z,31Z)-tetratriaconta-16,19,22,25,28,31-hexaenoic acid.

Another aspect of the disclosure encompasses embodiments of a composition comprising at least one elovanoid having at least 23 carbon atoms in its carbon chain.

In some embodiments of this aspect of the disclosure, the composition can further comprise a pharmaceutically-acceptable carrier and can be formulated for delivery of an amount of the at least one elovanoid effective in reducing a pathological condition of a tissue of a recipient subject.

In some embodiments of this aspect of the disclosure, the pathological condition can be an allergic inflammatory disease.

In some embodiments of this aspect of the disclosure, the at least one elovanoid can be selected from the group consisting of: a mono-hydroxylated elovanoid, a di-hydroxylated elovanoid, an alkynyl mono-hydroxylated elovanoid, and an alkynyl di-hydroxylated elovanoid, or any combination thereof.

In some embodiments of this aspect of the disclosure, the at least one elovanoid can be a combination of elovanoids, wherein the combination is selected from the group consisting of: a mono-hydroxylated elovanoid and a di-hydroxylated elovanoid; a mono-hydroxylated elovanoid and an alkynyl mono-hydroxylated elovanoid; a mono-hydroxylated elovanoid and an alkynyl di-hydroxylated elovanoid; a di-hydroxylated elovanoid and an alkynyl mono-hydroxylated elovanoid; a di-hydroxylated elovanoid and an alkynyl di-hydroxylated elovanoid; a mono-hydroxylated elovanoid, a di-hydroxylated elovanoid, and an alkynyl mono-hydroxylated elovanoid; a mono-hydroxylated elovanoid, a di-hydroxylated elovanoid, and an alkynyl di-hydroxylated elovanoid; and a mono-hydroxylated elovanoid, a di-hydroxylated elovanoid, and an alkynyl mono-hydroxylated elovanoid an alkynyl di-hydroxylated elovanoid, wherein each elovanoid is independently a racemic mixture, an isolated enantiomer, or a combination of enantiomers wherein the amount of one enantiomer greater than the amount of another enantiomer; and wherein each di-hydroxylated elovanoid is independently a diastereomeric mixture, an isolated diastereomer, or a combination of diastereomers wherein the amount of one diastereomer is greater than the amount of another diastereomer.

In some embodiments of this aspect of the disclosure, the composition can further comprise at least one very long-chain polyunsaturated fatty acid having at least 23 carbon atoms in its carbon chain.

In some embodiments of this aspect of the disclosure, the at least one very long chain polyunsaturated fatty acid can have from about 26 to about 42 carbon atoms in its carbon chain.

In some embodiments of this aspect of the disclosure, the at least one very long chain polyunsaturated fatty acid can have in its carbon chain five or six double bonds with cis geometry.

In some embodiments of this aspect of the disclosure, the at least one very long chain polyunsaturated fatty acid can be 14Z,17Z,20Z,23Z,26Z,29Z)-dotriaconta-14,17,20,23,26,29-hexaenoic acid or (16Z,19Z,22Z,25Z,28Z,31Z)-tetratriaconta-16,19,22,25,28,31-hexaenoic acid.

In some embodiments of this aspect of the disclosure, the mono-hydroxylated elovanoid can be selected from the group consisting of the formulas G, H, I or J:

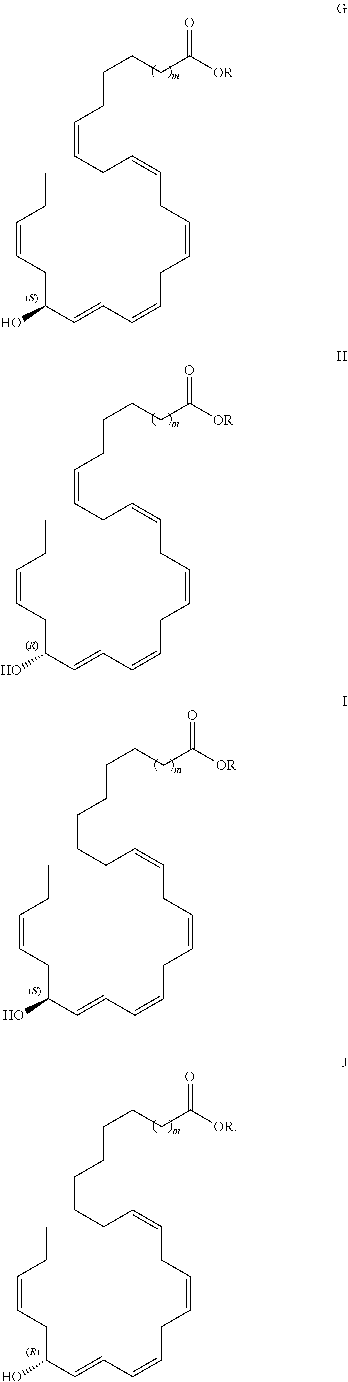

wherein: n can be 0 to 19 and —CO—OR can be a carboxylic acid group, or a salt or an ester thereof, and wherein: if —CO—OR can be a carboxylic acid group and the compound G, H, I or J can be a salt thereof, the cation of the salt can be a pharmaceutically acceptable cation, and if —CO—OR can be an ester, then R can be an alkyl group.

In some embodiments of this aspect of the disclosure, the pharmaceutically acceptable cation can be an ammonium cation, an iminium cation, or a metal cation.

In some embodiments of this aspect of the disclosure, the metal cation can be a sodium, potassium, magnesium, zinc, or calcium cation.

In some embodiments of this aspect of the disclosure, the composition can comprise equimolar amounts of the enantiomers G and H wherein the enantiomers have (S) or (R) chirality at the carbon bearing the hydroxyl group.

In some embodiments of this aspect of the disclosure, the composition can comprise amounts of the enantiomers I and J wherein the enantiomers have (S) or (R) chirality at the carbon bearing the hydroxyl group.

In some embodiments of this aspect of the disclosure, the composition can comprise one of the enantiomers of G or H in an amount exceeding the amount of the other enantiomer of G or H.

In some embodiments of this aspect of the disclosure, the composition can comprise one of the enantiomers of I or J in an amount exceeding the amount of the other enantiomer of I or J.

In some embodiments of this aspect of the disclosure, the mono-hydroxylated elovanoid can be selected from a group consisting of: methyl (S,14Z,17Z,20Z,23Z,25E,29Z)-27-hydroxydotriaconta-14,17,20,23,25,29-hexaenoate (G1), sodium (S,14Z,17Z,20Z,23Z,25E,29Z)-27-hydroxydotriaconta-14,17,20,23,25,29-hexaenoate (G2), methyl (S,16Z,19Z,22Z,25Z,27E,31Z)-29-hydroxytetratriaconta-16,19,22,25,27,31-hexaenoate (G3); and sodium (S,16Z,19Z,22Z,25Z,27E,31Z)-29-hydroxytetratriaconta-16,19,22,25,27,31-hexaenoate (G4) having the formulas, respectively:

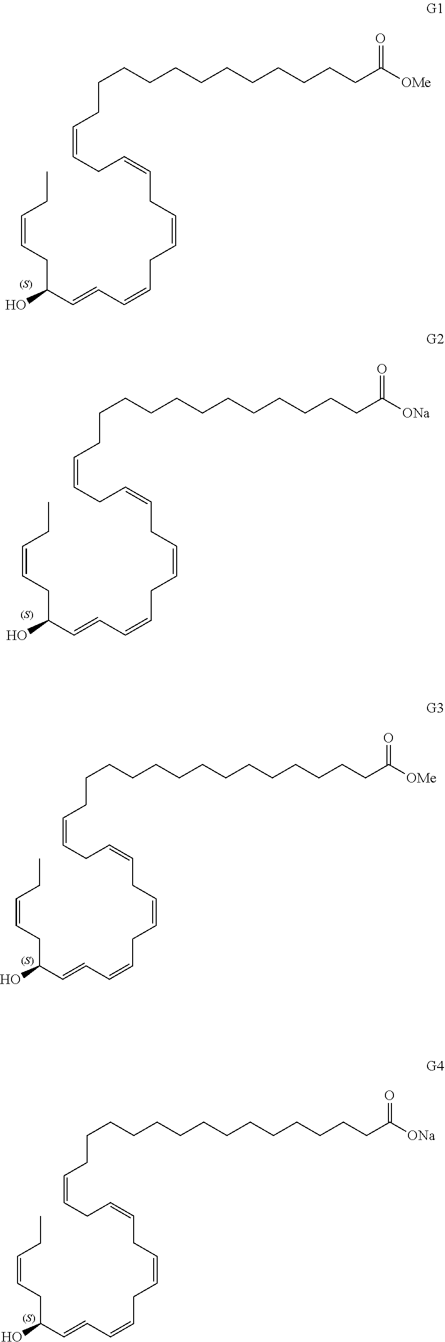

In some embodiments of this aspect of the disclosure, the di-hydroxylated elovanoid can be selected from the group consisting of the formulas K, L, M, and N:

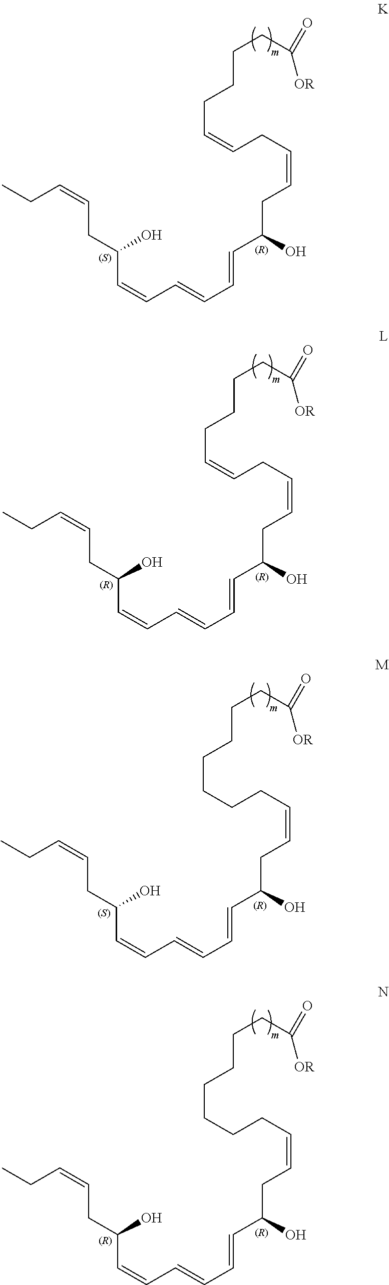

wherein: m can be 0 to 19 and —CO—OR can be a carboxylic acid group, or a salt or an ester thereof,
and wherein: if —CO—OR can be a carboxylic acid group and the compound K, L, M, or N can be a salt thereof, the cation of the salt can be a pharmaceutically acceptable cation, and if —CO—OR can be an ester, then R can be an alkyl group.

In some embodiments of this aspect of the disclosure, the pharmaceutically acceptable cation can be an ammonium cation, an iminium cation, or a metal cation.

In some embodiments of this aspect of the disclosure, the metal cation can be a sodium, potassium, magnesium, zinc, or calcium cation.

In some embodiments of this aspect of the disclosure, the composition can comprise equimolar amounts of the diastereomers K and L wherein the diastereomers have either (S) or (R) chirality at position n-6, and (R) chirality at position n-13.

In some embodiments of this aspect of the disclosure, the composition can comprise equimolar amounts of the diastereomers M and N wherein the diastereomers have either (S) or (R) chirality at position n-6, and (R) chirality at position n-13.

In some embodiments of this aspect of the disclosure, the composition can comprise one of the diastereomers of K or L in an amount exceeding the amount of the other diastereomer of K or L.

In some embodiments of this aspect of the disclosure, the composition can comprise one of the diastereomers of M or N in an amount exceeding the amount of the other diastereomer of M or N.

In some embodiments of this aspect of the disclosure, the di-hydroxylated elovanoid can be selected from the group consisting of: methyl (14Z,17Z,20R,21E,23E,25Z,27S,29Z)-20,27-dihydroxydotriaconta-14,17,21,23,25,29-hexaenoate (K1), sodium (14Z,17Z,20R,21E,23E,25Z,27S,29Z)-20,27-dihydroxydotriaconta-14,17,21,23,25,29-hexaenoate (K2), methyl (16Z,19Z,22R,23E,25E,27Z,29S,31Z)-22,29-dihydroxytetratriaconta-16,19,23,25,27,31-hexaenoate (K3), and sodium (16Z,19Z,22R,23E,25E,27Z,29S,31Z)-22,29-dihydroxytetratriaconta-16,19,23,25,27,31-hexaenoate (K4) having the formulas, respectively:

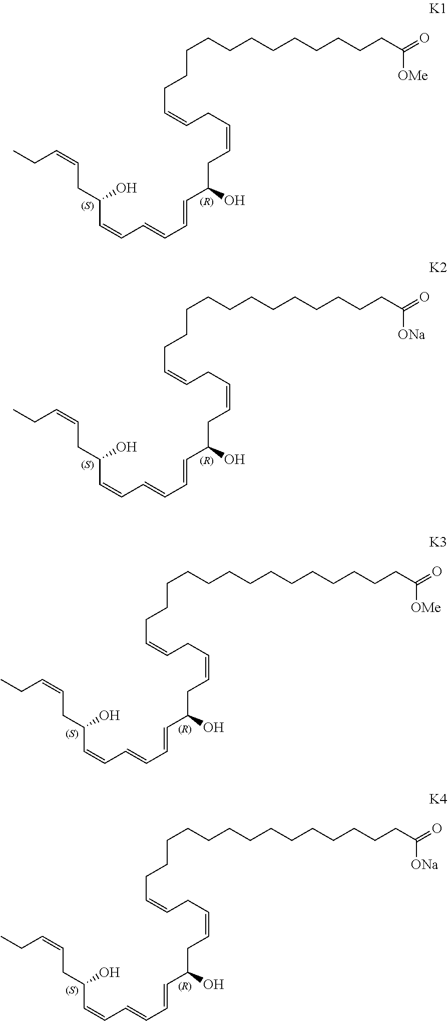

In some embodiments of this aspect of the disclosure, the alkynyl mono-hydroxylated elovanoid can be selected from the group consisting of the formulas 0, P, Q or R:

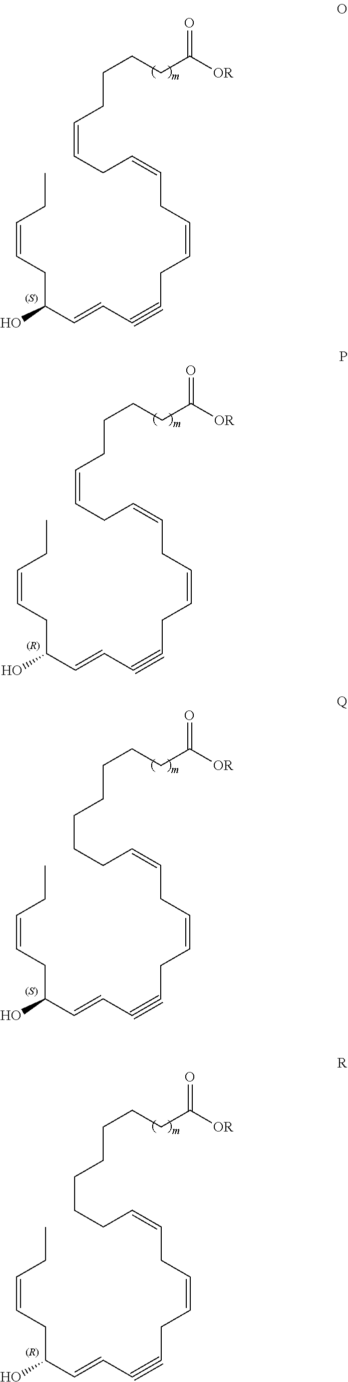

wherein: m can be 0 to 19 and —CO—OR can be a carboxylic acid group, or a salt or an ester thereof, and wherein: if —CO—OR can be a carboxylic acid group and the compound O, P, Q or R can be a salt thereof, the cation of the salt can be a pharmaceutically acceptable cation, and if —CO—OR can be an ester, then R can be an alkyl group, and wherein: compounds O and P each have a total from 23 to 42 carbon atoms in the carbon chain, with 4 cis carbon-carbon double bonds located at positions starting at n-3, n-12, n-15 and n-18; with a trans carbon-carbon double bond at position starting at n-7, and a carbon-carbon triple bond starting at position n-9; and compounds Q and R each have a total from 23 to 42 carbon atoms in the carbon chain, with 3 cis carbon-carbon double bond starting at positions n-3, n-12 and n-15, with a trans carbon-carbon double bond at position starting at n-7, and a carbon-carbon triple bond starting at position n-9.

In some embodiments of this aspect of the disclosure, the alkynyl mono-hydroxylated elovanoid can be selected from the group consisting of: methyl (S,14Z,17Z,20Z,25E,29Z)-27-hydroxydotriaconta-14,17,20,25,29-pentaen-23-ynoate (01); sodium (S,17Z,20Z,25E,29Z)-27-hydroxydotriaconta-17,20,25,29-tetraen-23-ynoate (O2); methyl (S,16Z,19Z,22Z,27E,31Z)-29-hydroxytetratriaconta-16,19,22,27,31-pentaen-25-ynoate (O3); and sodium (S,16Z,19Z,22Z,27E,31Z)-29-hydroxytetratriaconta-16,19,22,27,31-pentaen-25-ynoate (O4) and having the formulas, respectively:

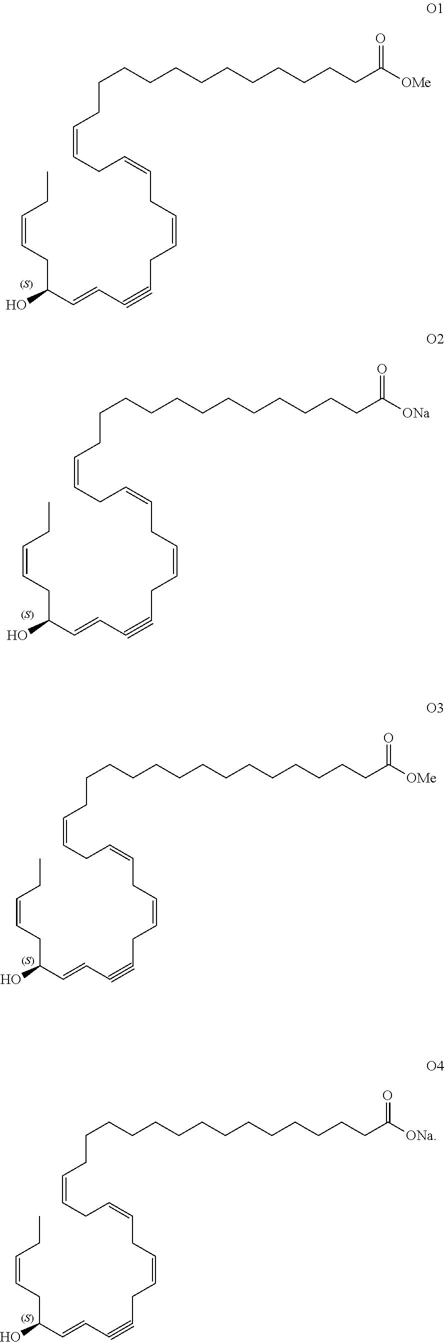

In some embodiments of this aspect of the disclosure, the pharmaceutically acceptable cation can be an ammonium cation, an iminium cation, or a metal cation.

In some embodiments of this aspect of the disclosure, the metal cation can be a sodium, potassium, magnesium, zinc, or calcium cation.

In some embodiments of this aspect of the disclosure, the composition can comprise equimolar amounts of the enantiomers 0 and P wherein the enantiomers have (S) or (R) chirality at the carbon bearing the hydroxyl group.

In some embodiments of this aspect of the disclosure, the composition can comprise equimolar amounts of the enantiomers Q and R wherein the enantiomers have (S) or (R) chirality at the carbon bearing the hydroxyl group.

In some embodiments of this aspect of the disclosure, the composition can comprise one of the enantiomers of O or P in an amount exceeding the amount of the other enantiomer of O or P.

In some embodiments of this aspect of the disclosure, the composition can comprise one of the enantiomers of Q or R in an amount exceeding the amount of the other enantiomer of Q or R.

In some embodiments of this aspect of the disclosure, the elovanoid can be an alkynyl di-hydroxylated elovanoid selected from the group consisting of the formulas S, T, U or V:

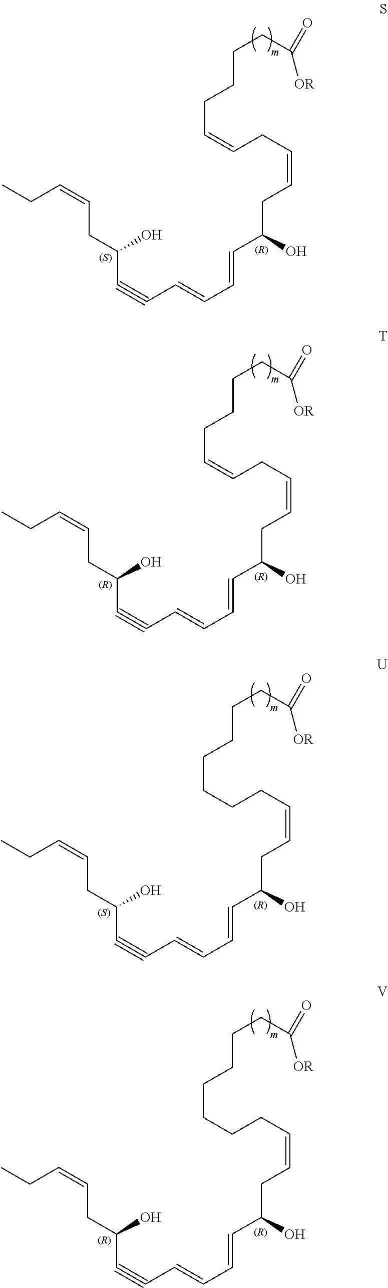

wherein: m can be 0 to 19 and —CO—OR can be a carboxylic acid group, or a salt or an ester thereof, and wherein: if —CO—OR can be a carboxylic acid group and the compound S, T, U or V can be a salt thereof, the cation of the salt can be a pharmaceutically acceptable cation, and if —CO—OR can be an ester, then R can be an alkyl group, and wherein: compounds S and T each have a total from 23 to 42 carbon atoms in the carbon chain, with 3 cis carbon-carbon double bonds starting at positions n-3, n-15 and n-18; 2 trans carbon-carbon double bonds starting at positions n-9, n-11; and a carbon-carbon triple bond starting at position n-7; and compounds U and V each have a total from 23 to 42 carbon atoms in the carbon chain, with 2 cis carbon-carbon double bond starting at positions n-3, and n-15; 2 trans carbon-carbon double bonds starting at positions n-9 and n-11; and a carbon-carbon triple bond starting at position n-7.

In some embodiments of this aspect of the disclosure, the pharmaceutically acceptable cation is an ammonium cation, an iminium cation, or a metal cation.

In some embodiments of this aspect of the disclosure, the metal cation is a sodium, potassium, magnesium, zinc, or calcium cation.

In some embodiments of this aspect of the disclosure, the alkynyl mono-hydroxylated elovanoid can be selected from the group consisting of: methyl (14Z,17Z,20R,21E,23E,27S,29Z)-20,27-dihydroxydotriaconta-14,17,21,23,29-pentaen-25-ynoate (S1); sodium (14Z,17Z,20R,21E,23E,27S,29Z)-20,27-dihydroxydotriaconta-14,17,21,23,29-pentaen-25-ynoate (S2); methyl (16Z,19Z,22R,23E,25E,29S,31Z)-22,29-dihydroxytetratriaconta-16,19,23,25,31-pentaen-27-ynoate (S3); and sodium (16Z,19Z,22R,23E,25E,29S,31Z)-22,29-dihydroxytetratriaconta-16,19,23,25,31-pentaen-27-ynoate (S4), and having the formula, respectively:

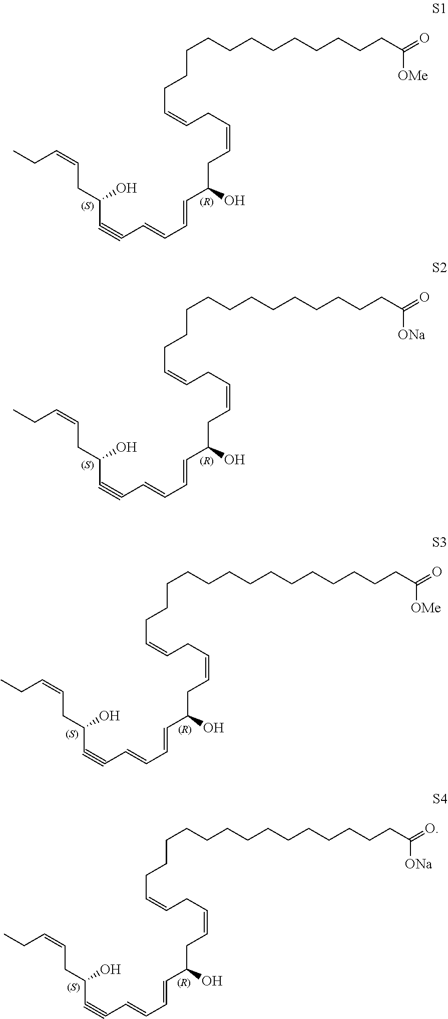

In some embodiments of this aspect of the disclosure, the composition can comprise equimolar amounts of the diastereomers S and T wherein the diastereomers have (S) or (R) chirality at the carbons bearing the hydroxyl groups.

In some embodiments of this aspect of the disclosure, the composition can comprise equimolar amounts of the diastereomers U and V wherein the diastereomers have either (S) or (R) chirality at position n-6, and (R) chirality at position n-13.

In some embodiments of this aspect of the disclosure, the composition can comprises one of the diastereomers of S or T in an amount exceeding the amount of the other diastereomer of S or T.

In some embodiments of this aspect of the disclosure, the composition can comprise one of the diastereomers of U or V in an amount exceeding the amount of the other diastereomer of U or V.

Other compositions, compounds, methods, features, and advantages of the present disclosure will be or become apparent to one having ordinary skill in the art upon examination of the following drawings, detailed description, and examples. All such additional compositions, compounds, methods, features, and advantages can be can included within this description, and be within the scope of the present disclosure.

Compositions and Methods for Modulating Elovanoid Bioactivity and Availability

Cellular senescence is a form of cell cycle arrest linked to aging and diseases. Cellular senescence is a proinflammatory cell fate associated with age-related diseases, including AD and AMD. The senescence phenotype expresses in cells undergoing terminal, replicative arrest that displays cell enlargement, chromatin alterations, SASP, and cell cycle regulatory proteins (cyclins and cyclin-dependent kinases). Persistent accumulation of senescence is associated with age-related diseases and functional decay. The clearance of senescent cells from tissue alleviates pathologies related to aging because they propagate degenerative and proinflammatory events in their microenvironment.

In the brain, the senescence signature program is triggered in astrocytes, microglia, and neurons (despite being post-mitotic). Senescent cell phenotype consequences (e.g., chronic inflammation) are also key in AMD. Cellular senescence is a defense event against cancer, and plays a role in aging and age-related diseases. Senescent cells help create a microenvironment that facilitates tumor progression. These events involve depletion of stem and progenitor cells and the cell deranging consequences of the expression of SASP, including proinflammatory homeostatic-perturbing cytokines and chemokines, growth factors, and matrix metalloproteinases.

Moreover, senescent cells have beneficial effects in injury repair and tissue remodeling as well. Thus, without being bound by theory, senescence, besides being a driver of age-dependent diseases and of metabolic syndrome, can remove healthy cells in addition to others that are damaging to the organ/organisms through senescent cell clearance. For example, a positive effect of senescent cells and SASP is the acceleration of skin wound healing by early secretion of SASP triggered by PDGF-AA, which is secreted by senescence cells. Wounding induces senescence in local fibroblasts and endothelial cells. As a consequence, myofibroblast differentiation, granulation tissue formation, and completion of wound healing takes place. ELVs modify expression (and protein abundance) of p16INK4a (also known as cyclin-dependent kinase inhibitor 2A, Cyclin-Dependent Kinase 4 Inhibitor A), a tumor suppressor protein. This protein is encoded by the Ink4a/Arf locus or Cdkn2a. p16 plays a role in cell cycle regulation by decelerating cell progression from the G1 phase to the S phase.

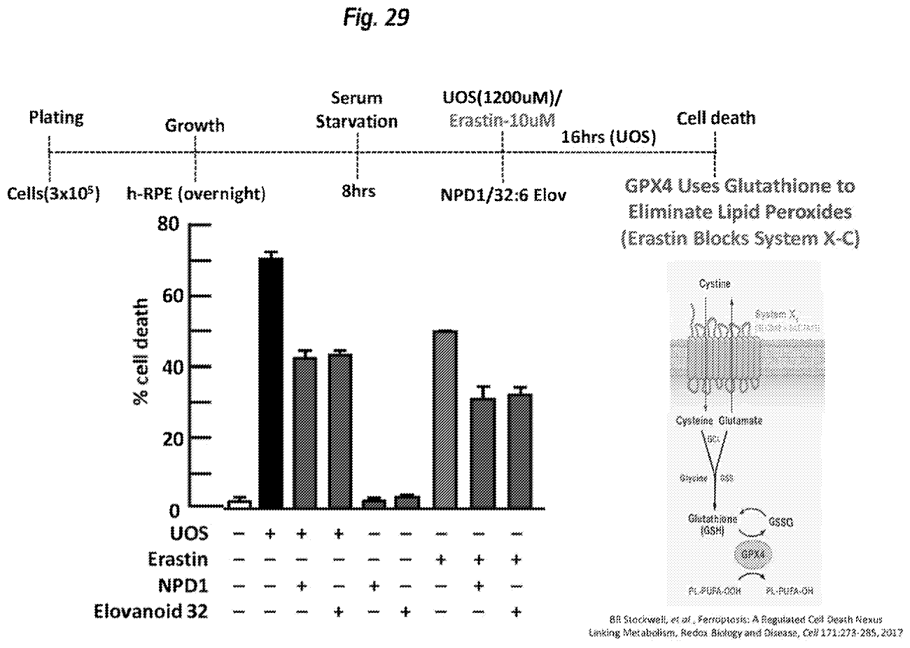

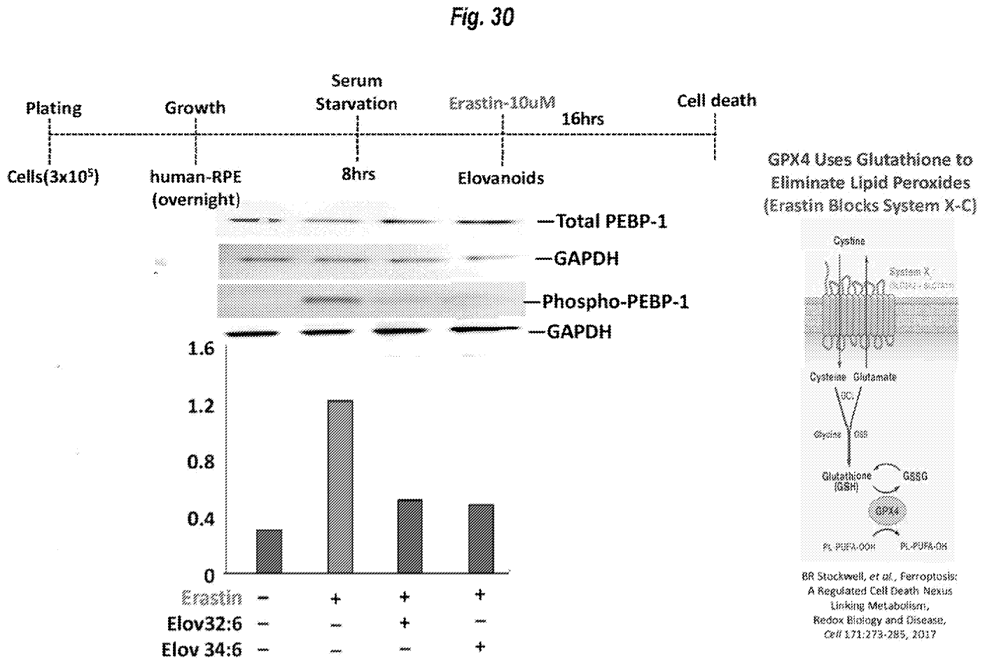

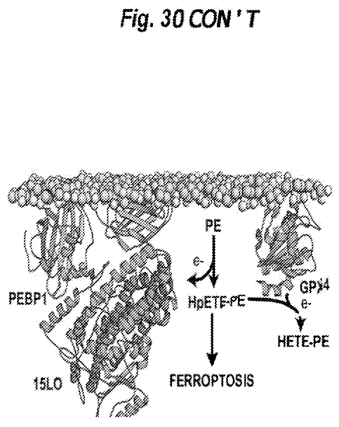

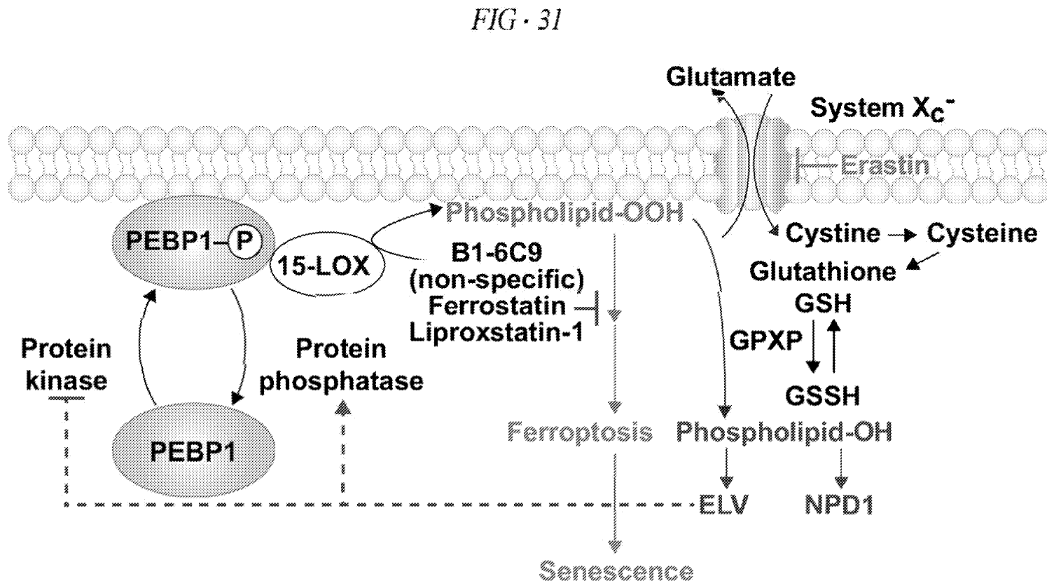

Referring to the figures, ELVs target upstream ferroptosis, a form of programed cell death that engages senescence. The inventors uncovered a new molecular target of ELVs that demonstrated inhibition of cell death by blocking phosphorylation of scaffold protein PEBP-1 (FIG. 29-31). As a result, peroxidized lipids are not formed, and ferroptosis is blocked. Iron, ferritin and markers of oxidative stress are enhanced in senescent cells. These cells display aberrant iron homeostasis and influence iron content in aging tissues. Iron itself induces senescence of microglia, whereas iron chelator decrease can reduce and prevent the accumulation of iron and ferritin of cellular senescence. Without wishing to be bound by theory, senescence-associated secretory phenotype (SASP) can drive ferritin expression in neurons and glia as an acute phase response, which can enhance its susceptibility to the iron-mediated cell death process, ferroptosis.

For example, the following are specific converging mechanisms:

Ferroptosis is at the interphase with autophagy and engages senescence (FIGS. 2-4)

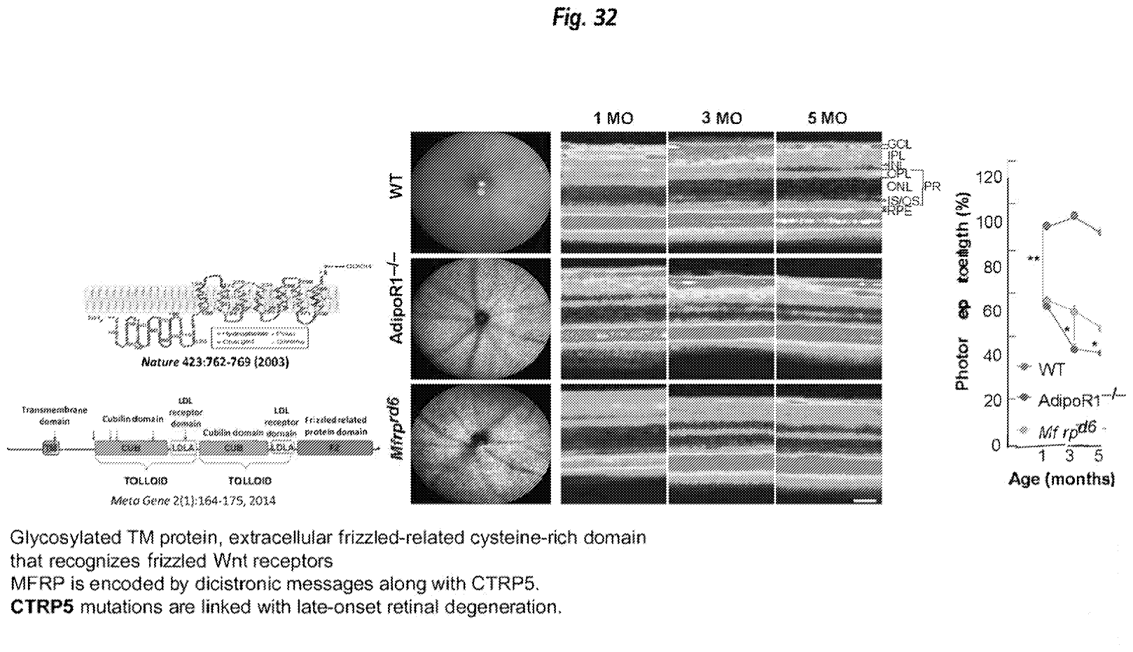

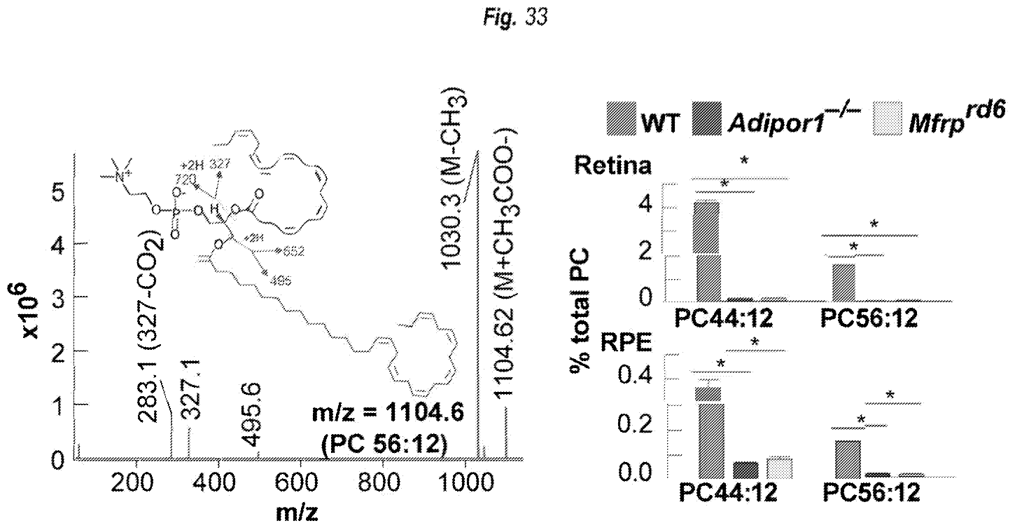

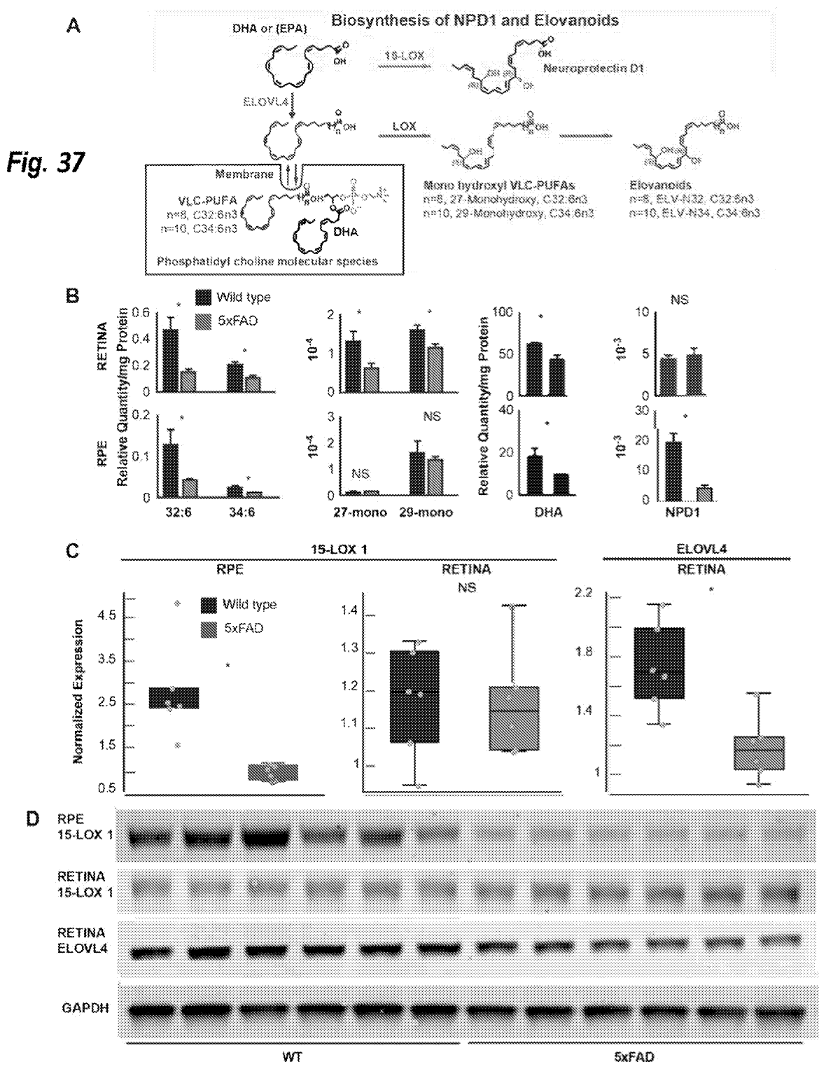

An AdipoR1 receptor subtype enhances DHA cellular uptake/retention and availability of the VLC-PUFAs' ELV precursors. After uptake/retention, this receptor subtype facilitates building in phosphatidylcholine of membrane reservoirs of the VLC-PUFAs that enter into that pathway of ELV biosynthesis upon release by a PLAL. The membranes containing VLC-PUFAs are released upon uncompensated oxidative stress (UOS) challenges, trauma, ischemia, and the onset of neurodegenerative diseases. 5×FAD on pathways failing before PRC death (FIG. 32 and FIG. 37).

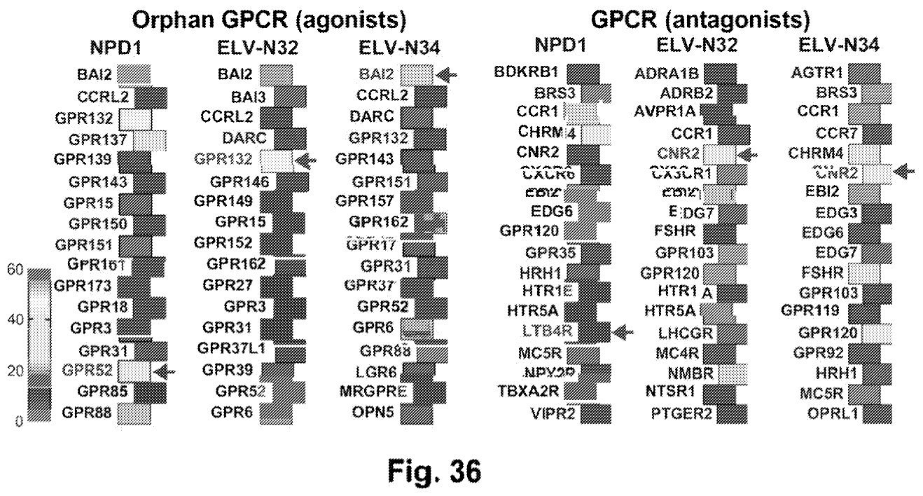

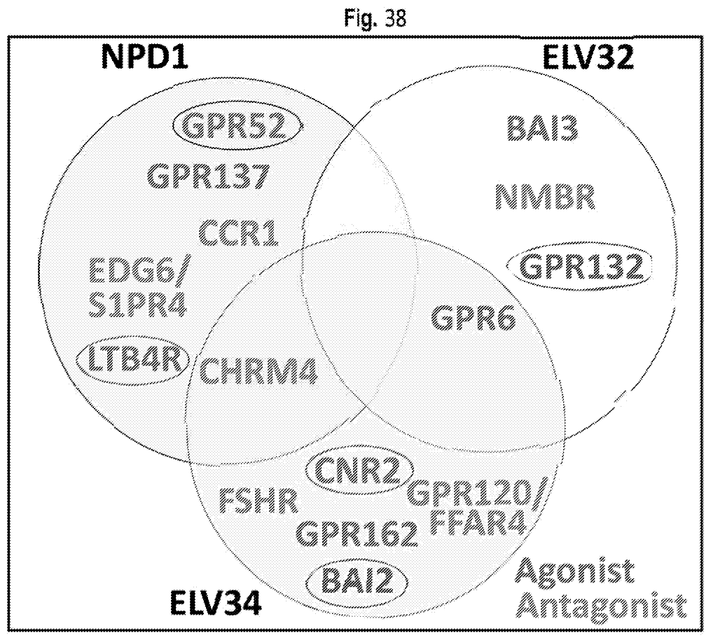

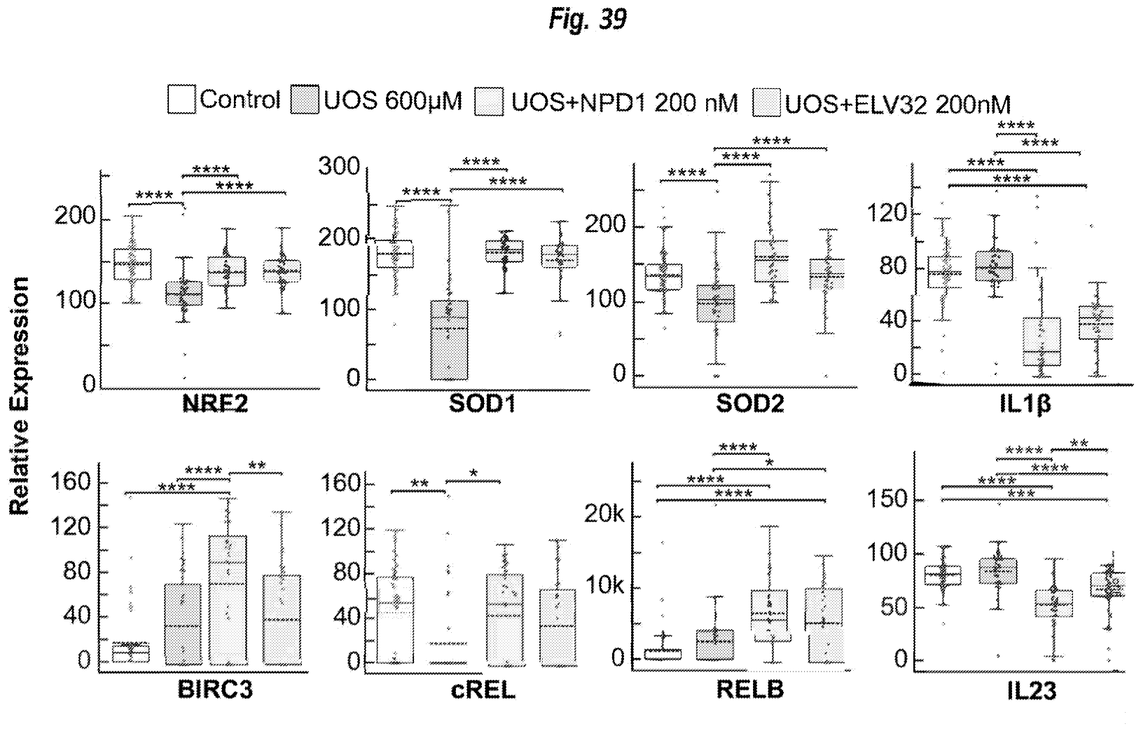

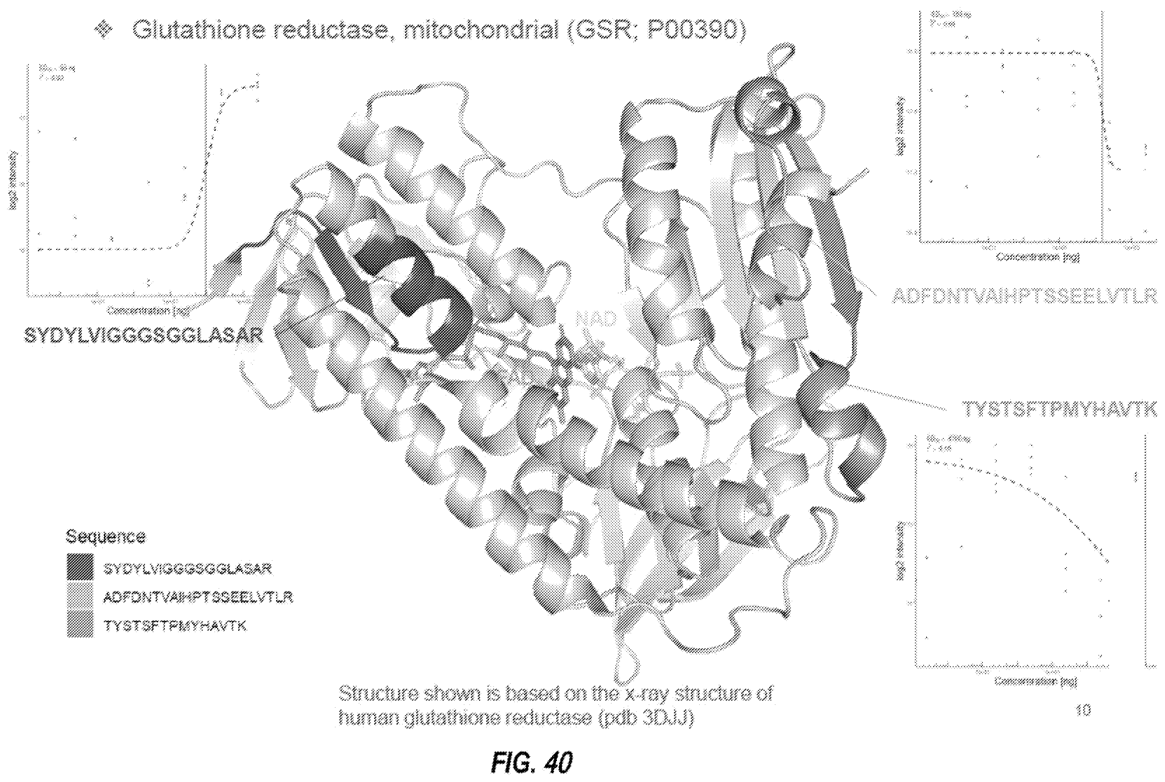

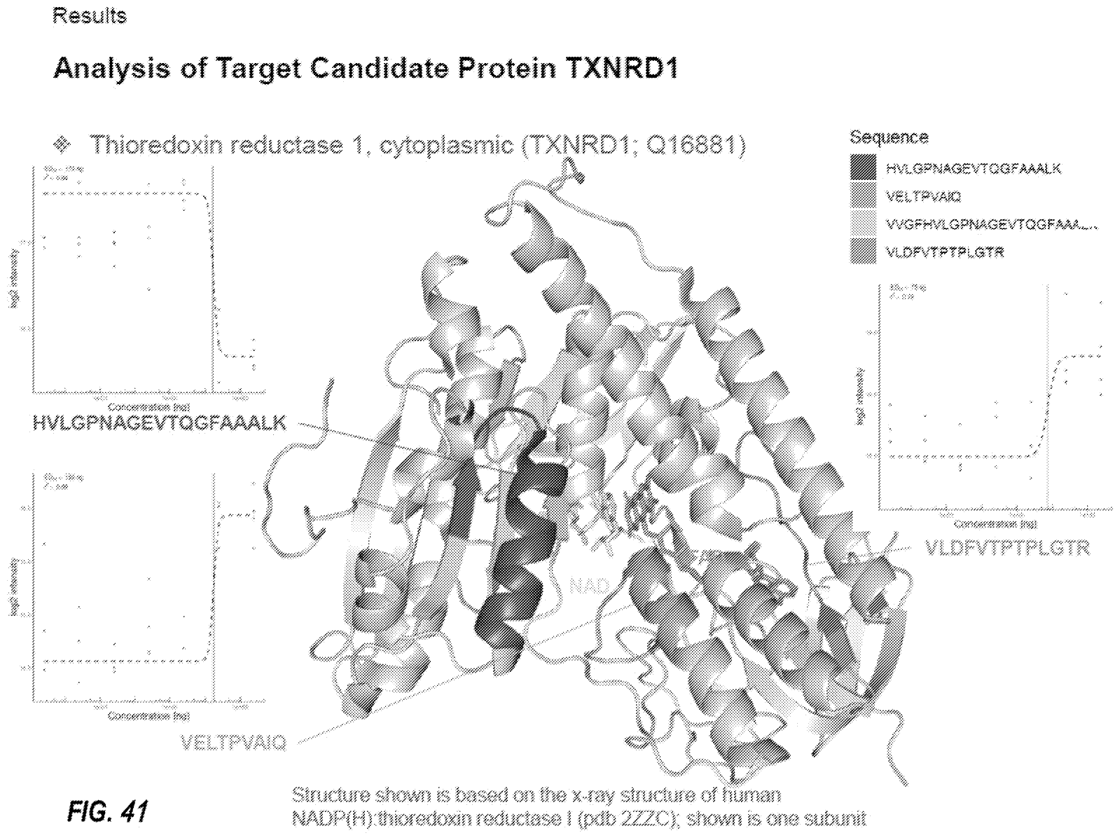

Specific GPCRs for ELV and NPD1 are the base to develop synthetic ligands (peptides small molecules or others) interaction of MFRP with AdipoR1 target to peptide that mimic ELV action by targeting specific receptor/s. GPCR data (FIG. 36 and FIG. 38).

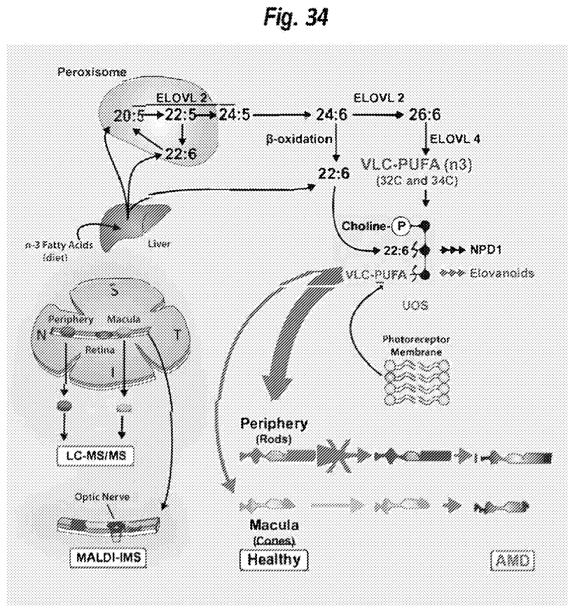

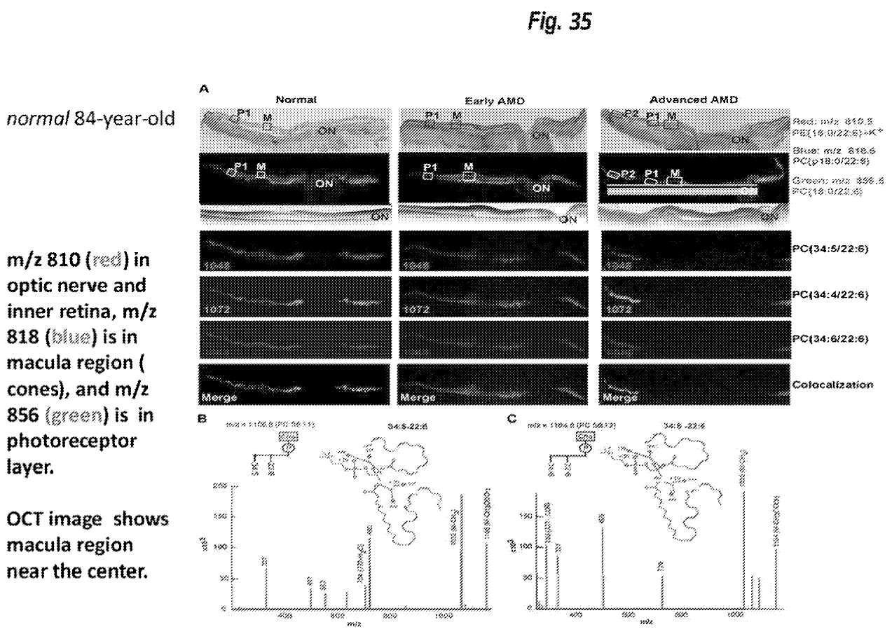

Intracellular proteins targeted by ELV as regulatory sites to enhance bioactivity. Identification of cell penetrant (or tissue penetrant) peptides or other small molecules that target GPCRs. Examples can include vivo MO-(contain bradykinin analog). Example of no toxicity to retina (FIG. 34).

Enzyme for signal termination by degrading ELV as a target for new, small molecules that would enhance the availability of ELV by blocking/attenuating its degradation.

ELVs beneficial role in GBM (see Figs).

TBI (see Figs)

Thus, embodiments of the invention comprise compositions and methods that result in the elimination of senescent cells. For example, embodiments of the invention are drawn to compositions and methods that modulate availability of senescence cells in cancer (chemotherapy, brain (neurodegenerative diseases)), wound healing (diabetes, cornea-keratinocytes, decubital ulcers), and neurodegenerative diseases, such as AMD and AD.

Embodiments of the invention are also drawn to compositions and methods that are neuroprotective and/or neurorestorative. For example, embodiments are neuroprotective in prodromal targeting of MCA and/or sight disturbances preceding blindness in AMD or retinitis pigmentosa (RP) or other retinal degenerative diseases. Further, embodiments are neuroprotective or neurorestorative in other diseases, such as AD, AM, CV diseases, metabolic syndrome, obesity, type 2 diabetes, myocardial infarction, stroke, TBI, GBM. In cancer, for example, senescent cells help create a microenvironment that facilitates tumor progression.

Aspects of the invention are drawn to a set of converging mechanisms of elovanoids (ELVs) that regulate ferroptosis and senescence and have beneficial consequences in diseases. For example, aspects of the invention are drawn to compositions and methods for preventing, treating, ameliorating the symptoms of, or slowing the progression of cancer (such as glioblastoma multiform or GBM), age-related macular degeneration (AMD), Alzheimer's disease (AD), other neurodegenerative diseases, metabolic syndrome, obesity, type 2 diabetes, neurotrauma, skin and corneal wound healing.

As used herein, the term “senescence” can refer to a halt in cell division. In embodiments, the halt can be permanent or temporary. As used herein, the term “senolytic” can refer to an agent that can kill or suppress a senescent cell. For example, the senolytic agent can induce the death of at least 10%, 15%, 20%, 25%, 30%, 35%, 40%, 45%, 50%, 55%, 60%, 65%, 70%, 75%, 80%, 85%, 90%, 95%, or more of a population of senescent cells, in vitro or in vivo. In embodiments, the senolytic can induce apoptosis of a senescent cell. In embodiments, the senolytic can suppress expression of senescence promoting genes.

In embodiments, aging is indicated by the presence or absence of senescent cells, the presence or absence of inflammation, or both.

The term “senescent cell” can refer to a cell that has lost the ability to divide. A cell can stop dividing due to a variety of factors such as, for example, aging, cellular damage, DNA damage, and/or toxins. A senescent cell can be a cultured cell or a cell in the body, in vivo. For example, embodiments of the invention can reduce the number or activity of senescent cells. For example, embodiments of the invention can reduce the number of senescent cells by at least 10%, 15%, 20%, 25%, 30%, 35%, 40%, 45%, 50%, 55%, 60%, 65%, 70%, 75%, 80%, 85%, 90%, 95%, or more after administration.

The term “inflammation” can refer to the term for local accumulation of fluids, plasma proteins, and/or white blood cells initiated by an autoimmune response, physical injury, infection, vascular disease, chemical exposure, radiation or a local immune response. Inflammation can be characterized by one or more signs, including, for example, redness, pain, heat, swelling and/or loss of function. Inflammation can be associated with chronic (long term) inflammatory diseases or disorders or acute (short term) inflammatory diseases or disorders. For example, the presence or absence of pro-inflammatory cytokines and chemokines are indicative of aging. Non-limiting examples of pro-inflammatory cytokines and chemokines include pro-inflammatory cytokines and chemokines comprise at least one of IL-6, IL-1β, IL-8/CXCL8, CCL2/MCP-1, CXCL1/KC/GRO, VEGF, ICAM1(CD54).

The elovanoids used in embodiments herein are described in PCT/US2016/017112, PCT/US2018/023082, each of which are incorporated by reference herein in their entireties. For example, the elovanoid comprises ELV-N-34 or ELV-N-32, or derivatives thereof. Lipoxygenation of n-3-VLC-PUFA, 3 leads to the formation of enzymatically-hydroxylated derivatives of n-3-VLC-PUFA, termed elovanoids, which can include monohydroxy compounds (e.g. ELV-27S and ELV-29S, 4, and dihydroxy derivatives, e.g. ELV-N-32 and ELV-N-34, 5. Elovanoid ELV-N-32 is the 20R,27S-dihydroxy 32:6 derivative (32-carbon, 6 double bond elovanoid with a neuroprotectin-like 20(R),27(S)-dihydroxy pattern). Elovanoid ELV-N-34 is the 22R,29S-dihydroxy 34:6 derivative (34-carbon, 6 double bond elovanoid with a 22(R),29(S)-dihydroxy pattern).

Peptide Analogs

Further, aspects of the invention are drawn to the development of new synthetic non-lipidic analogs to mimic the bioactivity of the lipid mediators, such as elovanoids. The term “analog” can refer to a second organic or inorganic molecule which possesses a similar or identical function as a first organic or inorganic molecule. The analog can be structurally similar to the first organic or inorganic molecule. In embodiments, the first molecule is a lipid, and the second molecule (i.e., analog) is a non-lipidic molecule. For example, the first molecule is an elovanoid, and the second molecule is a peptide. In such embodiments, the peptide can be referred to as a “peptide analog”.

In embodiments, the peptide analog can be considered a therapeutic peptide. The term “therapeutic peptide” can refer to a peptide or fragment or variant thereof having one or more therapeutic and/or biological activities.

The term “peptide” can refer to a molecule comprising two or more amino acid residues linked together by a peptide bond. These terms can include, for example, natural and artificial proteins, protein fragments of protein sequences and polypeptide mimetics (such as muteins, variants, and fusion proteins) as well as post-translationally or otherwise covalently or non-covalently modified peptides. The peptide may be monomeric or polymeric. In certain embodiments, a “peptide” is a chain of amino acids in which the alpha carbon can be linked through a peptide bond. The terminal amino acid at one end (amino terminus) of the chain thus has a free amino group, while the terminal amino acid at the other terminus (carboxy terminus) of the chain has a free carboxyl group. As used herein, the term “amino terminal” (abbreviated N-terminal) can refer to the free amino group on the amino acid at the amino terminus of the peptide or the Amino group of the amino acid at any other position in the peptide. Similarly, the term “carboxy terminus” can refer to the free carboxyl group on the carboxy terminus of the peptide or the carboxyl group of the amino acid in any other position within the peptide. Peptides also can include essentially any polyamino acids, including, but not limited to, amino acids linked by an ether as opposed to a peptide mimetic, such as an amide bond.

In embodiments, the peptide analogs are peptides comprising at least four amino acids linked through peptide bonds or other covalent bonds as described herein. In an embodiment, the peptide or peptide analog is from about 4 to about 50 amino acids in length. All integer subranges of 4 to 50 amino acids are useful for the peptides herein. In an embodiment, the peptide or peptide analog is an amino acid of about 5 to about 35 amino acids, about 5 to about 30 amino acids in length, about 5 to about 25 amino acids in length, or about 5 to about 20 amino acids in length to be. In an embodiment, the peptide or peptide analog is an amino acid of about 6 to about 35 amino acids, about 7 to about 30 amino acids in length, about 6 to about 25 amino acids in length, or about 6 to about 20 amino acids in length to be. In an embodiment, the peptide or peptide analog is an amino acid of about 7 to about 35 amino acids, about 7 to about 30 amino acids in length, about 7 to about 25 amino acids in length, or about 7 to about 20 amino acids in length to be. In an embodiment, the peptide or peptide analog is an amino acid of about 8 to about 35 amino acids in length, about 8 to about 30 amino acids in length, about 8 to about 25 amino acids in length, or about 8 to about 20 amino acids in length to be. In an embodiment, the peptide is an amino acid of about 8 to about 17 or 18 or about 9 to about 16 or 17 amino acids in length. In an embodiment, the peptide is from about 10 to about 17 or about 12 to about 16 or 17 or about 14 to about 16 amino acids in length. In some embodiments, the peptide is selected from the group consisting of 5-mer, 6-mer, 7-mer, 8-mer, 9-mer-10-mer, 16-mer, 17-mer, 18-mer, 19-mer, or 20-mer.

In embodiments, the elovanoid and/or the peptide analog can regulate/modulate ferroptosis, wherein modulation of ferroptosis treats or prevents a disease in a subject. As used herein, “ferroptosis” means regulated cell death that is iron-dependent. Ferroptosis is characterized by the overwhelming, iron-dependent accumulation of lethal lipid reactive oxygen species. Ferroptosis is distinct from apoptosis, necrosis, and autophagy. Assays for ferroptosis are as disclosed, for instance, in Dixon et al., 2012.

In other embodiments, the elovanoid and/or the peptide analog can regulate/modulate cellular senescence, wherein modulation of cellular senescence treats a disease in a subject.

The terms “modulate”, “modulating” and grammatical variations thereof mean to change, such as increasing or decreasing a biological activity.

Molecular Targets

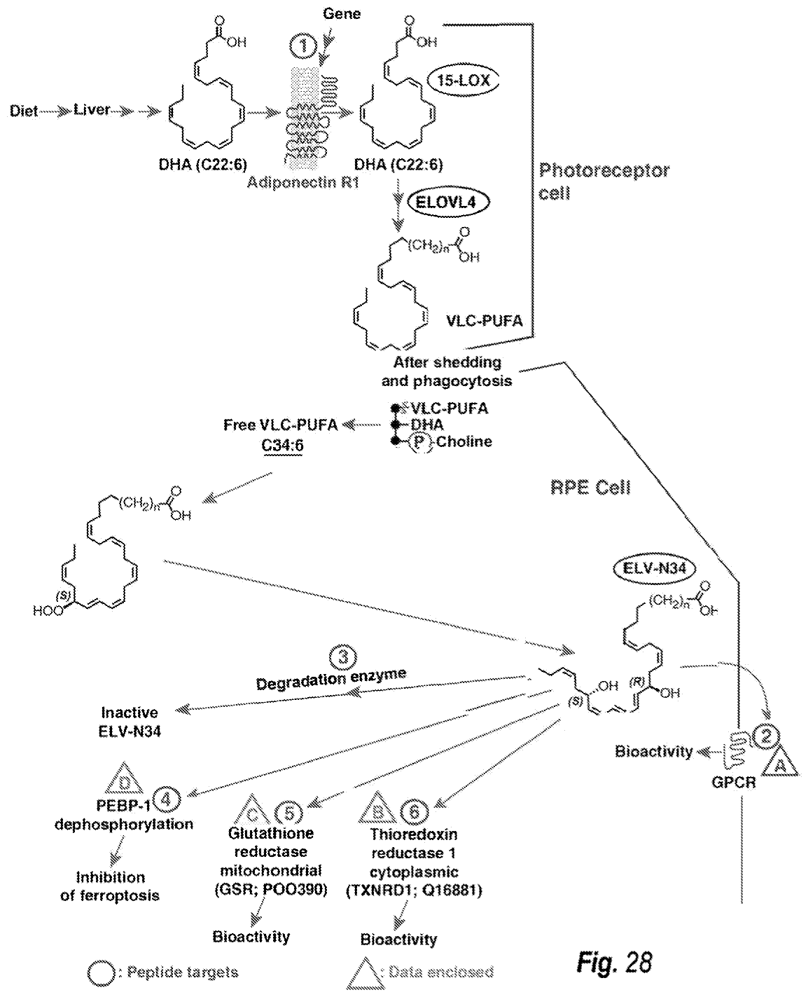

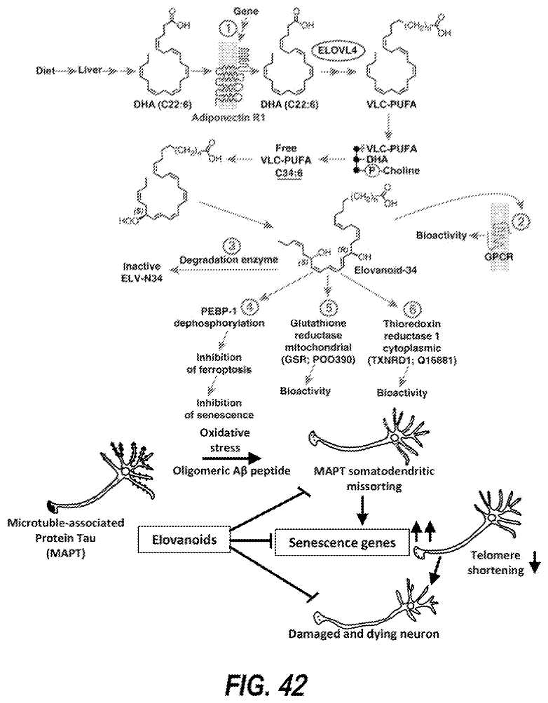

In embodiments, the elovanoid and/or peptide analog can bind to an epitope on one or more of the molecular targets, such as those as identified in FIG. 28 and FIG. 42.

In embodiments, the elovanoid and/or peptide analog can bind to an epitope on one or more of the molecular targets as identified in the table below (for example, to a contiguous or non-contiguous amino acid sequence depicted by the protein NCBI reference number listed in the Table below):

| | human | | | | |
|---|---|---|---|---|---|
| | | | gene | protein | protein |
| | GPCR | mRNA | ID | uniprot | NCBI |
| NPD1 | LTB4R | NM_001143919.3 | 1241 | Q15722 | NP_001137391.1 |
| | GPR37 | NM_00502.5 | 2861 | O15354 | NP_005293.1 |
| | GPR52 | NM_005684.5 | 9293 | P0C5J4 | NP_005675.3 |
| ELV32 | GPR132 | NM_001278696.2 | 29933 | Q9UNW8 | NP_037477.1 |
| ELV34 | CNR2 | NM_001841.3 | 1269 | P34972 | NP_001832.1 |
| | BAI2 | NM_001364857.1 | 576 | O60241 | NP_001281264.1 |
| | Interacting | | | | |
| | proteins | | | | |
| ELV34 | TXNRD1 | NM_001093771.3 | 7296 | Q16881 | NP_877393.1 |
| | PEBP1 | NM_002567.4 | 5037 | P30086 | NP_002558.1 |
| | PTBP1 | NM_002819.5 | 5725 | P26599 | NP_002810.1 |
| ELV34 | GSR | NM_001195104.2 | 2936 | P00390 | NP_000628.2 |
| | (mithochondrial) | | | | |

In embodiments, the elovanoids and the peptide analogs interact with the same or similar epitope on the molecule target. In embodiments, the elovanoids and the peptide analogs can interact with different epitopes on the molecular target.

As used herein, the term “epitope” can refer to a portion of the molecular target, such as those identified in the Table can included herein, to which an elovanoid and/or peptide analog specifically binds.

For example, in embodiments, an elovanoid or peptide analog thereof can target an epitope on LTB4R, GPR37, GPR52, GPR132, CNR2, BAI2, TXNRD1, PEBP1, and/or GSR to modulate cellular senescence and/or ferroptosis. In embodiments, the epitope can include a “target site” or “target sequence”, which can refer to a sequence that is bound by the binding partner, such as the elovanoid or the peptide analog. For example, the target site can comprise one or more amino acids.

The proteins in Table herein can be referred to as “molecular targets”. The term “target” or “molecular target” can refer to any molecule, for example a molecule within a cell or associated with a cell membrane, that is being examined for interaction with a candidate compound (e.g., a drug, an elovanoid, or a peptide analog thereof). Non-limiting examples of molecular targets can include DNA, RNA and proteins such as receptors (e.g., cell surface, membrane-bound or nuclear), components of signal transduction pathways, transcription factors or functional fragments thereof. A molecular-targets can also comprise a macromolecule such as a protein, nucleic acid, carbohydrate, lipid, glycoprotein, lipoprotein, polysaccharide, any modified derivative of the molecules described herein, or any complex comprising one or more of the molecules described herein. A compound, elovanoid, or peptide analog “interacts” with a molecular target when it affects, directly or indirectly, the molecular target. The compound can act directly on the molecular target, for example when the molecular target is a protein, the compound may directly interact with the protein by binding to it or may directly regulate expression of the protein via action on transcriptional regulatory elements. Similarly, the compound can also act indirectly on the molecular target, for example by blocking or stimulating a separate molecule that in turn acts on the molecular target. Indirect action of a compound on a molecular target can occur, for example, when the target is a non-protein molecule and the compound interacts with a protein involved in the production, stability, activity, maintenance and/or modification of the non-protein molecular target.

The term “binding” can refer to the determination by standard assays, including those described herein, that a binding polypeptide recognizes and binds reversibly to a given target. Such standard assays can include, but are not limited to, equilibrium dialysis, gel filtration, and the monitoring of spectroscopic changes that result from binding.

In embodiments, the elovanoid or peptide analog can have specificity for a molecular target in Table 1. The term “specificity” can refer to a binding polypeptide having a higher binding affinity for one target over another. Binding specificity may be characterized by a dissociation equilibrium constant (K_{D}) or an association equilibrium constant (Kₐ) for the two tested target materials.

### EXAMPLES

### Example 1—Primary Human Nasal Epithelial Cells (HNEpC) as Used in Examples Herein

Cryopreserved human nasal epithelial cells HNEpC were purchased from PromoCell GmbH, Heidelberg, Germany. (Catalog #C-1260, Lot #436Z028).

The cells used for our experiments are primary nasal epithelial cells obtained from the nasal mucosa of a 50-year-old Caucasian male.

Cells were received in passage (P1) and were sub-culture to passage (P3), which were used for all the experiments.

HNEpC were grown to 80% confluency in Promocell's Airway epithelial cell growth medium (Catalog #C-21060) which was supplemented with airway epithelial cell growth medium supplement pack (Catalog #C-39160) and Penicillin/Streptomycin.

### Example 2—HNEpC were Challenged Using Several Stressors—Aeroallergens

Lipopolysaccharide (LPS) from Escherichia coli serotype 0111:B4 (Catalog #L4391) were obtained from Sigma-Aldrich. LPS is the principal component of Gram-negative bacteria that activates the innate immune system through its recognition by Toll-like receptor 4 (TLR4). This leads to a signaling cascade that ultimately results in the activation of NF-κB and the production of proinflammatory cytokines. LPS we used for our experiments is a preparation of smooth (S)-form LPS purified from the Gram-negative E. coli 0111:B4 that was used at 30 μg/mL to challenge HNEpC.
Polyinosinic-polycytidylic acid (abbreviated as poly(I:C) or poly(rI):poly(rC)) is a synthetic analog of double-stranded viral RNA (dsRNA), a molecular pattern associated with viral infection such as loss of epithelial integrity, increased production of mucus and inflammatory cytokines. Poly(I:C), a TLR3 agonist activates the antiviral pattern recognition receptors TLR3, RIG-I/MDA5 and PKR, thereby inducing signaling via multiple inflammatory pathways, including NF-κB and IRF. High Molecular Weight Poly(I:C) comprises long strands of inosine poly(I) homopolymer annealed to strands of cytidine poly(C) homopolymer. The average size of Poly(I:C) HMW is from 1.5 kb to 8 kb. Poly(I:C)(Catalog #P1530) were obtained from Sigma-Aldrich and used at 100 μg/mL to challenge HNEpC.
House Dust Mite extract from Dermatophagoides pteronyssinus (D.P.) (Catalog #3033)—pure lyophilized extract were obtained from Chondrex, Inc.
D.P. was used at 30 μg/mL to challenge HNEpC. Allergen—Der p1, Der p2.
House Dust Mite extract from Dermatophagoides farinae (D.F.) (Catalog #3040)—pure lyophilized extract were obtained from Chondrex, Inc.
D.F. and used at 30 μg/mL to challenge HNEpC. Allergen—Der f1, Der f2
House Dust Mite extract—HDM a mixture of both (D.P.) & (D.F.) was used at (15 g/mL+15 μg/mL) to challenge HNEpC.

### Example 3—(HNEpC) were Challenged Using Several Stressors (Aeroallergans) and the Following Assays were Performed

LDH cytotoxicity assay—using CyQuant LDH Cytotoxicity Assay Kit from Invitrogen (Catalog #C20301).
Cell viability assay—using PrestoBlue HS Cell Viability Assay Kit from Invitrogen (Catalog #C50201).
Sandwich ELISA assays from Chondrex, Abcam and R&D Systems:
1) Human IL-6 detection Kit from Chondrex (Catalog #6802)
2) Human IL-1β detection Kit from Chondrex (Catalog #6805)
3) Human IL-8/CXCL8 Quantikine ELISA Kit from R&D Systems (Catalog #D8000C)
4) Human CCL2/MCP-1 detection Kit from Chondrex (Catalog #6821)
5) Human CXCL1/KC/GRO detection Kit from Chondrex (Catalog #6825)
6) Human VEGF detection Kit from Chondrex (Catalog #6810)
7) Human ICAM1(CD54) ELISA Kit from Abcam (Catalog #ab100640)
8) Human IL-10 detection Kit from Chondrex (Catalog #6806)

### Example 4—Cell Viability Assay Using Presto Blue HS Reagent

The PrestoBlue HS Cell Viability Reagent is a complete add and read, nontoxic reagent that does not require cell lysis. The highly purified resazurin that is used for PrestoBlue HS results in a reagent with a >50% decrease in background fluorescence and a >100% increase in the signal to background ratio.

On entering live cells, the cellular reducing environment reduces resazurin to resorufin a compound that is red and highly fluorescent.

Viable cells continuously convert resazurin to resorufin increasing the overall fluorescence and color of the media surrounding the cells. Also, the conversion of resazurin to resorufin results in a pronounced color change, therefore cell viability can be detected using absorbance-based plate readers.

Fluorescence is read using a fluorescence excitation wavelength of 560 nm (excitation range is 540-570 nm) and an emission of 590 nm (emission range is 580-610-nm).

### Example 5—Conclusion

Results of the cytotoxicity assay (LDH) shows that upon addition of the stressors—LPS, poly(I:C), HDM extracts—there is pronounced increase in the formation of red formazan indicating cytotoxicity, which are reduced by the addition of ELVs. (FIG. 17A and FIG. 17B).

Cell viability assay using PrestoBlue HS reagent also shows more resorufin production in control cells as compared to cells challenged with the different stressors—LPS, poly(I:C), HDM extracts; addition of ELVs increases cell viability and gives protection to the HNEpC (FIG. 18A and FIG. 18B).

When HNEpC were challenged with the different stressors—LPS, poly(I:C), HDM extracts, there is a pronounced production of pro-inflammatory cytokines and chemokines—IL-6, IL-1β, IL-8/CXCL8, CCL2/MCP-1, CXCL1/KC/GRO, VEGF, ICAM1(CD54) compared to controls. This increased production of pro-inflammatory cytokines and chemokines are abrogated by the addition of ELVs at a concentration of 500 nM, 30 min post challenge with the respective stressor (FIG. 19A and FIG. 19B).

Conversely, when HNEpC were challenged with the different stressors—LPS, poly(I:C), HDM extracts, there is a pronounced decrease in the release of anti-inflammatory cytokine—IL-10 compared to controls. This decreased production of anti-inflammatory cytokine are reversed by the addition of ELVs at a concentration of 500 nM, 30 min post challenge with the respective stressor (FIG. 26A and FIG. 26B).

### Example 6—Elovanoids for Allergic Rhinitis, Allergic Conjunctivitis, Allergic Dermatitis and Asthma

The following are the experimental conditions that trigger inflammation/allergy in the human nasal mucosa (in primary culture) and that elovanoids contracted, protecting the integrity of these cells.
a) Polyinosinic-polycytidylic acid (poly(I:C) or poly(rI):poly(rC)), a synthetic analog of double-stranded viral RNA (dsRNA), a molecular pattern associated with viral infection such as loss of epithelial integrity, increased production of mucus and inflammatory cytokines;
b) LPS, the principal component of Gram-negative bacteria that activates the innate immune system through its recognition by Toll-like receptor 4 (TLR4). This leads to a signaling cascade that ultimately results in the activation of NF-κB and the production of proinflammatory cytokines. The LPS that we used for our experiments is a preparation of smooth (S)-form LPS purified from the Gram-negative E. coli 0111:B4 that was used at 30 μg/mL to challenge HNEpC;
c) House Dust Mite extract from Dermatophagoidespteronyssinus (D.P.) (Catalog #3033)—pure lyophilized extract were obtained from Chondrex, Inc. D.P. was used at 30 μg/mL to challenge HNEpC. Allergen—Der p1, Der p2;
d) House Dust Mite extract from Dermatophagoides farina (D.F.) (Catalog #3040)—pure lyophilized extract were obtained from Chondrex, Inc. D.F. and used at 30 μg/mL to challenge HNEpC. Allergen—Der f1, Der f2;
e) House Dust Mite extract—HDM a mixture of both (D.P.) & (D.F.) was used at (15 μg/mL+15 μg/mL) to challenge HNEpC.

Allergy treatments for itchiness, difficulty breathing, etc., remain inconsistently effective with many experiencing drowsiness, dry mouth, and other side effects that make daily functioning difficult. Elovanoids can be delivered intranasally to treat allergic rhinitis, allergic conjunctivitis, allergic dermatitis and asthma, halting these conditions in their tracks, providing an effective alternative to most over-the-counter medications as well as their inhibiting side effects.

Results of the cytotoxicity assay (LDH) shows that upon addition of the stressors—LPS, poly(I:C), HDM extracts—there is pronounced increase in the formation of red formazan indicating cytotoxicity, which are reduced by the addition of ELVs. (FIG. 17A and FIG. 17B).

Cell viability assay using PrestoBlue HS reagent also shows more resorufin production in control cells as compared to cells challenged with the different stressors—LPS, poly(I:C), HDM extracts; addition of ELVs increases cell viability and gives protection to the HNEpC (FIG. 18A and FIG. 18B).

When HNEpC were challenged with the different stressors—LPS, poly(I:C), HDM extracts, there is a pronounced production of pro-inflammatory cytokines and chemokines—IL-6, IL-10, IL-8/CXCL8, CCL2/MCP-1, CXCL1/KC/GRO, VEGF, ICAM1(CD54) compared to controls. This increased production of pro-inflammatory cytokines and chemokines are abrogated by the addition of ELVs at a concentration of 500 nM, 30 min post challenge with the respective stressor (FIG. 19A and FIG. 19B).

Conversely, when HNEpC were challenged with the different stressors—LPS, poly(I:C), HDM extracts, there is a decrease in the release of anti-inflammatory cytokine—IL-10 compared to controls. This decreased production of anti-inflammatory cytokine are reversed by the addition of ELVs at a concentration of 500 nM, 30 min post challenge with the respective stressor (FIG. 26A and FIG. 26B).

### Example 7

(1) can be a target in prodromal conditions before disease is evident. Structure and function of the 5×FAD retina. (A) V log I plot showing maximum ERG b-wave amplitudes for light flashes from 0 to 0.075 cd·s/m2. The 5×FAD mice achieved maximum amplitude of about 100 μV, approximately half that recorded for the wild-type mice. (B) Electron microscopy of 5-month-old 5×FAD retinas illustrating morphological similarity to the wild-type retina. i. Basal side of a 5×FAD RPE cell showing membrane infoldings along Bruch's membrane (Br). ii. Disk synthesis region (arrow) at the basal portion of a wild-type rod outer segment showing newly formed disks from the connecting cilium (CC) membrane. iii. Similar region in a 5×FAD retina showing new disk formation (arrow). iv. The outer limiting membrane (OLM, arrow) at the scleral edge of the cell body layer (N, photoreceptor nucleus) in the 5×FAD retina. The cytoplasm of the Müller cells (M) are lighter than that of the photoreceptors (PR). v. The interface between a 5×FAD RPE cell and a rod photoreceptor tip (PR). Two phagosomes (Ph) are visible just within the RPE cytoplasm; the lower Ph is held within the RPE apical processes, while the upper, darker Ph is older and just entering the RPE cell body, illustrating normal phagocytic function. vi. Inner segment mitochondria (M) of 5×FAD retinas retain the very elongate form of healthy photoreceptors. (C) Five-month-old wild-type and 5×FAD retinal sections illustrating normal photoreceptor profiles within the 5×FAD retina. (D) Fluorescent staining of the retina from WT and 5×FAD. The blue (DAPI) is the nuclei and red Aβ in the RPE layer at 6 months old in 5×FAD. (*P<0.05, using student t-test comparison).

### Example 8

ELVs restore RPE morphology and reduce gene expression after subretinal injection of OAβ in WT mice. (A) Mice were divided into 7 groups: non-injected, PBS, OAβ only, OAβ+ELV-N-32, OAβ+ELV-N-34, ELV-N-32 only and ELV-N-34 only. On day 3, mRNA were isolated for RT-PCR. On day 7, mice were subjected to OCT and then eyes were enucleated and processed to whole mount RPE staining and Western blot. (B) Whole flat mount of RPE. OAβ disrupted RPE morphology. However, RPE were less damage in the ELVs treatment group as well as PBS, ELVs alone did non induce changes. (C) Evaluation the OAβ effects on retina and RPE by OCT. (D) The thickness of PRC was thin in the OAβ injected group. OAβ cause the cell death of PRC, as the thinner in OCT measurement. (E) RPE gene expression after OAβ (1-42) injection and treatment with ELVs. 3 days after injection, the RNAs from RPE were isolated, reverse transcribed into cDNA and subjected to RT-PCR with different primers. Genes in the same functional group were plotted in the same chart, including senescence- and AMD-related genes (E), and collagenases, gelatinase, stromelysins and others matrix metalloproteinases (MMP) (F) and autophagy (G). (H) p16INK4a western blots of RPE/Choroid. ELV-N-32 and ELV-N-34 down regulated the expression of the key senescence marker, p16INK4a, which was elevated by OAβ injection. (I) Retina gene expression after OAβ (1-42) injection and treatment with ELVs. OAβ activates apoptosis genes in the retina. With ELVs co-injection, these genes were down-regulated. (*P<0.05, using student t-test comparison).

### Example 9

OAβ toxicity is counteracted by ELVs in primary hRPE. (A) Primary hRPE cells were treated with 10 μM OAβ with or without adding ELVs. After 3 days, the total RNA was isolated and q-PCR analyzed. After 7 days, cells were subjected to β-Galactosidase staining. (B) Live cell images of primary hRPE under bright field microscope imaging after 7 days. (C) β-Galactosidase staining of primary hRPE, +/−ELVs. Quantitation of % for the j-Gal positive cells. ELVs decreased positive senescent cells. (D) Transcription of senescence genes, AMD-related genes and autophagy genes in primary hRPE under OAβ (1-42) exposure and treatment with ELVs. (*P<0.05, using student t-test comparison).

### Example 10

Working model of ELVs in OAβ-induced RPE and PRC damage. (A) OAβ induces senescence and disrupts the tight junction of RPE. Next, OAβ penetrates the retina, causing cell death of photoreceptors reflected in less cell body layer (CBL) nuclei. The Elovanoids restore the morphology of the RPE layer upon OAβ exposure and, as a consequence, the retina structure is preserved. (B) OAβ induces the senescence, autophagy, matrix metalloproteinases, and AMD-related genes in the RPE and apoptosis genes in retina. ELVs downregulated the OAβ-gene inductions. Pathways for the synthesis of ELVs are depicted.

### Example 11—Downregulation of Senescence Programming in the Hypothalamus and Adipose Tissue by Elovanoids Counteracts Diabetes Onset and Progression

1) Research Plan

a) Specific Aims

The incidence of type 2 diabetes (a consequence of obesity) is rapidly increasing, for example during aging, and is a risk factor for kidney dysfunction, cardiovascular disease, stroke, impaired wound healing, infections, depression, anxiety, and cognitive decline. Despite advances in our understanding of the pathogenesis of diabetes, metabolic syndrome, and comorbidities, there is no effective therapy available. Cellular senescence has been implicated in age-related chronic inflammatory diseases, including metabolic syndrome (hypertension, obesity, and atherosclerosis), in the pathogenesis of type 2 diabetes by targeting pancreatic beta cell function and by triggering adipose tissue dysfunctions. Senescent programming forms a diabetes loop—the cause and consequence of cellular dysfunctions. This new class of lipid mediators, the Elovanoids (ELVs), will be studied as down regulators of senescence programming to counteract diabetes onset and progression. We offer herein validating a new therapeutic approach utilizing specific new compounds supported by compelling evidence in experimental models of diabetes and by our recent discovery of a mechanism.

ELVs are dihydroxylated derivatives of the very long-chain polyunsaturated fatty acids (VLC-PUFAs) 32:6n-3 and 34:6n-3. As precursors of ELVs, VLC-PUFAs are biosynthesized by elongation of a 22:6n-3 fatty acid and catalyzed by ELOVL4 (elongation of very-long-chain fatty acids-4). Our lab reported the discovery of the ELVs, including their detailed structure and stereochemistry, as established by stereocontrolled total organic synthesis 1,2. Recently, our lab revealed that ELVs are low-abundance, high-potency, neuroprotective, pro-homeostatic mediators that arrest senescence gene programming and the senescence-activated secretory phenotype (SASP) in neural cells upon homeostasis disruptions 3.

Without wishing to be bound by theory, Elovanoids downregulate slow-going inflammation (inflammaging) in the adipose tissue (AT) and hypothalamus (HT), primarily through a senescence program (SP) that involves senescence transcriptome and SASP. This is validated by data using human adipocytes from diabetic patients, a genetic diabetic mouse model, human brain cells in culture and a state-of-the-art approach to addressing specific functional issues in the HT. In embodiments, inflammaging can refer to a low-grade, sterile, chronic pro-inflammatory status. For example, inflammaging can develop with aging and contribute to clinical manifestations of age-related pathologies.

The HT is also a target because, although it is comprised of terminally differentiated cells and originates from the neuro-epithelium, senescent neurons in aged mice, models of AD 4, and astrocytes 5,6, also expresses senescence and develops secretory SASP that fuels neuroinflammation in nearby cells 7-9. Our recent study shows neighboring cells are targeted by neurotoxic actions of SASP, inducing retinal paracrine senescence 3.

Aim 1) Validate that Elovanoids counteract in human adipocytes from diabetic patients' senescence programming activation upon induction by TNFα, IL1β or other inducers.

Active brown/beige AT plasticity increases energy expenditure and is linked to reduced hyperglycemia and hyperlipidemia; on the other hand, its atrophy and inactivation are associated with obesity and aging. Thus, a chronic slow-going local inflammatory condition would disrupt the regulation of internal signals as well as connections with the HC.

Aim 2) Validate that the HT of genetically diabetic mice develops SP that in turn impairs synaptic connectivity and neuronal dysfunctions. This is supported by data that demonstrate that HC of genetically diabetic mice displays perturbed electrophysiological activities (in our newly developed Maestro system). Thus, we have a new experimental model to utilize and mechanisms in addition to a model to assess therapeutic Elovanoids.

Aim 3) Test the experimental therapeutic efficacy of Elovanoids when administered systemically and/or intranasally in diabetic mice.

b) Significance and Innovation

This example is based on the therapeutic use for diabetes of new pro-homeostatic and neuroprotective mediators, the Elovanoids (ELVs). These compounds sustain neural cell integrity 1,10, counteract senescence programing 3 and, as supported by our current data, arrest signaling disturbances in adipose tissue (human diabetics) and in hypothalamus (genetic diabetic mouse model). ELVs mediate protection in neuronal cultures undergoing either oxygen/glucose deprivation or N methyl-D-aspartate receptor-mediated excitotoxicity, as well as in experimental ischemic stroke 10. The methyl ester or sodium salt of ELV-N-32 and ELV-N-34 resulted in reduced infarct volumes, promoted cell survival, and diminished neurovascular unit disruption when administered 1 hour following 2 hours of ischemia by middle cerebral artery occlusion.

Elovanoids as a Therapy for Diabetes and Obesity

It has recently been found that lean mice that became obese due to a high-fat diet display enhanced senescent cells abundance in their brain and anxiety behavior 11. This study also provides the first evidence that obesity-driven anxiety is arrested by new senolytic drugs that dissipated the senescent cells. Senescent cells release a senescence-associated secretory phenotype (SASP) that induces nearby healthy cells to join in the dysfunction.

Transplanting senescent cells into young mice triggers weakness, frailty, and persistent dysfunctions lessened by administering a senolytic cocktail that can includes Dasatinib (an anti-leukemia drug) and Quercetin (a plant flavonol) that set in motion programmed cell death of the senescence cells, extending both life and health span in aging mice. Several publications have reported that senescent cells accumulate in obesity. Obese mice display enhanced senescent cells abundance in the white matter adjacent to the lateral ventricles 12-17.

Elovanoids as a Therapy is Supported by the Following:
1) Our data on AT and HT (see herein)
2) Our data on human neurons in culture demonstrate that SASP activation is blocked by ELVs (see herein). ELVs counteract senescence in human neural cells.
3) We have reported that oligomeric A-beta peptide activates the SP, SASP followed by retinal cell death, and that Elovanoids arrest the expression of the SP genes p16INK4a, MMP1, p53, p21, p27, Il-6, and MMP1 as well as of the SASP secretome and of p16 protein by Western blot 3.
4) Additionally, we found that Elovanoids inhibit the expression of autophagy genes (ATG3, ATG5, ATG7, and Beclin-1) upon oligomeric A-beta peptide challenge in retinal cells 3. Autophagy is a key event of brown/beige adipocytes plasticity by regulating intracellular remodeling during brown/beige adipogenesis, thermogenic activation, and inactivation. This can include autophagic degradation of mitochondria critical for the inactivation of brown adipocytes and the transition from beige-to-white adipose tissue 3.
5) We also found that Elovanoids modulate the matrix metalloproteinases transcriptome (MMP1a, MMP2, MMP3, MMP8, MMP9, MMP12, and MMP13), and we indicated that, with SASP, this mechanism contributes to alter the extracellular matrix 3. So, in both AT and HT, SASP is autocrine and paracrine, modifying the homeostasis of the extracellular matrix microenvironment in both the AT and the HT as a consequence, creating an inflammatory milieu that contributes to impaired function in insulin sensitivity. Elovanoids regulate slow-going, chronic, sterile inflammation (i.e., inflammaging). This is even a tenant of the rationale described herein.
6) Additionally, another target of Elovanoids is unresolved oxidative stress and inflammation in neurons and neural injury models 1,2. These alterations, such as unresolved inflammation, evolve in dysfunctional adipocytes and are among the best consequences of studied proinflammatory signaling in AT and insulin resistance. Produced by immune cells, TNF-α directly prevents insulin action in the adipocyte by downregulating the major insulin-responsive glucose transporter GLUT4 and inhibits insulin-dependent tyrosine phosphorylation of the insulin receptor and IRS-1 through ceramide production in addition to IL-6, IFN-7, and CCL2.
7) Translation innovation. Biomimetic therapeutic approach using synthetically produced molecules of endogenously generated Elovanoids that:
   Restore homeostasis and counteract diabetes—Arrest senescence programming and neural cell damage in metabolic syndrome/obesity
   Use innovative medicinal chemistry

c) Research Approach

Hypothalamus is a Target of Metabolic Syndrome and Critical for Obesity and Diabetes Type 2.

Various aspects of physiological deterioration including obesity and diabetes type 2 are controlled by the hypothalamus, a critical brain region that connects the neuroendocrine system to physiological functions. In addition, functional alterations in a set of agouti-related peptide/neuropeptide Y (AgRP/NPY) and proopiomelanocortin (POMC) neurons, a set of growth hormone-releasing hormone (GHRH) and somatostatin (SST) neurons, a set of arginine vasopressin (AVP) and vasoactive intestinal peptide (VIP) neurons, and a set of gonadotropin-releasing hormone (GnRH) and kisspeptin/neurokinin B/dynorphin (KNDy) neurons contribute to age-related physiological decline in energy metabolism, hormone regulation, circadian rhythm, and reproduction. The underlying cellular mechanism for the hypothalamus-mediated dysfunctions progression comprises dysregulation of nutrient sensing, altered intercellular communication, stem cell exhaustion, senescence programming activation, loss of proteostasis, and epigenetic alterations.

One of the functions of the arcuate (ARC) hypothalamic neurons is to appropriately respond to hormones and neuropeptides both locally and peripherally and involved in energy homeostasis. Arcuate hypothalamic neurons have projections to the PVN and upon stimulation of the ARC, there is a co-release of dopamine and GABA to the neurons in the PVN where dopamine excited orexigenic neurons synthesize AgRP and NPY and inhibit anorexigenic neurons that synthesize POMC. The Ventromedial nucleus of the hypothalamus, (VMH) is involved in detecting hypoglycemic events and initiating the physiological counter-regulatory responses to overcome it. Hence, to evaluate the hypothalamus as a target of Elovanoids, we have use ex vivo organotypic culture of hypothalamic slices from brains of C57BL/6J (WT) mice and age matched Leprdb (db/db diabetic) mice and found changes in synaptic circuitry activity and firing trains of action potentials using innovative Maestro microelectrode arrays (MEA) performed with MaestroPro MEA, Axion Biosystems, GA.

Microelectrode Array (MEA) Measurements to Assess Hypothalamus as a Target of Elovanoids in Diabetes.

We determined whether ex vivo organotypic culture of hypothalamic slices (200 m in thickness) obtained from brains of C57BL/6J (WT) mice and age matched Leprdb (db/db diabetic) mice were electrically active and capable of firing trains of action potentials using Maestro microelectrode arrays (MEA) performed with MaestroPro MEA system from Axion Biosystems, GA.

MEA measurements were carried out using a 48-well Microelectrode array (MEA) plate (M768-tMEA-48W) from Axion Biosystems, GA. Each well of the MEA plate, contained a 4×4 grid of 30 nm circular nanoporous PEDOT electrodes embedded in the cell culture substrate, with a pole-to-pole electrode spacing of 200 μM. In preparation for seeding hypothalamic slices, the wells were treated with 0.1% polyethylenimine (PEI) in sodium borate buffer, pH 8.4. Wells were then pre-coated in laminin (6 μg/mL) and the hypothalamic slices (200 m in thickness) were plated on the electrode grid. Hypothalamus slices were plated and maintained for 4 days in culture in complete neurobasal medium supplemented with B27™ and N2 supplements along with GlutaMax™ and Pen Strep (Thermo Fisher, Gibco™) at 37° C. and 5% CO2.

Extracellular recordings of spontaneous action potentials were performed in culture medium at 37° C. using the standard neural setting of the MaestroPro MEA system and AxIS software version 1.5.1.12. (Axion Biosystems). Data were sampled at rate of 12.5 kHz with a hardware frequency bandwidth of 200-5000 Hz and filtered again in software using a 200-2500 Hz single-order Butterworth band-pass filer to remove high frequency noise before spike detection. The threshold for spike detection was set to 6 times the rolling standard deviation of the filtered field potential on each electrode. Five-minutes recording spans were used to calculate average spike rate for the well, and the number of active electrodes in a well (“Active Electrodes”), which was defined as the number with spike rates≥0.5/min. Recorded spike number per electrode were averaged after disregarding noisy electrodes from analysis. Spike time stamps were exported to Neuroexplorer 5.0 (NEX Technologies) for creation of spike raster plots.

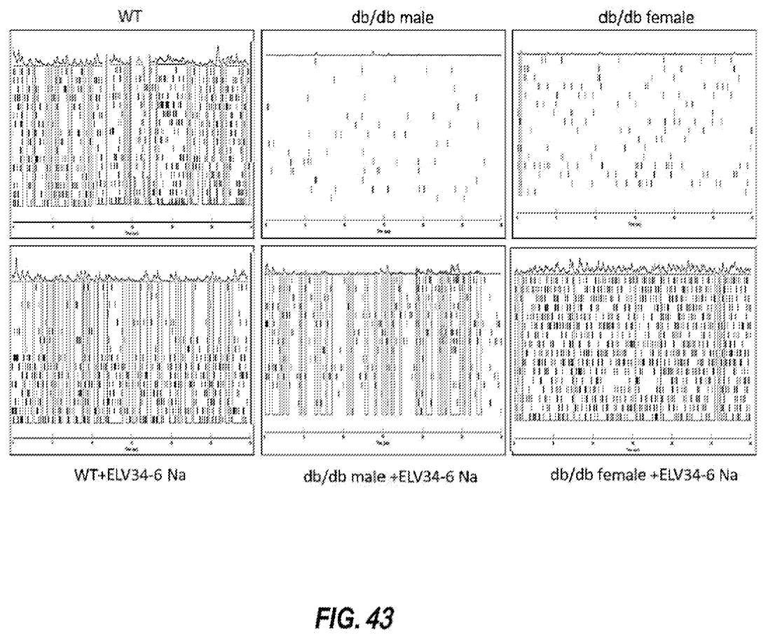

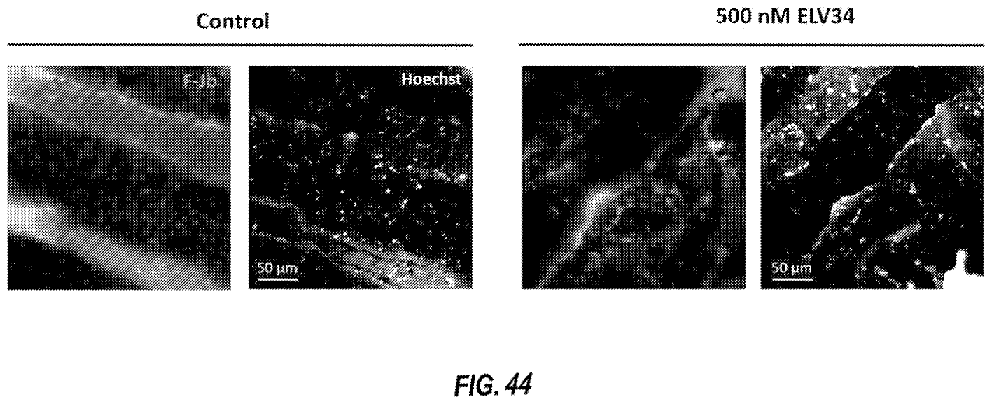

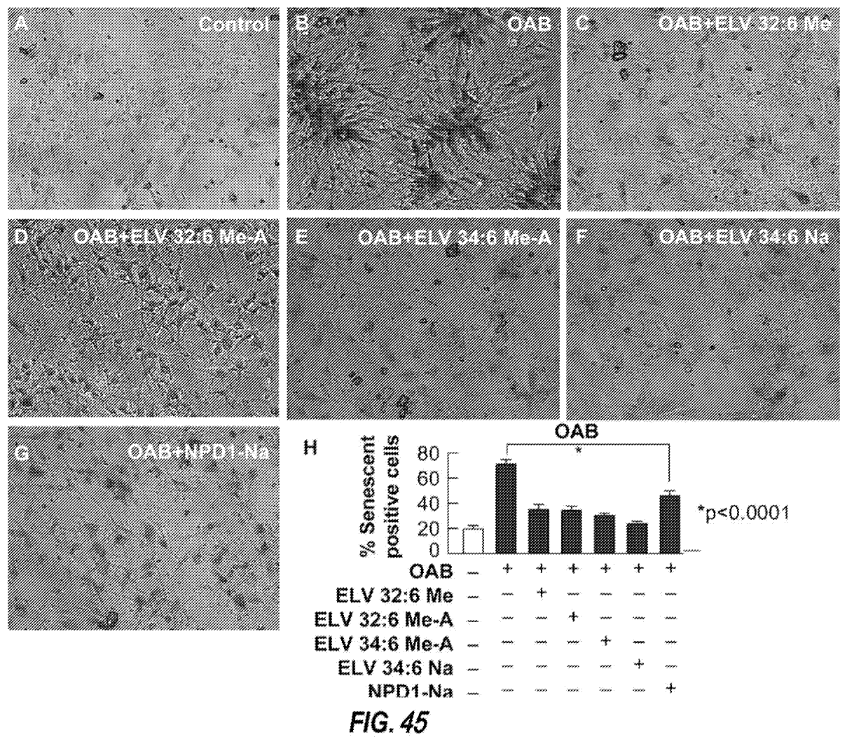

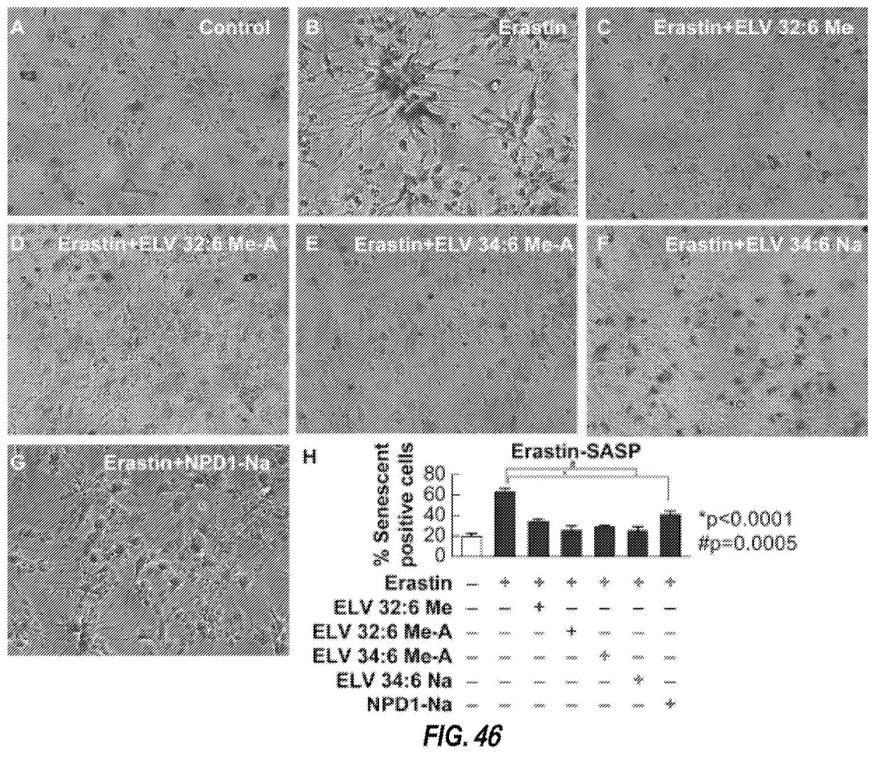

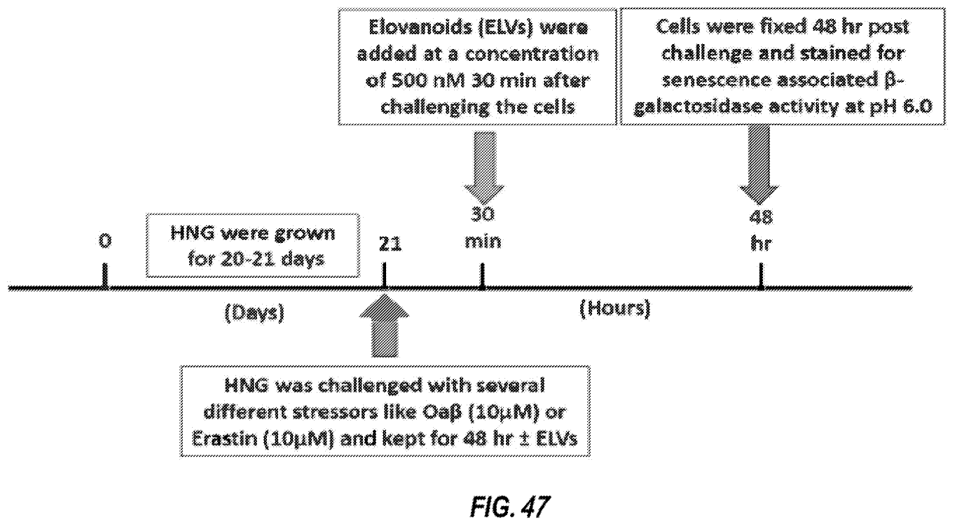

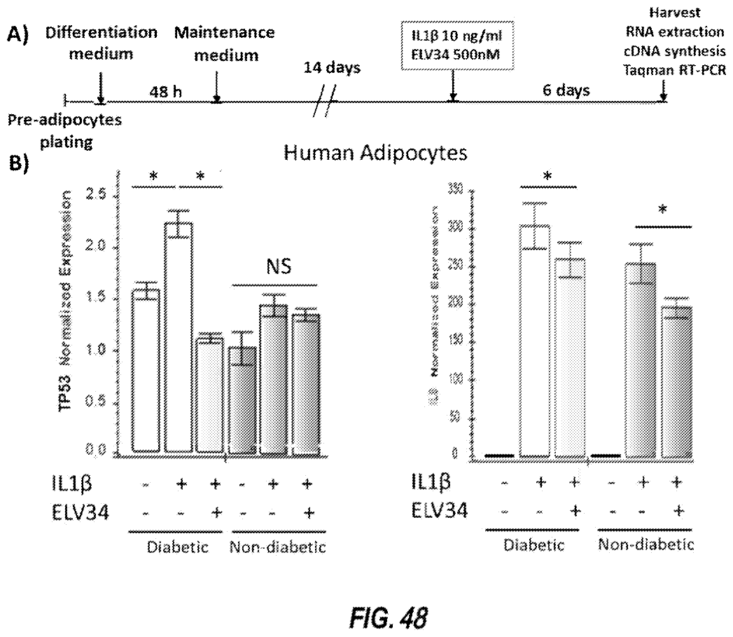

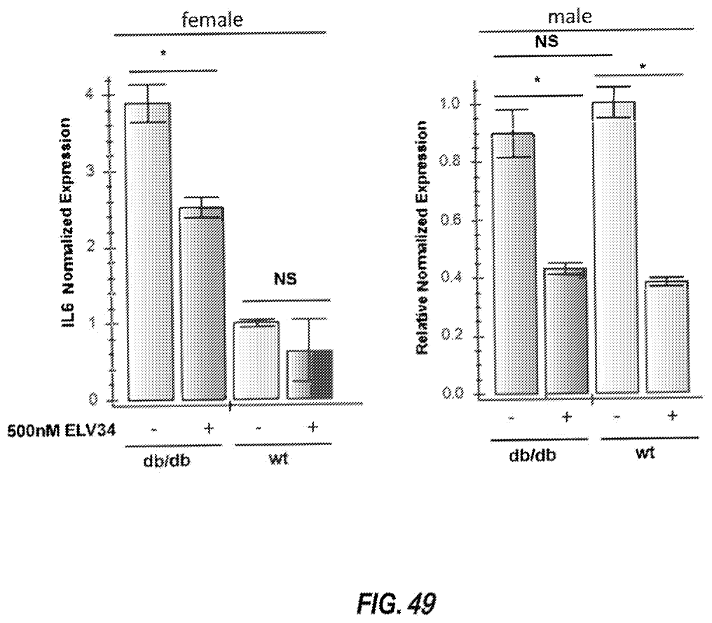

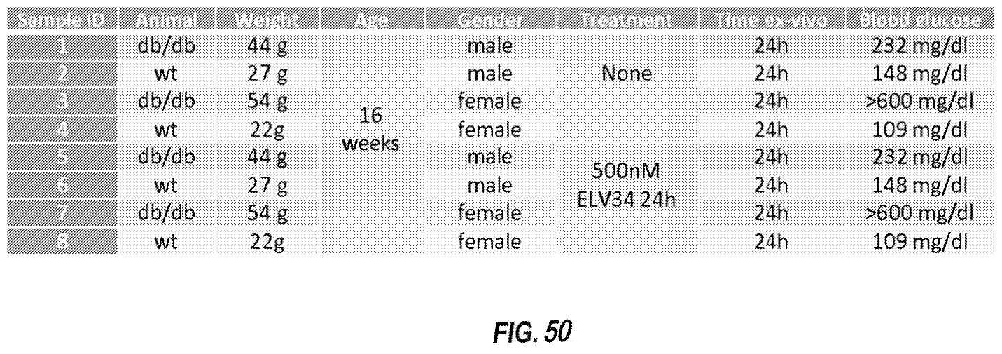

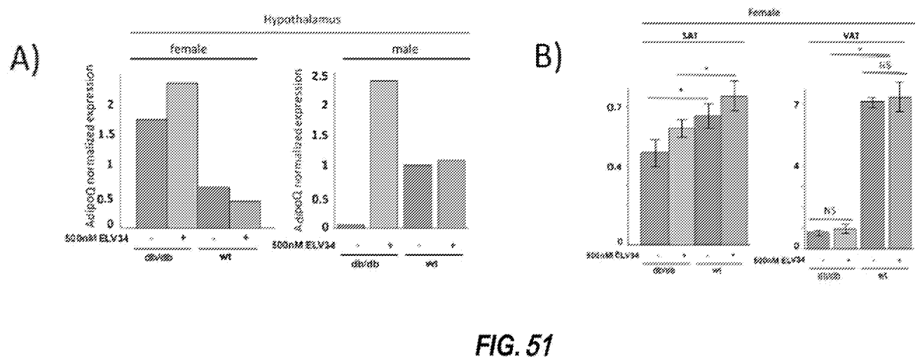

Using the MEA system, population level electrical activity was recorded from different hypothalamic neurons. Referring to FIG. 43, on the left top panel, we have the representative raster plots showing burst activity and spike histograms of C57B16 (WT) male mouse and on the left bottom panel, the raster plots for C57B16 (WT) male mouse treated with ELV (500 nM). A raster trace of each of the wells showed measurable firing by the hypothalamic neurons for the electrodes in contact with an active neuron. Each horizontal row represented an electrode in the well. Spontaneous activity (isolated single spikes and multiple-spike bursts) was evident in hypothalamic slices from normal C57B16 WT controls, while the middle and right top panels, show the raster plots for the Leprdb (db/db diabetic) male and female mice respectively. These plots show asynchronous field potential and spike/burst activity. The low-level spiking could arise from either a cell-autonomous deficiency in excitability or a lack of synaptic drive from neighboring cells. All these spikes were induced with the addition of Dopamine (50 nM) followed by GABA-a antagonist, bicuculline (10 μM). Upon addition of NMDA (10 μM), there was no induction of big spikes, rather it was like baseline. The middle and right bottom panels show that in the Leprdb (db/db diabetic) male and female age matched control mice hypothalamic sections, the asynchronous activity is overcome by the addition of ELV-34-6 Na (500 nM) and synchronous spiking is restored. ELV34 may protect hypothalamus from neurodegeneration. Here, we have shown that we can model CNS regulated disorders and delineate differences between control/diseased condition using the HTS screening method with the multi-electrode array (MEA) system and Elovanoids have therapeutic usefulness in reversing the ill-effects of diabetes and may play a critical role in the CNS regulation of glucose metabolism.

Protection by Elovanoids of Hypothalamic Neuronal Cell Death (by Fluoro-Jade B Staining) in Adult Obese Diabetic Mice (db/db)

Fluoro-Jade b (F-Jb) stains degenerating neurons. The adult obese diabetic mice (db/db) showed increased FJb signal in organotypic slices of Hypothalamus while the wild type mice depicted negligible amounts indicating that db/db mice hypothalamus is undergoing neurodegeneration. 48 h incubation with 500 nM ELV34 elicited neuroprotection that was reflected in the decrease of F-Jb signal (a).

Validation Experiments on Human Brain Neurons/Astrocytes Demonstrating that Elovanoids Block Senescence Associated Secretory Phenotype (SASP): β-Galactosidase Staining for Senescent Neurons

Senescense associated β-galactosidase activity measured in human neuronal-glial (HNG) cells exposed to oligomeric amyloid beta (Oaβ) (10 μM). (A-G) SA-β-Gal activity in HNG cells treated with Oaβ (10 μM) and different Elovanoids (ELVs) or neuroprotectin D1 (NPD1) at a concentration of 500 nM. Micrographs were obtained with bright field microscopy. (H) Quantification of SA-β-Gal+ cells shown in (A-G). SA-β-Gal+ cells were scored in 3 random fields of at least 150 total cells. Results are expressed as percentage of stained SA-β-Gal+ cells (mean±SEM). Statistical analysis were done using Graphpad Prism software 8.3. Results compared with one-way ANOVA, followed by Holm's Sidak post hoc tests and p<0.05 was considered statistically significant.

Similarly, senescense associated β-galactosidase activity measured in human neuronal-glial (HNG) cells exposed to Erastin (10 μM). (A-G) SA-β-Gal activity in HNG cells treated with Erastin (10 μM) and different Elovanoids (ELVs) or neuroprotectin D1 (NPD1) at a concentration of 500 nM. Micrographs were obtained with bright field microscopy. (H) Quantification of SA-β-Gal+ cells shown in (A-G). SA-β-Gal+ cells were scored in 3 random fields of at least 150 total cells. Results are expressed as percentage of stained SA-β-Gal+ cells (mean±SEM). Statistical analysis were done using Graphpad Prism software 8.3. Results compared with one-way ANOVA, followed by Holm's Sidak post hoc tests and p<0.05 was considered statistically significant. Experimental design: Human neuronal glial (HNG) cells were challenged using Oaβ or Erastin.

The following aims will test the components described herein:

Aim 1) Test the prediction that Elovanoids counteract in human adipocytes from diabetic patients' senescence programming activation upon induction by TNFα, IL1β or other inducers.

Active brown/beige AT plasticity increases energy expenditure and is linked to reduced hyperglycemia and hyperlipidemia; on the other hand, its atrophy and inactivation are associated with obesity and aging.

Aim 2) Test that the HT of genetically diabetic mice develops SP that in turn impairs synaptic connectivity and neuronal dysfunctions. This is supported by data that demonstrate that HC of genetically diabetic mice displays perturbed electrophysiological activities (in our newly developed Maestro system). Thus, we have a new experimental model to test mechanisms in addition to a model to assess therapeutic Elovanoids.

Aim 3) Test the experimental therapeutic efficacy of Elovanoids when administered systemically and/or intranasally in diabetic mice.

Data Demonstrating Elovanoids Counteracting Senescence Programming and Inflammation
ELV34 brought down the levels of TP53 mRNA down to non-diabetic control levels in human adipocytes from diabetic patient treated with IL1β. IL1β is a cytokine that induce insulin resistance in adipocytes 18
Similarly, IL8 was elevated by IL1P and brought down around 40 folds by 500 nM ELV34 in both diabetic and non-diabetic adipocytes. Even though the mechanisms are unknown ELV34 can be a therapeutic agent to stop or halt damaging signaling observed in diabetic patients.

ELV34 revert the effect of IL1p in human diabetic adipocytes. A) experimental design. B) expression levels of TP53 and IL8 in human diabetic and non-diabetic adipocytes by the means of Tagman Real time PCR.

ELV34 reduced the levels of IL6 (marker of SASP) induced by IL1P in Diabetic db/db mice hypothalamus indicating that hypothalamic neurons and astrocytes undergo SP. Different effects have been observed in female and male mice.

ELV34 treatment increased the levels of Adiponectin an anti-diabetic systemic hormone secreted by adipocytes and other tissues (hypothalamus) that promotes insulin sensitivity. A) Diabetic hypothalamus treated with ELV34 showed a trend of increase in adiponectin in female and males. B) Differential effect of ELV34 in subcutaneous adipose tissue (SAT), and visceral adipose tissue (VAT). SAT and VAT possess differential ability to browning 19.

Approach

Specific Aim 1. Test the Prediction that Elovanoids Counteract in Human Adipocytes from Diabetic Patients' Senescence Programming Activation Upon Induction by TNFα, IL1β or Other Inducers.

Rationale. TNFα is circulating in obese patients and has an important role in insulin resistance pathogenesis20,21. In addition, Interleukin 1β signaling mediates the effects of macrophages on the adipose tissue 18. Circulating IL1β induce disruption in the function of adipose tissue 22. Without wishing to be bound by theory, systemic IL1β induce senescence in human adipocytes. The loss of functionality of the adipocytes exposed to cytokines can include the incapability of the cells for browning. Active brown/beige AT plasticity increases energy expenditure and is linked to reduced hyperglycemia and hyperlipidemia; on the other hand, its atrophy and inactivation are associated with obesity and aging. Thus, a chronic slow going local inflammatory condition would disrupt the regulation of internal signals as well as connections with the HC. In addition, other hormones produced and secreted by fat tissue, like adiponectin, may be reduced when the adipocytes undergo SP. Without wishing to be bound by theory, adiponectin has anti-diabetic and anti-inflammatory effects, and it also functions as an insulin sensitizer 23. Results showed that Elovanoid 34 (ELV34) can revert several pathological features in diabetic mice (db/db) and in human diabetic adipocytes. Based on these results we can halt the SP and SASP on human diabetic adipocytes.

Experimental Design.

Experiment 1: To determine whether IL1β induces senescence in adipocytes we will 1) test for the markers of senescence: p16, p21, p27, p53 expression and activity; 2) measure β-galactosidase activity and 3) Senescence-Associated Secretory Phenotype (SASP) in human adipocytes from diabetic and non-diabetic patients exposed to IL1β and/or TNFα.

To test for the expression of senescence markers, differentiated adipocytes from diabetic and non-diabetic patients will be exposed to IL1β and/or TNFα for 6 days plus and minus ELV34, harvested and their RNA will be extracted and used as template for cDNA synthesis. The expression levels of p16, p21, p27 and p53 will be assessed by Real time PCR using Taqman probes. The results will be normalized by the expression of housekeeping genes: PPIA, GAPDH, β-actin, B2M, TBP and TFRC. The activity of these markers will be measured by western blot assay to detect phosphorylation and increase in the total protein content.

The β-galactosidase activity assay is based on the overexpression and accumulation of the endogenous lysosomal beta-galactosidase specifically in senescent cells 24. Similarly to the detection of the expression of the senescence markers, human diabetic and non-diabetic adipocytes will be exposed to IL1β and/or TNFα for 6, 9 and 12 days plus and minus ELV34 and then stained using a β-galactosidase substrate that produces fluorescent signal when hydrolyzed by the endogenous enzyme. The samples will be imaged by two methods: confocal microscopy and flow cytometry.

The third part consists on determining the Senescence-Associated Secretory Phenotype. The senescence of the adipocytes will be induced as mentioned described herein in the presence or absence of EL34 and the resulting culture medium will be collected, concentrated and tested for candidates via western blot or ELISA assays. Some of the candidates to be tested are: IL-6, IL-7, IL-1α, -1β, IL-13, IL-15, IL-8, GRO-α, -β, -γ, MCP-2, MIP-1a, Eotaxin, Eotaxin-3, TECK, ENA-78, I-309 and MMP-1, -3, -10, -12, -13, -14.

Experiment 2: to assess the effects of SP and/or SASP in the content of brown and white adipocytes, the cells from experiment 1 will be 1) tested for markers of brown tissue such as CD137, TMEM26 (transmembrane protein 26) and TBX1 (T-box 1) which are found enriched in human brown fat (BAT). PGC-1α, PPARγ, C/EBPα and PRDM16 will also be evaluated in diabetic and non-diabetic adipocytes exposed to IL1β and/or TNFα using western blot to establish the variations in their content and their activity will be measured via luciferase reporter assay. The adipocytes undergoing SP and or SASP will be then treated with ELV34 and the parameters described herein will be tested.

Experiment 3: to assess the ability of diabetic adipocytes exposed to IL1β and/or TNFα for 6, 9 and 12 days to synthesize and release Adiponectin. Adipocytes treated with IL1β and/or TNFα will be tested for expression of adiponectin mRNA via real time PCR (Figure X), western blot assay and the culture medium will be collected and subjected to ELISA and/or western blot assay. The effects of the ELV34 on the production of Adiponectin will be tested using the procedures described herein.

Without wishing to be bound by theory, we will detect senescence and the SASP in human adipocytes from diabetic patients exposed to IL10 and/or TNFα but not in non-diabetic adipocytes. However, when a senescent-like phenotype is triggered in cells that overexpress cell-cycle inhibitors such as p16 or p21, cells undergo a growth arrest with many characteristics of senescent cells, but not a SASP 25. Thus, without wishing to be bound by theory, SASP will not be observed in the presence of increased p16 or p21.

Without wishing to be bound by theory, the diabetic adipocytes that become senescent will show lower expression of BAT markers; CD137, TMEM26 (transmembrane protein 26) and TBX1 (T-box 1) concomitantly with low activity of the main transcription factors responsible for the genetic signature observed in brown adipocytes.

In parallel with the SP, and without wishing to be bound by theory, decreased expression and secretion of adiponectin in diabetic adipocytes will be observed.

ELV34 will halt SP and SASP in diabetic adipocytes in culture and favoring the browning of the cells and the synthesis of AdipoQ.

Specific Aim 2. Validate that the HT of genetically diabetic mice develops SP that in turn impairs synaptic connectivity and neuronal dysfunctions. This is supported by data that demonstrate that HC of genetically diabetic mice displays perturbed electrophysiological activities (in our newly developed Maestro system). Thus, we have an experimental model to validate embodiments described herein and mechanisms in addition to a model to assess therapeutic Elovanoids.

Rationale. The neuro-inflammation of the hypothalamus induces dysregulation of the neurons, which then enter senescence, leading to metabolic syndrome.

Experimental Design. We will evaluate our findings with BKS.Cg-Dock7m+/+ Leprdb/J diabetic mice and controls C57BLKS/J. Samples from both genders will be included. This strain of mice is used to model phases I to III of diabetes type II and obesity. We will dissect out the brain of these animals and slice it to isolate the hypothalamus, the hippocampus and the cortex. Each organotypic slice will be set up in an individual well with Neurobasal medium supplemented with B27. 48 h post-plating medium will be supplemented with 500 nM ELV and 24 h later, the organotypic slices will be recorded. Neuronal activity will be determined using the Axion BioSystems' Maestro multielectrode array (MEA) technology. Due to the heterogeneity of neurons composing the hypothalamus, we will validate the hypothalamic neurons identity that we are testing by adding 50 nM dopamine and 10 μM bicuculine, which will indicates the presence of the ventro-medium (VTM) and arcuate nuclei that are important for satiety and feeding. Comparison of the neuronal activity of control and diabetic mice hypothalamus with and without ELV will provide us the baseline activity of this brain structure and help us evaluate the functionality of the neurons. After the recording, the hypothalamus slice will be retrieved and total RNA will be extracted. First-strand cDNA will be reversed transcribed, and the expression of genes involved in senescence programming, as well as insulin signaling and sensitivity and glucose metabolism will be examined. The up- and downregulation of candidates (p53, p21, p16ink4a, and Bmi-1) will be further confirmed by Western Blot or capillary Western Blot for a larger panel of targets.

Outcome. Neuronal activity of the HT of diabetic mice has a decreased sensitivity for dopamine and bicculine, indicating malfunctioning neurons. By investigating the causality, an upregulation of markers for SP and SASP in diabetic mice as compared to control will be observed.

Specific Aim 3. Validate the Therapeutic Efficacy of Elovanoids when Administered Systemically and/or Intranasally in Diabetic Mice.

Rationale. Routes of administration affect bioavailability by changing the number of biologic barriers a drug must cross or by changing the exposure of drug to pumping and metabolic mechanisms. We will validate various routes of administration including iv and intranasal lungs serve as an effective route of administration of drugs. The pulmonary alveoli represent a large surface and a minimal barrier to diffusion. The lungs also receive the total cardiac output as blood flow. Thus, absorption from the lungs can be very rapid and complete. Elovanoids dissolved in 0.9% saline (vehicle) are nonirritating and from previous experiments have been seen to be delivered very effectively intranasally. The intended effects can be systemic.

Experimental Design. We will evaluate our findings with BKS.Cg-Dock7m+/+ Leprdb/J mice and controls C57BLKS/J. This strain of mice is used to model phases I to III of diabetes type II and obesity. Samples from both genders will be included. All animal experiments will be carried out in accordance with the approved IACUC protocol issued by Louisiana State University Health Sciences Center. Intranasal administration will be performed on lightly anesthetized mice. Each mouse will be placed on a sterile surgical pad and lightly stretched out to better hold the scruff. With a firm grip on the scruff, the mouse will be turned on its back while still allowing the mouse to breathe and be comfortable. With the neck and chin flat and parallel to the pad, the tip of the pipettor containing the Elovanoids dispersed in 0.9% saline (vehicle) will be placed near the left nostril of the mouse at a 45-degree angle, and about 5 μL of the drug will be administered to the nostril with a 2-3 sec interval in between for a total of 10 μL/nostril. The mouse will be held in this position for 5 sec or until it regained consciousness, then the administration step will be repeated for the other nostril for a total of 20 μL/mouse. After the mouse receives all drops, the animal will be kept restrained on its back until the material disappears into the nares and then will be returned to its cage. After 4-, 24-, 48-, 72- and 120-hours mice will be sacrificed. We will collect visceral and subcutaneous adipose tissues (VAT and SAT), as well as the brain to dissect out the hypothalamus and create organotypic slices. VAT and SAT will be dissociated, and adipocytes will be plated in 6 well-plates and the brain dissected out to collect the hypothalamus, hippocampus and cerebral cortex. With the tissue we will test the parameters explained in the experiments designed for Specific Aims 1 and 2.

Outcome. Without wishing to be bound by theory, SP and SASP will halt in VAT, SAT and HT of Leprdb/J mice. Also, treatment of db/db mice with ELV-34 intranasally will restore the spontaneous electrical activity of the HT of these mice. Moreover, VAT and SAT will synthesize and release Adiponectin, and restore the SP reprogramming.

Outcomes

1—Establish a solid foundation for Elovanoids as a therapeutic in obesity and diabetes type 2, although there is a case for the use of Elovanoids as a therapeutic for diabetes type 1 as well.

### REFERENCES CITED IN EXAMPLES HEREIN

1. Bhattacharjee S, Jun B, Belayev L, et al. Elovanoids are a novel class of homeostatic lipid mediators that protect neural cell integrity upon injury. Sci Adv. 2017; 3(9):e1700735. doi:10.1126/sciadv.1700735
2. Jun B, Mukherjee P K, Asatryan A, et al. Elovanoids are novel cell-specific lipid mediators necessary for neuroprotective signaling for photoreceptor cell integrity. Sci Rep. 2017; 7(1):5279. doi:10.1038/s41598-017-05433-7
3. Do K V, Kautzmann M-A I, Jun B, et al. Elovanoids counteract oligomeric β-amyloid-induced gene expression and protect photoreceptors. Proc Natl Acad Sci. 2019; 116(48):24317-24325. doi:10.1073/pnas.1912959116
4. Musi N, Valentine J M, Sickora K R, et al. Tau protein aggregation is associated with cellular senescence in the brain. Aging Cell. 2018; 17(6). doi:10.1111/acel.12840
5. Das M M, Svendsen C N. Astrocytes show reduced support of motor neurons with aging that is accelerated in a rodent model of ALS. Neurobiol Aging. 2015; 36(2):1130-1139. doi:10.1016/j.neurobiolaging.2014.09.020
6. Turnquist C, Horikawa I, Foran E, et al. p53 isoforms regulate astrocyte-mediated neuroprotection and neurodegeneration. Cell Death Differ. 2016; 23(9):1515-1528. doi:10.1038/cdd.2016.37
7. Appel S H, Zhao W, Beers D R, Henkel J S. The microglial-motoneuron dialogue in ALS. Acta MyolMyopathies Cardiomyopathies Off J Mediterr Soc Myol. 2011; 30(1):4-8.
8. Komine O, Yamanaka K. Neuroinflammation in motor neuron disease. Nagoya J Med Sci. 2015; 77(4):537-549.
9. Lunyak V V, Amaro-Ortiz A, Gaur M. Mesenchymal Stem Cells Secretory Responses: Senescence Messaging Secretome and Immunomodulation Perspective. Front Genet. 2017; 8:220. doi:10.3389/fgene.2017.00220
10. Bazan N G. Docosanoids and elovanoids from omega-3 fatty acids are pro-homeostatic modulators of inflammatory responses, cell damage and neuroprotection. Mol Aspects Med. 2018; 64:18-33. doi:10.1016/j.mam.2018.09.003
11. Ogrodnik M, Zhu Y, Langhi L G P, et al. Obesity-Induced Cellular Senescence Drives Anxiety and Impairs Neurogenesis. Cell Metab. 2019; 29(5):1061-1077.e8. doi:10.1016/j.cmet.2018.12.008
12. Bussian T J, Aziz A, Meyer C F, Swenson B L, van Deursen J M, Baker D J. Clearance of senescent glial cells prevents tau-dependent pathology and cognitive decline. Nature. 2018; 562(7728):578-582. doi:10.1038/s41586-018-0543-y
13. Justice I N, Nambiar A M, Tchkonia T, et al. Senolytics in idiopathic pulmonary fibrosis: Results from a first-in-human, open-label, pilot study. EBioMedicine. 2019; 40:554-563. doi:10.1016/j.ebiom.2018.12.052
14. Kirkland J L. ClinicalTrials.gov [Internet]. Bethesda (MD): National Library of Medicine (US). 2018 Sep. 18-. Identifier NCT03675724, Alleviation by Fisetin of Frailty, Inflammation, and Related Measures in Older Adults. ClinicalTrials.gov. https://clinicaltrials.gov/ct2/show/NCT03675724. Published Dec. 28, 2018. Accessed Dec. 16, 2019.
15. Hickson L J. ClinicalTrials.gov [Internet]. Bethesda (MD): National Library of Medicine (US). 2016 Jul. 28-. Identifier NCT02848131, Senescence in Chronic Kidney Disease. ClinicalTrials.gov. https://clinicaltrials.gov/ct2/show/NCT02848131. Published Nov. 8, 2019. Accessed Dec. 16, 2019.
16. Hickson L J. ClinicalTrials.gov [Internet]. Bethesda (MD): National Library of Medicine (US). 2017 Oct. 30-. Identifier NCT03325322, Inflammation and Stem Cells in Diabetic and Chronic Kidney Disease. ClinicalTrials.gov. https://clinicaltrials.gov/ct2/show/NCT03325322. Published Oct. 17, 2019. Accessed Dec. 16, 2019.
17. Hickson U. ClinicalTrials.gov [Internet]. Bethesda (MD): National Library of Medicine (US). 2016 Jan. 11-. Identifier NCT02652052, Hematopoietic Stem Cell Transplant Survivors Study (HTSS Study). ClinicalTrials.gov. https://clinicaltrials.gov/ct2/show/NCT02652052. Published Oct. 15, 2019. Accessed Dec. 16, 2019.
18. Bing C. Is interleukin-1β a culprit in macrophage-adipocyte crosstalk in obesity?Adipocyte. 2015; 4(2):149-152. doi:10.4161/21623945.2014.979661
19. He P, Hou B, Li Y, et al. Lipid Profiling Reveals Browning Heterogeneity of White Adipose Tissue by B3-Adrenergic Stimulation. Biomolecules. 2019; 9(9):444. doi:10.3390/biom9090444
20. Xu H, Uysal K T, Becherer J D, Arner P, Hotamisligil G S. Altered Tumor Necrosis Factor-α (TNF-α) Processing in Adipocytes and Increased Expression of Transmembrane TNF-α in Obesity. Diabetes. 2002; 51(6):1876. doi:10.2337/diabetes.51.6.1876
21. Ruan H, Lodish H F. Insulin resistance in adipose tissue: direct and indirect effects of tumor necrosis factor-α. Cytokine Growth Factor Rev. 2003; 14(5):447-455. doi:10.1016/S1359-6101(03)00052-2
22. Pellegrinelli V, Rouault C, Veyrie N, Clément K, Lacasa D. Endothelial Cells From Visceral Adipose Tissue Disrupt Adipocyte Functions in a Three-Dimensional Setting: Partial Rescue by Angiopoietin-1. Diabetes. 2014; 63(2):535. doi:10.2337/db13-0537
23. Kadowaki T, Yamauchi T, Kubota N, Hara K, Ueki K, Tobe K. Adiponectin and adiponectin receptors in insulin resistance, diabetes, and the metabolic syndrome. J Clin Invest. 2006; 116(7):1784-1792. doi:10.1172/JC129126
24. Lee B Y, Han J A, Im J S, et al. Senescence-associated β-galactosidase is lysosomal β-galactosidase. Aging Cell. 2006; 5(2):187-195. doi:10.1111/j.1474-9726.2006.00199.x
25. Coppé J-P, Desprez P-Y, Krtolica A, Campisi J. The senescence-associated secretory phenotype: the dark side of tumor suppression. Annu Rev Pathol. 2010; 5:99-118. doi:10.1146/annurev-pathol-121808-102144

### Example 12—Elovanoids Counteract Oligomeric β-Amylid-Induced Gene Expression and Protect Photoreceptors

Abstract

The onset of neurodegenerative diseases activates inflammation that leads to progressive neuronal cell death and impairments in cognition (Alzheimer's disease, AD) and sight (age-related macular degeneration, AMD). How neuroinflammation can be counteracted is not known. In AMD, amyloid β-peptide (Aβ) accumulates in subretinal drusen. In the 5×FAD retina, we found early functional deficiencies (ERG) without photoreceptor cell (PRC) death and identified early insufficiency in biosynthetic pathways of pro-homeostatic/neuroprotective mediators, neuroprotectin D1 (NPD1) and elovanoids (ELVs). To mimic an inflammatory milieu in wild-type (WT) mouse, we triggered retinal pigment epithelium (RPE) damage/PRC death by subretinally injected oligomeric β-Amyloid (OAβ) and observed that ELVs administration counteracted their effects, protecting these cells. In addition, ELVs prevented OAβ-induced changes in gene expression engaged in senescence, inflammation, autophagy, extracellular matrix remodeling and AMD. Moreover, since OAβ target the RPE, we used primary human RPE cell cultures and demonstrated that OAβ caused cell damage, while ELVs protected and restored gene expression as in mouse. Our data show OAβ activates senescence as reflected by enhanced expression of p16INK4a, MMP1, p53, p21, p²⁷ and Il-6 and of senescence-associated secretory phenotype (SASP) secretome, followed by RPE and PRC demise and that elovanoids 32 and 34 blunt these events and elicits protection. In addition, ELVs counteracted OAβ-induced expression of genes engaged in AMD, autophagy and extracellular matrix (ECM) remodeling. Overall, our data uncovered that ELVs downplay OAβ-senescence program induction and inflammatory transcriptional events and protect RPE cells and PRC, and therefore have utility as a therapeutic avenue for AMD.

This example uncovers biosynthetic pathway insufficiencies of prohomeostatic/neuroprotective mediators, neuroprotectin D1 and elovanoids, in the retina during early pathology expression in transgenic Alzheimer's disease 5×FAD mouse. These changes correlate with photoreceptor cell functional impairments preceding their loss. Amyloid beta (Aβ) peptide accumulates in drusen in AMD. Thus, injecting oligomeric Abeta in wild-type mice behind the retina leads to photoreceptor cell degeneration and to gene expression disruptions that can include upregulation of a senescence program and of SASP. Similar changes take place in human retinal pigment epithelium cells in culture. The new lipid mediators, the elovanoids, restore Aβ-peptide-induced gene expression changes and SASP secretome and in turn protect these cells. This study opens avenues of therapeutic assessments of elovanoids for AMD.

Introduction

The onset of the neuroinflammatory response encompasses synthesis of endogenous mediators aiming to counteract brain and/or retina damage. Neuroprotectin D1 (NPD1), a docosanoid derived from an omega-3 essential fatty acid, is neuroprotective by arresting inflammation initiation and thus sustaining photoreceptor cell (PRC) integrity (1) and is deficient in the hippocampus CA1 area of early Alzheimer's disease (AD) (2). Aβ accumulates in AD. In the 5×FAD retina, Aβ also accumulates and, although Aβ in AMD sets in motion homeostasis disturbances that can include inflammation and contribute to PRC death (3, 4), it is not known how to limit Aβ-mediated cell damage. In PRCs, very long-chain PUFAs (VLC-PUFAs, C>28) are synthesized by ELOVL4 (elongation of very long chain fatty acid-4) (5, 6) and are necessary for rhodopsin function (7). Mutations on the ELOVL4 gene (5) cause Stargardt macular dystrophy type 3 with central vision loss. Recessive ELOVL4 mutations cause seizures, mental retardation, and spastic quadriplegia, indicating the importance of VLC-PUFAs in brain development and physiology as well (8). Once VLC-PUFAs are incorporated into specific phosphatidylcholine molecular species (PCs) of the photoreceptor cells outer segments, they arrive to the retinal pigment epithelium (RPE) after daily PRC disk shedding and phagocytosis. ELVs with 32 and 34 C are enzymatically synthesized in RPE cells from PC-released VLCPUFAs by a phospholipase A1 (9, 10). These new lipid mediators have the ability to protect RPE cells from uncompensated oxidative stress by upregulating pro-homeostatic and prosurvival protein abundance with attenuation of apoptosis in photoreceptor cells (9, 10) as well as in neurons (11).

Aβ42 is a component of drusen in AMD and of senile plaques of AD (12, 13). In AMD, Abeta contributes to inflammation, perturbed RPE morphology and function, and PRC integrity (14, 15). The 5×FAD transgenic mouse carries mutations associated with early-onset familial AD, and although it displays several unspecific changes, it shows PRC degeneration (16, 17). We first investigated the 5×FAD retinal phospholipid profile seeking to understand the availability of precursors of lipid mediators preceding the expression of PRC degeneration in the 5×FAD mice. Next, we studied the consequences of subretinal administration of oligomeric Aβ (OAβ), one of the most cytotoxic forms of Abeta, in wild-type (WT) mice on RPE and PRC as well as on the expression of genes involved in senescence, autophagy, AMD, extracellular matrix (ECM) remodeling and apoptosis. Also, we exposed human RPE cells in culture to OAβ and assessed similar endpoints. Finally, we evaluated whether or not elovanoids modify OAβ-induced gene expression, including the senescence program and senescence-associated secretory phenotype (SASP) to, in turn, protect the RPE and sustain photoreceptor cell integrity.

Results

5×FAD Mouse Retina and RPE Reveal Deficits in the Pathways Leading to NPD1 and ELV Biosynthesis.

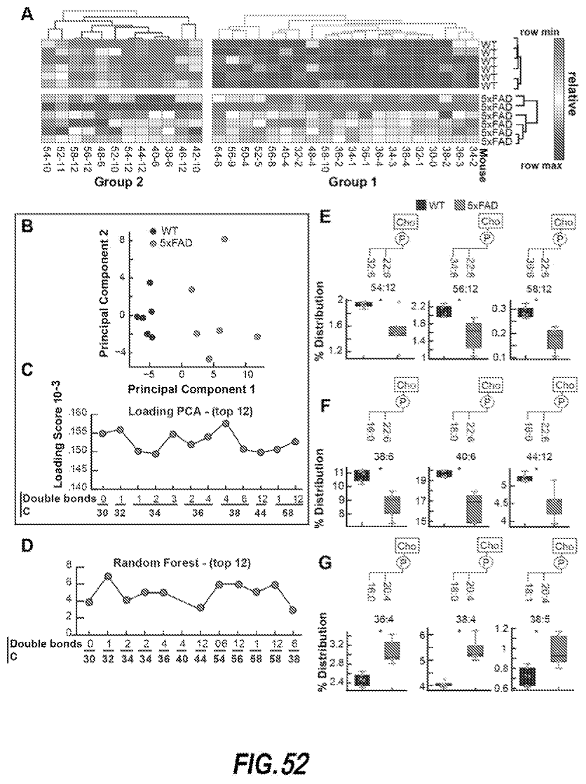

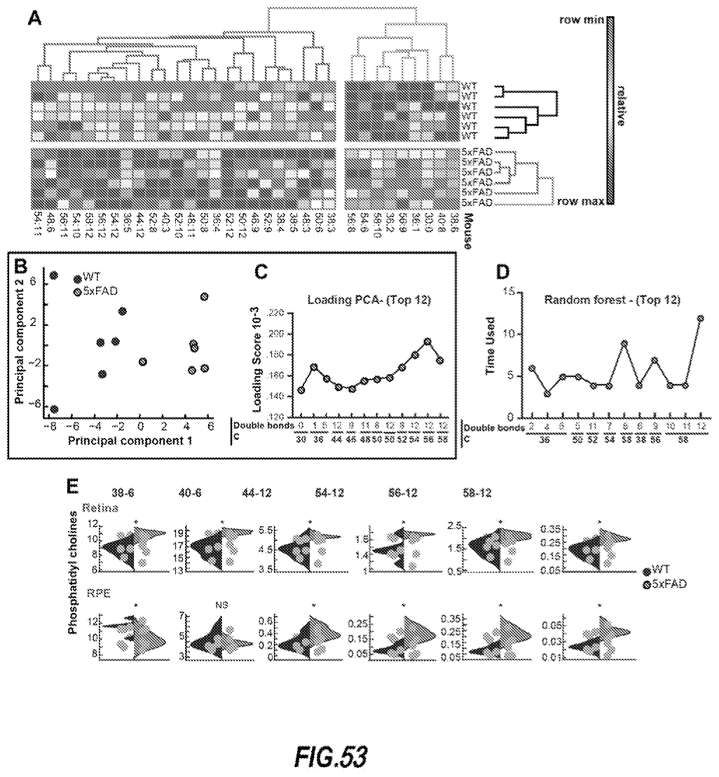

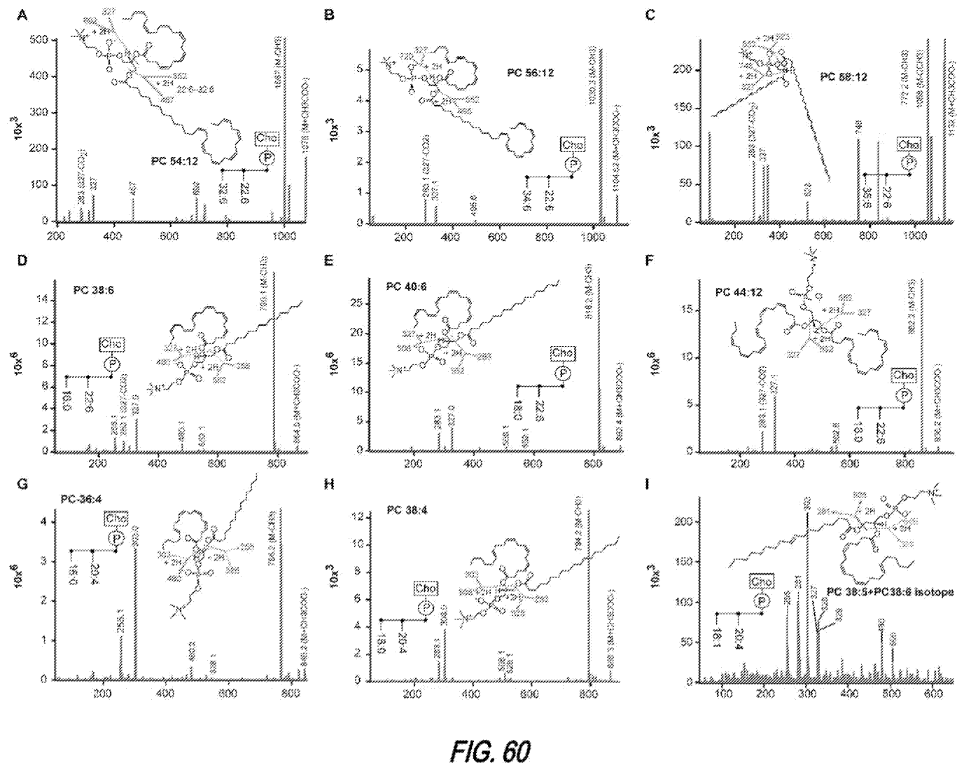

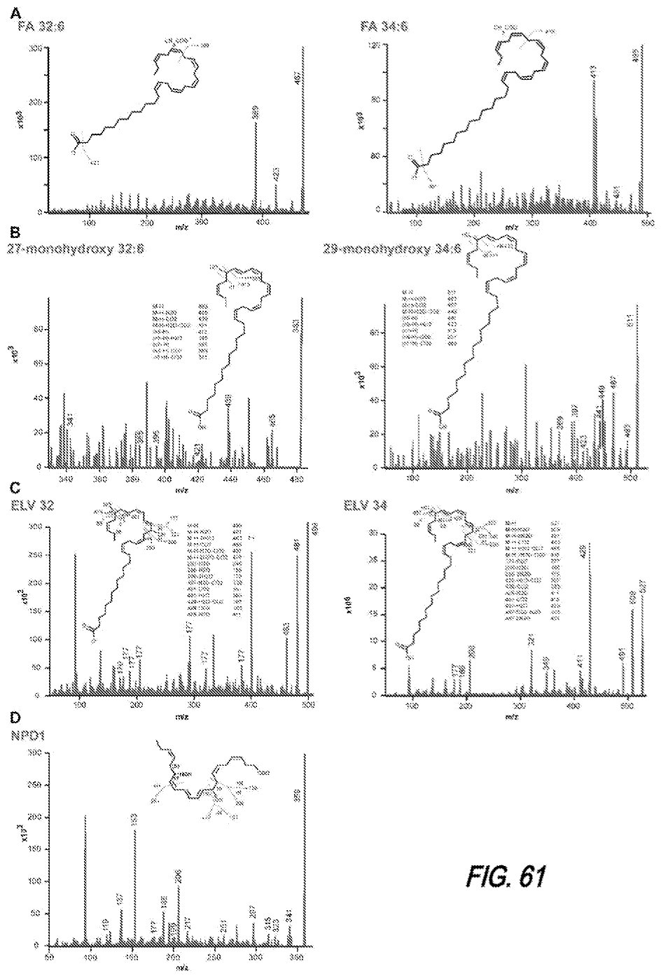

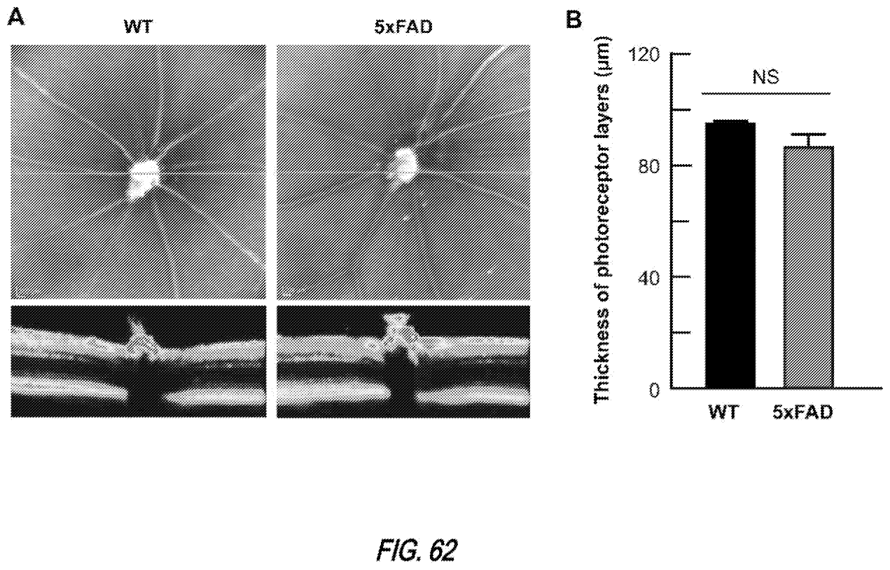

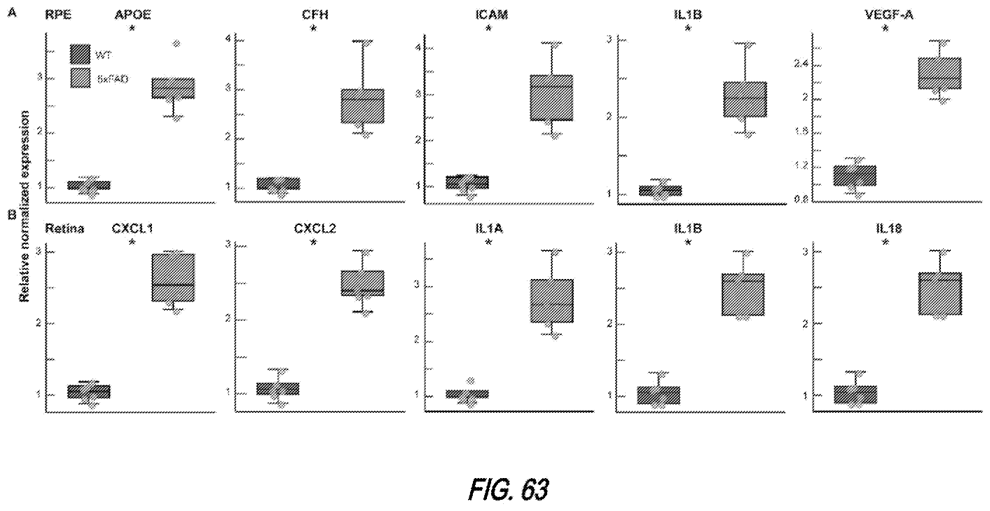

When cleaved by PLA2 and PLA1, acyl chains of a phosphatidylcholine with DHA (at sn2) and VLC-PUFAs,n-3 (at sn1) lead to the synthesis of NPD1 and elovanoids, respectively (10). To ascertain the availability of these PCs in the 5×FAD retinas and RPE, heatmap analyses were performed. Two PC clusters emerged from these analyses: short chain (<48 C) and saturated (<6 doubles bonds) (group 1) and a less abundant cluster (group 2) when comparing the 5×FAD vs. WT (FIG. 52, panel A). This means that 5×FAD retina has relatively less PC containing VLC-PUFAs. However, principal component analysis (PCA) did not reveal any sensible difference, since all discriminable makers for 5×FAD and WT mouse were short chain-containing PCs (FIG. 52, panel B and C). Hence, we performed a random forest classification with the criteria that the higher time used for the phosphatidylcholines, the more PC contribution to the variation of 5×FAD to WT would be highlighted. As a result, we found a dense distribution of high time used PCs in the VLC-PUFA containing PC area (FIG. 52, panel D), which supported the heatmap analysis observation. Therefore, PCs were presented in three groups: (i) DHA and VLC-PUFA containing PCs, (ii) DHA containing PCs, and (iii) AA containing PCs. Structures and m/z of PCs (FIG. 60). The 5×FAD depicted decreases of both DHA and VLC-PUFA containing PCs, including PC54:12, PC56:12, and PC58:12 (FIG. 52, panel E), and DHA containing PCs, including PC36:8, PC38:8, and PC44:12 (FIG. 52, panel F). In contrast, PCs containing AA, including PC36:4, PC38:4, and PC36:5, were increased in the 5×FAD retina (FIG. 52, panel G), indicating that the balance of n-6/n-3 (AA, DHA, and VLC-PUFA) was altered. Next, we observed that DHA and VLC-PUFA contained in PCs were deficient in 5×FAD retina (FIG. 1A-G) unlike in RPE (FIG. 53). The PC38:6 content was higher in the RPE of 5×FAD (contrast to the retina—FIG. 2E), and the PC40:6 was similar in 5×FAD and WT (FIG. 2E). PC44:12, however, was lower in the 5×FAD RPE as in the retina. Furthermore, the relative abundance of PCs differs in retina and RPE. In retina, VLC-PUFA containing PCs amounted to 3% of total PC while these PCs were less than 0.3% in the RPE. Similarly, PC44:12 was 5% in the retina and less than 0.5% in the RPE. Thus, PCs containing DHA and VLC-PUFA are more abundant in photoreceptor cells than RPE. Despite the small contribution of these PCs in RPE, our results clearly unveiled a deficiency of VLC-PUFA containing PCs in the 5×FAD RPE.

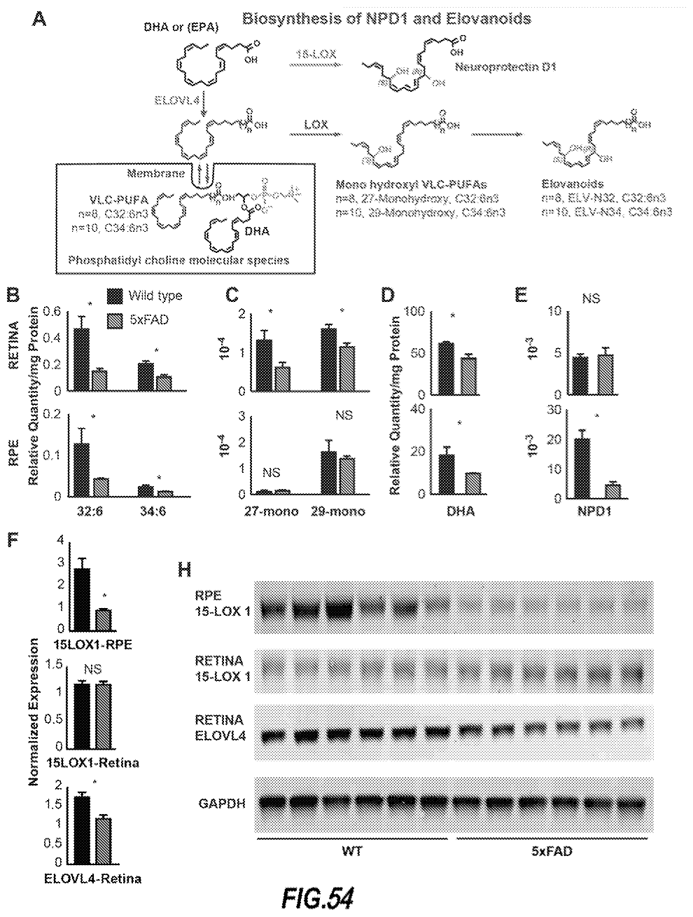

Elovanoids are generated from VLC-PUFA stored in PC54-12 and PC56-12, present in limited amounts in the 5×FAD retina and RPE (FIGS. 1 and 2). We found that the free pool size of 32:6n-3 and 34:6n-3 are increased, reflecting release from the sn-1 position of the PC54-12 and PC56-12 respectively. We next explore the subsequent, lipoxygenase-catalyzed enzymatic epoxidation to form the epoxide intermediate, followed by the hydrolase-catalyzed enzymatic hydrolysis, resulting in synthesis of di-hydroxylated ELV-N-32 or ELV-N-34 with the Z,E,E triene moiety (FIG. 54, panel A). Thus, the expression of 15-lipoxygenase-1 in 5×FAD RPE is less than in WT, whereas, in the retina, there are no differences between the two genotypes (FIG. 54, panels C and D) in agreement with NPD1 abundance that is lower in 5×FAD RPE and unchanged in the retina (FIG. 54, panel B). On the other hand, ELOVL4, an enzyme that elongates EPA or DHA, is only expressed in PRC and is lower in 5×FAD, correlating with the smaller pool size of 32:6n-3 and 34:6n-3 as well as of the monohydroxy stable derivatives of elovanoid hydroperoxide precursors (FIG. 54, panels B-D). Therefore, lipids as well as the expression of two enzymes involved in the ELV and NPD1 pathways are markedly depressed in retina and RPE in early ages of retinal pathology development in the 5×FAD.

Early Abnormal Retina Function Preceding PRC Loss in 5×FAD.

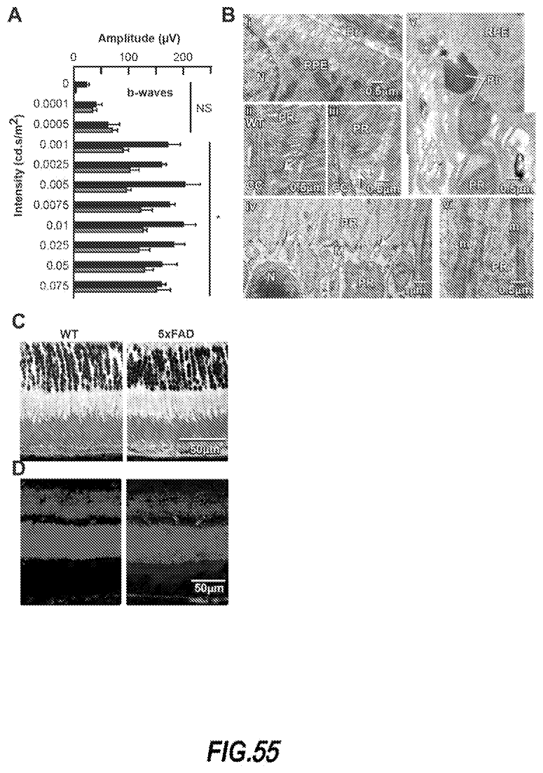

The b-wave ERG analysis of 6-month-old 5×FAD mice discloses a loss of visual sensitivity (FIG. 55, panel A). However, retina ultrastructure, the RPE cell/Bruch's membrane interface, the outer segment basal region of disk synthesis, the integrity of the outer limiting membrane (OLM), elongate inner segment mitochondria (no fission profiles), and PRC tip release and phagocytosis by the RPE (FIG. 55, panel B), demonstrated lack of abnormalities. Furthermore, histology did not show PRC loss of 5×FAD (FIG. 55, panel C). On the other hand, immunofluorescence microscopy showed that in 5×FAD the Aβ is mainly accumulated in the retina under the RPE as in AMD phenotype of drusen (FIG. 55, panel D).

ELVs Protect RPE and PRC Against OAβ-Induced Toxicity.

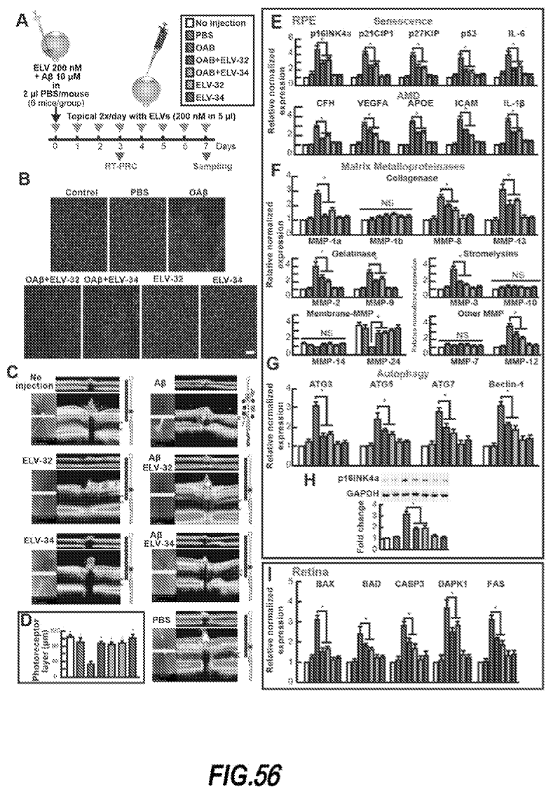

Because of the early deficits in the pro-homeostatic pathways leading to elovanoids in 5×FAD retina and the ensuing retinal degeneration, we next asked if ELVs would protect against the effect of OAβ, a most cytotoxic Aβ peptide (18). Six-month-old WT mice subretinally injected with OAβ demonstrated PRC degeneration (FIG. 56, panels A, C). Fundus (left side) and corresponding optical coherence tomography (OCT) (right side) images are depicted. The PRC layer underwent cell loss, from 105 μm thickness for the non-injected retina to 35 μm, for the OAβ-injected retina. Non-injected, PBS-injected and ELV-32, ELV-34-injected mice did not yield PRC degeneration (FIG. 56, panels C, D). The ZO-1 staining of flat mounted RPE revealed that oligomeric β-Amyloid disrupted tight junctions and triggered cell damage. We co-injected ELV32 or ELV34 with OAβ, followed by topical application of the elovanoids during 7 days (FIG. 56, panel A) resulting in restoration of RPE morphology (FIG. 56, panel B) and protection of PRC (FIG. 56, panels C and D). The mice injected with PBS or ELVs alone showed a small reduction of ONL, due to mechanical stress following subretinal injection (FIG. 56, panels C and D). These results demonstrate that elovanoids preserve the integrity of PRC, which denotes the ability of these lipid mediators to counteract cellular injury sustained by OAβ toxicity.

ELVs counteract OAβ-induced senescence, autophagy, AMD and ECM remodeling gene expression disruptions in RPE and of apoptotic gene expression in retina. To search for mechanism(s) involved in the ELV protection against OAβ-mediated damage, isolated RPE and retina were subjected to quantitative PCR (qPCR). We selected to survey genes involved in senescence (19, 20), autophagy (21), AMD (22, 23) and ECM remodeling (24) on day 3 post-injection in the RPE (FIG. 56, panels E-G). In addition, we explored cell death-related genes Bax, Bad, Casp3, Dapk1 and Fas in the retina (FIG. 56, panel I). The OAβ-mediated upregulation of senescence, autophagy, AMD and some ECM remodeling gene expression was counteracted by elovanoids (FIG. 56). Certain matrix metalloproteinases (1b,10,14 and 7) were not affected by OAβ. In addition, in the RPE, the protein abundance of the key senescence p16INK4a (FIG. 56, panel H) correlates with those on its gene expression (FIG. 56, panel E).

ELVs Protect Human RPE Cells from OAβ-Induced Senescence and Other Gene Transcription Disruptions.

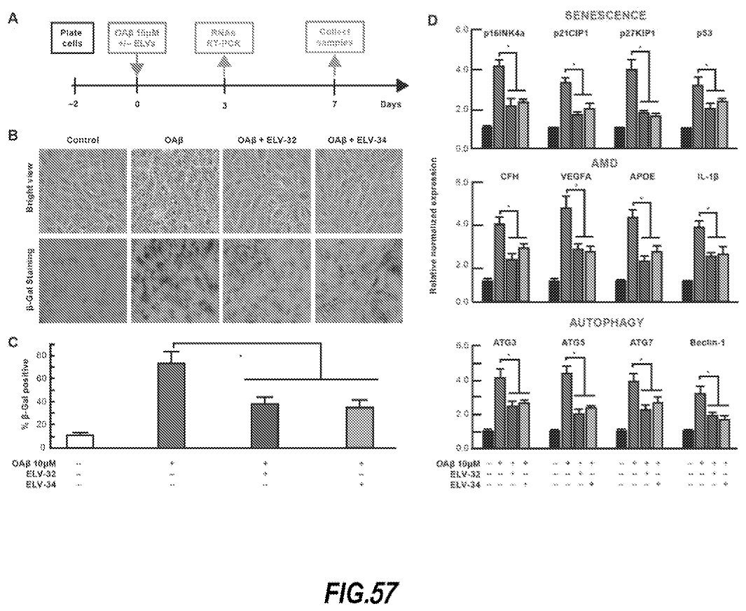

Since 5×FAD mice display RPE tight junction disruptions upon Aβ accumulation (16), we used a primary human RPE cell in culture challenged with OAβ to assess damage and to evaluate ELV-N-32 or ELV-N-34 protection (FIG. 57, panel A). After 7 days incubation, oligomeric β-Amyloid altered RPE cell morphology and activated SASP, as revealed by the SA-β-Gal staining (FIG. 57, panel B and C), as well as enhanced the expression of a set of senescence genes (FIG. 57, panel E), AMD, matrix metalloproteinases and autophagy-related genes (FIG. 57, panel D). A point of interest is that some matrix metalloproteinases were affected, but not all expressed in RPE cells. In other cells, SASP is primarily pro-inflammatory and has been shown to comprise chemokines, metalloproteinases, proteases, cytokines (e.g., TNF-α, IL-6, and IL-8), and insulin-like growth factor binding proteins. The senescence genes studied are P16 INK4a (Cdkn2a), p21CIP1(Cdkn1A), p27 KIP (Cdkn1B), p53 (Tp53 or TRP53), IL6 and MMP1. ELV-N-32 and ELV-N-34 reverted these effects (FIG. 57, panels B-D.

DISCUSSION AMD and Alzheimer's disease display accumulation of Abeta in the retina and brain, respectively. AP-based antibody as well as anti-inflammatory therapies for AD have been largely unsuccessful, therefore there is a need to understand mechanisms and identify specific agents that limit Aβ neurotoxicity (25-28). RPE sustains PRC integrity and its dysfunction sets in motion PRC death in retinal degenerative diseases, including AMD. Here we show that Oaβ drives RPE and PRC pathology, both in vivo in a rodent and in primary human RPE cell culture. Early in the pathogenesis of 5×FAD PRC degeneration, we report deficits in precursors and pathways for NPD1 and ELV biosynthesis. These deficits precede ECM and histology signs of PRC damage while ERG already displays impairments. These findings uncover prodromal alterations of key pro-homeostatic lipid signaling during onset and early disease progression. Aside from being used as biomarkers, they can also be explored as therapeutic targets for AMD.

There is not clear evidence in genetic animal models that blocking Aβ formation results in reduced AMD pathology. However, there are studies aiming to inhibit Aβ in the eye experimentally to protect PRC. For example, Liu et al. showed that 10 months of Aβ vaccination inhibits retinal deposits but causes retinal amyloid angiopathy characterized by microglial infiltration and astrogliosis in AD-transgenic mice (29). A drawback to this is the fact that active immunization can cause severe side effects.

It is not clear how many AD patients develop AMD, nor vice versa. However, there is a correlation between AD and eye diseases besides AMD that can includes glaucoma and susceptibility to diabetic retinopathy (30). Evolving key signaling disease mechanisms, can includes CFH, APOE (31-33) and the matrix metalloproteinase pathway (34). Our data shows that subretinal OAβ injection in mice triggers RPE cell damage and PRC loss after 7 days. To test the soundness of the Aβ deleterious effects on the RPE, we used human RPE cells in primary cultures and showed that it sets in motion similar damage as in the in vivo rodent. Moreover, both in the rodent model in vivo and in human cells in vitro, the changes in gene expression profiles were similar. Aβ synthesis takes place in the RPE (35-38) and accumulates in drusen, it is becoming evident that amyloid precursor protein processing dysfunctions lead as well to accumulation of the peptide within the retina also adjacent to ganglion cells, to the inner nuclear layer (39-41) and its synthesis, abundance, secretion, and aggregation increases in an age-dependent fashion (39). Our subretinal injection of Aβ here recapitulates some conditions associated with pathology of AMD targeting the RPE.

The finding that OAβ-induced RPE and photoreceptor cell death in wild-type mice in vivo was counteracted by elovanoids uncovers an additional bioactivity of these specific downstream mediators from omega-3 fatty acids. Mechanistically, neuroinflammatory disruptions are involved in early stages of AMD pathology and several studies have used dietary supplementation with omega-3 fatty acids (42-46), which have not yielded clear benefits, due to the supply of these critical fatty acids to the PRC and synapses involves complex steps that can include gut, liver, blood stream transport, cellular uptake, etc. (47, 48). A rational therapeutic approach for AMD can be to use mediators from omega-3 fatty acids that have neuroprotective bioactivity.

The study identifies the ELVs 32 and 34 as downregulatory mediators of OAβ-evoked senescence, as shown by SASP and the expression of senescence-related genes in RPE. Under these conditions, the upregulated expression of autophagy- and AMD-related genes, including human complement factor (49) and extracellular matrix-genes, were beneficially targeted by elovanoids as well. Thus, the similarities on the oligomeric β-Amyloid elicited effects in RPE cells in culture and in RPE and PRC in vivo, including the ELV-targeted protection, indicate relevance to the human retina. Surprisingly, we observed that OAβ injection caused apoptosis-related cell death signaling in photoreceptor cells not senescence. However, it is important to note that elovanoids prevented both OAβ-induced senescence in RPE and OAβ-induced PRC apoptosis.

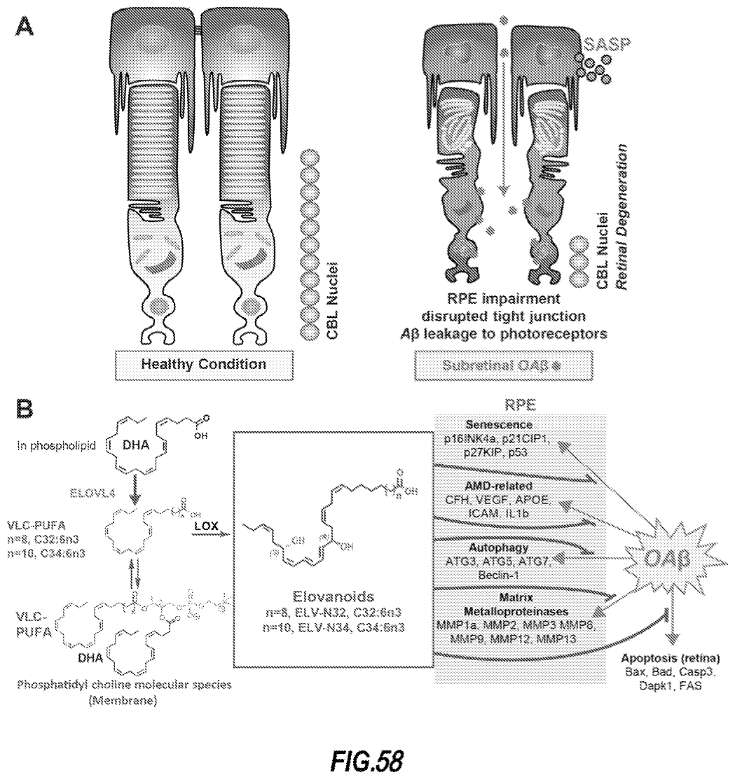

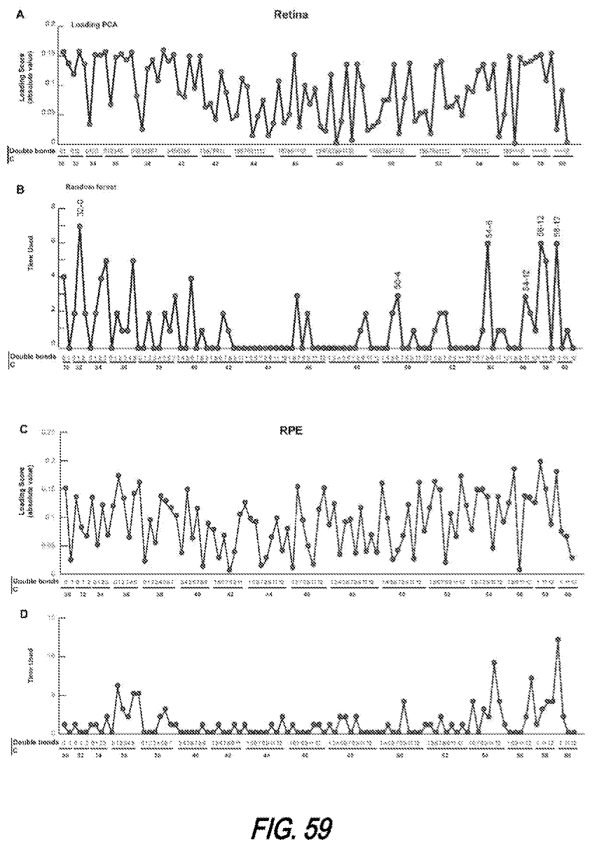

In conclusion, we uncovered early deficits of pro-homeostatic pathways before PRC death in the 5×FAD mice, highlighted by decreased abundance of PC molecular species in RPE (for example, those containing VLC-PUFAs) and in retina (those containing DHA and VLCPUFAs). Also, the pool size of free VLC-PUFAs and stable derivatives of precursors 27- and 29-monohydoxy and of ELV-32 and ELV-34, respectively, were found to be depleted. Moreover, the retina displays deficiencies in key enzymes of the pathways for the synthesis of pro homeostatic/neuroprotective NPD1 and ELVs without overt PRC damage or loss but shows functional impairments. Elovanoids counteracted the cytotoxicity of OAβ subretinally administered in WT mice leading to RPE tight junction disruptions followed by PRC cell death. Our data show that OAβ activates a senescence program reflected by enhanced gene expression of p16INK4a, MMP1, p53, p21, p27, Il-6, MMP1 and SASP secretome, followed by RPE and PRC demise, and that ELV-N-32 and ELV-N-34 blunt these events and elicit protection to both cells. The RPE cell is terminally differentiated and originated from the neuroepithelium. In this connection, senescent neurons in aged mice and models of AD (50) and astrocytes (51,52) also express senescence and develop secretory SASP that fuel neuroinflammation in nearby cells (53-55). Our study shows neighbor cells can be targeted by SASP neurotoxic actions, inducing photoreceptor paracrine senescence. Therefore, SASP from RPE cells may be autocrine and paracrine, altering the homeostasis of the interphotoreceptor matrix microenvironment as a consequence and creating an inflammatory milieu the contributes to loss of function associated with ageing (56), age-related pathologies (56) and AMD. Furthermore, ELVs restore expression of ECM remodeling matrix metalloproteinases altered by OAβ treatment, pointing to an additional disturbance in the interphotoreceptor matrix. The inflammation set in motion can be a low-grade, sterile, chronic pro-inflammatory condition similar to inflammaging that is also linked to senescence of the immune system (56, 57). In addition, elovanoids counteracted OAβ-induced expression of genes engaged in AMD and autophagy. Without wishing to be bound by theory, the elovanoids targeted event(s) on gene transcription (FIG. 58) to inform new unifying regulatory mechanisms to sustain health span during aging and neurodegenerative diseases (56, 58). Although further research is needed, our results, overall, show ELVs as a therapeutic avenue of exploration for AMD.

Materials and Methods

Materials and Methods. This information can include animals, lipid extraction and LC-MS/MS-based lipidomic analysis, primary human RPE culture, Aβ (1-42) oligomerization, SA-β-Gal staining, protein extraction and western blot analysis, RNA isolation and quantitative PCR analysis, immunofluorescence and confocal microscopy, and statistics.

### REFERENCES CITED IN THIS EXAMPLE

1. N. G. Bazan, Cell survival matters: docosahexaenoic acid signaling, neuroprotection and photoreceptors. Trends Neurosci. 29, 263-271 (2006).
2. W. J. Lukiw, et al., A role for docosahexaenoic acid-derived neuroprotectin D1 in neural cell survival and Alzheimer disease. J. Clin. Invest. 115, 2774-2783 (2005).
3. L. V. Johnson, et al., The Alzheimer's A beta-peptide is deposited at sites of complement activation in pathologic deposits associated with aging and age-related macular degeneration. Proc. Natl. Acad. Sci. U.S.A. 99, 11830-11835 (2002).
4. D. H. Anderson, et al., Characterization of beta amyloid assemblies in drusen: the deposits associated with aging and age-related macular degeneration. Exp. Eye Res. 78, 243-256 (2004).
5. M.-P. Agbaga, et al., Role of Stargardt-3 macular dystrophy protein (ELOVL4) in the biosynthesis of very long chain fatty acids. Proc. Natl. Acad. Sci. U.S.A. 105, 12843-12848 (2008).
6. R. Harkewicz, et al., Essential role of ELOVL4 protein in very long chain fatty acid synthesis and retinal function. J. Biol. Chem. 287, 11469-11480 (2012).
7. M. I. Aveldaño, Phospholipid species containing long and very long polyenoic fatty acids remain with rhodopsin after hexane extraction of photoreceptor membranes. Biochemistry 27, 1229-1239 (1988).
8. M. A. Aldahmesh, et al., Recessive mutations in ELOVL4 cause ichthyosis, intellectual disability, and spastic quadriplegia. Am. J. Hum. Genet. 89, 745-750 (2011).
9. B. Jun, et al., Elovanoids are novel cell-specific lipid mediators necessary for neuroprotective signaling for photoreceptor cell integrity. Scientific Reports 7, 5279 (2017).
10. N. G. Bazan, Docosanoids and elovanoids from omega-3 fatty acids are pro-homeostatic modulators of inflammatory responses, cell damage and neuroprotection. Mol. Aspects Med. 64, 18-33 (2018).
11. S. Bhattacharjee, et al., Elovanoids are a novel class of homeostatic lipid mediators that protect neural cell integrity upon injury. Science Advances 3, e1700735 (2017).
12. T. Dentchev, A. H. Milam, V. M.-Y. Lee, J. Q. Trojanowski, J. L. Dunaief, Amyloid-beta is found in drusen from some age-related macular degeneration retinas, but not in drusen from normal retinas. Mol. Vis. 9, 184-190 (2003).
13. M. T. Heneka, et al., Neuroinflammation in Alzheimer's disease. Lancet Neurol 14, 388-405 (2015).
14. T. Prasad, et al., Amyloid R peptides overexpression in retinal pigment epithelial cells via AAV-mediated gene transfer mimics AMD-like pathology in mice. Sci Rep 7, 3222 (2017).
15. K. H. Kurji, et al., Microarray analysis identifies changes in inflammatory gene expression in response to amyloid-beta stimulation of cultured human retinal pigment epithelial cells. Invest. Ophthalmol. Vis. Sci. 51, 1151-1163 (2010).
16. S. W. Park, et al., Dry age-related macular degeneration like pathology in aged 5×FAD mice: Ultrastructure and microarray analysis. Oncotarget 8, 40006-40018 (2017).
17. C. Criscuolo, et al., The retina as a window to early dysfunctions of Alzheimer's disease following studies with a 5×FAD mouse model. Neurobiol. Aging 67, 181-188 (2018).
18. B. A. Yankner, T. Lu, Amyloid beta-protein toxicity and the pathogenesis of Alzheimer disease. J. Biol. Chem. 284, 4755-4759 (2009).
19. R. Salama, M. Sadaie, M. Hoare, M. Narita, Cellular senescence and its effector programs. Genes Dev. 28, 99-114 (2014).
20. P. Zhang, et al., Senolytic therapy alleviates Aβ-associated oligodendrocyte progenitor cell senescence and cognitive deficits in an Alzheimer's disease model. Nat. Neurosci. 22, 719-728 (2019).
21. S. K. Mitter, et al., Dysregulated autophagy in the RPE is associated with increased susceptibility to oxidative stress and AMD. Autophagy 10, 1989-2005 (2014).
22. C. B. Toomey, L. V. Johnson, C. Bowes Rickman, Complement factor H in AMD: Bridging genetic associations and pathobiology. Prog Retin Eye Res 62, 38-57 (2018).
23. M. M. DeAngelis, et al., Genetics of age-related macular degeneration (AMD). Hum. Mol. Genet. 26, R45-R50 (2017).
24. B. Budiene, et al., The association of matrix metalloproteinases polymorphisms and interleukins in advanced age-related macular degeneration. Ophthalmic Genet. 39, 463-472 (2018).
25. F. Panza, M. Lozupone, G. Logroscino, B. P. Imbimbo, A critical appraisal of amyloid-β-targeting therapies for Alzheimer disease. Nat Rev Neurol 15, 73-88 (2019).
26. P. L. McGeer, E. G. McGeer, The amyloid cascade-inflammatory hypothesis of Alzheimer disease: implications for therapy. Acta Neuropathol. 126, 479-497 (2013).
27. A. C. Cuello, Early and Late CNS Inflammation in Alzheimer's Disease: Two Extremes of a Continuum? Trends Pharmacol. Sci. 38, 956-966 (2017).
28. A. Crane, et al., Peripheral complement interactions with amyloid β peptide in Alzheimer's disease: 2. Relationship to amyloid β immunotherapy. Alzheimers Dement 14, 243-252 (2018).
29. B. Liu, et al., Amyloid-peptide vaccinations reduce {beta}-amyloid plaques but exacerbate vascular deposition and inflammation in the retina of Alzheimer's transgenic mice. Am. J Pathol. 175, 2099-2110 (2009).
30. C. S. Lee, et al., Associations between recent and established ophthalmic conditions and risk of Alzheimer's disease. Alzheimers Dement 15, 34-41 (2019).
31. L. Biscetti, et al., Associations of Alzheimer's disease with macular degeneration. Front Biosci (Elite Ed) 9, 174-191 (2017).
32. M. A. Williams, et al., Age-Related Macular Degeneration-Associated Genes in Alzheimer Disease. Am J Geriatr Psychiatry 23, 1290-1296 (2015).
33. S. Frost, et al., Alzheimer's Disease and the Early Signs of Age-Related Macular Degeneration. Curr Alzheimer Res 13, 1259-1266 (2016).
34. A. A. Hussain, Y. Lee, J.-J. Zhang, P. T. Francis, J. Marshall, Disturbed Matrix Metalloproteinase Pathway in Both Age-Related Macular Degeneration and Alzheimer's Disease. J Neurodegener Dis 2017, U.S. Pat. No. 4,810,232 (2017).
35. S. A. Lynn, et al., The complexities underlying age-related macular degeneration: could amyloid beta play an important role? Neural Regen Res 12, 538-548 (2017).
36. J. A. Ratnayaka, L. C. Serpell, A. J. Lotery, Dementia of the eye: the role of amyloid beta in retinal degeneration. Eye (Lond) 29, 1013-1026 (2015).
37. T. Yoshida, et al., The potential role of amyloid beta in the pathogenesis of age-related macular degeneration. J. Clin. Invest. 115, 2793-2800 (2005).
38. J. Wang, K. Ohno-Matsui, I. Morita, Elevated amyloid § production in senescent retinal pigment epithelium, a possible mechanism of subretinal deposition of amyloid β in age related macular degeneration. Biochem. Biophys. Res. Commun. 423, 73-78 (2012).
39. J. Wang, et al., Development and expression of amyloid-β peptide 42 in retinal ganglion cells in rats. AnatRec (Hoboken) 294, 1401-1405 (2011).
40. R. M. Dutescu, et al., Amyloid precursor protein processing and retinal pathology in mouse models of Alzheimer's disease. Graefes Arch. Clin. Exp. Ophthalmol. 247, 1213-1221 (2009).
41. J. Hoh Kam, E. Lenassi, G. Jeffery, Viewing ageing eyes: diverse sites of amyloid Beta accumulation in the ageing mouse retina and the up-regulation of macrophages. PloS ONE 5 (2010).
42. M. Fiala, G. Kooij, K. Wagner, B. Hammock, M. Pellegrini, Modulation of innate immunity of patients with Alzheimer's disease by omega-3 fatty acids. FASEB J. 31, 3229-3239 (2017).
43. F. La Rosa, et al., The Gut-Brain Axis in Alzheimer's Disease and Omega-3. A Critical Overview of Clinical Trials. Nutrients 10 (2018).
44. N. M. D'Cunha, et al., Effect of long-term nutraceutical and dietary supplement use on cognition in the elderly: a 10-year systematic review of randomised controlled trials. Br. J Nutr. 119, 280-298 (2018).
45. M. Belkouch, et al., The pleiotropic effects of omega-3 docosahexaenoic acid on the hallmarks of Alzheimer's disease. J. Nutr. Biochem. 38, 1-11 (2016).
46. J. G. Devassy, S. Leng, M. Gabbs, M. Monirujjaman, H. M. Aukema, Omega-3 Polyunsaturated Fatty Acids and Oxylipins in Neuroinflammation and Management of Alzheimer Disease. Adv Nutr 7, 905-916 (2016).
47. N. G. Bazan, M. F. Molina, W. C. Gordon, Docosahexaenoic acid signalolipidomics in nutrition: significance in aging, neuroinflammation, macular degeneration, Alzheimer's, and other neurodegenerative diseases. Annu. Rev. Nutr. 31, 321-351 (2011).
48. D. S. Rice, et al., Adiponectin receptor 1 conserves docosahexaenoic acid and promotes photoreceptor cell survival. Nature Communications 6, 6228 (2015).
49. M. Landowski, et al., Human complement factor H Y402H polymorphism causes an age related macular degeneration phenotype and lipoprotein dysregulation in mice. Proc. Natl. Acad. Sci. U.S.A. 116, 3703-3711 (2019).
50. N. Musi, et al., Tau protein aggregation is associated with cellular senescence in the brain. Aging Cell 17 (2018).
51. M. M. Das, C. N. Svendsen, Astrocytes show reduced support of motor neurons with aging that is accelerated in a rodent model of ALS. Neurobiol. Aging 36, 1130-1139 (2015).
52. C. Turnquist, et al., p53 isoforms regulate astrocyte-mediated neuroprotection and neurodegeneration. Cell Death Differ. 23, 1515-1528 (2016).
53. S. H. Appel, W. Zhao, D. R. Beers, J. S. Henkel, The microglial-motoneuron dialogue in ALS. Acta Myol 30, 4-8 (2011).
54. O. Komine, K. Yamanaka, Neuroinflammation in motor neuron disease. Nagoya J Med Sci 77, 537-549 (2015).
55. V. V. Lunyak, A. Amaro-Ortiz, M. Gaur, Mesenchymal Stem Cells Secretory Responses: Senescence Messaging Secretome and Immunomodulation Perspective. Front Genet 8, 220 (2017).
56. J. Campisi, et al., From discoveries in ageing research to therapeutics for healthy ageing. Nature 571, 183-192 (2019).
57. S. He, N. E. Sharpless, Senescence in Health and Disease. Cell 169, 1000-1011 (2017). 58. D. J. Baker, et al., Naturally occurring p16(Ink4a)-positive cells shorten healthy lifespan. Nature 530, 184-189 (2016).

Materials and Methods

Animals

All animal experiments were performed according to ARVO Statement for the Use of Animals in Ophthalmic and Vision Research, and the protocol was approved by LSU Health New Orleans' Institutional Animal Care and Use Committee (IACUC). 6-month-old 5×FAD mice (stock number: 34848-JAX, The Jackson Laboratory, Bar Harbor, ME, USA), co-overexpress FAD mutant forms of human amyloid precursor protein (the Swedish mutation: K670N, M671L; the Florida mutation: I716V; and the London mutation: V7171) and presenilin 1 (PS1, encoded by Psen1: M146L, L286V) transgenes under transcriptional control of the neuron-specific mouse Thy1 promoter. 5×FAD mice were hemizygotes with respect to the transgenes and non-transgenic WT littermates. Genotyping was performed by PCR of tail DNA. All analyses were carried out blind with respect to the mice genotype. For the subretinal injection, 6-month-old C57BL/6J mice were anesthetized by an intraperitoneal injection of ketamine/xylazine, pupils dilated with 1.0% tropicamide (Akorn, IL, USA); and 0.5% Proparacaine Hydrochloride (Akorn) was applied for topical anesthesia. Eyes were punctured with a 30-gauge needle between the corneoscleral junction and the ora serrata into the vitreous cavity without disturbing the lens. Compounds were delivered to the subretinal region using a 33-gauge blunt needle attached to a 5 μl Hamilton syringe (Hamilton Company, Reno, NV, USA) under a dissecting microscope. Non-injected mice were used as negative control while the PBS injected mice were used for sham. The injection volume was 2 μl containing PBS, 10 μM of OAβ, 10 μM of OAβ+200 ng ELV-N-32, 200 ng ELV-N-32 alone, 10 μM of OAβ+200 ng ELV-N-34 or 200 ng ELV-N-34 alone (n=12/group). All groups received topical drops: PBS only or ELV-N-32 (200 nM) or ELV-N-34 (200 nM), twice a day for 3 or 7 days.

Lipid Extraction and LC-MS/MS-Based Lipidomic Analysis

Retina or RPE/choroid were homogenized in 3 ml of MeOH followed by adding 6 ml of CHCl3 and 5 μl of an internal standard mixture of deuterium-labeled lipids (AA-d8 (5 ng/μl), PGD2-d4 (1 ng/μl), EPA-d5 (1 ng/μl), 15-HETE-d8 (1 ng/μl), and LTB4-d4 (1 ng/μl)). Samples were sonicated for 30 min and stored at −80° C. overnight. Then supernatant collected, pellet washed with 1 ml of CHCl3/MeOH (2:1) and centrifuged, and supernatants combined. Two ml of distilled water, pH 3.5, was added to the supernatant, vortexed, and centrifuged, and then the pH of the upper phase adjusted to 3.5-4.0 with 0.1 N HCl. The lower phase was dried down under N2 and then resuspended in 1 ml of MeOH. LC-MS/MS analysis was performed in a Xevo TQ equipped with Acquity I class UPLC with a flow-through needle (Waters, Milford, MA, USA). For PC and PE molecular species analysis, samples were dried under N2 and then resuspended in 20 μl of the sample solvent (acetonitrile/chloroform/methanol, 90:5:5 by volume). The Acquity UPLC BEH HILIC 1.7-μm, 2.1×100-mm column was used with a mixture of solvent A (acetonitrile/water, 1:1; 10 mM ammonium acetate, pH 8.3) and solvent B (acetonitrile/water, 95:5; 10 mM ammonium acetate, pH 8.3) as the mobile phase (0.5 ml/min). Solvent B (100%) was isocratically run for the first 5 min and then run in a gradient to 20% of solvent A for 8 min, increased to 65% of solvent A for 0.5 min, run isocratically at 65% of solvent A for 3 min, and then returned to 100% of solvent B for 3.5 min for equilibration. The column temperature was set to 30° C. The amount for each PC and PE species was calculated as % of the total PCs and PEs/sample. For analysis of fatty acids and their derivatives, six retinas or six RPE/Choroid were pooled and homogenized as described herein. Samples (in 1 ml of MeOH) were mixed with 9 ml of H2O at pH 3.5, loaded onto C18 columns (Agilent, Santa Clara, CA, USA), and then eluted with 10 ml of methyl formate, dried under N2, resuspended in 50 μl of MeOH/H2O (1:1), and injected into an Acquity UPLC HSS T3 1.8-μm 2.1×50-mm column. Mobile phase 45% solvent A—H2O+0.01% acetic acid and 55% solvent B—MeOH+0.01% acetic acid-, 0.4 ml/min flow initially, and then a gradient to 15% solvent A for the first 10 min, a gradient to 2% solvent A for 18 min, 2% solvent A run isocratically until 25 min, and then a gradient back to 45% solvent A for re-equilibration until 30 min. Lipid standards (Cayman, Ann Arbor, MI, USA) were used for tuning and optimization, as well as to create calibration curves for each compound.

Primary Human RPE Culture

All experiments with primary human RPE cells were approved by the Institutional Review Board of LSUHNO and conducted in accordance with NIH guidelines. Cells were collected from anonymous donors provided by eye banks. Briefly, globes of a 19-year-old Caucasian male, without eye pathology were obtained from NDRI within 24 hours after death from head trauma. Globes were opened, and RPE cells harvested and cultured (1, 2) and grown in MEM medium supplemented with 10% FBS, 5% NCS, non-essential amino acids, Penicillin-Streptomycin (100 U/mL), human fibroblast growth factor 10 ng/ml and incubated at 37° C. with a constant supply of 5% CO₂. Cells integrity was validated as in previous study (3). For oligomeric Aβ treatment, cells were seeded in the 6-well plates, 30.000 cells/cm2. After 2 days, sub-confluent cells were treated with 10 μM OAβ or with PBS (vehicle control).

Aβ (1-42) Oligomerization

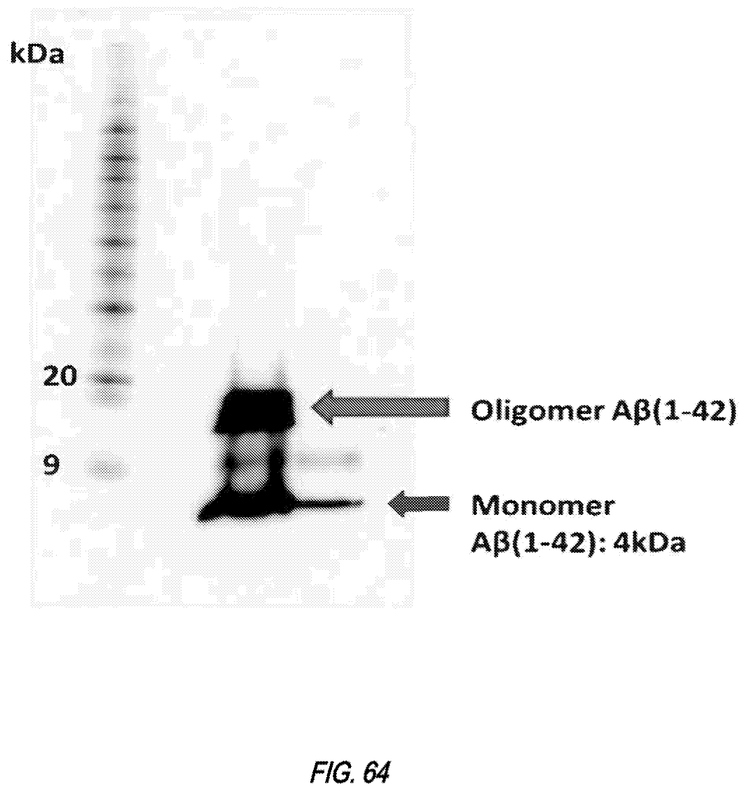

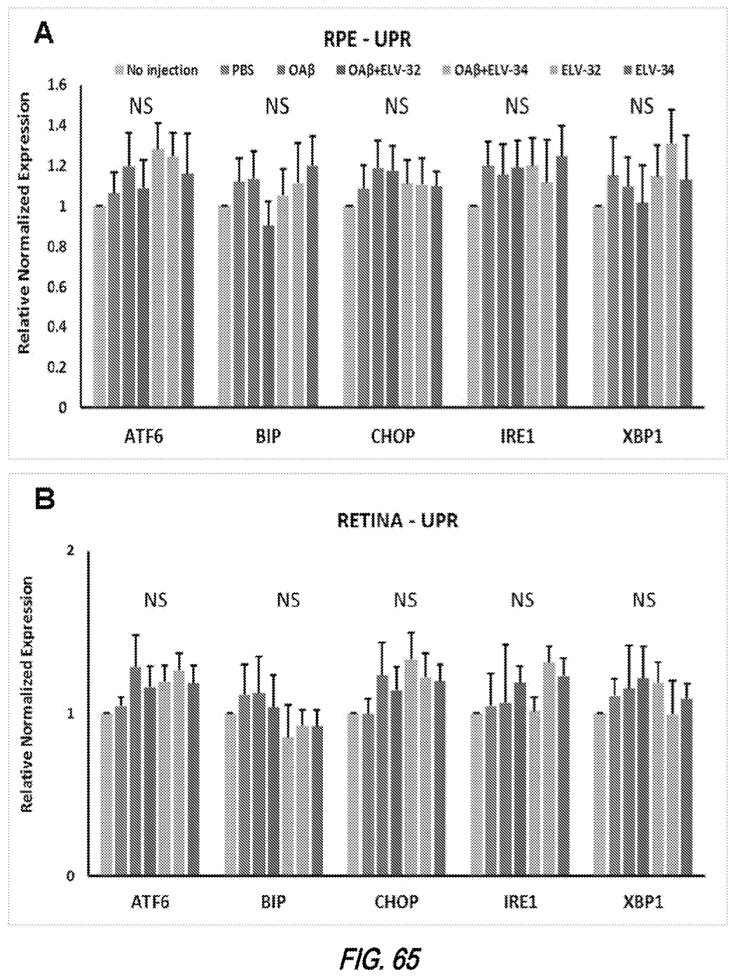

Aβ (1-42) (HFIP-treated, ANASPEC Company, Fremont, CA, USA, Cat AS-64129) was resuspended by adding 1% NH4OH/Water and DMSO to obtain a concentration 500 μM and sonicated for 10 min. Then oligomerization was performed by diluting t Aβ (1-42) with sterile phosphate buffer in low-binding polypropylene micro-centrifuge tube for 24 h at 4° C. Oligomerization was verified by Western blot using mouse monoclonal 6E10 antibody (FIG. 64).

Senescence-Associated β-Galactosidase (SA-β-Gal) Staining

Cells were visualized using SA-β-Gal staining kit (Cat 9860, Cell Signaling Technology, MA, USA). Briefly, RPE cells were washed with PBS, fixed with 4% paraformaldehyde (PFA) for 15 min, then washed again with PBS and incubated in staining solution mix overnight at 37° C. (no CO2), the presence of CO2 can cause changes to the pH which may affect staining results. Pictures were taken under brightfield microscope (Nikon Eclipse TS100) 200× magnification after the development of blue color, and cells counted in 10 different random fields per well.

Protein Extraction and Western Blot Analysis

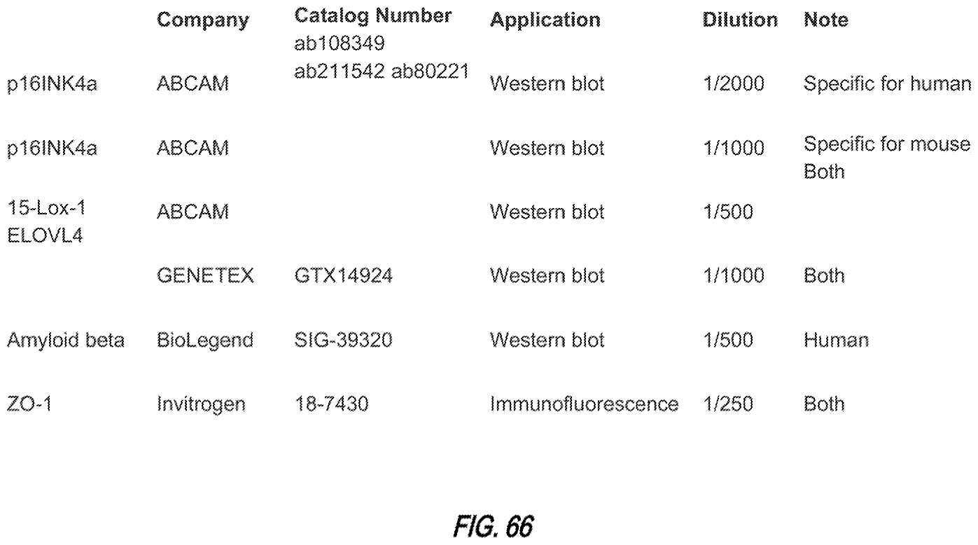

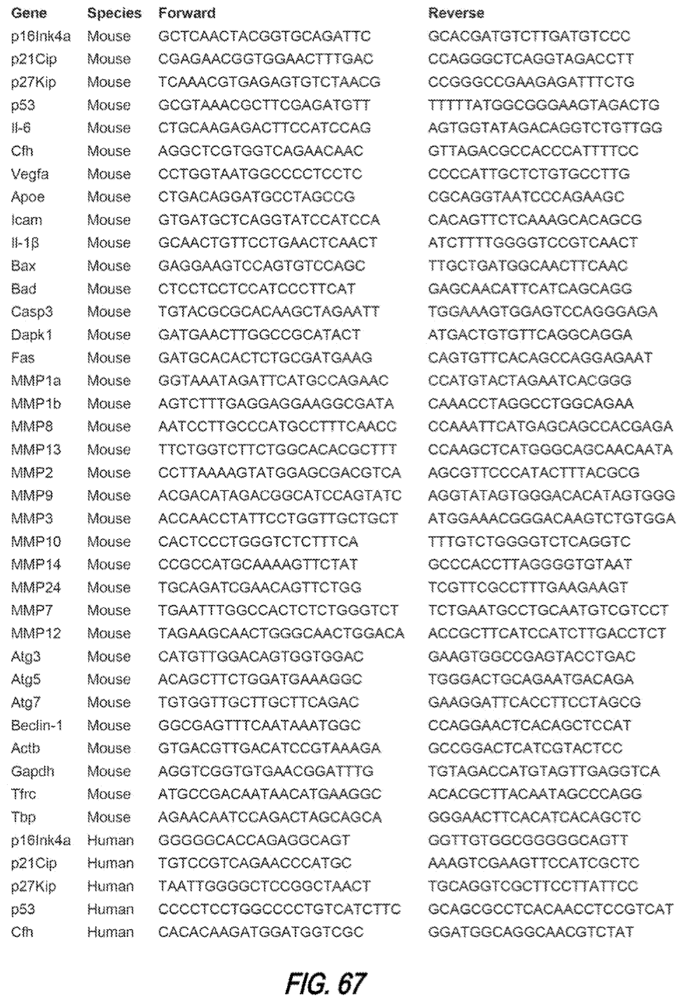

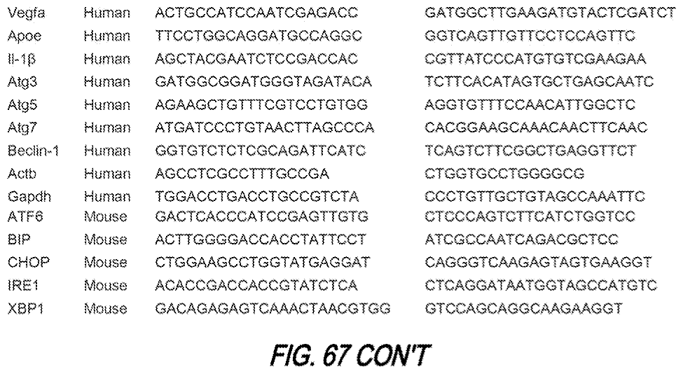

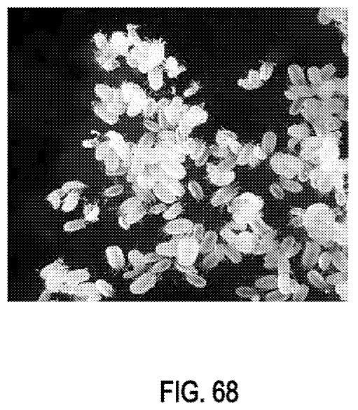

Samples were lysed by RIPA buffer and protein determined by Bradford assay (Bio-Rad, Hercules, CA, USA). After denaturation, 20 μl of each medium sample or 30 g of total protein for cell/tissue sample was separated by SDS-PAGE (4-12% gradient) gel (Thermo Fisher Scientific, Waltham, MA, USA) and transferred to nitrocellulose membranes (Bio-Rad). The membranes were blocked by 5% non-fat dry milk in PBST, probed with primary antibodies (FIG. 66) for 1 h, washed 3× by PBST, probed with secondary antibodies (GE Healthcare, Chicago, IL, USA) for 1 h, and washed 3× by PBST. Proteins bands were visualized using a LAS 4000 imaging system (GE Healthcare). Densitometry data were statistically analyzed at 95% confidence level.

RNA Isolation and qPCR Analysis

Cell culture media was removed, cells were wash with PBS 1× and samples were collected using cell scraper. Total RNA was isolated using RNeasy Plus Mini Kit (Qiagen, Hilden, Germany).

For the in vivo experiments, eyeballs were enucleated and anterior segment containing the cornea, lens and iris removed and the retina separated from the rest of the eyecup (RPE/choroid). Total retinal and eyecup (RPE/choroid) RNA were isolated using RNeasy Plus Mini Kit (Qiagen). One g of total RNA was reverse transcribed using an iScript cDNA Synthesis Kit (Bio-Rad) and the reaction carried out with BrightGreen 2× qPCR MasterMix (Applied Biological Materials Inc., Richmond, BC, Canada) and validated primers (SI Appendix, Table S2). Quantitative PCR was performed in a CFX-384 Real-Time PCR system (Bio-Rad). The expression of target genes was normalized to the geometric mean of housekeeping genes and relative expression was calculated by the comparative threshold cycle method (ΔΔCT).

Immunofluorescence and Confocal Microscopy

For the whole mount RPE staining, eyeballs were enucleated and pre-fixed in 4% PFA for 15 min. Then the eye cup containing RPE sheet were fixed in 4% PFA for 30 min, washed in PBS 3× following the blocking step for 1 h at room temperature. The immunostaining was performed by incubating primary antibody (ZO-1) for 48 h at 4° C. Then the eye cups were washed 3× with PBS and incubated with the secondary antibody for 12 h at 4° C. The primary human RPE cells as well as mouse eye cups were embedded in ProLong™ Gold Antifade Mounting medium (Thermo Fisher Scientific) between two glass coverslips. Pictures were taken with Olympus FV1200 microscope (Olympus, Japan). Images were analyzed by software ImageJ (rsb.info.nih.gov/ij/).

Spectral Domain-Optical Coherence Tomography Imaging and Analysis

7 days post-injection, mice were anesthetized with ip ketamine/xylazine, pupil dilated by topical 1.0% tropicamide and placed in a custom-built holder for OCT imaging (body temperature maintained at 38° C. with a heat pad). Retinas were imaged along the horizontal meridian through the optic nerve head using a Heidelberg Spectralis HRA OCT system (Heidelberg Engineering, Heidelberg, Germany). Axial resolution is 7 mm optical and 3.5 mm digital. The raw OCT B-scans cross-sectional images were exported with the scale in m and opened in ImageJ (http//imagej.nih.gov/ij). The PRC layer thickness was defined as the width from the tip of the outer nuclear layer, right after the outer plexiform layer, to the outer segments of PRC. Three measurements were made on the same scan and averaged. Mean and standard error of the mean (SEM) were calculated (n=4/group). Students' T-test was used to calculate statistical significance and a P-value less than 0.05 was considered significant.

Statistics

Data are expressed as mean±SEM of three or more independent experiments. The data were analyzed by one-way ANOVA followed by Tukey HSD post-hoc test at 95% confidence level to compare the different groups and considered significant with a P<0.05. The Pearson relation analysis was used to analyze the relationship between factors. Statistical analysis was performed by using BioVinci software (Bioturing INC., San Diego, CA, USA).

### REFERENCES

1. S. Sonoda, et al., A protocol for the culture and differentiation of highly polarized human retinal pigment epithelial cells. Nat Protoc 4, 662-673 (2009).
2. M. Ishida, G. M. Lui, A. Yamani, I. K. Sugino, M. A. Zarbin, Culture of human retinal pigment epithelial cells from peripheral scleral flap biopsies. Curr. Eye Res. 17, 392-402 (1998).
3. B. Jun, et al., Elovanoids are novel cell-specific lipid mediators necessary for neuroprotective signaling for photoreceptor cell integrity. Scientific Reports 7, 5279 (2017).

### Example 13

Elovanoids are Senolytics in Human Nasal Mucosal Cells Confronted with Allergens: Evidence for Therapeutics in Allergic Disease

Non-limiting, exemplary model of ELVs in OAβ-induced RPE and PRC damage.

OAβ induces senescence and disrupts the tight junction of RPE. Next, OAβ penetrates the retina, causing cell death of photoreceptors reflected in less cell body layer (CBL) nuclei. The Elovanoids restore the morphology of the RPE layer upon OAβ exposure and, as a consequence, the retina structure is preserved.

OAβ induces the senescence, autophagy, matrix metalloproteinases, and AMD-related genes in the RPE and apoptosis genes in retina. ELVs downregulated the OAβ-gene inductions.

Targets to tested: House Dust Mites (HDM)

HDM, indoor allergen, 1-2% suffers from HDM allergy.

HDM domestic source for allergic diseases (allergic rhinitis, asthma and atopic dermatitis).

HDM are arachnoids, of the Pyroglyphidae family: Dermatophagoidespteronyssinus and Dermatophagoides farinae (in drier areas) and Euroglyphus maynei (in humid areas).

Der p1 is an abundant allergen from mite fecal pellets and also an inducer of IgE response in patients with HDM allergy.

Dust mites thrive at 20-25 degrees Celsius and humidity of 70-80%.

There are at least 13 mites species adapted to the environment in our homes. They feed on flakes of human skin that we shed daily.

Allergy and House Dust Mites (HDM)
HDM are in dust, textile, carpets, beds, upholster furniture and curtain drapes. By dusting, vacuuming, and bed-making, mite fecal pellets and other aeroallergens become suspended in air, and inhaled.
Dust mite allergy symptoms comprise—Sneezing, Runny nose, Itchy, red or watery eyes, Stuffy and itchy nose, Itchy mouth or throat, Itchy skin, Postnasal drip (flow of mucus into your throat) and Cough.
HDM allergen/innate immune system—Toll-like receptors, protease activated receptors and DC-SIGN activated by HDM allergens, upregulation of proinflammatory cytokines.
Mite fecal pellets, Der p 1 accumulates at the apical surface of airway mucosa and trigger:
(1) degradation of antiprotease-based lung defenses (α1-antitrypsin, elafin, and secretory leukocyte protease inhibitor) and surfactant proteins (SPs; SP-A and SP-D);
(2) cleavage of tight junction proteins (occludin, zonula occludens-1, and cadherins) to facilitate the access of HDM allergens to immune cells (i.e., type 2 innate lymphoid cells [ILC2s] and dendritic cells [DCs]) in subepithelial tissue; and (3) stimulation of production of innate proinflammatory cytokines/chemokines/alarmins (i.e., IL-6, IL-8, GM-CSF, CCL2, and CCL20)

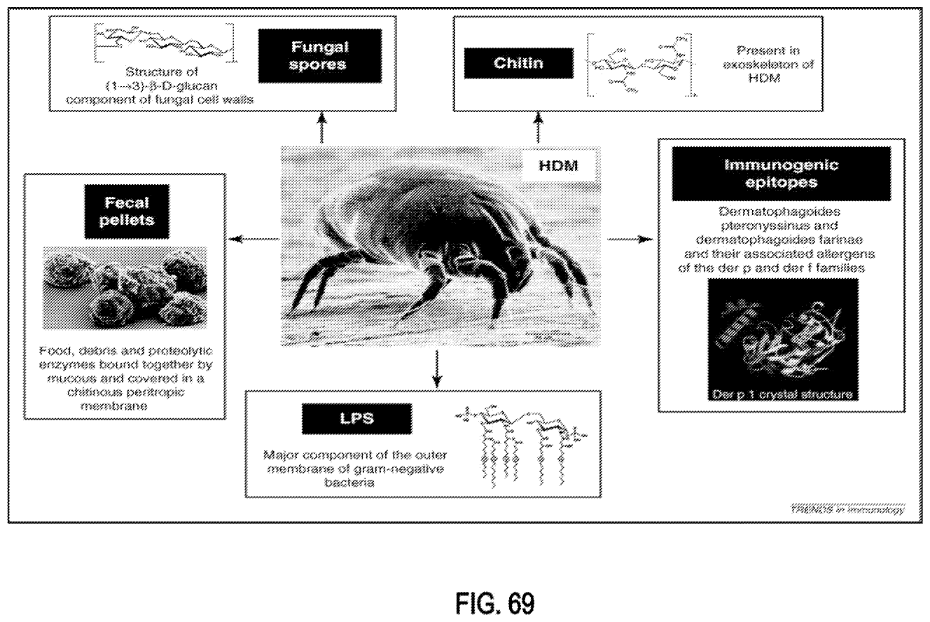

HDM Allergenicity: components, associated fecal pellets and dust, activator of the immune system to initiate inflammatory responses (FIG. 69, See Gregory 2011).

House dust mite allergenicity. The components of HDM, and their fecal pellets and dust, which activate the immune system to initiate an inflammatory response, are illustrated.

Non-limiting, exemplary experimental model: Human Nasal Epithelial Cells (from a 50-year-old Caucasian male)

Challenged by Aeroallergens (I)

Dermatophagoides pteronyssinus (D.P.) (Chondrex, Inc.)
D.P. (30 μg/mL)

Dermatophagoides farina (D.F.) 30 μg/mL
HDM mixture of both (D.P.) & (D.F.) (15 μg/mL)

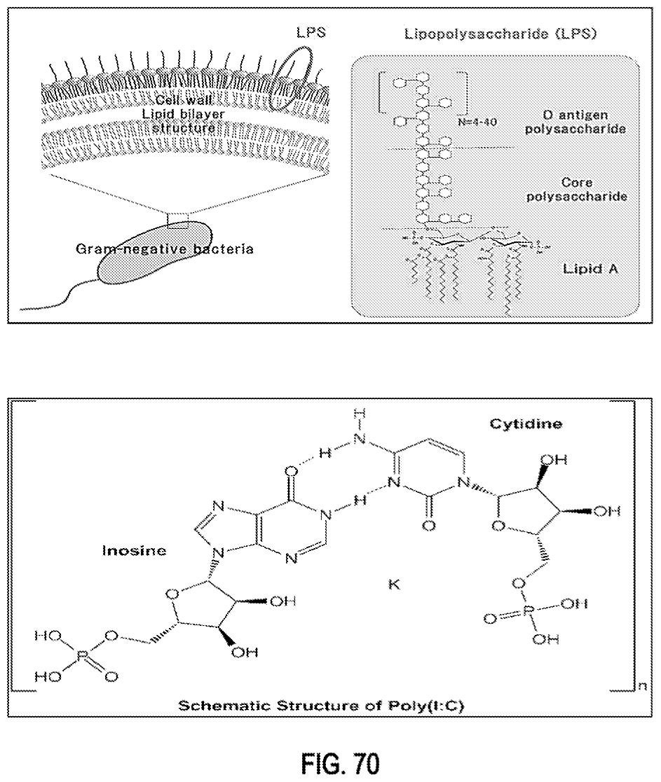

LPS and Poly(I:C) (FIG. 70)
Lipopolysaccharide (LPS), Gram-negative E. coli serotype 0111:B4 (Sigma-Aldrich) a preparation of smooth (S)-form LPS, 30 μg/mL.

LPS, principal component of Gram-negative bacteria, activates the innate immune system through its recognition by Toll-like receptor 4 (TLR4).

That leads to a signaling cascade that activates NF-κB and the production of cytokines engaged in senescence.
Polyinosinic-polycytidylic acid, poly(I:C) synthetic analog of double-stranded viral RNA, High Molecular Weight Poly(I:C) long strands of inosine poly(I), homopolymer annealed to strands of cytidinepoly(C) homopolymer (1.5 kb-8 kb). (Sigma-Aldrich) 100 g/mL.

A molecular pattern which can be activated with viral infection, loss of epithelial integrity, increased production of mucus and senescence programming cytokines.

A TLR3 agonist that activates the antiviral pattern recognition receptors TLR3, RIG-I/MDA5 and PKR, thereby inducing signaling via inflammatory pathways, including NF-κB and IRF.

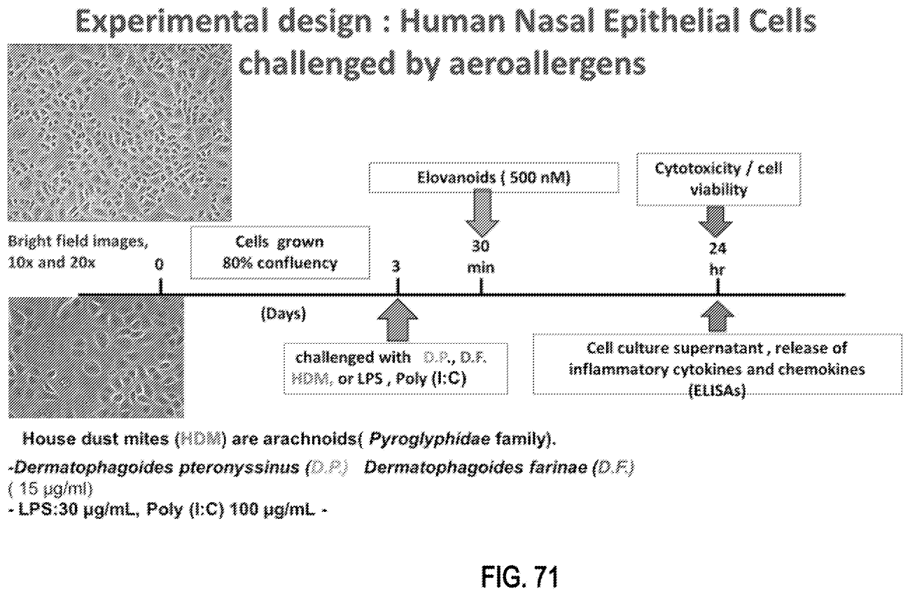

Non-limiting, exemplary experimental design: Human Nasal Epithelial Cells challenged by aeroallergens (FIG. 71).

House dust mites (HDM) are arachnoids (Pyroglyphidae family).

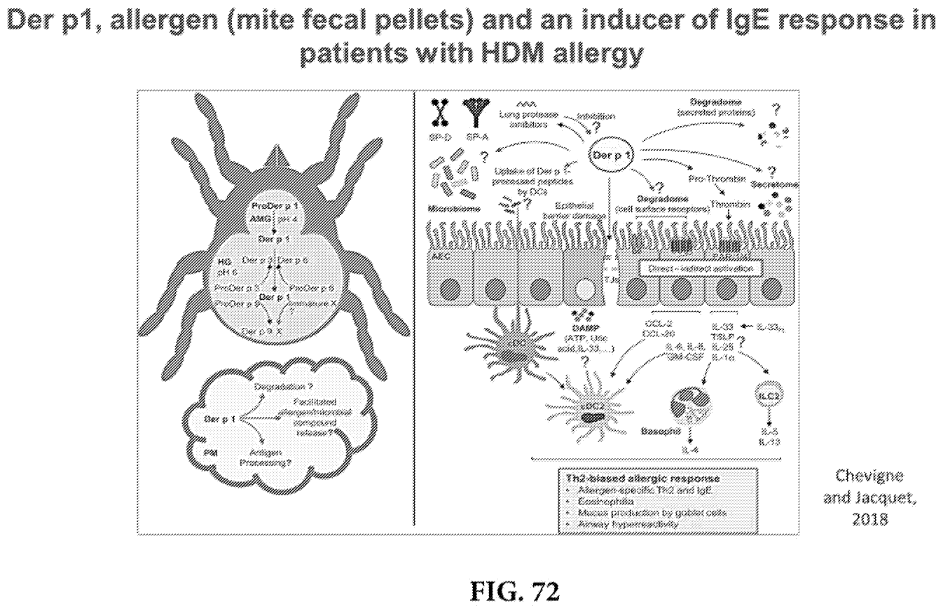

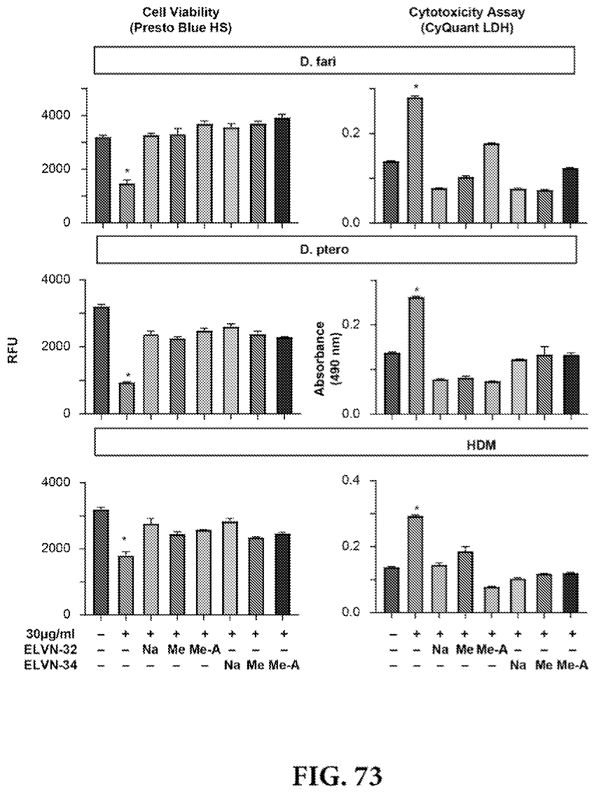

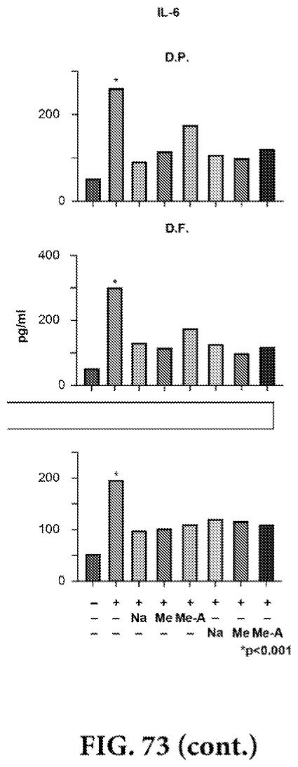

FIG. 72 shows a schematic representation of the reported and other functions of Der p 1 in the HDM induced inflammatory response. Der p 1 triggers a proteolytic activation cascade in the digestive tract of the mite Dermatophagoides pteronyssinus, leading to maturation of the serine protease allergens Der p 3, Der p 6, and Der p 9 and without wishing to be bound by theory, other unknown mite protein targets.

Without wishing to be bound by theory:

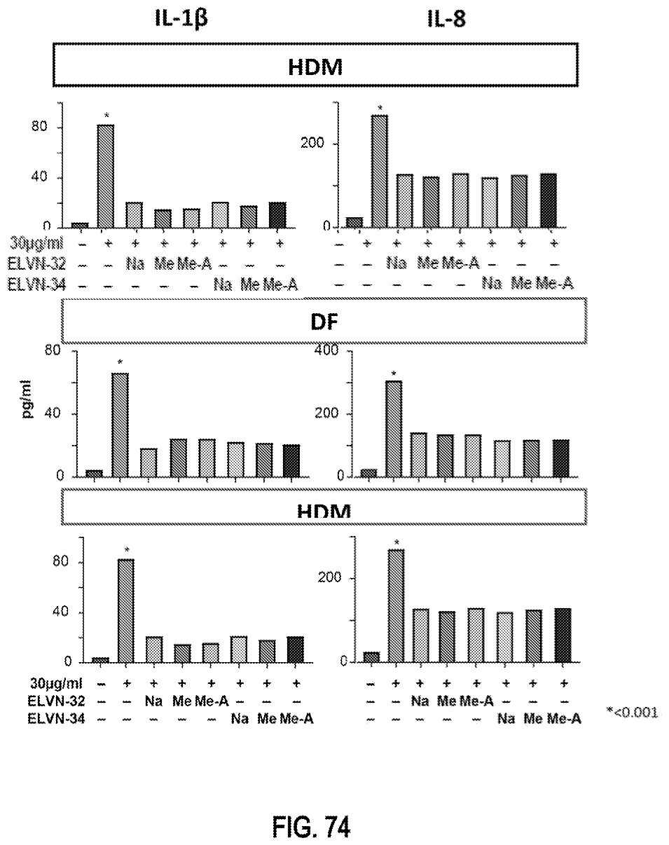

Within mite fecal pellets, Der p 1 can cleave and/or degrade several proteins, including HDM allergens and endosymbiotic bacteria. These activities can modulate the respective stability of HDM allergens and facilitate their release from the peritrophic matrix, as well as affecting generation of peptides for antigen processing. When fecal pellets reach the airway mucosa, elution of the microbial compounds and intact and fragmented allergens leads to initiation of the HDM-induced allergic response. Active Der p 1 can exert direct and indirect pleiotropic effects at the level of the airway epithelium, lung microbiome, and secretome, leading to release of proinflammatory cytokines (IL-6, IL-8, GM-CSF, thymic stromal lymphopoietin, and IL-25), alarmins (IL-lan and IL-33), and chemo-attractants (CCL2 and CCL20), which activate DCs, basophils, and ILC2s to initiate the TH2-biased HDM-induced allergic response. Red and black arrows or parentheses can represent the different effects of active Der p 1, respectively. AEC, Airway epithelial cell; AMG, anterior midgut; cDC, conventional DC; cDC2c, type 2 conventional DC; HG, hindgut; IL-33FL, full length IL-33; PM, peritrophic matrix; TJ, tight junction protein.
HDMs, D. ptero (Dermatophagoides pteronyssinus) or D. fari (Dermtophagoides farina) on Cell Survival and IL-6 (senescence activator) (FIG. 74).

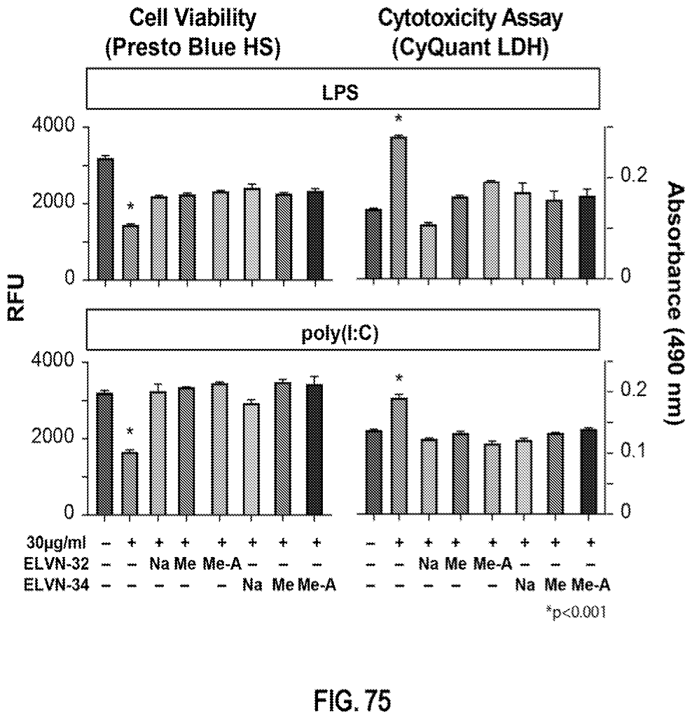

LPS or Poly(I:C) on cell survival (FIG. 75)

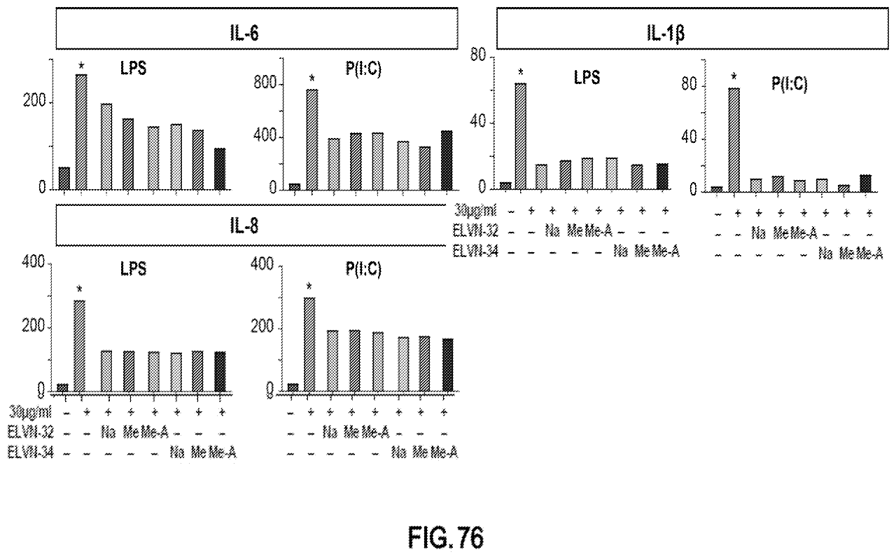

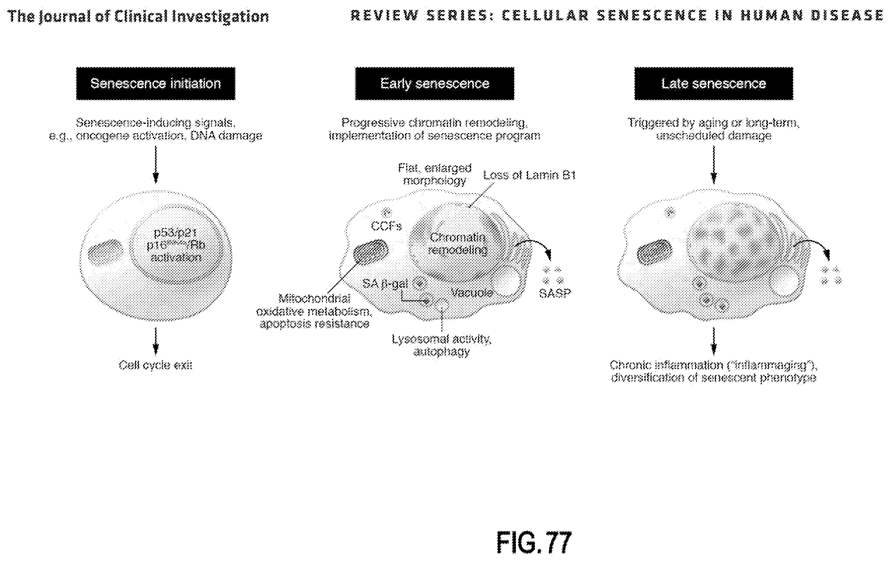

Effects of LPA or Poly (I:C) (FIG. 76)

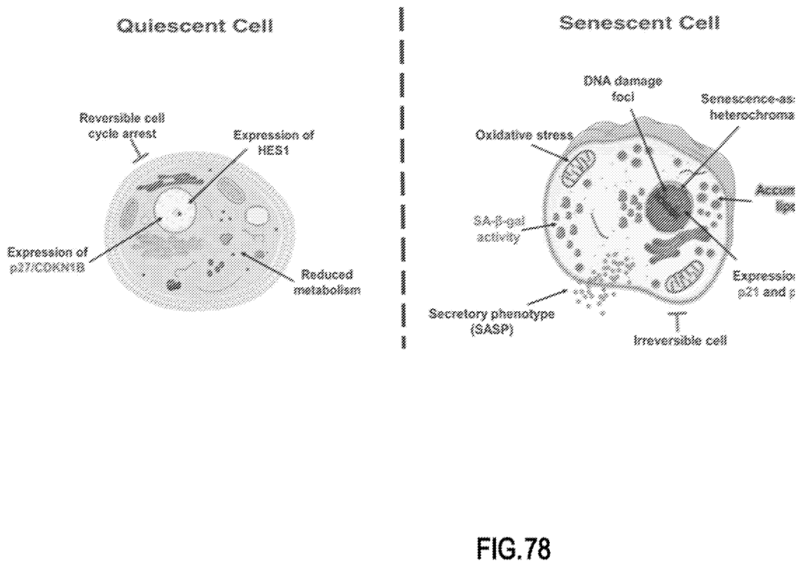

Senescence-Associated Secretory Phenotype (SASP) (FIG. 78)

Amyloid β peptide SASP disrupts intercellular matrix triggering ‘inflammaging’ (low-grade, sterile, chronic pro-inflammatory status) that sets in motion neuronal damage.

SASP: Immune surveillance of senescent cells
induce paracrine senescence in normal cells

SASP:
Chemokines/Inflammatory cytokines (TNF-α IL-1, -6, -8)
matrix-remodeling proteases (MMP-1, MMP-3)

Growth factors (insulin-like growth factor bp)
secreted proteins (fibronectin & collagen)

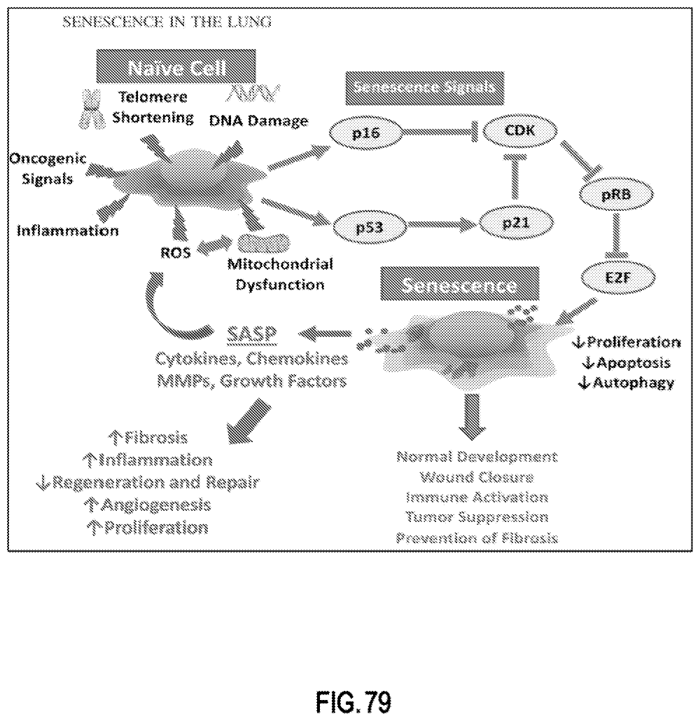

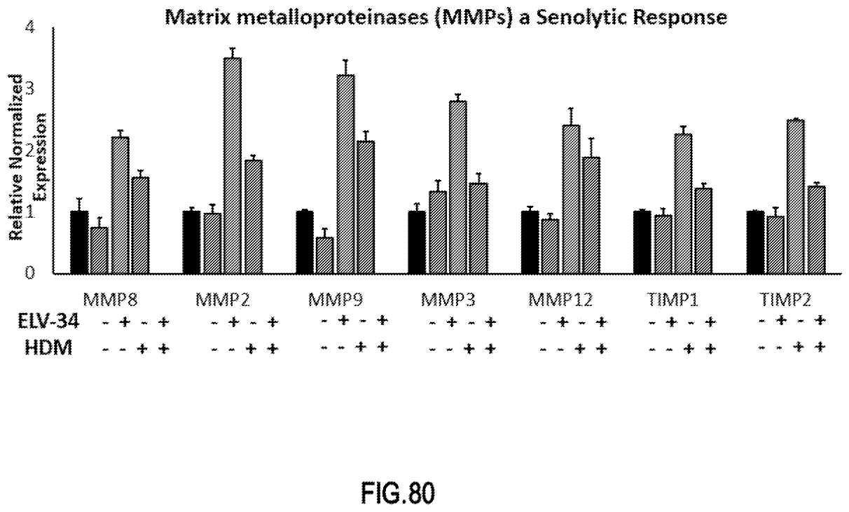

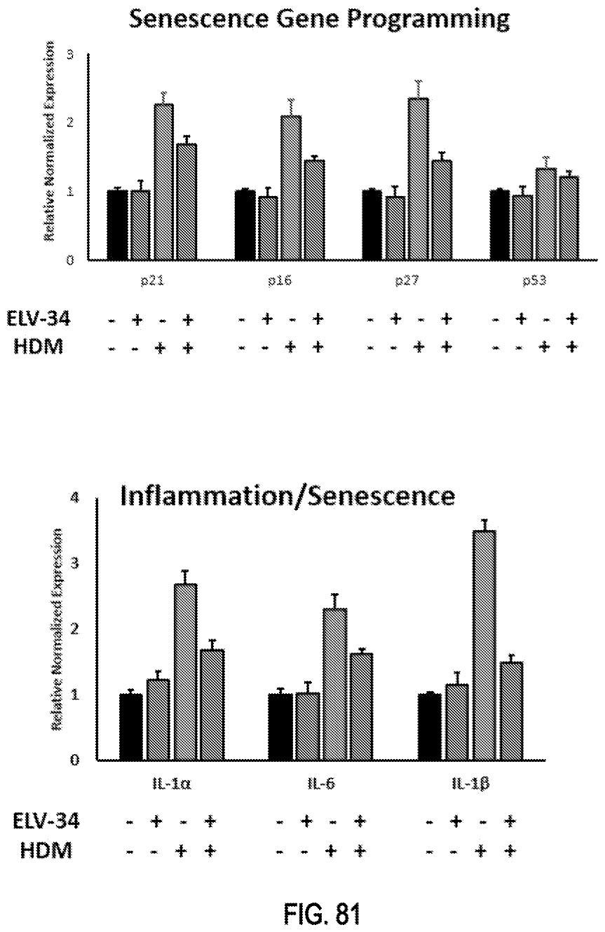

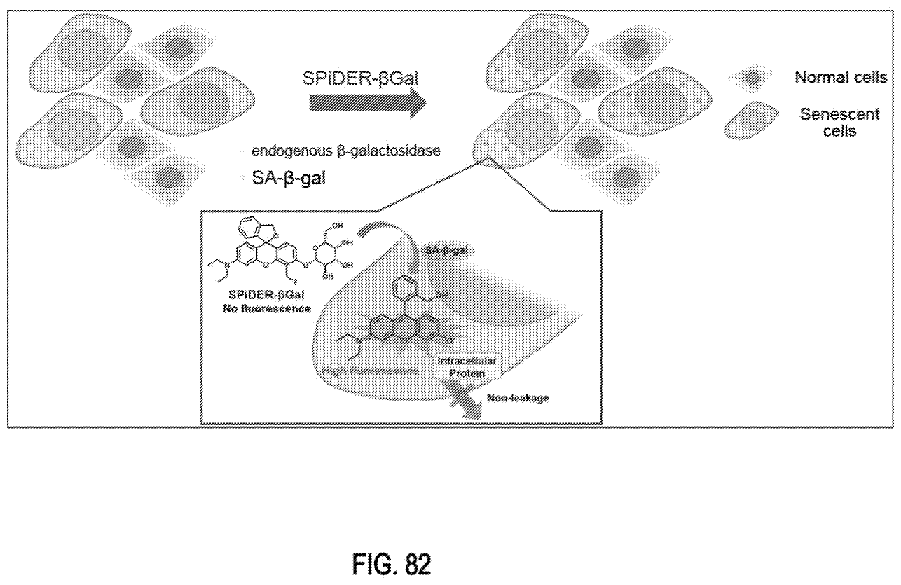

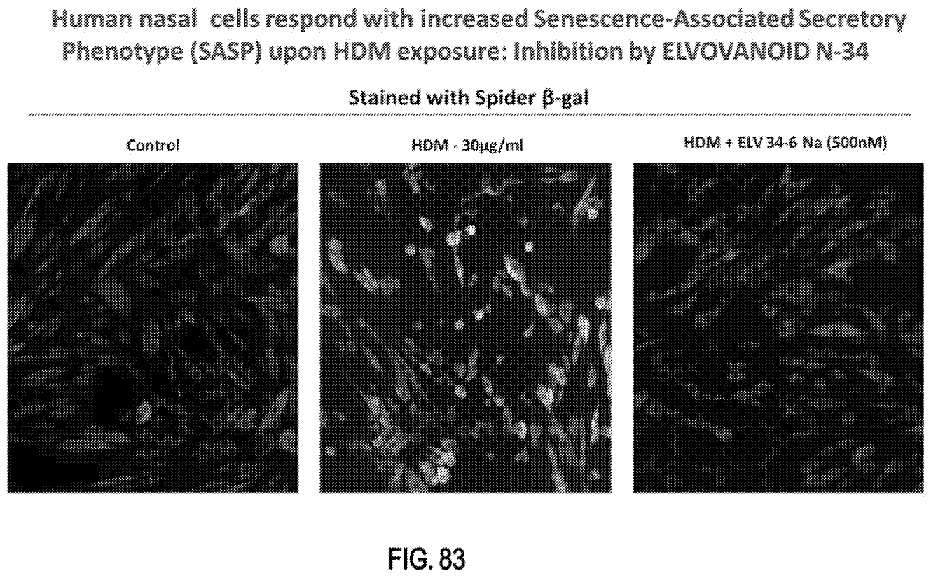

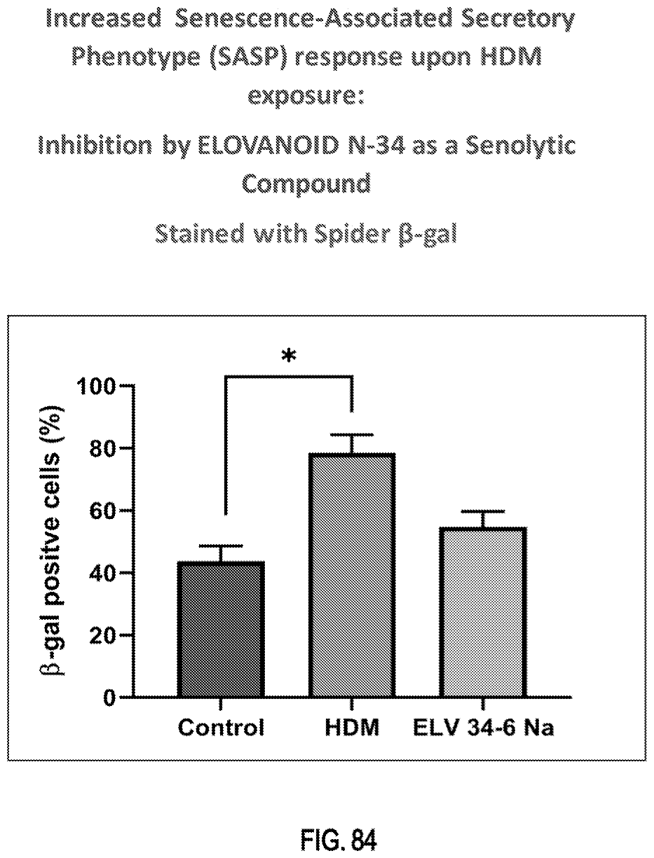

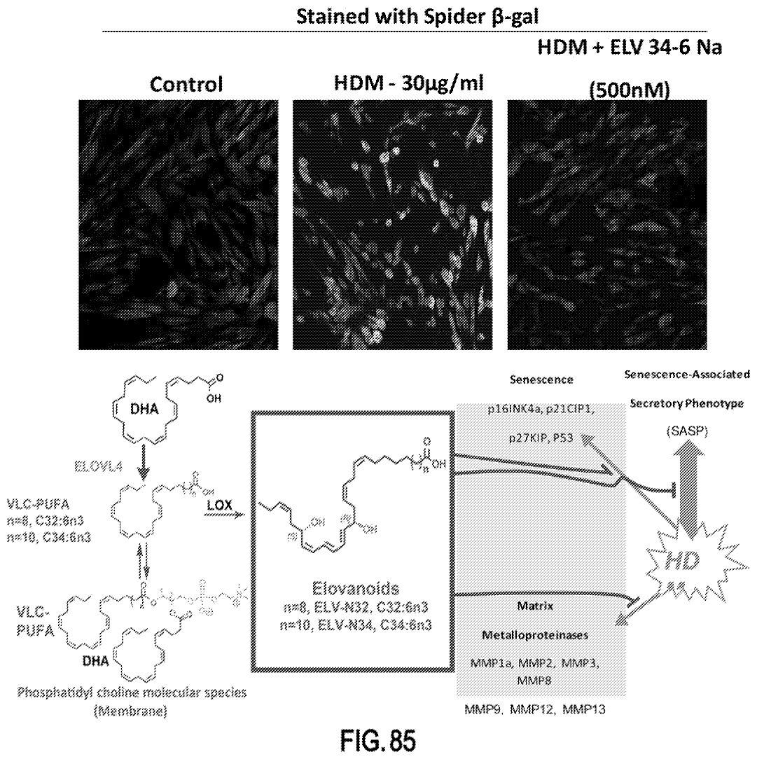

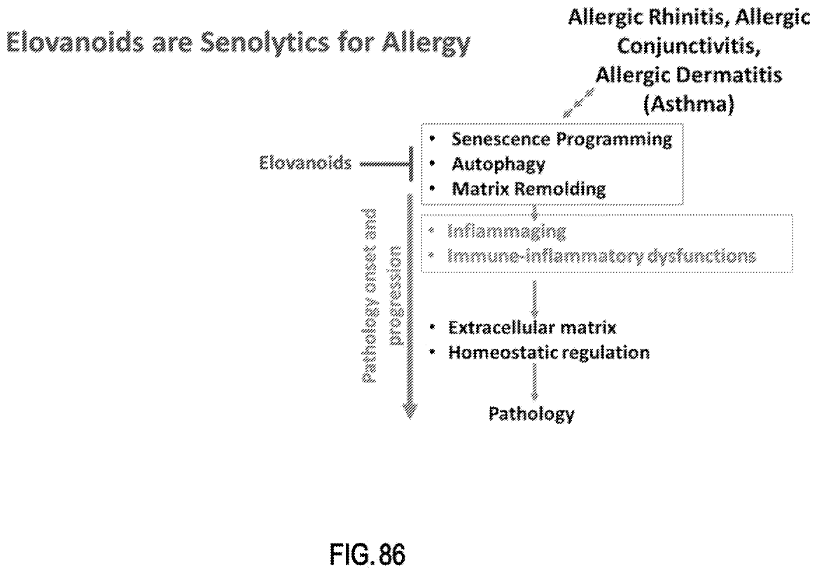

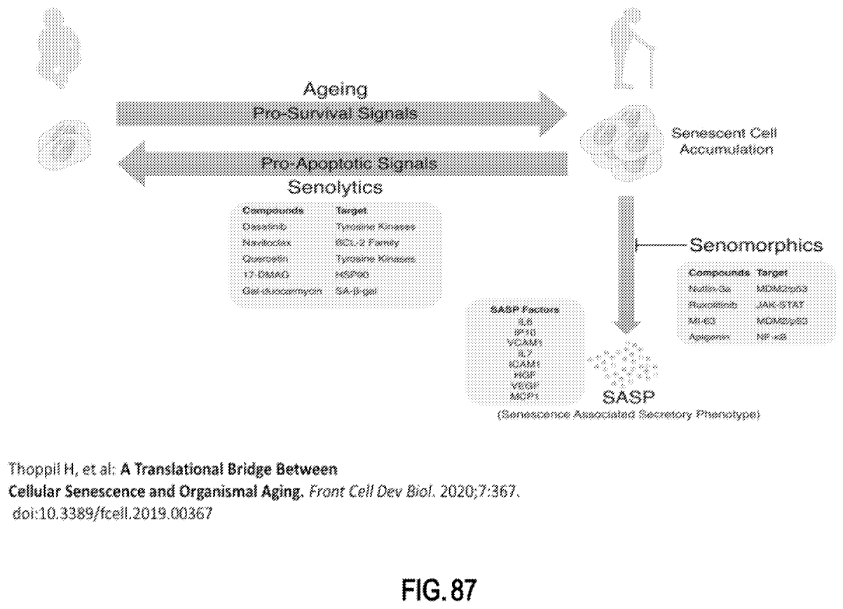

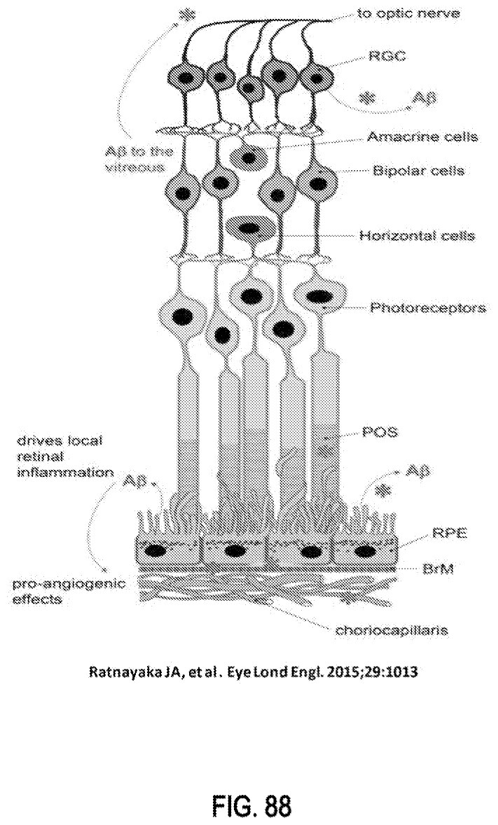

Cellular senescence and the senescence associated secretory phenotype (SASP) (FIG. 79)

Cellular senescence and the senescence associated secretory phenotype (SASP): naive cells can be exposed to a number endogenous or exogenous stimuli that initiate an intracellular signaling cascade that induces senescence (FIG. 79). Factors such as DNA damage from radiation, chemotherapy etc., aging-associated telomere shortening, oncogenic signals, and the influence of inflammation, reactive oxygen species (ROS), and/or mitochondrial dysfunction can all serve as triggers. Without wishing to be bound by theory, interplay between these factors is temporally involved. Senescent cells do not proliferate, but they are also resistant to apoptosis and autophagy and are thus long-living. Senescent cells secrete cytokines, chemokines, matrix-modulating factors (e.g., matrix metalloproteinases, MMPs), and growth factors that can have paracrine influences on surrounding naive cells to promote inflammation, mitochondrial dysfunction, and other disease-promoting pathways.

HDM induces MMPs gene expression: ELV 34 inhibits these effects

Conclusions: Effects of HDMs, D. ptero (Dermatophagoides pteronyssinus) or D. fari (Dermatophagoides farina)
cytotoxicity assay (LDH): red formazan=cytotoxicity, reduced by ELVs.
cell viability assay (PresetoBlue HS): resorufin; ELVs increases cell viability and cell protection
enhanced senescence cytokines and chemokines—IL-6, IL-1β, IL-8/CXCL8. ELVs (500 nM), 30 min post challenge blocks these effects.

Conclusions: LPS, poly(I:C)
Cytotoxicity assay (LDH): PLUS LPS, poly(I:C) increase red formazan indicating cytotoxicity, reduced by ELVs.
Cell viability assay (PrestoBlue HS) shows resorufin production, ELVs increases cell viability and cell protection.
Enhanced pro-inflammatory cytokines and chemokines—IL-6, IL-1β, IL-8/CXCL8.

ELV (500 nM), 30 min post challenge blocks these effects
Cytotoxicity assay (LDH): LPS, poly(I:C), HDM increase red formazan indicating cytotoxicity, reduced by ELVs.
Cell viability assay (PrestoBlue HS) shows resorufin production in cells challenged with LPS, poly(I:C), HDM; ELVs increases cell viability and cell protection.
Cell challenged with LPS, poly(I:C), HDM, display enhanced pro-inflammatory cytokines and chemokines, IL-6, IL-10, IL-8/CXCL8, CCL2/MCP-1, CXCL1/KC/GRO, VEGF, ICAM1(CD54). ELVs (500 nM), 30 min post challenge blockes these effects.
Conversely, LPS, poly(I:C), HDM, display a decrease in the release of anti-inflammatory cytokine, IL-10. Reversed by ELVs (500 nM), 30 min post challenge.

Elovanoids are Senolytics in Nasal Mucosal Cells

Exemplary model of ELVs in Oaβ-induced RPE and PRC damage.

(A) OAβ induces senescence and disrupts the tight junction of RPE. OAβ penetrates the retina, causing cell death of photoreceptors reflected in less cell body layer (CBL) nuclei. The Elovanoids restore the morphology of the RPE layer upon OAβ exposure and, as a consequence, the retina structure is preserved.

(B) OAβ induces the senescence, autophagy, matrix metalloproteinases, and AMD-related genes in the RPE and apoptosis genes in retina. ELV downregulated the OAβ-gene inductions. Pathways for the synthesis of ELVs are depicted.

### Example 14

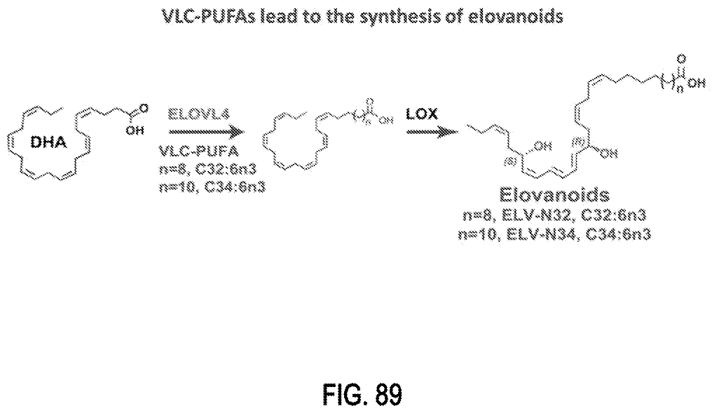

Are Elovanoids senolytics?
experimental models
A) Allergy
B) Amyloid-β peptide in PRC degeneration
senescence gene programming and inflammaging in photoreceptor cell survival
Outcome: RPE cell integrity and PRC survival
Photoreceptors and Brain Neurons selectively express the evolutionarily-conserved fatty acid elongase ELOVL4 that catalyzes the Synthesis of VLC-PUFAs (>28 carbons) (FIG. 89).

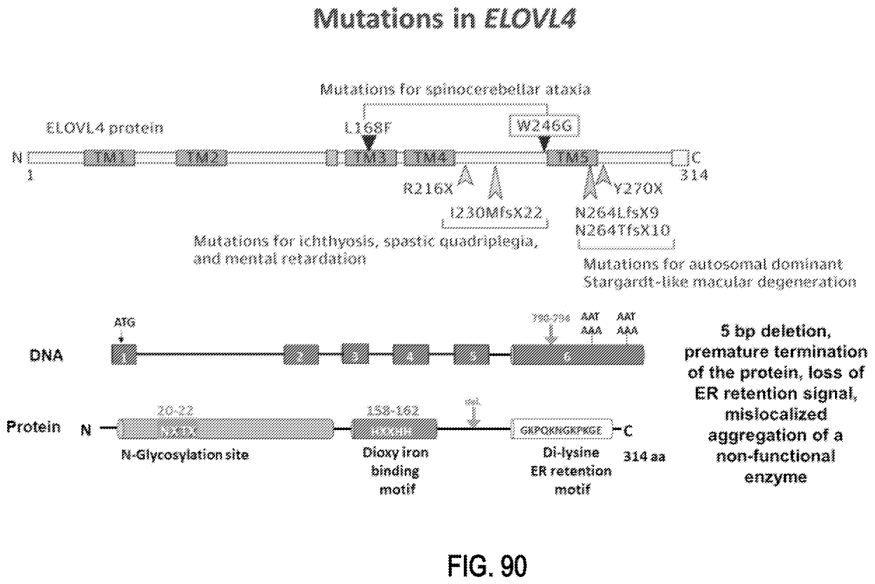

Mutations in ELOVL4 (FIG. 90)

Impaired neural development, neuronal hyper-excitability and seizures, mental retardation, neuroichthyotic disorders, and spinocerebellar ataxia.

3 Genetic Data on 2 Japanese Families With the Novel Mutation (p.W246G) in ELOVL4

The mutation c.736T>G, p.W246G in ELOVL4 was detected in the affected member, as indicated by the arrowhead. The affected amino acid residue is highly conserved from zebrafish to humans, as indicated by the red rectangle.

The brown boxes indicate transmembrane domains as predicted in previous reports and the Uniprot Knowledgebase (see Magrane M; UniProt Consortium. UniProt Knowledgebase: a hub of integrated protein data. Database. 2011; 2011:bar009 and Zhang K, Kniazeva M, Han M, et al. A 5-bp deletion in ELOVL4 is associated with two related forms of autosomal dominant macular dystrophy. Nat Genet. 2001; 27(1):89-93); the green box indicates the dioxy iron-binding motif (HXXHH); the yellow box indicates the dilysine motif for the retention of transmembrane proteins in the endoplasmic reticulum. The mutations (p.W246G and p.L168F) lead to spinocerebellar ataxia (black arrowheads). The p.W246G mutation was identified in our study (the red rectangle). Two recessive mutations (p.R216X and p.I230MfsX22 [blue arrowheads]) were reported to cause ichthyosis, spastic quadriplegia, and mental retardation (Aldahmesh M A, Mohamed J Y, Alkuraya H S, et al. Recessive mutations in ELOVL4 cause ichthyosis, intellectual disability, and spastic quadriplegia. Am J Hum Genet. 2011; 89(6):745-750). Three mutations (N264LfsX9, N264TfsX10, and Y270X [orange arrowheads]) cause autosomal dominant Stargardt-like macular dystrophy (Vasireddy V, Wong P, Ayyagari R. Genetics and molecular pathology of Stargardt-like macular degeneration. Prog Retin Eye Res. 2010; 29(3):191-207). (K Ozaki et al., JAMA Neurol. 2015; 72:797-805)

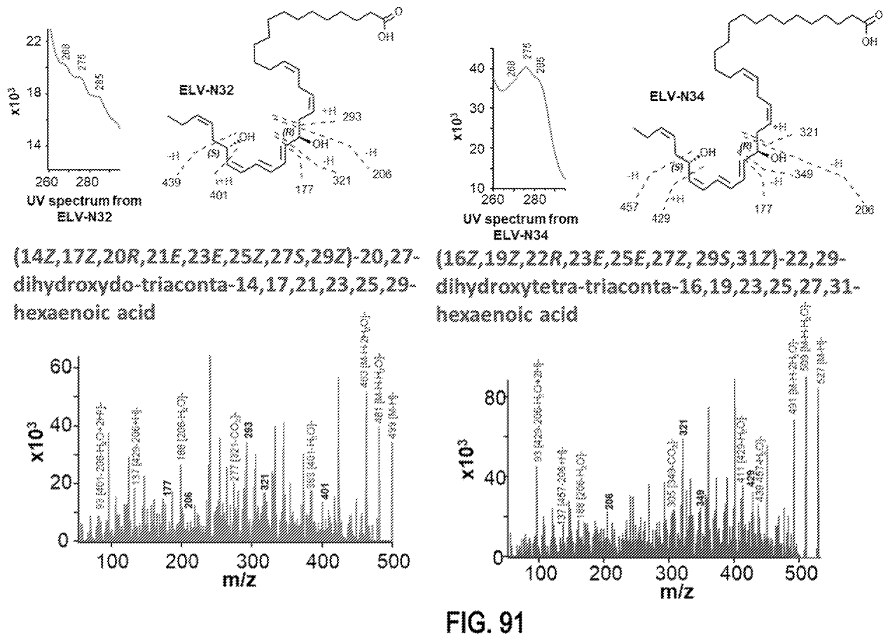

Elovanoids are neuroprotective and pro-homeostatic in (FIG. 91)

Oxygen/glucose deprivation or NMDA receptor-mediated excitotoxicity

Stroke: reduces volume, induces cell survival, and attenuates neurovascular unit disruption, injected 1 h after 2 hrs of middle cerebral artery occlusion.

Upregulate anti-apoptotic and downregulate pro-apoptotic protein abundance

Retinal pigment epithelial cells, sustain photoreceptor cell integrity and function (see, Jun, B. Et al. Sci. Rep. 2017; 7:5279:doi:10.1038, Bhattacharjee S, et al. Sci. Adv. 2017:3:e1700735, and Bazan N. Mol. Aspects Med. 2018; 64:18-33.doi:10.1016).

ELVs restore RPE morphology after subretinal injection of Oligomeric Amyloid-β peptide.

ELVs restore RPE morphology and reduce gene expression after subretinal injection of OAβ in WT mice.

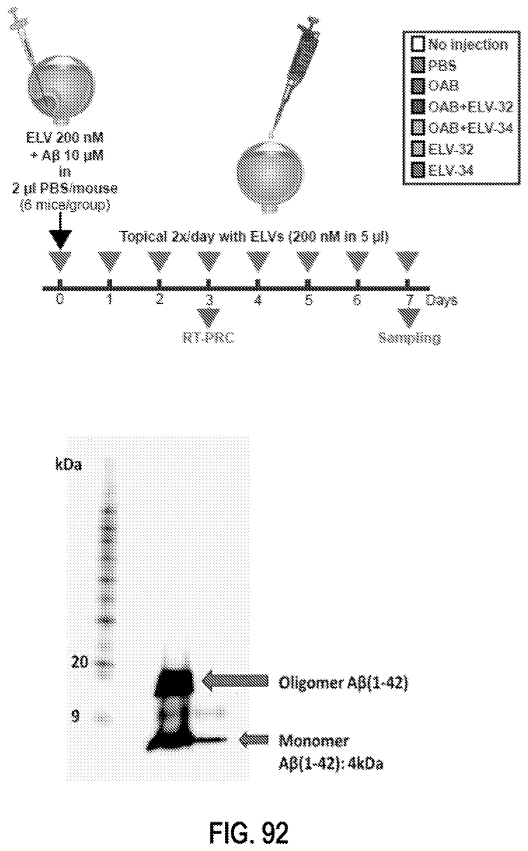

Mice were divided into 7 groups: non-injected, PBS, OAβ only, OAβ+ELV-N32, OAβ+ELV-N34, ELV-N32 only and ELV-N34 only. On day 3, mRNA were isolated for RT-PCR. On day 7, mice were subjected to OCT and then eyes were enucleated and processed to whole mount RPE staining and Western blot (FIG. 92).

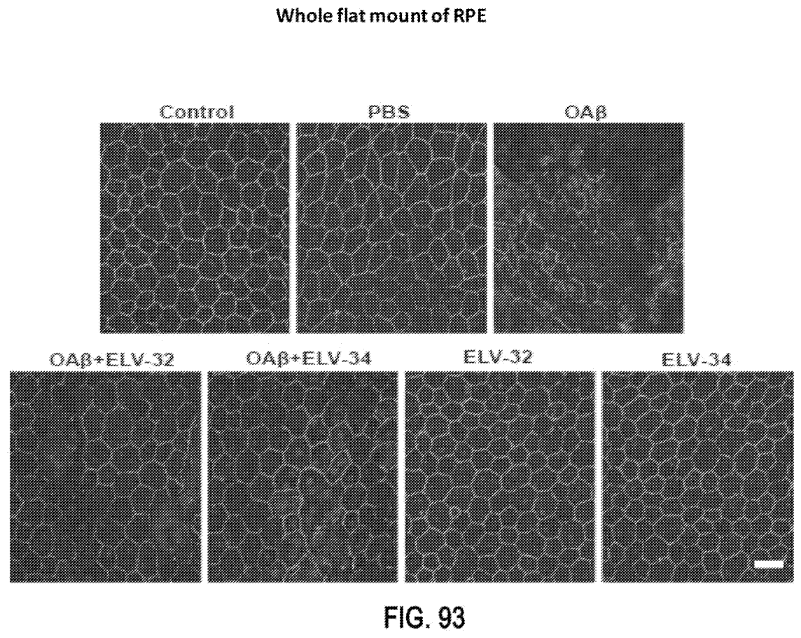

Whole flat mount of RPE (FIG. 93). OAβ disrupted RPE morphology. However, RPE were less damage in the ELVs treatment group as well as PBS, ELVs alone did non induce changes. (FIG. 93).

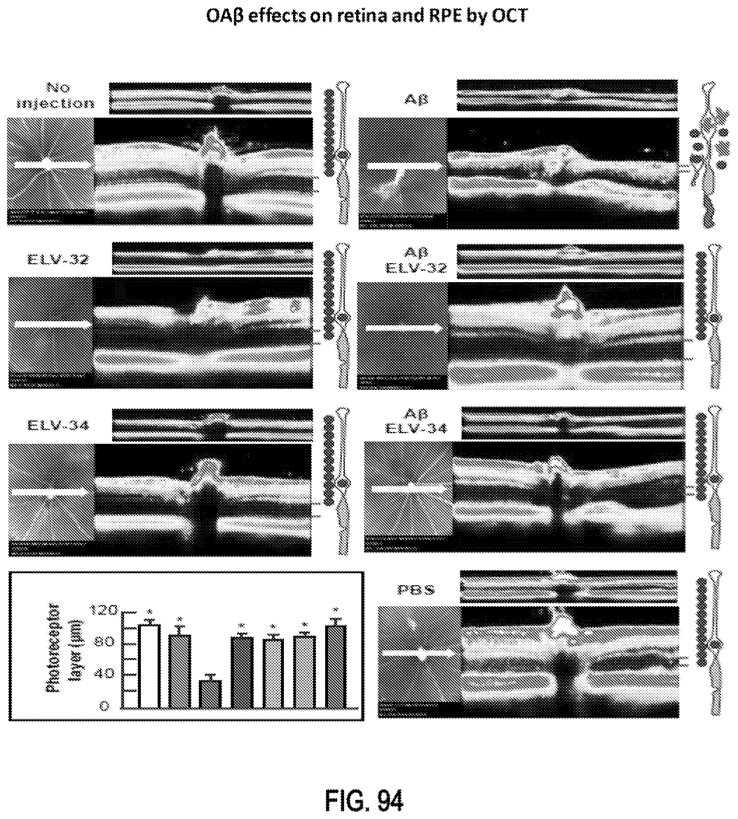

(FIG. 94) Evaluation the OAβ effects on retina and RPE by OCT. The thickness of PRC was thin in the OAβ injected group. OAβ cause the cell death of PRC, as the thinner in OCT measurement.

Cellular Senescence: A homeostatic response to prevent propagation of damaged cells and neoplastic transformation.

Stress-induced premature senescence due to uncompensated oxidative stress, impaired autophagy, mitochondrial dysfunction

Unlike apoptosis, senescent cells remain viable and metabolically active.

Detrimental effects in age-associated neurodegenerations.

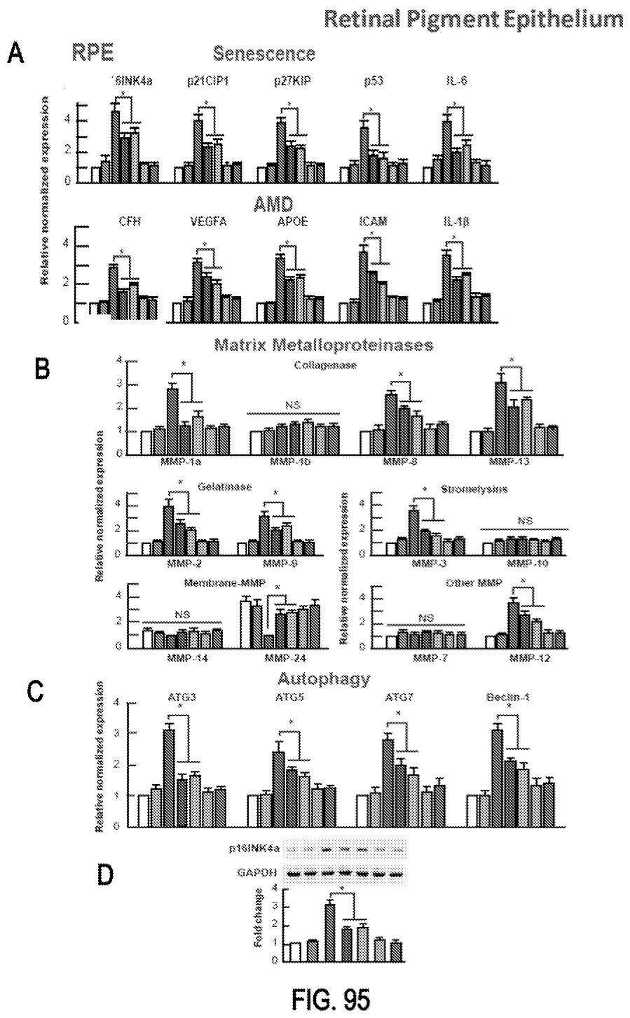

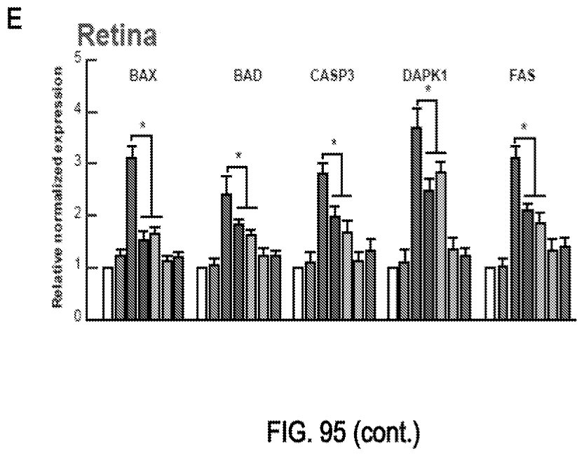

ELVs restore senescence gene expression modified by Oligomeric Amyloid-0. (FIG. 95)

RPE gene expression after OAβ (1-42) injection and treatment with ELVs. 3 days after injection, the RNAs from RPE were isolated, reverse transcribed into cDNA and subjected to RT-PCR with different primers. Genes in the same functional group were plotted in the same chart, including senescence- and AMD-related genes (Panel A), and collagenases, gelatinase, stromelysins and others matrix metalloproteinases (MMP) (Panel B) and autophagy (Panel C).

(Panel D) p16INK4a western blots of RPE/Choroid. ELV-N32 and ELV-N34 down regulated the expression of the key senescence marker, p16INK4a, which was elevated by OAβ injection (Panel E) Retina gene expression after OAβ (1-42) injection and treatment with ELVs. OAβ activates apoptosis genes in the retina. With ELVs co-injection, these genes were down-regulated. (*P<0.05, using student t-test comparison).

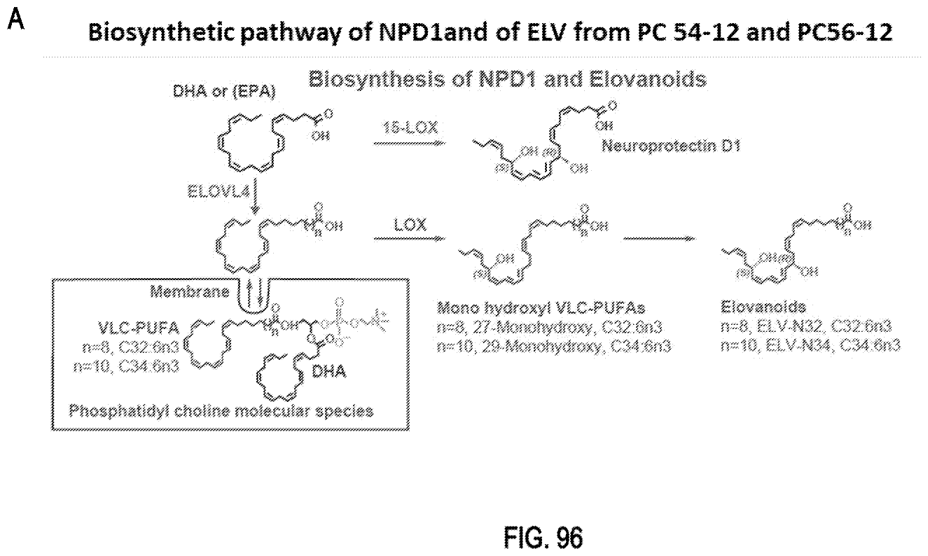

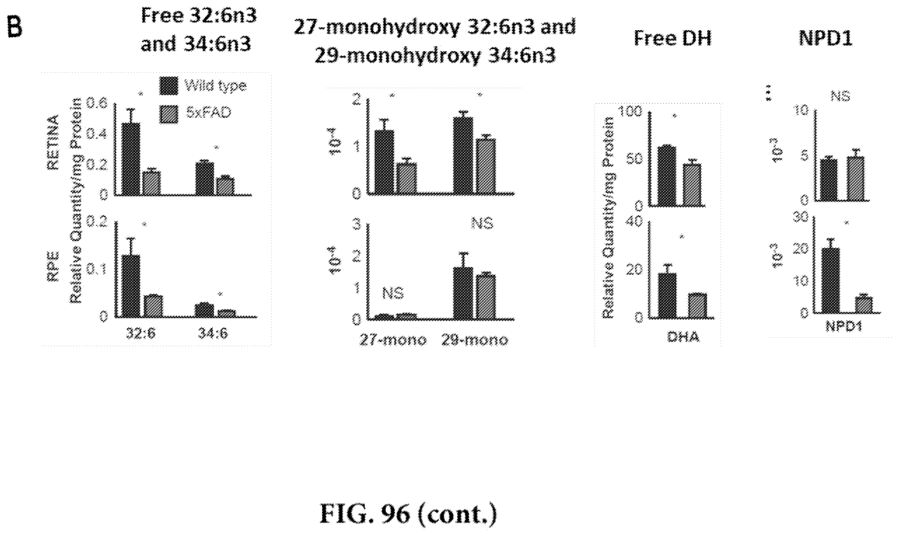

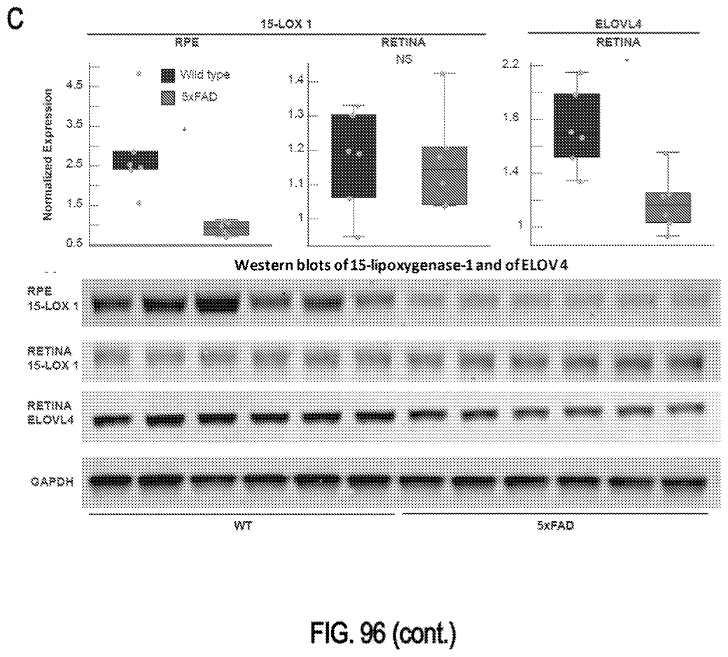

Deficiencies in precursors and intermediates of lipid mediators pathways and of ELOVL-4 during the early development of retina pathology in the 5×FAD mouse. (FIG. 96). The biosynthetic pathway of NPD1, 32:6n3 and 34:6n3 elovanoids from their PC 54-12 and PC56-12 precursors (Panel A). For the mass spectrometry detection, the stable monohydroxy products of VLC-PUFAs are used. (Panel B) The bar chart for free 32:6n3 and 34:6n3 VLC-PUFAs, 27-monohydroxy 32:6n3 and 29-monohydroxy 34:6n3 VLC-PUFAs, free DHA, and NPD1 in the retina (top) and RPE layers (bottom). (Panel C) Western blots and quantification of 15-lypoxygenase-1 expression in RPE and Retina. In RPE, the expression of 15-lypoxygenase-1 in 5×FAD is less than WT RPE. In the retina, there is no difference between two groups. This explains why the level of NPD1 is lower in 5×FAD, but no change in the retina. ELOVL4 is only express in retina and its expression is lower in 5×FAD. As a consequence, there was less free 32:6n3 and 34:6n3 as well as less abundant monohydroxy molecules. (NS: non-significant, *P<0.05, using student t-test comparison).

Senescence-Associated Secretory Phenotype (SASP)

SASP disrupts interphotoreceptor matrix triggering ‘inflammaging’ (low-grade, sterile, chronic pro-inflammatory status) that sets in motion RPE/PRC damage.

SASP:
Chemokines/Inflammatory

cytokines (TNF-α IL-1, -6, -8)
Matrix-remodeling proteases

(MMP-1, MMP-3)
Growth factors (insulin-like growth factor)
Fibronectin & collagen)

SASP:

Immune surveillance of senescent cells
induce paracrine senescence in normal cells

Senescence-associated secretory phenotype induced by OA-β is counteracted by ELVs in RPE cells

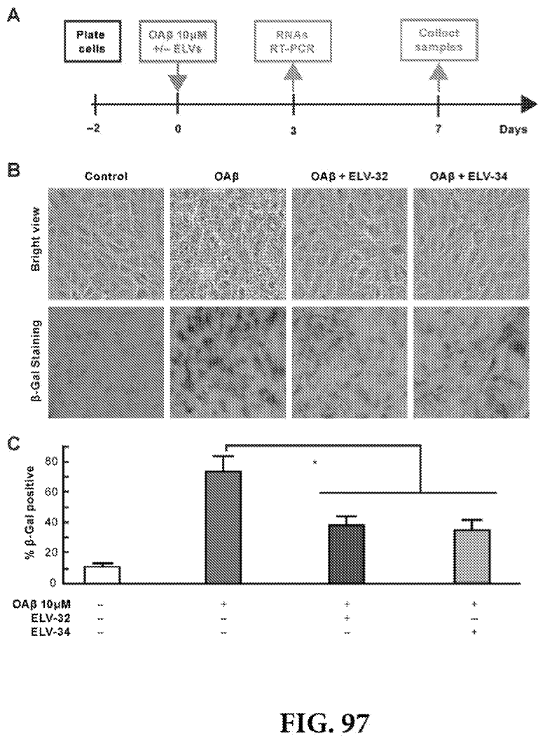

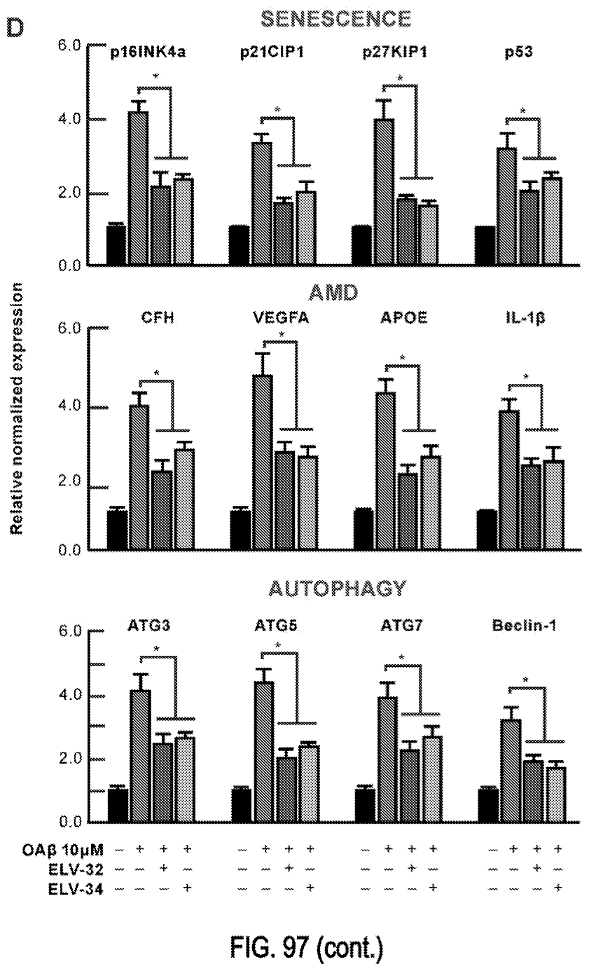

OA-β-mediated activation of senescence-associated secretory phenotype (0-galactosidase, SA-β-Gal) and of gene expression in human RPE cells in primary culture are counteracted by ELVs (FIG. 97).

(Panel A) In vitro experimental design: Primary human RPE cells were treated with 10 μM OAβ+/−ELVs. After 3 days, RNA was isolated and qPCR analyzed. After 7 days, cells were subjected to β-Galactosidase staining. (Panel B) Live cell images under bright field microscopy after 7 days. (Panel C) β-Gal staining+/−ELVs. Quantitation of % for the β-Gal positive cells. ELVs decreased positive senescent cells. (Panel D) Gene transcription of senescence, AMD-related and autophagy genes after OAβ (1-42) exposure+/−ELVs. (*P<0.05, student t-test).

Loss of intercellular matrix integrity is a hallmark of aging.
Matrix molecules exhibit long half-lives
Drive phenotypes that increase cell integrity early in life but become detrimental in an aged organism.
Placing senescent cells into a ‘young’ matrix can rejuvenate them, highlighting how the cellular microenvironment feeds into cell ageing.

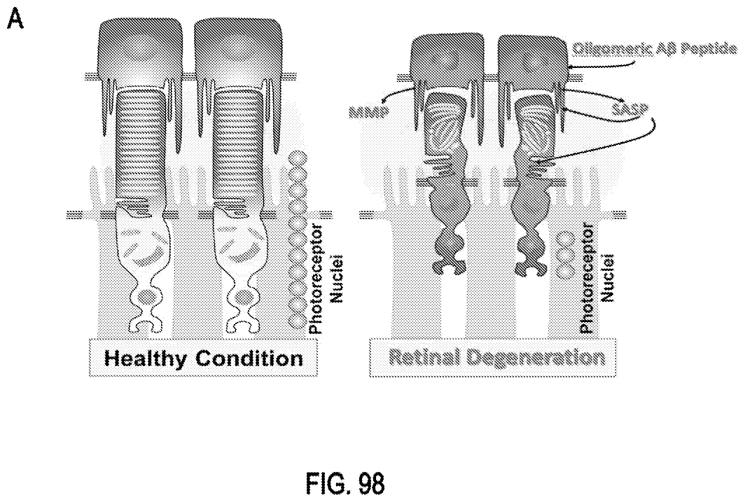

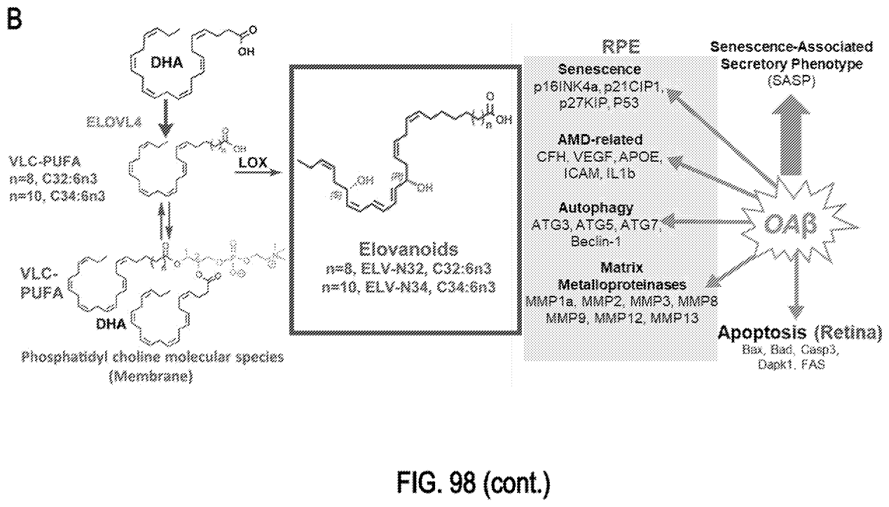

Elovanoids have Senolytic Bioactivity: Targets senescence-associated Secretory phenotype alters the interphotoreceptor matrix (FIG. 98).

(Panel A) OAβ induces senescence and disrupts the tight junction of RPE. Next, OAβ penetrates the retina, causing cell death of photoreceptors reflected in less cell body layer (CBL) nuclei. The Elovanoids restore the morphology of the RPE layer upon OAβ exposure and, as a consequence, the retina structure is preserved.

(Panel B) OAβ induces the senescence, autophagy, matrix metalloproteinases, and AMD-related genes in the RPE and apoptosis genes in retina. ELVs downregulated the OAβ-gene inductions. Pathways for the synthesis of ELVs are depicted.

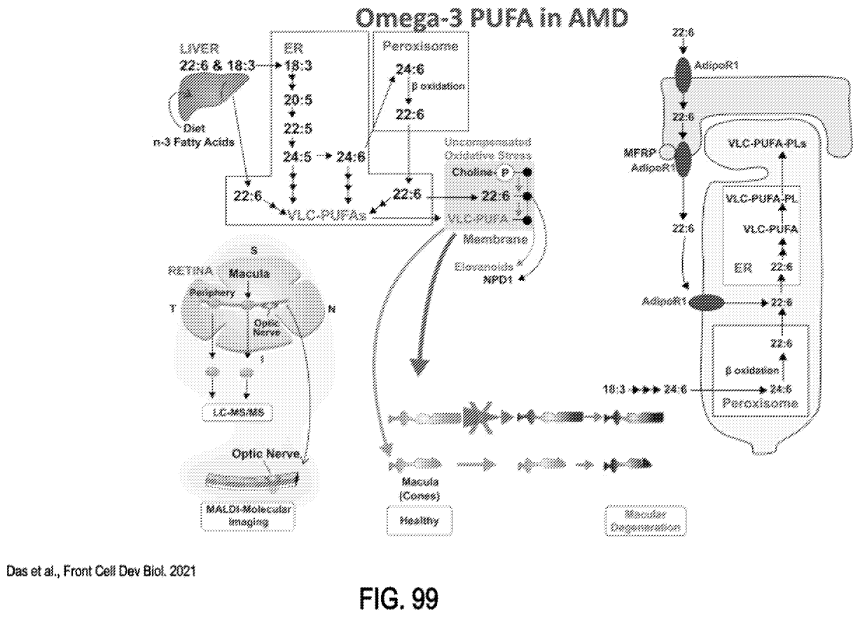

Omega-3 PUFA in AMD (FIG. 99) Das Y, et al Peroxisomal Multifunctional Protein 2 Deficiency Perturbs Lipid Homeostasis in the Retina and Causes Visual Dysfunction in Mice. (Front Cell Dev Biol. 2021).

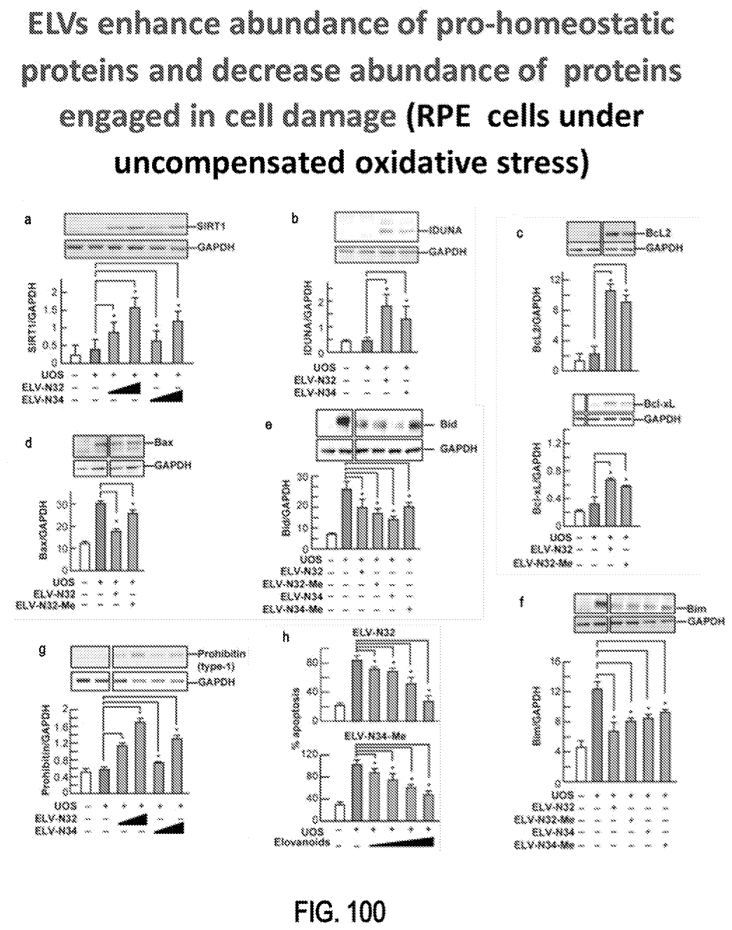

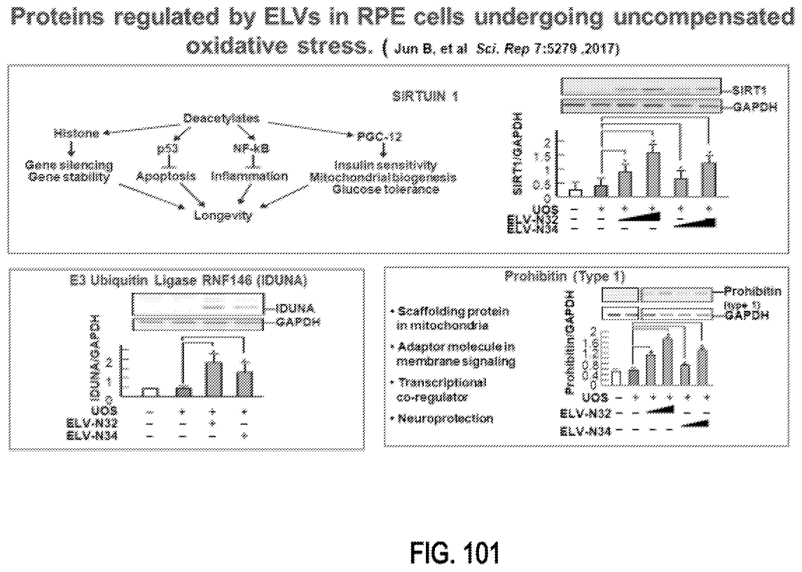

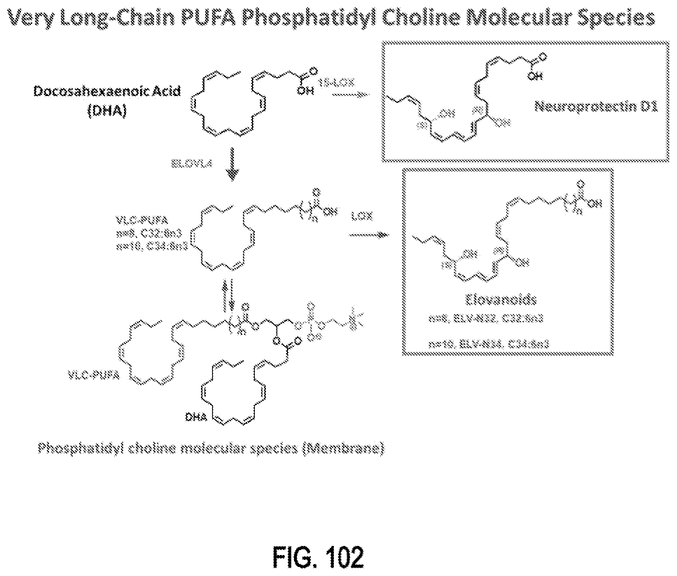

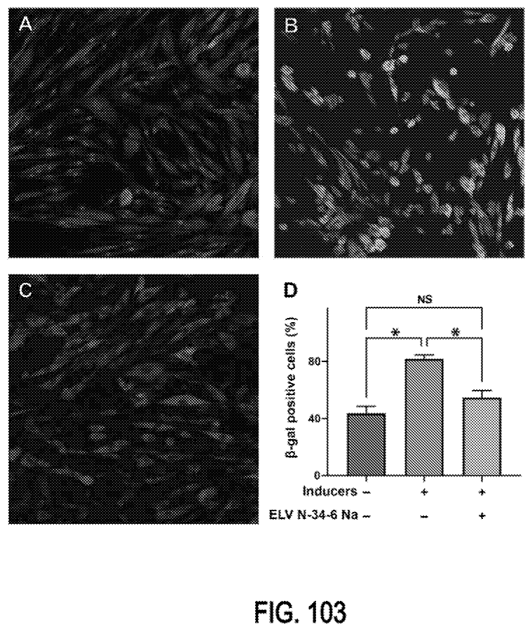

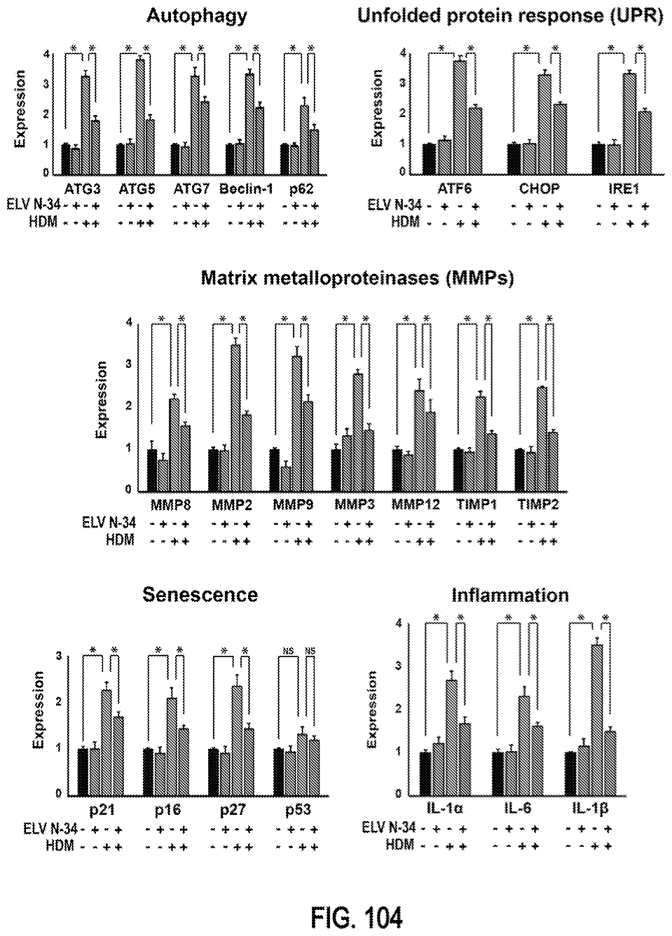

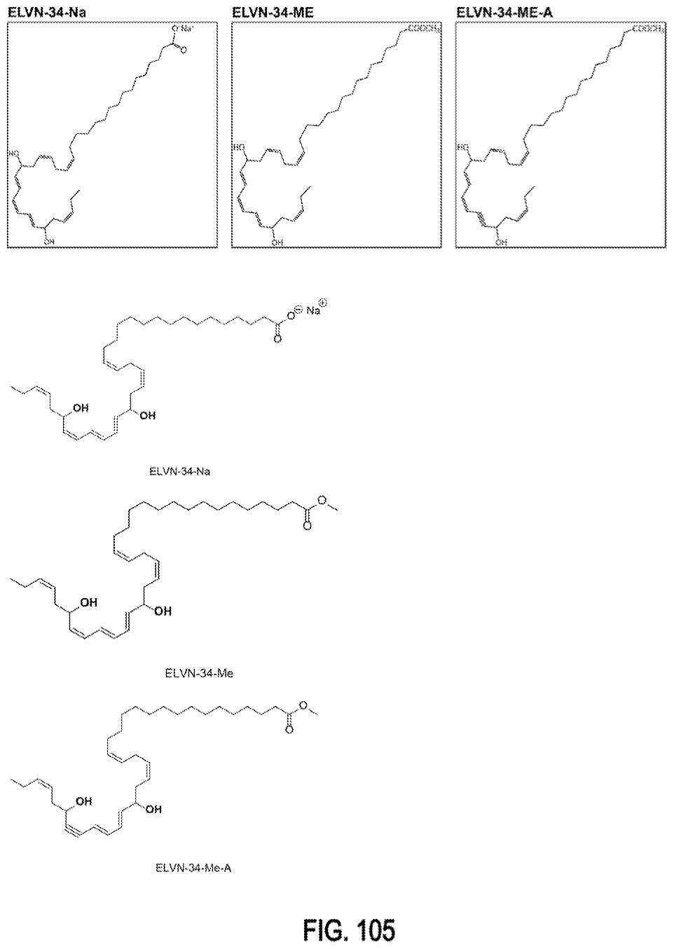

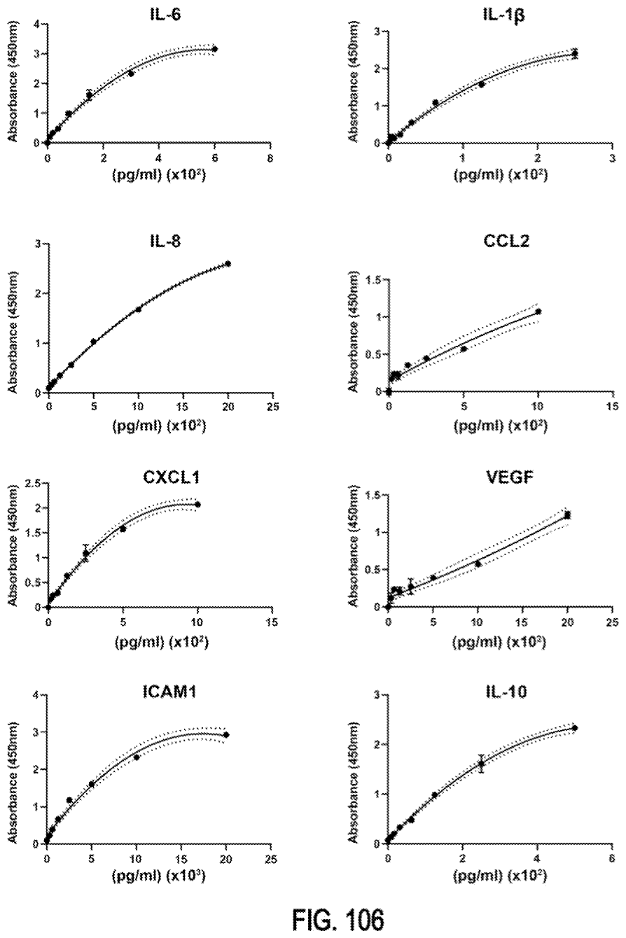

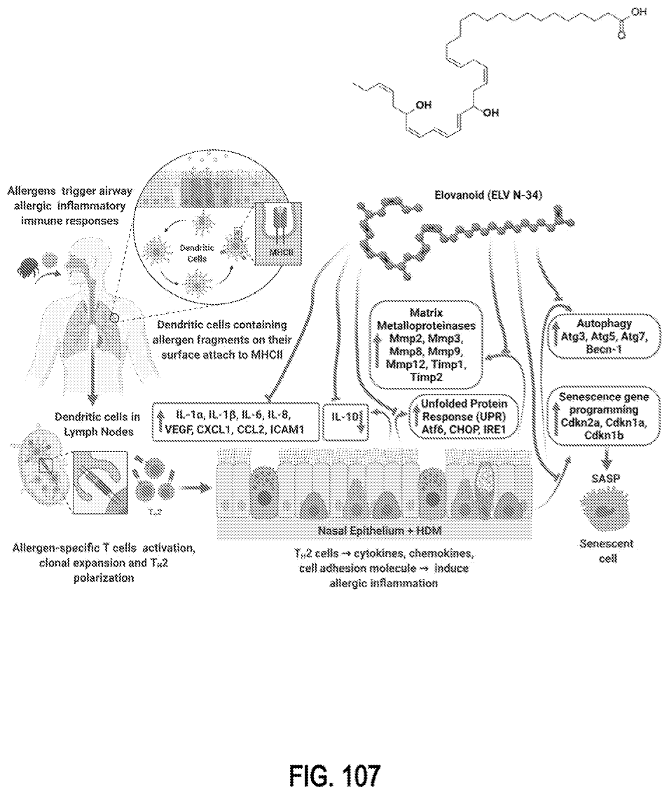

ELV-N32 and ELV-N34 enhance abundance of pro-homeostatic proteins and decrease abundance of cell damaging proteins in RPE cells under UOS (FIG. 100). ELV-N32 or ELV-N34 indicates the sodium salt forms, and ELV-N34-Me or ELV-N32-Me indicates the methyl ester forms. ELVs induces the following effects in ARPE-19 cells undergoing UOS: (panel a) Concentration-dependent (100 and 250 nM) upregulation of SIRT1. The results are the averages of three independent experiments; (panel b) upregulation of Iduna abundance; (panel c) increased abundance of anti-apoptotic proteins Bcl-2 and Bcl-xL; (panel d) decreased abundance of pro-apoptotic proteins Bax, (panel e) Bid and (panel f) Bim. (panel g) Concentration-dependent (100 and 250 nM) upregulation of Prohibitin (type-1) by ELVs takes place. (panel h) Concentration-dependent (50, 100, 250, and 500 nM) reduction of UOS-induced apoptosis. Error bars, SEM; *p<0.05.

Elovanoids: Senolytics Molecular Guardians of PRC Integrity

Telomeres: A ‘mitotic clock’ in neurodegeneration fueled by inflammation

Telomerase: involved in senescence, DNA damage and repair

Non-telomeric functions of telomerase
TERT (telomerase reverse transcriptase)
TTl1 (TEL02-interacting protein1
RAP1 (repressor/activator protein1

Cell and Interphotoreceptor Matrix Protection

Homeostasis Restoration

Maintenance of a balanced DHA PRC lipidome relies on MFRP and ADIPORI

Control acquisition and distribution of key lipids from the RPE to the PRC,

ELVs act as epigenetic regulators contributing to RPE cell-survival/senescence decisions.

Cellular Senescence: A homeostatic response to prevent propagation of damaged cells and neoplastic transformation.

Hayflick and Moorhead (1961), observed that cultured fibroblasts, passaged serially underwent stable cell cycle arrest and phenotypic changes

Replicative senescence is associated with telomere shortening.

Stress-induced premature senescence due to uncompensated oxidative stress, impaired autophagy, mitochondrial dysfunction

When cell's repair mechanisms become overwhelmed, a DNA damage response elicits cellular senescence via p53 phosphorylation.

Unlike apoptosis, senescent cells remain viable and metabolically active.

Detrimental effects in age-associated neurodegenerations.

Loss of intercellular matrix integrity is a hallmark of ageing: Elovanoids counteract this as a senolytic.
Matrix molecules exhibit long half-lives
Drive phenotypes that increase cell integrity early in life but become detrimental in an aged organism.
Collagen mass declines at 1%/year in tissues such as skin.
Placing senescent cells into a ‘young’ matrix can rejuvenate them, highlighting how the cellular microenvironment feeds into cell ageing.
Ageing of tissue matrix is accelerated by: skin photo-ageing that alters the matrix proteome such that resembles that of the aged skin.
Senesence-Associated secretory phenotype alters the interphotoreceptor matrix.
Inflammaging (chronic low-grade inflammation occurring with advanced age and also during other conditions).

Amyloid-β peptide in PRC degeneration

Senescence gene programming and inflammaging in photoreceptor cell survival
Does O A-β triggers

RPE damage leading to PRC death
Elovanoids blocks O A-β induction of senescence gene programs, in mice (in vivo) and in RPE cells
Outcome: RPE cell integrity and PRC survival.

### Example 15

Allergens trigger airway allergic inflammatory responses which induce production of inflammatory cytokines, chemokines, cell adhesion molecule (ICAM1) resulting in induction of autophagy and SASP which are attenuated by Elovanoid (ELV N-34).

### Example 16

Elovanoids are Senolytic: Downregulation of Senescence Programming in the Hypothalamus and Adipose Tissue by Elovanoids Counteracts Diabetes Onset and Progression

The incidence of type 2 diabetes (a consequence of obesity) is rapidly increasing, particularly during aging and is a risk factor for kidney dysfunction, cardiovascular disease, stroke, impaired wound healing, infections, depression, anxiety and cognitive decline.⁴⁻⁷ Despite advances in our understanding of the pathogenesis of diabetes, metabolic syndrome, and comorbidities, there is no effective therapy available. Cellular senescence has been implicated in age-related chronic inflammatory diseases, including metabolic syndrome (hypertension, obesity, and atherosclerosis), in the pathogenesis of type 2 diabetes by targeting pancreatic beta-cell function and by triggering adipose tissue dysfunction. Senescent programming forms a diabetes loop—the cause and consequence of cellular dysfunctions. We study a class of lipid mediators, the Elovanoids (ELVs), as downregulators of senescence programming to counteract diabetes onset and progression.

ELVs are dihydroxylated derivatives of the very long-chain polyunsaturated fatty acids (VLC-PUFAs), for example, 32:6n3 and 34:6n3. As precursors of ELVs, VLC-PUFAs are biosynthesized by elongation of a 22:6n3 fatty acid and catalyzed by ELOVL4 (elongation of very-long-chain fatty acids-4). We have reported the discovery of ELVs and their detailed structure and stereochemistry, as established by stereocontrolled total organic synthesis^{1,2}. We reported that ELVs are low-abundance, high-potency, neuroprotective, pro-homeostatic mediators that arrest senescence gene programming and the senescence-activated secretory phenotype (SASP) in neural cells upon homeostasis disruptions³.

Herein, we describe Elovanoids and their ability to downregulate slow-going inflammation (inflammaging) in the adipose tissue (AT) and hypothalamus (HT) through a senescence program (SP) that involves senescence transcriptome and the senescence-activated secretory phenotype (SASP). This can be supported, for example, by data using human adipocytes from diabetic patients, a genetic diabetic mouse model, and a state-of-the-art approach to addressing specific functional issues in the HT.

The HT is also a target because, although it is comprised of terminally differentiated cells and originates from the neuroepithelium senescent neurons in aged mice, models of AD ⁴, and astrocytes^{5,6}, it also expresses senescence and develops secretory SASP that fuels neuroinflammation in nearby cells 7-9. We observe neighboring cells can be targeted by neurotoxic actions of SASP, inducing retinal paracrine senescence 3.

Human adipocytes from diabetic patients activate an SP upon induction by TNF alpha. Active brown/beige AT plasticity increases energy expenditure and is linked to reduced hyperglycemia and hyperlipidemia; on the other hand, its atrophy and inactivation are can cause obesity and aging.

Elovanoids counteract SP expression in AT and in the HT.

ELVs mediate protection in neuronal cultures undergoing either oxygen/glucose deprivation or N-methyl-D-aspartate receptor-mediated excitotoxicity, as well as in experimental ischemic stroke. The methyl ester or sodium salt of ELV-N32 and ELV-N34 resulted in reduced infarct volumes, promoted cell survival, and diminished neurovascular unit disruption when administered 1 hour following 2 hours of ischemia by middle cerebral artery occlusion. Herein we describe the pro-homeostatic and neuroprotective lipid-signaling function that sustains neural cell integrity.

Elovanoids a Senolytic Therapy for Diabetes and Obesity

It has been found that lean mice that became obese due to a high-fat diet display enhanced senescent cells abundance in their brain and anxiety behavior 1°. This study also provides the evidence that obesity-driven anxiety is arrested by senolytic drugs that dissipated the senescent cells. Senescent cells release a senescence-associated secretory phenotype (SASP) that induces nearby healthy cells to join in the dysfunction.

Transplanting senescent cells into young mice triggers weakness, frailty, and persistent dysfunctions lessened by administering a senolytic cocktail that includes dasatinib (an anti-leukemia drug) and quercetin (a plant flavonol) that set in motion programmed cell death of the senescence cells, extending both life and healthspan in aging mice (Nature Medicine Jul. 9, 2018). It has been reported that senescent cells accumulate in obesity. Obese mice display enhanced senescent cells abundance in the white matter adjacent to the lateral ventricles¹¹⁻¹⁶.

We reported that oligomeric A-beta peptide activates the SP, SASP followed by retinal cell death, and that Elovanoids arrest the expression of the SP genes p16INK4a, MMP1, p53, p21, p27, Il-6, and MMP1 as well as of the SASP secretome and of p16 protein by Western blot.

Additionally, we found that Elovanoids inhibit the expression of autophagy genes (ATG3, ATG5, ATG7, and Beclin-1) upon oligomeric A-beta peptide challenge in retinal cells³. Autophagy is an event of brown/beige adipocytes plasticity by regulating intracellular remodeling during brown/beige adipogenesis, thermogenic activation, and inactivation. This includes autophagic degradation of mitochondria for the inactivation of brown adipocytes and the transition from beige-to-white adipose tissue.

We also found that Elovanoids modulate the matrix metalloproteinases transcriptome (MMP1a, MMP2, MMP3, MMP8, MMP9, MMP12, and MMP13), and indicated that, with SASP, this mechanism contributes to alter the extracellular matrix 3. So in both AT and HT, SASP is autocrine and paracrine, modifying the homeostasis of the extracellular matrix microenvironment in both the AT and the HT as a consequence, creating an inflammatory milieu that contributes to impaired function in insulin sensitivity. We indicated that Elovanoids regulate slow-going, chronic, sterile inflammation (i.e., inflammaging). Additionally, another target of Elovanoids is unresolved oxidative stress and inflammation in neurons and neural injury models^{1,2}. These alterations, for example unresolved inflammation, evolve in dysfunctional adipocytes and are among the consequences of studied proinflammatory signaling in AT and insulin resistance. Produced by immune cells, TNF-α prevents insulin action in the adipocyte by downregulating the major insulin-responsive glucose transporter GLUT4 and inhibits insulin-dependent tyrosine phosphorylation of the insulin receptor and IRS-1 through ceramide production in addition to IL-6, IFN-γ, and CCL2.

Biomimetic therapeutic approach using synthetically produced molecules of endogenously generated Elovanoids that:
Restore homeostasis
Neuroprotective bioactivity in experimental disease models (e.g., ischemic stroke)
Arrest senescence programming and neural cell damage against oligomeric Aβ peptide damage³

Innovative medicinal chemistry

### REFERENCES CITED IN THIS EXAMPLE

1. Bhattacharjee S, Jun B, Belayev L, et al. Elovanoids are a novel class of homeostatic lipid mediators that protect neural cell integrity upon injury. Sci Adv. 2017; 3(9):e1700735. doi:10.1126/sciadv.1700735
2. Jun B, Mukherjee P K, Asatryan A, et al. Elovanoids are novel cell-specific lipid mediators necessary for neuroprotective signaling for photoreceptor cell integrity. Sci Rep. 2017; 7(1):5279. doi:10.1038/s41598-017-05433-7
3. Do K V, Kautzmann M-A I, Jun B, et al. Elovanoids counteract oligomeric (3-amyloid-induced gene expression and protect photoreceptors. Proc Natl Acad Sci. November 2019:201912959. doi:10.1073/pnas.1912959116
4. Musi N, Valentine J M, Sickora K R, et al. Tau protein aggregation is associated with cellular senescence in the brain. Aging Cell. 2018; 17(6). doi:10.1111/acel.12840
5. Das M M, Svendsen C N. Astrocytes show reduced support of motor neurons with aging that is accelerated in a rodent model of ALS. Neurobiol Aging. 2015; 36(2):1130-1139. doi:10.1016/j.neurobiolaging.2014.09.020
6. Turnquist C, Horikawa I, Foran E, et al. p53 isoforms regulate astrocyte-mediated neuroprotection and neurodegeneration. Cell Death Differ. 2016; 23(9):1515-1528. doi:10.1038/cdd.2016.37
7. Appel S H, Zhao W, Beers D R, Henkel J S. The microglial-motoneuron dialogue in ALS. Acta Myol Myopathies Cardiomyopathies Off J Mediterr Soc Myol. 2011; 30(1):4-8.
8. Komine O, Yamanaka K. Neuroinflammation in motor neuron disease. Nagoya J Med Sci. 2015; 77(4):537-549.
9. Lunyak V V, Amaro-Ortiz A, Gaur M. Mesenchymal Stem Cells Secretory Responses: Senescence Messaging Secretome and Immunomodulation Perspective. Front Genet. 2017; 8:220. doi:10.3389/fgene.2017.00220
10. Ogrodnik M, Zhu Y, Langhi L G P, et al. Obesity-Induced Cellular Senescence Drives Anxiety and Impairs Neurogenesis. Cell Metab. 2019; 29(5):1061-1077.e8. doi:10.1016/j.cmet.2018.12.008
11. Bussian T J, Aziz A, Meyer C F, Swenson B L, van Deursen J M, Baker D J. Clearance of senescent glial cells prevents tau-dependent pathology and cognitive decline. Nature. 2018; 562(7728):578-582. doi:10.1038/s41586-018-0543-y
12. Justice I N, Nambiar A M, Tchkonia T, et al. Senolytics in idiopathic pulmonary fibrosis: Results from a first-in-human, open-label, pilot study. EBioMedicine. 2019; 40:554-563. doi:10.1016/j.ebiom.2018.12.052
13. Kirkland J L. ClinicalTrials.gov [Internet]. Bethesda (MD): National Library of Medicine (US). 2018 Sep. 18-. Identifier NCT03675724, Alleviation by Fisetin of Frailty, Inflammation, and Related Measures in Older Adults. ClinicalTrials.gov. https://clinicaltrials.gov/ct2/show/NCT03675724. Published Dec. 28, 2018. Accessed Dec. 16, 2019.
14. Hickson L J. ClinicalTrials.gov [Internet]. Bethesda (MD): National Library of Medicine (US). 2016 Jul. 28-. Identifier NCT02848131, Senescence in Chronic Kidney Disease. ClinicalTrials.gov. https://clinicaltrials.gov/ct2/show/NCT02848131. Published Nov. 8, 2019. Accessed Dec. 16, 2019.
15. Hickson U. ClinicalTrials.gov [Internet]. Bethesda (MD): National Library of Medicine (US). 2017 Oct. 30-. Identifier NCT03325322, Inflammation and Stem Cells in Diabetic and Chronic Kidney Disease. ClinicalTrials.gov. https://clinicaltrials.gov/ct2/show/NCT03325322. Published Oct. 17, 2019. Accessed Dec. 16, 2019.
16. Hickson U. ClinicalTrials.gov [Internet]. Bethesda (MD): National Library of Medicine (US). 2016 Jan. 11-. Identifier NCT02652052, Hematopoietic Stem Cell Transplant Survivors Study (HTSS Study). ClinicalTrials.gov. https://clinicaltrials.gov/ct2/show/NCT02652052. Published Oct. 15, 2019. Accessed Dec. 16, 2019.

### OTHER REFERENCES INCORPORATED HEREIN

Aging, Cell Senescence, and Chronic Disease: Emerging Therapeutic Strategies. Tchkonia T, Kirkland J L. JAMA. 2018 Oct. 2; 320(13):1319-1320.
Fulop T, Witkowski J M, Olivieri F, Larbi A. The integration of inflammaging in age-related diseases. Semin Immunol. 2018 December; 40:17-35. Epub 2018 Oct. 2. PMID: 30287177.
Zhang, P., Kishimoto, Y., Grarnmatikakis, I et al. Senolytic therapy alleviates Aβ-associated oligodendrocyte progenitor cell senescence and cognitive deficits in an Alzheimer's disease model. Nat Neurosci 22, 719-728 (2019).
Van Deursen, J., Senolytic therapies for healthy longevity, Science, vol. 364, No. 6441 (2019).
Simpson, D. J., Olova, N. N. & Chandra, T, Cellular reprogramming and epigenetic rejuvenation. Clin Epigenet 13, 170 (2021).
Hill, S. (2021 Sep. 1). What are senolytics? A Summary of Senotherapeutics. lifespan.io/news/senolytics/
Quick, D. (2015 Mar. 10). Senolytics: A new class of drugs with the potential to slow the aging process. newatlas.com/senolytics-drug-slow-aging/36472/

We claim:

1. A method of delaying or preventing aging of a subject, the method comprising administering to the subject a therapeutically effective amount of a senolytic agent, wherein the senolytic agent comprises a VLC-PUFA or a hydroxylated derivative thereof.

2. The method of claim 1, wherein aging is not skin aging.

3. The method of claim 1, wherein aging is indicated by the presence or absence of senescent cells, the presence or absence of inflammation, or both.

4. The method of claim 1, wherein preventing aging comprises reducing the number or activity of senescent cells.

5. The method of claim 3, wherein the presence or absence of pro-inflammatory cytokines and chemokines is indicative of inflammation.

6. The method of claim 5, wherein the pro-inflammatory cytokines and chemokines comprise at least one of IL-6, IL-1β, IL-8/CXCL8, CCL2/MCP-1, CXCL1/KC/GRO, VEGF, ICAM1(CD54).

7. The method of claim 5, wherein the VLC-PUFA abrogates the production of pro-inflammatory cytokines and chemokines.

8. The method of claim 1 wherein the VLC-PUFA can be selected from the group consisting of the formula A or B:

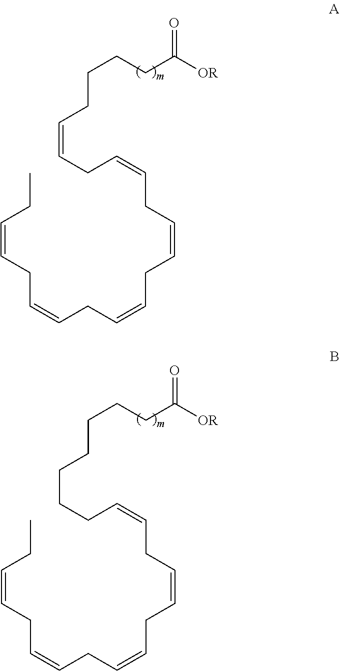

9. The method of claim 1, wherein the hydroxylated derivative compound can be selected from the group consisting of.

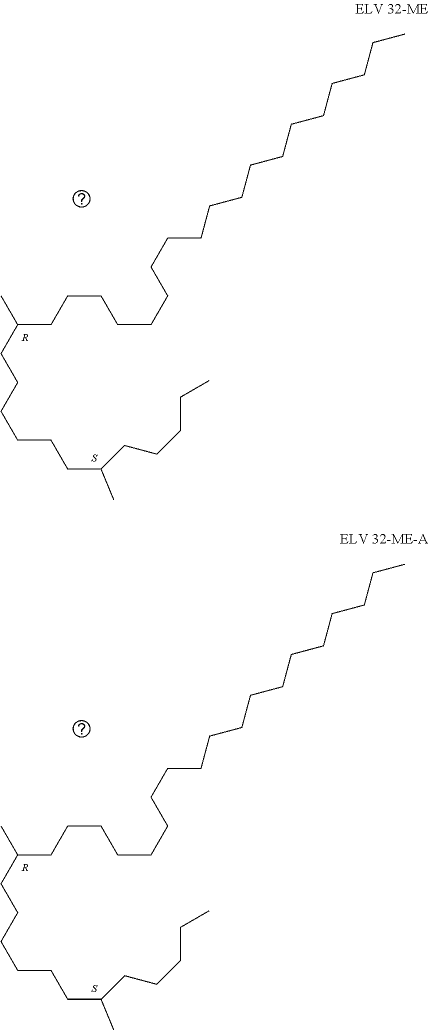

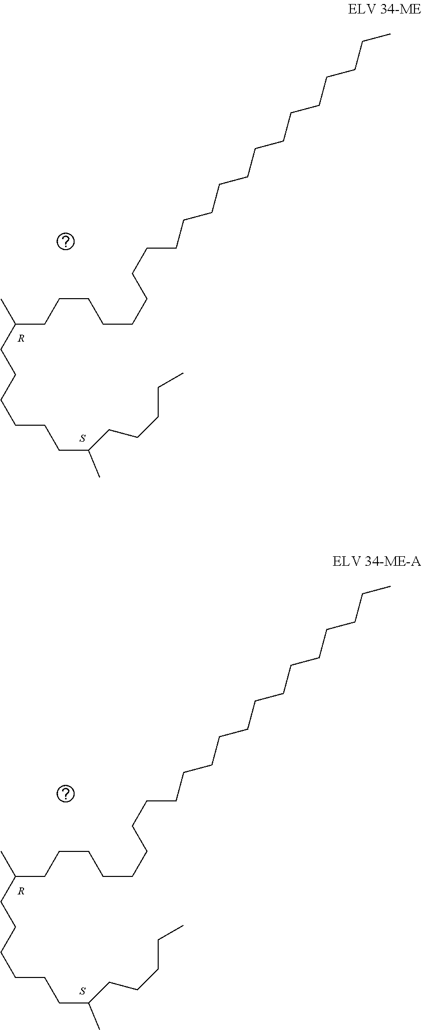

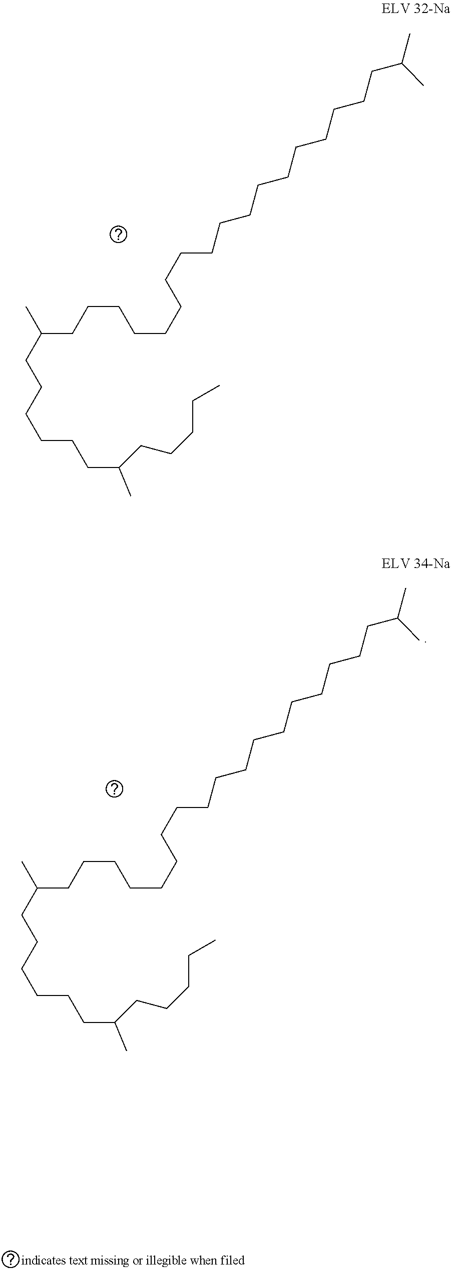

10. The method of claim 1, wherein the VLC-PUFA or hydroxylated derivative thereof is provided as a pharmaceutical composition.

11. The method of claim 1, wherein the therapeutically effective amount comprises about 500 nM concentration, greater than about 500 nM concentration, or less than about 500 nM concentration.

12. The method of claim 1, wherein the subject is a human.
